(19) United States
(12) Patent Application Publication
(10) Pub. No.: US 2025/0129348 A1
(43) Pub. Date: Apr. 24, 2025

(54) CIRCULAR PERMUTANTS, COMPOSITIONS COMPRISING THE SAME, AND METHODS OF USE THEREOF

(71) Applicant: Pairwise Plants Services, Inc., Durham, NC (US)

(72) Inventors: David Gerhard Schwark, Apex, NC (US); Sharon Leigh Guffy, Durham, NC (US)

(21) Appl. No.: 18/909,178

(22) Filed: Oct. 8, 2024

Related U.S. Application Data
(60) Provisional application No. 63/588,833, filed on Oct. 9, 2023.

(51) Int. Cl. C12N 9/22; C12N 9/12; C12N 9/78; C12N 15/62

(52) U.S. Cl. CPC C12N 9/22; C12N 9/1276; C12N 9/78; C12N 15/62; C12Y 207/07049; C12Y 301/21001; C12Y 305/04001; C12Y 305/04002; C12N 2310/20

(57) ABSTRACT

Described herein are circular permutants along with compositions and systems comprising the circular permutants. Fusion proteins including a circular permutant are also described such as fusion proteins that include a circular permutant fused to a polypeptide of interest (e.g., a deaminase or reverse transcriptase). Also described herein are methods of using and/or producing a circular permutant such as methods of using a circular permutant for modifying or editing a target nucleic acid.

### STATEMENT REGARDING ELECTRONIC FILING OF A SEQUENCE LISTING

A Sequence Listing in XML format, entitled 1499-132WO_ST26.xml, 3,956,249 bytes in size, generated on Oct. 3, 2024, and filed herewith, is hereby incorporated by reference in its entirety for its disclosures.

### FIELD

This invention relates to circular permutants along with compositions and systems comprising the same, and methods of use thereof such as methods of using a circular permutant for modifying or editing a target nucleic acid.

### BACKGROUND

Cas12a and base editors including a Cas12a domain have a limited editing window. New enzymes may be advantageous.

### SUMMARY OF THE INVENTION

A first aspect of the present invention is directed to a polypeptide comprising a circular permutant of a Cas12a, wherein the circular permutant binds to a nucleic acid. In some embodiments, the nucleic acid comprises a target nucleic acid. In some embodiments, the circular permutant has nuclease activity. In some embodiments, the polypeptide comprises a polypeptide of interest, optionally wherein the polypeptide of interest is fused to the N- or C-terminus of the circular permutant. In some embodiments, the polypeptide of interest is a deaminase and/or reverse transcriptase that is optionally fused to the N- or C-terminus of the circular permutant.

A further aspect of the present invention is directed to a polypeptide comprising a circular permutant of an engineered protein that comprises a Cas12a polypeptide, wherein the circular permutant binds to a nucleic acid. In some embodiments, the nucleic acid comprises a target nucleic acid. In some embodiments, the circular permutant has nuclease activity. In some embodiments, the polypeptide comprises a polypeptide of interest, optionally wherein the polypeptide of interest is fused to the N- or C-terminus of the circular permutant. In some embodiments, the polypeptide of interest is a deaminase and/or reverse transcriptase that is optionally fused to the N- or C-terminus of the circular permutant.

Another aspect of the present invention is directed to a polypeptide comprising an amino acid sequence having at least 70%, 75%, 80%, 85%, 90%, 95%, 96%, 97%, 98%, 99%, or 100% sequence identity to one or more of SEQ ID NOs: 234-245, 299-310, 390-399, 537-560, 702-704, 735-740, and 757-760. In some embodiments, the polypeptide binds to a nucleic acid and/or has nuclease activity. In some embodiments, the polypeptide comprises a polypeptide of interest, optionally wherein the polypeptide of interest is fused to the N- or C-terminus of the polypeptide having at least 70%, 75%, 80%, 85%, 90%, 95%, 96%, 97%, 98%, 99%, or 100% sequence identity to one or more of SEQ ID NOs: 234-245, 299-310, 390-399, 537-560, 702-704, 735-740, and 757-760. In some embodiments, the polypeptide of interest is a deaminase and/or reverse transcriptase that is optionally fused to the N- or C-terminus of the polypeptide having at least 70%, 75%, 80%, 85%, 90%, 95%, 96%, 97%, 98%, 99%, or 100% sequence identity to one or more of SEQ ID NOs: 234-245, 299-310, 390-399, 537-560, 702-704, 735-740, and 757-760. In some embodiments, a fusion protein of the present invention comprises a deaminase and/or reverse transcriptase that is optionally fused to the N- or C-terminus of a circular permutant of an engineered protein that comprises a Cas12a polypeptide. In some embodiments, a fusion protein of the present invention comprises a sequence having at least 70%, 75%, 80%, 85%, 90%, 95%, 96%, 97%, 98%, 99%, or 100% sequence identity to one or more of SEQ ID NOs: 351-362, 450-461, 486-497, and 609-639.

A further aspect of the present invention is directed to a polypeptide comprising a first amino acid sequence having at least 70%, 75%, 80%, 85%, 90%, 95%, 96%, 97%, 98%, 99%, or 100% sequence identity to a portion of one of SEQ ID NOs: 39-65, 69-132, 159-182, 281-284, 298, 400-401, 741, 761, and 850-861, wherein the portion of the one of SEQ ID NOs: 39-65, 69-132, 159-182, 281-284, 298, 400-401, 741, 761, and 850-861 includes the N-terminus of the one of SEQ ID NOs: 39-65, 69-132, 159-182, 281-284, 298, 400-401, 741, 761, and 850-861 and is less than the entire amino acid sequence of SEQ ID NOs: 39-65, 69-132, 159-182, 281-284, 298, 400-401, 741, 761, and 850-861. In some embodiments, the N-terminus of the one of SEQ ID NOs: 39-65, 69-132, 159-182, 281-284, 298, 400-401, 741, 761, and 850-861 is at or after amino acid residue 70 of the polypeptide. In some embodiments, the polypeptide binds to a nucleic acid and/or has nuclease activity. In some embodiments, the polypeptide comprising the first amino acid sequence further comprises a polypeptide of interest, optionally wherein the polypeptide of interest is fused to the N- or C-terminus of the polypeptide comprising the first amino acid sequence. In some embodiments, the polypeptide of interest is a deaminase and/or reverse transcriptase that is optionally fused to the N- or C-terminus of the polypeptide comprising the first amino acid sequence.

An additional aspect of the present invention is directed to a complex comprising a circular permutant of the present invention and a guide nucleic acid. In some embodiments, the complex comprises a deaminase and/or a reverse transcriptase. In some embodiments, the circular permutant is fused to a polypeptide of interest, optionally wherein the polypeptide of interest is fused to the N- or C-terminus of the circular permutant. In some embodiments, the polypeptide of interest is a deaminase and/or reverse transcriptase that is optionally fused to the N- or C-terminus of the circular permutant.

Another aspect of the present invention is directed to a method of modifying a target nucleic acid, the method comprising contacting the target nucleic acid with a circular permutant of the present invention and a guide nucleic acid, thereby modifying the target nucleic acid. In some embodiments, the method further comprises contacting the target nucleic acid with a deaminase and/or a reverse transcriptase. In some embodiments, the circular permutant is fused to a polypeptide of interest, optionally wherein the polypeptide of interest is fused to the N- or C-terminus of the circular permutant. In some embodiments, the polypeptide of interest is a deaminase and/or reverse transcriptase that is optionally fused to the N- or C-terminus of the circular permutant.

An additional aspect of the present invention is directed to a nucleic acid molecule encoding a circular permutant of the present invention. In some embodiments, the nucleic acid molecule has at least about 70%, 75%, 80%, 85%, 90%, 95%, 96%, 97%, 98%, 99%, or more sequence identity to one or more of SEQ ID NOs: 247-258, 286-297, 327-338, 364-375, 402-411, 462-473, 503-514, 561-584, 640-670, 706-708, 742-747, 763-766, 784, and 787. In some embodiments, the nucleic acid molecule encodes a polypeptide that has at least about 70%, 75%, 80%, 85%, 90%, 95%, 96%, 97%, 98%, 99%, or more sequence identity to one or more of SEQ ID NOs: 234-245, 299-310, 351-362, 390-399, 450-461, 486-497, 537-560, 609-639, 702-704, 735-740, and 757-760.

Another aspect of the present invention is directed to an expression cassette codon optimized for expression in an organism, the expression cassette comprising a polynucleotide encoding a promoter sequence, and a polynucleotide encoding a circular permutant of the present invention, which may be codon-optimized for expression in the organism. In some embodiments, the polynucleotide encoding the circular permutant has at least about 70%, 75%, 80%, 85%, 90%, 95%, 96%, 97%, 98%, 99%, or more sequence identity to one or more of SEQ ID NOs: 247-258, 286-297, 327-338, 364-375, 402-411, 462-473, 503-514, 561-584, 640-670, 706-708, 742-747, 763-766, 784, and 787. In some embodiments, the circular permutant has at least about 70%, 75%, 80%, 85%, 90%, 95%, 96%, 97%, 98%, 99%, or more sequence identity to one or more of SEQ ID NOs: 234-245, 299-310, 351-362, 390-399, 450-461, 486-497, 537-560, 609-639, 702-704, 735-740, and 757-760, which may be codon-optimized for expression in the organism.

A further aspect of the present invention is directed to a method of modifying a target nucleic acid in a cell, the method comprising introducing an expression cassette and/or vector of the present invention into the cell, thereby modifying the target nucleic acid in the cell.

An additional aspect of the present invention is directed to a method for producing a polypeptide of the present invention, the method comprising culturing a cell or group of cells that have been transformed with a nucleic acid encoding the polypeptide; and isolating the polypeptide, thereby producing the polypeptide.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1A is a schematic illustration of the domains of a Cas12a in the N- to C-terminus direction.

FIG. 1B is a schematic illustration of the domains of exemplary circular permutants of some embodiments of the present invention in the N- to C-terminus direction.

FIG. 2 is a graph of INDEL percentages for circular permutants of the present invention at three different target nucleic acids and for the control (LbCas12a; pWISE 121) at the three different target nucleic acids. The error bars represent standard deviation across two biological replicates and two technical replicates.

FIG. 3 shows graphs of the percentage of supporting next generation sequencing (NGS) reads vs. deletion lengths for the LbCas12a control (pWISE121) and each of the circular permutants (pWISE8345-pWISE8356) for three different target sites (PWsp143, PWsp449, and PWsp453) in HEK293T cells. The number of supporting reads for each deletion length (1-68 nt) were counted for each sample at each target site; SNPs and insertions were excluded from the data.

FIG. 4 shows graphs of the kernel density estimation vs. position in target loci for the control (pWISE121) and each of the circular permutants (pWISE8345-pWISE8356) at three different target sites (PWsp143, PWsp449, and PWsp453) in HEK293T cells. Kernel density estimation is used to fit a smooth curve to the observed distribution of deletion start sites. The black bar represents the spacer sequence, and the vertical dotted line represents where the control or circular permutant is expected to cut the non-target strand. While the density is not a direct measure of editing efficiency, the two are related. The area under the curve for each figure sums to 1.

FIG. 5 is a graph of indel percentages for the control LbCas12a (pWISE121) and control a REDRAW editor (RE2; pWISE6099) at three different target nucleic acids (PWsp143, PWsp449, and PWsp453). The error bars represent standard deviation across three biological replicates and two technical replicates.

FIG. 6A is a graph showing the precise editing percentages for fusion proteins (pWISE9459-9471) according to some embodiments of the present invention at the RNF2 target nucleic acid and for the control LbCas12a (pWISE121) and control REDRAW editor (RE2; pWISE6099) at the RNF2 target nucleic acid. The error bars represent standard deviation across three biological replicates and two technical replicates.

FIG. 6B is a graph showing the indel percentages for fusion proteins (pWISE9459-9471) according to some embodiments of the present invention at the RNF2 target nucleic acid and for the control LbCas12a (pWISE121) and control REDRAW editor (RE2; pWISE6099) at the RNF2 target nucleic acid. The error bars represent standard deviation across three biological replicates and two technical replicates.

FIG. 7A is a graph showing the precise editing percentages for fusion proteins (pWISE9459-9471) according to some embodiments of the present invention at the FANCF target nucleic acid and for the control LbCas12a (pWISE121) and control REDRAW editor (RE2; pWISE6099) at the FANCF target nucleic acid. The error bars represent standard deviation across three biological replicates and two technical replicates.

FIG. 7B is a graph showing the indel percentages for fusion proteins (pWISE9459-9471) according to some embodiments of the present invention at the FANCF target nucleic acid and for the control LbCas12a (pWISE121) and control REDRAW editor (RE2; pWISE6099) at the FANCF target nucleic acid. The error bars represent standard deviation across three biological replicates and two technical replicates.

FIG. 8A is a graph showing the precise editing percentages for fusion proteins (pWISE9459-9471) according to some embodiments of the present invention at the DNMT1 target nucleic acid and for the control LbCas12a (pWISE121) and control REDRAW editor (RE2; pWISE6099) at the DNMT1 target nucleic acid. The error bars represent standard deviation across three biological replicates and two technical replicates.

FIG. 8B is a graph showing the indel percentages for fusion proteins (pWISE9459-9471) according to some embodiments of the present invention at the DNMT1 target nucleic acid and for the control LbCas12a (pWISE121) and control REDRAW editor (RE2;
pWISE6099) at the DNMT1 target nucleic acid. The error bars represent standard deviation across three biological replicates and two technical replicates.

FIG. 9A is a 3D cartoon image showing a predicted protein structure of a circular permutant (CP02) according to some embodiments of the present invention; the spheres represent the new N- and C-termini of the circular permutant protein.

FIG. 9B is a 3D cartoon image showing a predicted protein structure of a circular permutant (CP12) according to some embodiments of the present invention; the spheres represent the new N- and C-termini of the circular permutant protein.

FIGS. 10A-10C shows graphs of the precise editing percentages and indel percentages ratio for fusion proteins (pWISE9459-9471) according to some embodiments of the present invention at three different target nucleic acids (RNF2, FIG. 10A; FANCF-002, FIG. 10B; and DNMT1-001, FIG. 10C) and for the control LbCas12a (pWISE121) and control REDRAW editor (RE2; pWISE6099) at the same three different target nucleic acids. The error bars represent standard deviation across three biological replicates and two technical replicates.

FIG. 11 is a graph showing the INDEL percentages for LbCas 12a (pWISE121) at each of the target genes FANCF (PWsp132 and PWsp449), RUNX1 (PWsp135), and AAVS1 (PWsp389) in HEK293T cells. The error bars represent standard deviation across a single biological replicate and two technical replicates.

FIG. 12 is a graph showing the INDEL percentages for EnAsCas12a circular permutants (pWISE9723-9734) of the present invention at the RNF2 (PWsp1956 and PWsp1959) and FANCF (PWsp1960) target nucleic acids and for the control EnAsCas12a (pWISE4494) at the RNF2 (PWsp1956 and PWsp1959) and FANCF (PWsp1960) target nucleic acids in HEK293T cells. The error bars represent standard deviation across three biological replicates and two technical replicates.

FIG. 13 is a graph showing the indel percentages for circular permutants (pWISE9517, pWISE9519, pWISE9521, pWISE9523, pWISE9525, and pWISE9527) according to some embodiments of the present invention at AHK4 target nucleic acid and for the control LbCas12a (pWISE3380) at the AHK4 target nucleic acid in soy. These circular permutants and control LbCas12a were optimized for expression in soy. Each dot represents the sum of the INDEL percentage for an individual soy plant, the thicker line is the average INDEL percentage across all plants for each tested circular permutant.

FIGS. 14-19 are graphs showing the INDEL percentages for LbCas12a (pWISE121) and circular permutants according to some embodiments of the present invention at each of the target genes FANCF (PWsp449), DNMT1 (PWsp143), and RNF2 (PWsp453) in HEK293T cells. The data is the average of two technical replicates.

FIG. 20 is a graph showing the sum of INDEL percentages for circular permutants including an arginine mutation (pWISE9516, pWISE9518, pWISE9520, pWISE9522, pWISE9524, and pWISE9526) according to some embodiments of the present invention at AHK4 target nucleic acid and for the control LbCas12a D156R (pWISE9528) at the AHK4 target nucleic acid in soy. Each dot represents the sum of the INDEL percentage for an individual soy plant, the thicker line is the average INDEL percentage across all plants for each tested protein.

FIG. 21 is a graph showing the sum of INDEL percentages for circular permutants including an arginine mutation (pWISE9516, pWISE9518, pWISE9520, pWISE9522, pWISE9524, and pWISE9526) compared to circular permutants without the arginine mutation (pWISE9517 (CP02), pWISE9519 (CP04), pWISE9521 (CP06), pWISE9523 (CP08), pWISE9525 (CP10), and pWISE9527 (CP12)) according to some embodiments of the present invention at AHK4 target nucleic acid and for the control LbCas 12a (pWISE3380) and control LbCas12a D156R (pWISE9528) at the AHK4 target nucleic acid in soy. Each dot represents the sum of the INDEL percentage for an individual soy plant, the thicker line is the average INDEL percentage across all plants for each tested protein.

FIG. 22 is a graph showing the sum of INDEL percentages for circular permutants (pWISE10428, pWISE10429, pWISE10430, pWISE10431, and pWISE10432) according to some embodiments of the present invention at AHK4 target nucleic acid and for a control LbCas12a (pWISE3380) and a control engineered enzyme (pWISE6345) at the AHK4 target nucleic acid in soy. Each dot represents the sum of the INDEL percentage for an individual soy plant, the thicker line is the average INDEL percentage across all plants for each tested protein.

FIG. 23 is a graph showing the INDEL percentages for enzymatically dead circular permutants fused to a cytosine deaminase or circular permutants comprising a catalytic nickase mutation fused to a cytosine deaminase at the target gene FANCF (PWsp132) in HEK293T cells. The error bars represent standard deviation across two technical replicates.

FIG. 24 is a graph showing the INDEL percentages for circular permutants including two arginine mutations (pWISE10850, pWISE10851, and pWISE10852) compared to a control LbCas12a (pWISE121) and a control Cas12a with two arginine mutations (pWISE3559) according to some embodiments of the present invention at the indicated PAM sites in the target genes AAVS1, DNMT1, EXM1, FANCF, HEK2, HEK3, and RNF2 in HEK293T cells. The data is the average of three biological replicates.

FIG. 25 is a graph showing the fold increase of the INDEL percentages over a control LbCas12a (pWISE121) for circular permutants including two arginine mutations (pWISE10850, pWISE10851, and pWISE10852) and a control Cas12a with two arginine mutations (pWISE3559) according to some embodiments of the present invention at the indicated PAM sites in the target genes AAVS1, DNMT1, EXM1, FANCF, HEK2, HEK3, and RNF2 in HEK293T cells. The data is the average of three biological replicates.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of #10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

A “heterologous nucleotide sequence” or a “recombinant nucleotide sequence” is a nucleotide sequence not naturally associated with a host cell into which it is introduced, including non-naturally occurring multiple copies of a naturally occurring nucleotide sequence.

A “native” or “wild-type” nucleic acid, nucleotide sequence, polypeptide or amino acid sequence refers to a naturally occurring or endogenous nucleic acid, nucleotide sequence, polypeptide or amino acid sequence. Thus, for example, a “native nucleic acid” is a nucleic acid that is naturally occurring in or endogenous to a reference organism. A “homologous” nucleic acid sequence is a nucleotide sequence naturally associated with a host cell into which it is introduced.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “recombinant nucleic acid,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions).

A polynucleotide, gene, or polypeptide may be “isolated” by which is meant a nucleic acid or polypeptide that is substantially or essentially free from components normally found in association with the nucleic acid or polypeptide, respectively, in its natural state. In some embodiments, such components include other cellular material, culture medium from recombinant production, and/or various chemicals used in chemically synthesizing the nucleic acid or polypeptide.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue.

As used herein, a “non-natural mutation” refers to a mutation that is generated through human intervention and differs from mutations found in the same gene or polypeptide that have occurred in nature (e.g., occurred naturally and not as a result of a modification made by a human).

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., “substantially complementary,” such as about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

A “portion” or “fragment” of a nucleotide sequence or polypeptide (including a domain) will be understood to mean a nucleotide sequence or polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., nucleotide(s) or peptide(s)) relative to a reference nucleotide sequence or polypeptide, respectively, and comprising, consisting essentially of and/or consisting of a nucleotide sequence or polypeptide of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleotide sequence or polypeptide. In some embodiments, a portion of a reference nucleotide sequence or polypeptide is about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or more of the full-length reference nucleotide sequence or polypeptide. Such a nucleic acid fragment or portion according to the invention may be, where appropriate, included in a larger polynucleotide of which it is a constituent. As an example, a repeat sequence of guide nucleic acid of this invention may comprise a portion of a wild-type CRISPR-Cas repeat sequence (e.g., a wild-type Type V CRISPR Cas repeat, e.g., a repeat from the CRISPR Cas system that includes, but is not limited to, Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c, and the like). Similarly, a portion of a polypeptide may be included in a larger polypeptide of which it is a constituent.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptides of this invention. “Orthologous” and “orthologs” as used herein, refers to homologous nucleotide sequences and/or amino acid sequences in different species that arose from a common ancestral gene during speciation. A homologue or ortholog of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCGR Wisconsin Package® (Accelrys Inc., San Diego, CA) as well as web-based alignment programs such as Clustal Omega, EMBOSS Needle, EMBOSS Stretcher, EMBOSS Water, LALIGN, GGSEARCH2SEQ, EMBOS Cons, Kalign, MAFFT, MUSCLE, and T-Coffee. In some embodiments, an “optimal alignment” of two sequences (e.g., two polypeptide sequences) is the highest scoring alignment, optionally from an alignment conducted by a tool such as the local homology algorithm of Smith and Waterman, the homology alignment algorithm of Needleman and Wunsch, the search for similarity method of Pearson and Lipman, GAP, BESTFIT, FASTA, and TFASTA available as part of the GCGR Wisconsin Package® (Accelrys Inc., San Diego, CA), Clustal Omega, EMBOSS Needle, EMBOSS Stretcher, EMBOSS Water, LALIGN, GGSEARCH2SEQ, EMBOS Cons, Kalign, MAFFT, MUSCLE, and/or T-Coffee. In some embodiments, an “optimal alignment” of two sequences (e.g., two polypeptide sequences) is the alignment that provides the highest percent sequence identity, optionally allowing for one or more gap(s) to be introduced into one or both sequences. An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” and/or optimal alignment may be determined using Basic Local Alignment Search Tool (BLAST) provided by the National Center for Biotechnology Information such as BLASTX, for translated nucleotide sequences, BLASTN for polynucleotide sequences, and BLASTP for polypeptide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention can be codon optimized for expression. In some embodiments, a polynucleotide, nucleic acid construct, expression cassette, and/or vector of the present invention (e.g., that comprises/encodes a polypeptide of the present invention (e.g., a circular permutant), a nucleic acid binding polypeptide (e.g., a DNA binding polypeptide such as a sequence-specific DNA binding domain from a polynucleotide-guided endonuclease, a zinc finger nuclease, a transcription activator-like effector nuclease (TALEN), an Argonaute protein, and/or a CRISPR-Cas effector protein), a guide nucleic acid, a deaminase, and/or a reverse transcriptase) may be codon optimized for expression in an organism (e.g., an animal such as a human, a plant, a fungus, an archaeon, or a bacterium). In some embodiments, the codon optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acid constructs, polynucleotides, expression cassettes, and/or vectors but which have not been codon optimized.

In any of the embodiments described herein, a polynucleotide or nucleic acid construct of the invention may be operatively associated with a variety of promoters and/or other regulatory elements for expression in an organism or cell thereof (e.g., a mammal and/or a mammalian cell, a plant and/or a cell of a plant, etc.). Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubil promoter and intron). In some embodiments, a promoter associated with an intron may be referred to as a “promoter region” (e.g., Ubil promoter and intron).

By “operably linked” or “operably associated” as used herein in reference to polynucleotides, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” or “fused” in reference to polypeptides, refers to the covalent attachment of one polypeptide to another. A polypeptide may be linked or fused to another polypeptide (e.g., at the N-terminus or the C-terminus) directly (e.g., via a peptide bond) or through a linker (e.g., a peptide linker). Two polypeptides being directly fused (e.g., a direct linkage) refers to the covalent attachment of one amino acid residue of a first polypeptide of the two polypeptides to an amino acid residue of a second polypeptide of the two polypeptides without an intervening element between the two amino acid residues. For example, first and second polypeptides may be directly linked via a peptide bond between the first and second polypeptides without an intervening element (e.g., a linker) between the first and second polypeptides. Two polypeptides being directly fused (e.g., an indirect linkage) refers to an intervening element (e.g., a linker such as a peptide linker) that is present between the two polypeptides and is covalently attached to each, optionally the intervening element may attach one end of a first polypeptide of the two polypeptides to an end of the second polypeptide of the two polypeptides.

A “fusion protein” as used herein refers to two or more polypeptides that are covalently attached (e.g., directly or indirectly) so that they are transcribed and translated as a single unit and thereby produce a single polypeptide comprising the two or more polypeptides. In some embodiments, the two or more polypeptides may naturally be encoded by separate genes, but, in the form of a fusion protein, are encoded by a single gene.

The term “linker” is art-recognized and refers to a chemical group, or a molecule linking two molecules or moieties, e.g., linking two polypeptides or domains of a fusion protein, such as, for example, a CRISPR-Cas effector protein and a peptide tag and/or a polypeptide of interest. A linker may be comprised of a single linking molecule (e.g., a single amino acid) or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide (e.g., a peptide linker).

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length)). In some embodiments, a peptide linker may be a GS linker. In some embodiments, the peptide linker is a GS linker having 2, 3, or 4 amino acid residues, optionally 2 or 4 amino acid residues. In some embodiments, the peptide linker has one of the amino acid sequences of SEQ ID NOs: 1-36, 313-314, 869-870, 883, and/or 885. In some embodiments, the peptide linker is encoded by a sequence of SEQ ID NO: 311 or 312. In some embodiments, the peptide linker may comprise an amino acid sequence of CA, CF, (GGS)ₙ, GS, SG, GSSG (SEQ ID NO:31), GSSGSS (SEQ ID NO:32), GSSGSSGS (SEQ ID NO:33), (GSS)ₙ (SEQ ID NO:34), (GSS)ₙGS (SEQ ID NO:35), S(GGS)ₙ (SEQ ID NO:25), SGGS (SEQ ID NO:26), (GSS)ₙG (SEQ ID NO:36), (GGGGS)ₙ (SEQ ID NO:27), (SGS)ₙ (SEQ ID NO:869), (SGGS)ₙ (SEQ ID NO:870), or GSPKKKRKVSGGS (SEQ ID NO:885), wherein n is an integer of 1-20 (e.g., 1, 2, 3, 4, 5, 6, 7, 8, 9, 10, 11, 12, 13, 14, 15, 16, 17, 18, 19, or 20). In some embodiments, the peptide linker may comprise the amino acid sequence: SGGSGGSGGS (SEQ ID NO:28). In some embodiments, the peptide linker may comprise the amino acid sequence: SGSETPGTSESATPES (SEQ ID NO:29), also referred to as the XTEN linker. In some embodiments, the peptide linker may comprise the amino acid sequence: SGGSSGGSSGSETPGTSESATPESSGGSSGGS (SEQ ID NO:30), also referred to as the GS-XTEN-GS linker or a BE4 linker. In some embodiments, the linker has a sequence of (GSS)ₙG (SEQ ID NO:36), wherein n is 3. In some embodiments, the linker has a sequence of (GSS)ₙG (SEQ ID NO:36), wherein n is 5. In some embodiments, the peptide linker may comprise the amino acid sequence STSQSDGSSVPADIDQSSDSDQSSSQGQPG (SEQ ID NO: 5), also referred to as the 5R linker. In some embodiments, the peptide linker may comprise the amino acid sequence: SGGSSGSETPGTSESATPESSGGS (SEQ ID NO:883), also referred to as the SGGS-XTEN-SGGS linker.

As used herein, the term “linked,” or “fused” in reference to polynucleotides, refers to the covalent attachment of one polynucleotide to another polynucleotide. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a direct covalent linkage or through one or more linking nucleotides. In some embodiments, a polynucleotide motif of a certain structure may be inserted within another polynucleotide sequence (e.g., extension of the hairpin structure in guide RNA). In some embodiments, the linking nucleotides may be naturally occurring nucleotides. In some embodiments, the linking nucleotides may be non-naturally occurring nucleotides. Two polynucleotides being indirectly fused (e.g., a direct linkage) refers to the covalent attachment of one nucleotide of a first polynucleotide of the two polynucleotides to a nucleotide of a second polynucleotide of the two polynucleotides without an intervening element between the two polynucleotides. For example, first and second polynucleotides may be directly linked via a phosphodiester bond between the first and second polynucleotides without an intervening element (e.g., a linker) between the first and second polynucleotides. Two polynucleotides being directly fused (e.g., an indirect linkage) refers to an intervening element (e.g., a linker such as a polynucleotide linker) that is present between the two polynucleotides and is covalently attached to each, optionally the intervening element attaches one end of a first polynucleotide of the two polynucleotides to an end of the second polynucleotide of the two polynucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). In some embodiments, a promoter region may comprise at least one intron (e.g., SEQ ID NO:37 or SEQ ID NO:38).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdcal) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdcal are inducible promoters. Pnr is induced by nitrate and repressed by ammonium (Li et al. Gene 403:132-142 (2007)) and Pdcal is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79:87-94), maize (Christensen et al., 1989. Plant Molec. Biol. 12:619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the European patent publication EP0342926. The ubiquitin promoter is suitable for the expression of the nucleotide sequences of the invention in transgenic plants, especially monocotyledons. Further, the promoter expression cassettes described by McElroy et al. (Mol. Gen. Genet. 231:150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP U.S. Pat. No. 255,378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, incorporated by reference herein for its disclosure of promoters. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); European patent EP 0452269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9 (5): 297-306 (2015)), ZmSTK2 USP from maize (Wang et al. Genome 60 (6): 485-495 (2017)), LAT52 and LAT59 from tomato (Twell et al. Development 109 (3): 705-713 (1990)), Zm13 (U.S. Pat. No. 10,421,972), PLA2-δ promoter from Arabidopsis (U.S. Pat. No. 7,141,424), and/or the ZmC5 promoter from maize (International PCT Publication No. WO 1999/042587).

Additional examples of plant tissue-specific/tissue preferred promoters include, but are not limited to, the root hair-specific cis-elements (RHEs) (KIM ET AL. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37 (8): 1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubil promoter and intron.

Non-limiting examples of introns useful with the present invention include introns from the ADHI gene (e.g., Adh1-S introns 1, 2 and 6), the ubiquitin gene (Ubil), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdcal), the psbA gene, the atpA gene, or any combination thereof.

An “editing system” as used herein refers to any site-specific (e.g., sequence-specific) nucleic acid editing system now known or later developed, which system can introduce a modification (e.g., a mutation) in a nucleic acid in target specific manner. For example, an editing system (e.g., a site- and/or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which may comprise one or more polypeptide(s) and/or one or more polynucleotide(s) that when present and/or expressed together (e.g., as a system) in a composition and/or cell can modify (e.g., mutate) a target nucleic acid in a sequence specific manner. In some embodiments, an editing system (e.g., a site- and/or sequence-specific editing system) can comprise one or more polynucleotide(s) and/or one or more polypeptide(s), including but not limited to a nucleic acid binding polypeptide (e.g., a DNA binding domain), a nuclease, another polypeptide, and/or a polynucleotide. In some embodiments, a CRISPR-Cas editing system is provided and/or is used that comprises a polypeptide of the present invention.

In some embodiments, an editing system comprises one or more sequence-specific nucleic acid binding polypeptide(s) (e.g., a DNA binding domain) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system comprises one or more cleavage polypeptide(s) (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN).

A “nucleic acid binding polypeptide” as used herein refers to a polypeptide or domain that binds and/or is capable of binding a nucleic acid (e.g., a target nucleic acid). A DNA binding domain is an exemplary nucleic acid binding polypeptide and may be a site- and/or sequence-specific nucleic acid binding domain. In some embodiments, a nucleic acid binding polypeptide may be a sequence-specific nucleic acid binding polypeptide such as, but not limited to, a sequence-specific binding domain from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, a nucleic acid binding polypeptide comprises a cleavage domain (e.g., a nuclease domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, the nucleic acid binding polypeptide associates with and/or is capable of associating with (e.g., forms a complex with) with one or more nucleic acid molecule(s) (e.g., forms a complex with a guide nucleic acid as described herein), which may direct and/or guide the nucleic acid binding polypeptide to a specific target nucleotide sequence (e.g., a gene locus of a genome) that is complementary to the one or more nucleic acid molecule(s) (or a portion or region thereof), thereby causing the nucleic acid binding polypeptide to bind to the nucleotide sequence at the specific target site. In some embodiments, the nucleic acid binding polypeptide is a CRISPR-Cas effector protein as described herein.

In some embodiments, an editing system comprises or is a ribonucleoprotein such as an assembled ribonucleoprotein complex (e.g., a ribonucleoprotein that comprises a CRISPR-Cas effector protein, a guide nucleic acid, and optionally a deaminase and/or reverse transcriptase). In some embodiments, a ribonucleoprotein of an editing system may be assembled together (e.g., a pre-assembled ribonucleoprotein including a CRISPR-Cas effector protein, a guide nucleic acid, and optionally a deaminase and/or reverse transcriptase) such as when contacted to a target nucleic acid or when introduced into a cell (e.g., a mammalian cell or a plant cell) (e.g., at the time of contacting the components of the ribonucleoprotein to a target nucleic acid and/or at the time of introducing the components of the ribonucleoprotein into a cell). In some embodiments, a ribonucleoprotein of an editing system may assemble into a complex (e.g., a non-covalently bound complex) while a portion of the ribonucleoprotein is contacting a target nucleic acid and/or may assemble after and/or during introduction into a plant cell. In some embodiments, an editing system may be assembled (e.g., into a non-covalently bound complex) when introduced into a plant cell. In some embodiments, a ribonucleoprotein of an editing system may be contacted a target nucleic acid and/or may be introduced into a plant cell. In some embodiments, an editing system may be assembled (e.g., into a non-covalently bound complex) when introduced into a plant cell. In some embodiments, a ribonucleoprotein may comprise a polypeptide of the present invention (e.g., a polypeptide comprising a circular permutant), a guide nucleic acid, and optionally a deaminase and/or reverse transcriptase. In some embodiments, a circular permutant of the present invention is used in place of (e.g., substituted for) a CRISPR-Cas effector protein (e.g., in a composition, complex, kit, method, and/or system such as an editing system described herein) and/or functions as a CRISPR-Cas effector protein, optionally in a composition, complex, ribonucleoprotein, kit, method, system, and/or editing system of the present invention.

In some embodiments, an editing system of the present invention comprises a reverse transcriptase, an extended guide nucleic acid, and a CRISPR-Cas effector protein and/or a polypeptide of the present invention. In some embodiments, the CRISPR-Cas effector protein and/or a polypeptide of the present invention, the reverse transcriptase, and the extended guide nucleic acid may form a complex or may be comprised in a complex that is capable of interacting with a target nucleic acid.

In some embodiments, a guide nucleic acid further comprises a reverse transcriptase template and may be referred to as an extended guide nucleic acid. An “extended guide nucleic acid” as used herein is a guide nucleic acid as described herein that further comprises a reverse transcriptase template (RTT) and/or a primer binding site (PBS). In some embodiments, an extended guide nucleic acid is an engineered prime editing guide RNA (pegRNA). An extended guide nucleic acid may be a targeted allele guide RNA (tagRNA) or a stabilized targeted allele guide RNA (stagRNA). A “tagRNA” as used herein refers to an extended guide nucleic acid that comprises a PBS and a RTT and has target strand complementarity. A “stagRNA” as used herein refers to a tagRNA that comprises a stabilization motif. A stabilization motif may be present at the 3′ and/or 5′ end of a tagRNA. In some embodiments, a stabilization motif is present at the 3′ end of a tagRNA. Exemplary stabilization motifs include, but are not limited to, recruiting motifs, RNA hairpins, pseudoknot sequences, and/or PP7 motifs (e.g., a PP7 RNA hairpin sequence). In some embodiments, a stagRNA is a tagRNA that comprises a PP7 RNA hairpin sequence. In some embodiments, a CRISPR-Cas effector protein (e.g., a Type II or Type V CRISPR-Cas effector protein), a reverse transcriptase, and an extended guide nucleic acid can form a complex or are comprised in a complex.

In some embodiments, an extended guide nucleic acid comprises an extended portion that includes a primer binding site and a reverse transcriptase template, wherein the reverse transcriptase template comprises the modification (e.g., edit) to be incorporated into a target nucleic acid. In some embodiments, an extended guide nucleic acid comprises, at its 3′ end, a primer binding site and a modification (e.g., an edit) to be incorporated into the target nucleic acid (e.g., a reverse transcriptase template). In some embodiments, an extended guide nucleic acid comprises: (1) a sequence that interacts (e.g., recruits and/or binds) with a CRISPR-Cas effector protein (e.g., a CRISPR-Cas nuclease), (2) a spacer having substantial complementary to a first site on a target nucleic acid (e.g., a CRISPR RNA (crRNA) (a first crRNA) and/or tracrRNA+crRNA (sgRNA)), and (3) a nucleic acid encoded repair template (e.g., an RNA encoded repair template) comprising a primer binding site and an RNA template (e.g., that encodes the modification to be incorporated into the target nucleic acid). In some embodiments, an extended guide nucleic acid (e.g., an extended guide RNA) may comprise, 5′-3′, a spacer sequence, a repeat sequence, and an extended portion, the extended portion comprising, 5′ to 3′, a reverse transcriptase template and a primer binding site. In some embodiments, an extended guide nucleic acid may comprise, 5′-3′, a spacer sequence, a repeat sequence and an extended portion, the extended portion comprising, 5′ to 3′, a primer binding site and a reverse transcriptase template. In some embodiments, an extended guide nucleic acid may comprise, 5′-3′, an extended portion, a spacer sequence, and a repeat sequence, wherein the extended portion comprises, 5′ to 3′, a reverse transcriptase template and a primer binding site. In some embodiments, an extended guide nucleic acid may comprise, 5′-3′, an extended portion, a spacer sequence, and a repeat sequence, wherein the extended portion comprises, 5′ to 3′, a primer binding site and a reverse transcriptase template.

According to some embodiments, an extended guide nucleic acid (e.g., a pegRNA) may have a structure and/or be designed as described in Anzalone et al., Nature, 2019 December; 576 (7785): 149-157. In some embodiments, an extended guide nucleic acid comprises a primer binding site (PBS) optionally having a sequence of 1, 2, 3, 4, or 5 to 6, 7, 8, 9, 10, 11, 12, 13, 14, or 15 nucleotides and a reverse transcriptase template (RT template) sequence optionally having a sequence of 65 nucleotides or more. In some embodiments, a PBS of an extended guide nucleic acid has a sequence of less than 15 nucleotides and has a sequence of 1, 2, 3, 4, 5, 6, 7, 8, 9, 10, 11, 12, 13, or 14 nucleotides (e.g., a sequence of 5 or 6 nucleotides in length). The RT template sequence may be after the PBS sequence in the 5′ to 3′ direction. In some embodiments, the RT template sequence of the extended guide nucleic acid has a length of greater than 65 nucleotides and may comprise about 50 or more nucleotides of heterology relative to the target site (e.g., target nucleic acid), followed by about 15 or more nucleotides of homology relative to the target site. In some embodiments, the RT template sequence of the extended guide nucleic acid is after the PBS sequence and the RT template sequence has a length of greater than 65 nucleotides with the sequence including more than 50 nucleotides of heterology relative to the target site, followed by more than 15 nucleotides of homology relative to the target site. Accordingly, in some embodiments, when the extended guide nucleic acid is reverse transcribed, the resulting newly transcribed sequence may hybridize and/or is configured to hybridize with the unnicked strand of the target site, which may thereby create a heteroduplex DNA with a large insertion into the newly synthesized strand. Upon repair of this mismatched DNA, the resultant repaired DNA may contain a large insertion (e.g., greater than 50 nucleotides) of DNA sequence. In some embodiments, the method may provide a large deletion (e.g., greater than 50 nucleotides) of DNA sequence. In some embodiments, the PBS and the 15 or more nucleotides of homology to the target site may comprise homology arms, which may serve to insert the heterology into the target site optionally using homology directed repair. The inserted DNA may correspond to any functional sequence of DNA such as, but not limited to: a functional transgene; a fragment of DNA that is inserted into a gene in a way that, when the gene is transcribed, would produce a hairpin RNA that is sufficient to silence homologous genes through RNAi; and/or one or more functional site-specific recombination sites, e.g. lox, frt, which could then be used in subsequent Cre or Flp mediated site-specific recombination processes. In some embodiments, an extended guide nucleic acid may be too large to produce using a PolIII promoter in vivo. In some embodiments, an extended guide nucleic acid may be operatively associated with and/or produced using a PolII promoter. In some embodiments, a DNA binding polypeptide (e.g., a DNA binding domain) and/or DNA endonuclease may have a structure and/or be designed as described in Anzalone et al., Nature, 2019 December; 576 (7785): 149-157. In some embodiments, a DNA binding domain and/or DNA endonuclease is a CRISPR Cas polypeptide such as a Cas9 nickase, a nicking variant of another CRISPR Cas polypeptide, or Cas12a.

In some embodiments, two extended guide nucleic acids (e.g., pegRNAs) may be used (e.g., an editing system may comprise two extended guide nucleic acids). One or both of the two extended guide nucleic acids may have a structure and/or be designed as described in Anzalone et al., Nature, 2019 December; 576 (7785): 149-157. The two extended guide nucleic acids may comprise a primer binding site (PBS) optionally having a sequence of 1, 2, 3, 4, or 5 to 6, 7, 8, 9, 10, 11, 12, 13, 14, or 15 nucleotides and a reverse transcriptase template (RT template) sequence optionally having a sequence of 50 nucleotides or more. The RT template sequences of the two extended guide nucleic acids may be complementary to each other and as such the polynucleotides that are respectively reverse transcribed from each the RT templates will be complementary to each other and will be able to hybridize with each other. This may allow for the intermediates that are produced by this system and/or method to join together two sections of DNA that are otherwise separated by more than 50 nucleotides, e.g., within a chromosome, or that are positioned on two separate pieces of DNA, e.g., on two different chromosomes. After repair of the intermediates, the resultant products may produce, depending on the design of the RT template, large deletions, large inversions, or inter-chromosomal recombinations. Since all of these products are produced by homology directed repair, the products may be predictably precise and/or reproducible. In some embodiments, a DNA binding polypeptide (e.g., a DNA binding domain) and/or DNA endonuclease may have a structure and/or be designed as described in Anzalone et al., Nature, 2019 December; 576 (7785): 149-157. In some embodiment, a DNA binding polypeptide and/or DNA endonuclease is a CRISPR Cas polypeptide such as a Cas9 nickase, a similar nicking variant of another CRISPR Cas polypeptide, or Cas12a. In some embodiments, a DNA binding polypeptide and/or DNA endonuclease is a Cas9 nuclease, a similar nuclease from another CRISPR Cas polypeptide, or Cas12a. Using a nuclease (rather than a nickase) may facilitate the intra- or interchromosomal recombination processes through single-strand annealing of the more than 50 nucleotide 3′ overhangs that would be produced at each of the two target sites corresponding to the two pegRNA target nucleic acids. In some embodiments, an editing system comprises one extended guide nucleic acid and a guide nucleic acid that is devoid of a reverse transcriptase template and/or primer binding site.

An extended guide nucleic acid may comprise a CRISPR nucleic acid (e.g., CRISPR RNA, CRISPR DNA, crRNA, crDNA) and/or a CRISPR nucleic acid and a tracr nucleic acid; and (b) an extended portion comprising a primer binding site and a reverse transcriptase template (RT template), wherein the RT template encodes a modification to be incorporated into the target nucleic acid. The CRISPR nucleic acid may be a Type II or Type V CRISPR nucleic acid and/or the tracr nucleic acid may be any tracr corresponding to the appropriate Type II or Type V CRISPR nucleic acid. In some embodiments, an extended guide nucleic acid comprises: (i) a Type V CRISPR nucleic acid or a Type II CRISPR nucleic acid (e.g., a Type II or Type V CRISPR RNA, Type II or Type V CRISPR DNA, Type II or Type V crRNA, or Type II or Type V crDNA) and/or a CRISPR nucleic acid and a tracr nucleic acid (e.g., a Type II or Type V tracrRNA, Type II or Type V tracrDNA); and (ii) an extended portion comprising a primer binding site and a reverse transcriptase template (RT template), wherein the Type V CRISPR nucleic acid or Type II CRISPR nucleic acid comprises a spacer that binds to a first strand (e.g., the target strand) of a target nucleic acid (e.g., the spacer is complementary to a portion of consecutive nucleotides in the first strand of the target nucleic acid) and the primer binding site binds to the first strand (e.g., target strand). In some embodiments, the extended portion can be fused to either the 5′ end or 3′ end of the CRISPR nucleic acid (e.g., from 5′ to 3′: repeat-spacer-extended portion or extended portion-repeat-spacer) and/or to the 5′ or 3′ end of the tracr nucleic acid. In some embodiments, the extended portion of an extended guide nucleic acid comprises, 5′ to 3′, an RT template (RTT) and a primer binding site (PBS) (e.g., 5′-crRNA-spacer-RTT (edit encoded)-PBS-3′) or comprises 5′ to 3′ a PBS and RTT, depending on the location of the extended portion relative to the CRISPR nucleic acid of the extended guide nucleic acid (e.g., 5′-crRNA-spacer-PBS-RTT (edit encoded)-3′). For example, in some embodiments, an extended portion of the extended guide nucleic acid may comprise, 5′ to 3′, an RT template and a primer binding site (when the extended guide is linked to the 3′ end of the CRISPR nucleic acid). In some embodiments, an extended portion of the extended guide may comprise, 5′ to 3′, a primer binding site and an RT template (when the extended guide is linked to the 5′ end of the CRISPR nucleic acid).

In some embodiments, a target nucleic acid is double stranded and comprises a first strand and a second strand and a primer binding site of an extended guide nucleic acid binds to the second strand (e.g., the non-target, top strand) of the target nucleic acid. In some embodiments, a target nucleic acid is double stranded and comprises a first strand and a second strand and a primer binding site of an extended guide nucleic acid binds to the first strand (e.g., binds to the target strand, optionally the same strand to which a CRISPR-Cas effector protein is recruited, bottom strand) of the target nucleic acid. In some embodiments, a target nucleic acid is double stranded and comprises a first strand and a second strand and the primer binding site of an extended guide nucleic acid binds to the second strand (e.g., the non-target strand, optionally the opposite strand from that to which the CRISPR-Cas effector protein is recruited) of the target nucleic acid. In some embodiments, a reverse transcriptase (RT) may add to the target strand of a target nucleic acid (e.g., the strand to which the spacer of the CRISPR nucleic acid of the extended guide nucleic acid is complementary and to which the CRISPR-Cas effector protein is recruited). In some embodiments, the reverse transcriptase (RT) adds to the non-target strand of a target nucleic acid (e.g., the strand that is complementary to the strand to which the spacer of the CRISPR nucleic acid is complementary and to which the CRISPR-Cas effector protein is recruited). Example methods and editing systems are described in International Patent Publication No. WO 2021/092130, International Patent Publication No. WO 2022/098993, and U.S. Patent Application Publication Nos. 2021/0147862, 2021/0130835, 2021/0147862, and 2022/0145334, each of which are incorporated herein by reference in their entirety.

The RT template of an extended guide nucleic acid may encode one or more modification(s) (e.g., edit(s)) to be incorporated into a target nucleic acid. The one or more modification(s) may be located in any position within an RT template (e.g., where the position location may be relative to the position of a protospacer adjacent motif (PAM) of the target nucleic acid). In some embodiments, an RT template has a modification at one or more positions from −1 to 23 (e.g., −1, 1, 2, 3, 4, 5, 6, 7, 8, 9, 10, 11, 12, 13, 14, 15, 16, 17, 18, 19, 20, 21, 22, or 23) relative to the position of a protospacer adjacent motif (PAM) (e.g., TTTG, TCCG, CCCC, and TTTV) in a target nucleic acid. In some embodiments, an RT template may comprise a modification located at nucleotide position-1, 1, 2, 3, 4, 5, 6, 7, 8, 9, 10, 11, 12, 13, 14, 15, 16, 17, 18, 19, 20, 21, 22 or 23. In some embodiments, an RT template may comprise a modification located at nucleotide position 4 to nucleotide position 17 (e.g., position 4, 5, 6, 7, 8, 9, 10, 11, 12, 13, 14, 15, 16, or 17) of the RT template relative to the position of a PAM of a target nucleic acid. In some embodiments, an RT template may comprise a modification located at nucleotide position 10 to nucleotide position 17 (e.g., position 10, 11, 12, 13, 14, 15, 16, or 17) of the RT template relative to the position of a PAM of a target nucleic acid. In some embodiments, an RT template may comprise a modification located at nucleotide position 12 to nucleotide position 15 (e.g., position 12, 13, 14, or 15) of the RT template relative to the position of a PAM of a target nucleic acid.

In some embodiments, an extended portion of an extended guide nucleic acid may comprise, 5′ to 3′, an RT template and a primer binding site (e.g., when the extended portion is linked to the 3′ end of a CRISPR nucleic acid). In some embodiments, an extended portion of an extended guide nucleic acid may comprise, 5′ to 3′, a primer binding site and an RT template (RTT) (e.g., when the extended portion is linked to the 5′ end of the CRISPR nucleic acid). In some embodiments, an RT template may have a length of about 1 nucleotide to about 100 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides, and any range or value therein), e.g., about 1 nucleotide to about 10 nucleotides, about 1 nucleotide to about 15 nucleotides, about 1 nucleotide to about 20 nucleotides, about 1 nucleotide to about 25 nucleotides, about 1 nucleotide to about 30 nucleotides, about 1 nucleotide to about 35, 36, 37, 38, 39 or 40 nucleotides, about 1 nucleotide to about 50 nucleotides, about 5 nucleotides to about 15 nucleotides, about 5 nucleotides to about 20 nucleotides, about 5 nucleotides to about 25 nucleotides, about 5 nucleotides to about 30 nucleotides, about 5 nucleotides to about 35, 36, 37, 38, 39 or 40 nucleotides, about 5 nucleotides to about 50 nucleotides, about 8 nucleotides to about 15 nucleotides, about 8 nucleotide to about 20 nucleotides, about 8 nucleotide to about 25 nucleotides, about 8 nucleotide to about 30 nucleotides, about 8 nucleotide to about 35, 36, 37, 38, 39 or 40 nucleotides, about 8 nucleotide to about 50 nucleotides in length, about 8 nucleotides to about 100 nucleotides, about 10 nucleotide to about 15 nucleotides, about 10 nucleotide to about 20 nucleotides, about 10 nucleotide to about 25 nucleotides, about 10 nucleotide to about 30 nucleotides, about 10 nucleotide to about 36 nucleotides, about 10 nucleotide to about 40 nucleotides, about 10 nucleotide to about 50 nucleotides, about 10 nucleotides to about 100 nucleotides in length and any range or value therein. In some embodiments, the length of an RT template may be at least 8 nucleotides, optionally about 8 nucleotides to about 100 nucleotides. In some embodiments, the length of an RT template is 36, 37, 38, 39 or 40 nucleotides or less (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length, or any value or range therein (e.g., about 1, 2, 3, 4, 5, 6, 7, 8, 9, 10, 11, 12, 13, 14, or 15 nucleotides in length to about 16, 17, 18, 19, 20, 21, 22, 23, 24, 25, 26, 27, 28, 29, 30, 31, 32, 33, 34, 35, 36, 37, 38, 39, or 40 nucleotides in length). In some embodiments, the length of an RT template may be at least 30 nucleotides, optionally about 30, 31, 32, 33, 34, 35, 36, 37, 38, 39, or 40 nucleotides in length to about to about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length, or any range or value therein. In some embodiments, the length of an RT template may be about 36, 40, 44, 47, 50, 52, 55, 63, 72 or 74 nucleotides. Within the length of the RTT one or more modification(s) may be present. The one or more modification(s) may be located anywhere within the RTT, wherein the position of the modification may be described relative to the position of a protospacer adjacent motif (PAM) of a target nucleic acid. In some embodiments, an RT template may comprise a modification located at nucleotide position-1, 1, 2, 3, 4, 5, 6, 7, 8, 9, 10, 11, 12, 13, 14, 15, 16, 17, 18, 19, 20, 21, 22 or 23. In some embodiments, an RT template may comprise a modification located at nucleotide position 4 to nucleotide position 17 (e.g., position 4, 5, 6, 7, 8, 9, 10, 11, 12, 13, 14, 15, 16, or 17) of the RT template relative to the position of a protospacer adjacent motif (PAM) of a target nucleic acid. In some embodiments, an RT template may comprise a modification located at nucleotide position 10 to nucleotide position 17 (e.g., position 10, 11, 12, 13, 14, 15, 16, or 17) of the RT template relative to the position of a protospacer adjacent motif (PAM) of a target nucleic acid. In some embodiments, an RT template may comprise a modification located at nucleotide position 12 to nucleotide position 15 (e.g., position 12, 13, 14, or 15) of the RT template relative to the position of a protospacer adjacent motif (PAM) of a target nucleic acid.

As used herein, a “primer binding site” (PBS) of an extended portion of an extended guide nucleic acid (e.g., a tagRNA) refers to a sequence of consecutive nucleotides that can bind to a region or “primer” on a target nucleic acid, e.g., is complementary to the target nucleic acid primer. As an example, a CRISPR Cas effector protein (e.g., a Type II or Type V, e.g., Cas 9 or Cas12a) may nick/cut the DNA and the 3′ end of the cut DNA acts as a primer for the PBS portion of the extended guide nucleic acid. The PBS may be complementary to the 3′ end of a strand of the target nucleic acid and may bind and/or may be configured to bind to either the target strand or non-target strand. A primer binding site can be fully complementary to the primer or it may be substantially complementary (e.g., at least 70% complementary (e.g., 70% or about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9% or more)) to the primer of a target nucleic acid. In some embodiments, the length of a primer binding site of an extended portion may be about 1 nucleotide to about 100 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides, or any value or range therein), or about 4 nucleotide to about 85 nucleotides, about 10 nucleotide to about 80 nucleotides, about 20 nucleotide to about 80 nucleotides, about 25 nucleotides to about 80 nucleotides about 30 nucleotide to about 80 nucleotides, about 40 nucleotide to about 80 nucleotides, about 45 nucleotide to about 80 nucleotides, about 45 nucleotide to about 75 nucleotides, or about 45 nucleotide to about 60 nucleotides, or any range or value therein. In some embodiments, the length of a PBS may be at least 30 nucleotides, optionally about 30, 31, 32, 33, 34, 35, 36, 37, 38, 39, or 40 nucleotides to about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length, or any range or value therein. In some embodiments, the length of a PBS may be about 8, 16, 24, 32, 40, 48, 56, 64, 72, or 80 nucleotides.

In some embodiments, an RTT may have a length of about 35 nucleotides to about 75 nucleotides and a PBS may have a length of about 30 nucleotides to about 80 nucleotides, optionally wherein the PBS may comprise a length of about 8, 16, 24, 32, 40, 48, 56, 64, 72, or 80 nucleotides and the RTT may comprise a length of about 36, 40, 44, 47, 50, 52, 55, 63, 72 or 74 nucleotides, or any combination thereof of the RTT length and/or PBS length.

In some embodiments, an extended portion of an extended guide nucleic acid may be fused to either the 5′ end or 3′ end of a Type II or a Type V CRISPR nucleic acid (e.g., 5′ to 3′: repeat-spacer-extended portion, or extended portion-repeat-spacer) and/or to the 5′ or 3′ end of the tracr nucleic acid. In some embodiments, when an extended portion is located 5′ of the crRNA, a Type V CRISPR-Cas effector protein is modified to reduce (or eliminate) self-processing RNase activity.

In some embodiments, the extended portion of an extended guide nucleic acid may be linked to the Type II or Type V CRISPR nucleic acid and/or the Type II or Type V tracrRNA via a linker. In some embodiments, a linker have a length of about 1 to about 100 nucleotides or more (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length, and any range therein (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, about 40 to about 100, about 50 to about 100, or about 2, 3, 4, 5, 6, 7, 8, 9, 10, 11, 12, 13, 14, 15, 16, 17, 18, 19, 20, 21, 22, 23, 24, 25 nucleotides to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length (e.g., about 105, 110, 115, 120, 130, 140 150 or more nucleotides in length).

A guide nucleic acid and/or an extended guide nucleic acid may comprise one or more recruiting motifs as described herein, which may be linked to the 5′ end and/or the 3′ end of the guide nucleic acid and/or it may be inserted into the guide nucleic acid (e.g., within a hairpin loop of the guide nucleic acid). In some embodiments, an extended guide nucleic acid may be linked to an RNA recruiting motif. An extended guide nucleic acid and/or guide nucleic acid may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion of an extended guide nucleic acid.

In some embodiments, an editing system comprises an extended guide nucleic acid that is linked to an RNA recruiting motif and a reverse transcriptase that is a reverse transcriptase fusion protein, wherein the reverse transcriptase fusion protein comprises a reverse transcriptase polypeptide fused to an affinity polypeptide that binds to the RNA recruiting motif, wherein the extended guide nucleic acid binds to a target nucleic acid and the RNA recruiting motif binds to the affinity polypeptide, thereby recruiting the reverse transcriptase fusion protein to the extended guide nucleic acid and contacting the target nucleic acid with the reverse transcriptase. In some embodiments, two or more reverse transcriptase fusion proteins may be recruited to an extended guide nucleic acid, thereby contacting the target nucleic acid with two or more reverse transcriptase fusion proteins.

The terms “transgene” or “transgenic” as used herein refer to at least one nucleic acid sequence that is taken from the genome of one organism or produced synthetically, and which is then introduced into a host cell (e.g., a plant cell) or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation into the genome and thus without inheritance of the respective at least one molecule introduced into the genome of a cell. The term “transgene-free” refers to a condition in which a transgene is not present or found in the genome of a host cell or tissue or organism of interest.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a nucleic acid construct of the invention (e.g., a polynucleotide encoding a polypeptide (e.g., a circular permutant) of the present invention, a polynucleotide encoding a nuclease, a polynucleotide encoding a reverse transcriptase, a polynucleotide encoding a reverse transcriptase fusion protein, a polynucleotide encoding a peptide tag, a polynucleotide encoding an affinity polypeptide, a polynucleotide encoding a glycosylase, and/or a polynucleotide comprising a guide nucleic acid), wherein the nucleic acid construct is operably associated with at least a control sequence (e.g., a promoter). Thus, some embodiments of the invention provide expression cassettes designed to express, for example, a nucleic acid construct of the invention. When an expression cassette comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). Thus, for example, a polynucleotide encoding a polypeptide of the present invention, and a polynucleotide comprising a guide nucleic acid comprised in an expression cassette may each be operably associated with a single promoter or one or more of the polynucleotide(s) may be operably associated with separate promoters (e.g., two or three promoters) in any combination, which may be the same or different from each other.

In some embodiments, an expression cassette comprising the polynucleotides/nucleic acid constructs of the invention may be optimized for expression in an organism (e.g., an animal, a plant, a bacterium and the like).

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to a gene encoding a CRISPR-Cas effector protein, or a gene encoding a polypeptide of the present invention, may be native to a host cell, or may be native to another source (e.g., foreign or heterologous to the promoter, to a gene encoding the CRISPR-Cas effector protein, to a host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

The expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector may comprise a nucleic acid construct comprising one or more nucleotide sequence(s) to be transferred, delivered or introduced into a cell. Vectors for use in transformation of host organisms are well known in the art. Non-limiting examples of general classes of vectors include viral vectors (e.g., Adeno-associated virus (AAV) vectors), plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally, included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid construct of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). Thus, for example, a target nucleic acid may be contacted with a nucleic acid construct of the invention comprising, for example, a polynucleotide encoding a polypeptide of the present invention and optionally a guide nucleic acid, under conditions whereby the polypeptide (e.g., comprising a circular permutant) is expressed, and the polypeptide forms a complex with a guide nucleic acid, the complex hybridizes to the target nucleic acid, and optionally the polypeptide of the present invention may recruit one or more additional component(s) (e.g., a reverse transcriptase, glycosylase inhibitor, and/or deaminase) to it (and thus, to the target nucleic acid) or one or more additional component(s) is/are fused to the polypeptide, thereby modifying the target nucleic acid. In some embodiments, a polypeptide of the present invention localizes at a target nucleic acid, optionally through non-covalent interactions. Methods for recruiting one or more additional component(s) (e.g., a reverse transcriptase and/or deaminase) may be used that take advantage of other protein-protein interactions, RNA-protein interactions, and/or chemical interactions. In some embodiments, a target nucleic acid may be contacted with a ribonucleoprotein comprising a polypeptide of the present invention (e.g., comprising a circular permutant) and optionally a guide nucleic acid and the ribonucleoprotein hybridizes to the target nucleic acid, and optionally the polypeptide of the present invention may recruit one or more additional component(s) (e.g., a reverse transcriptase, glycosylase inhibitor, and/or deaminase) to it (and thus, to the target nucleic acid) or one or more additional component(s) is/are fused to the polypeptide, thereby modifying the target nucleic acid.

In some embodiments, a target nucleic acid may be contacted with a nucleic acid construct of the invention encoding a polypeptide of the present invention and optionally a guide nucleic acid, under conditions whereby the polypeptide is expressed, or a target nucleic acid may be contacted with a polypeptide of the present invention and optionally a guide nucleic acid. The polypeptide of the present invention can form a complex with the guide nucleic acid, and the complex can hybridize to the target nucleic acid, and/or a polypeptide of the present invention may be recruited to a target nucleic acid, which may result in modifying the target nucleic acid.

As used herein, “modifying” or “modification” in reference to a target nucleic acid includes editing (e.g., mutating), covalent modification, exchanging/substituting nucleic acids/nucleotide bases, deleting, cleaving, and/or nicking of a target nucleic acid to thereby provide a modified nucleic acid and/or altering transcriptional control of a target nucleic acid to thereby provide a modified nucleic acid. In some embodiments, a modification may include an insertion and/or deletion of any size and/or a single base change (SNP) of any type. In some embodiments, a modification comprises a SNP. In some embodiments, a modification comprises exchanging and/or substituting one or more (e.g., 1, 2, 3, 4, 5, or more) nucleotides. In some embodiments, an insertion or deletion may be about 1 base to about 30,000 bases or more in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000 bases in length or more, or any value or range therein). Thus, in some embodiments, an insertion or deletion may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 bases in length, or any range or value therein; about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 bases to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 bases or more in length, or any value or range therein; about 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 bases to about 2500, 3000, 3500, 4000, 4500, 5000, 5500, 6000, 6500, 7000, 7500, 8000, 8500, 9000, 9500, or 10,000 bases or more in length, or any value or range therein; or about 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 bases to about 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, or 5000 bases or more in length, or any value or range therein. In some embodiments, an insertion or deletion may be about 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, or 10,000 bases to about 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, or 30,000 bases or more in length, or any value or range therein.

“Recruit,” “recruiting” or “recruitment” as used herein refer to attracting one or more polypeptide(s) or polynucleotide(s) to another polypeptide or polynucleotide (e.g., to a particular location in a genome) using protein-protein interactions, nucleic acid protein interactions (e.g., RNA-protein interactions), and/or chemical interactions. Protein-protein interactions can include, but are not limited to, peptide tags (epitopes, multimerized epitopes) and corresponding affinity polypeptides, RNA recruiting motifs and corresponding affinity polypeptides, and/or chemical interactions. Example chemical interactions that may be useful with polypeptides and polynucleotides for the purpose of recruitment can include, but are not limited to, rapamycin-inducible dimerization of FRB-FKBP; Biotin-streptavidin interaction; SNAP tag (Hussain et al. Curr Pharm Des. 19 (30): 5437-42 (2013)); Halo tag (Los et al. ACS Chem Biol. 3 (6): 373-82 (2008)); CLIP tag (Gautier et al. Chemistry & Biology 15:128-136 (2008)); DmrA-DmrC heterodimer induced by a compound (Tak et al. Nat Methods 14 (12): 1163-1166 (2017)); and/or bifunctional ligand approaches (e.g., chemically induced dimerization (Voß et al. Curr Opin Chemical Biology 28:194-201 (2015) (e.g. dihyrofolate reductase (DHFR) (Kopyteck et al. Cell Cehm Biol 7 (5): 313-321 (2000)). In some embodiments, a recruiting method and/or system of the present invention uses a protein-protein interaction and/or nucleic acid-protein interaction (e.g., RNA-protein interactions) to attract a polypeptide or polynucleotide to another polypeptide or polynucleotide (e.g., to a particular location in a genome).

“Introducing,” “introduce,” “introduced” (and grammatical variations thereof) in the context of a polynucleotide of interest or editing system means presenting a nucleotide sequence of interest (e.g., polynucleotide, a nucleic acid construct, and/or a guide nucleic acid) and/or editing system (e.g., a polynucleotide, polypeptide, and/or ribonucleoprotein) to a host organism or cell of said organism (e.g., host cell; e.g., a plant cell) in such a manner that the nucleotide sequence and/or editing system gains access to the interior of a cell. Thus, for example, a nucleic acid construct of the invention encoding a polypeptide of the present invention and/or a guide nucleic acid may be introduced into a cell of an organism, thereby transforming the cell with the polypeptide, CRISPR-Cas effector protein, guide nucleic acid, and reverse transcriptase. In some embodiments, a polypeptide of the present invention and/or a guide nucleic acid may be introduced into a cell of an organism, optionally wherein the polypeptide and guide nucleic acid may be comprised in a complex (e.g., a ribonucleoprotein). In some embodiments, the organism is a eukaryote (e.g., a mammal such as a human).

The term “transformation” as used herein refers to the introduction of a nucleic acid, polypeptide, and/or ribonucleoprotein (e.g., a heterologous nucleic acid, polypeptide, and/or ribonucleoprotein) into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a nucleic acid construct, a polypeptide, and/or a ribonucleoprotein of the invention.

“Transient transformation” in the context of a polynucleotide, polypeptide, and/or ribonucleoprotein means that a polynucleotide, polypeptide, and/or ribonucleoprotein is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a mammal, plant, etc.). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention may be transiently introduced into a cell with a guide nucleic acid and as such, no DNA maintained in the cell.

A nucleic acid construct, polypeptide, and/or ribonucleoprotein of the invention can be introduced into a cell by any method known to those of skill in the art. In some embodiments, transformation methods include, but are not limited to, transformation via bacterial-mediated nucleic acid delivery (e.g., via Agrobacteria), viral-mediated nucleic acid delivery, silicon carbide and/or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the cell (e.g., a plant cell or an animal cell), including any combination thereof. In some embodiments of the invention, transformation of a cell comprises nuclear transformation. In some embodiments, transformation of a cell comprises plastid transformation (e.g., chloroplast transformation). In some embodiments, a recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

A nucleotide sequence, polypeptide, and/or ribonucleoprotein therefore can be introduced into a host organism or its cell in any number of ways that are well known in the art. The methods of the invention do not depend on a particular method for introducing one or more nucleotide sequence(s), polypeptide(s), and/or ribonucleoprotein(s) into the organism, only that they gain access to the interior of at least one cell of the organism. Where more than one nucleotide sequence, polypeptide, and/or ribonucleoprotein is to be introduced, they can be assembled as part of a single nucleic acid construct, or as separate nucleic acid constructs, and can be located on the same or different nucleic acid constructs. Accordingly, a nucleotide sequence, polypeptide, and/or ribonucleoprotein can be introduced into the cell of interest in a single transformation event, and/or in separate transformation events, or, alternatively, where relevant, a nucleotide sequence can be incorporated into a plant, for example, as part of a breeding protocol. In some embodiments, the cell is a eukaryotic cell (e.g., a plant cell or a mammalian such as a human cell).

In some embodiments, a nucleic acid construct of the invention (e.g., a polynucleotide encoding a polypeptide of the present invention, and/or a guide nucleic acid and/or expression cassettes and/or vectors comprising the same) may be operably linked to at least one regulatory sequence, optionally, wherein the at least one regulatory sequence may be codon optimized for expression in a plant. In some embodiments, the at least one regulatory sequence may be, for example, a promoter, an operon, a terminator, or an enhancer. In some embodiments, the at least one regulatory sequence may be a promoter. In some embodiments, the regulatory sequence may be an intron. In some embodiments, the at least one regulatory sequence may be, for example, a promoter operably associated with an intron or a promoter region comprising an intron. In some embodiments, the at least one regulatory sequence may be, for example a ubiquitin promoter and its associated intron (e.g., Medicago truncatula and/or Zea mays and their associated introns). In some embodiments, the at least one regulatory sequence may be a terminator nucleotide sequence and/or an enhancer nucleotide sequence.

In some embodiments, a nucleic acid construct of the invention may be operably associated with a promoter region, wherein the promoter region comprises an intron, optionally wherein the promoter region may be a ubiquitin promoter and intron (e.g., a Medicago or a maize ubiquitin promoter and intron, e.g., SEQ ID NO:37 or SEQ ID NO:38). In some embodiments, the nucleic acid construct of the invention that is operably associated with a promoter region comprising an intron may be codon optimized for expression in a plant.

In some embodiments, a nucleic acid construct of the invention may encode one or more (e.g., 1, 2, 3, 4, or more) polypeptide(s) of interest. The one or more polypeptides of interest may be codon optimized for expression in a eukaryote (e.g., a human or a plant). In some embodiments, a polypeptide of the present invention may comprise to one or more (e.g., 1, 2, 3, 4, or more) polypeptide(s) of interest.

A polypeptide of interest useful with this invention can include, but is not limited to, a polypeptide or protein domain having deaminase activity, nickase activity, recombinase activity, transposase activity, methylase activity, glycosylase (DNA glycosylase) activity, glycosylase inhibitor activity (e.g., uracil-DNA glycosylase inhibitor (UGI)), a reverse transcriptase, a peptide tag (e.g., a GCN4 peptide tag), demethylase activity, transcription activation activity, transcription repression activity, transcription release factor activity, histone modification activity, nuclease activity, single-strand RNA cleavage activity, double-strand RNA cleavage activity, restriction endonuclease activity (e.g., Fok1), nucleic acid binding activity, methyltransferase activity, DNA repair activity, DNA damage activity, dismutase activity, alkylation activity, depurination activity, oxidation activity, pyrimidine dimer forming activity, integrase activity, transposase activity, polymerase activity, ligase activity, helicase activity, a nuclear localization sequence or activity, an affinity polypeptide, a peptide tag, and/or photolyase activity. In some embodiments, the polypeptide of interest is a Fok1 nuclease, or a uracil-DNA glycosylase inhibitor. In some embodiments, the polypeptide of interest is a cytosine deaminase (e.g., apolipoprotein B mRNA editing enzyme, catalytic polypeptide-like 3A (A3A)). In some embodiments, the polypeptide of interest is an adenine deaminase (e.g., TadA8e). When encoded in a nucleic acid (polynucleotide, expression cassette, and/or vector) the encoded polypeptide or protein domain may be codon optimized for expression in an organism. In some embodiments, a polypeptide of interest may be linked to a CRISPR-Cas effector protein domain to provide a CRISPR-Cas fusion protein. In some embodiments, a CRISPR-Cas fusion protein that comprises a CRISPR-Cas effector protein domain linked to a peptide tag may also be linked to a polypeptide of interest (e.g., a CRISPR-Cas effector protein domain may be, for example, linked to both a peptide tag (or an affinity polypeptide) and, for example, a polypeptide of interest.

In some embodiments, an editing system of the present invention comprises a CRISPR-Cas effector protein and/or a polypeptide of the present invention (e.g., a circular permutant). As used herein, a “CRISPR-Cas effector protein” is a protein or polypeptide that cleaves, cuts, or nicks a nucleic acid; binds a nucleic acid (e.g., a target nucleic acid and/or a guide nucleic acid); and/or that identifies, recognizes, or binds a guide nucleic acid as defined herein. In some embodiments, a CRISPR-Cas effector protein may be an enzyme (e.g., a nuclease, endonuclease, nickase, etc.) and/or may function as an enzyme. In some embodiments, a CRISPR-Cas effector protein refers to a CRISPR-Cas nuclease. In some embodiments, a CRISPR-Cas effector protein comprises nuclease activity and/or nickase activity, comprises a nuclease domain whose nuclease activity and/or nickase activity has been reduced or eliminated, comprises single stranded DNA cleavage activity (ss DNase activity) or which has ss DNase activity that has been reduced or eliminated, and/or comprises self-processing RNase activity or which has self-processing RNase activity that has been reduced or eliminated. A CRISPR-Cas effector protein may bind to a target nucleic acid. A CRISPR-Cas effector protein may be a Type I, II, III, IV, V, or VI CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein may be from a Type II CRISPR-Cas system or a Type V CRISPR-Cas system. In some embodiments, a CRISPR-Cas effector protein may be a Type II CRISPR-Cas effector protein, for example, a Cas9 effector protein. In some embodiments, a CRISPR-Cas effector protein may be Type V CRISPR-Cas effector protein, for example, a Cas12 effector protein. In some embodiments, a CRISPR-Cas effector protein may be Cas12a and optionally may have an amino acid sequence of any one of SEQ ID NOs: 39-65, 298, and 850-852 and/or a nucleotide sequence of any one of SEQ ID NOs: 66-68, 246, and 949. In some embodiments, a CRISPR-Cas effector protein may be an active Cas 12a and optionally may have an amino acid sequence of SEQ ID NO:46, 55 or 56. In some embodiments, a CRISPR-Cas effector protein may be an inactive (i.e., dead) Cas12a and optionally may have an amino acid sequence of SEQ ID NO: 59, 60, 61, or 850-852.

Exemplary CRISPR-Cas effector proteins include, but are not limited to, a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site and/or nuclease domain (e.g., a RuvC, HNH, e.g., a RuvC site of a Cas12a nuclease domain; e.g., a RuvC site and/or HNH site of a Cas9 nuclease domain). A CRISPR-Cas effector protein having a mutation in its nuclease active site and/or nuclease domain that causes the protein to not have nuclease activity, is commonly referred to as “inactive” or “dead,” e.g., dCas9. In some embodiments, a CRISPR-Cas effector protein having a mutation in its nuclease active site and/or nuclease domain may have impaired activity or reduced activity (e.g., nickase activity) as compared to the same CRISPR-Cas effector protein without the mutation. In some embodiments, a CRISPR-Cas effector protein having a mutation in its nuclease active site may comprise an arginine to alanine mutation in its nuclease active site. In some embodiments, a CRISPR-Cas effector protein having a mutation in its nuclease active site may be a Cas12a nickase comprising an R1138A mutation and/or may have the amino acid sequence of SEQ ID NO:62. In some embodiments, a CRISPR-Cas effector protein may be a non-target strand catalytic nickase.

A CRISPR Cas9 effector protein or Cas9 useful with this invention may be any known or later identified Cas9 nuclease. In some embodiments, a Cas9 may be a protein from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. In some embodiments, a CRISPR-Cas effector protein may be a Cas9 and optionally may have a nucleotide sequence of any one of SEQ ID NOs: 141-155 and/or an amino acid sequence of any one of SEQ ID NOs: 133-140 and 156-157.

In some embodiments, the CRISPR-Cas effector protein may be a Cas9 derived from Streptococcus pyogenes and/or may recognize the PAM sequence motif NGG, NAG, NGA (Mali et al, Science 2013; 339 (6121): 823-826). In some embodiments, the CRISPR-Cas effector protein may be a Cas9 derived from Streptococcus thermophiles and/or may recognize the PAM sequence motif NGGNG and/or NNAGAAW (W=A or T) (See, e.g., Horvath et al, Science, 2010; 327 (5962): 167-170, and Deveau et al, J Bacteriol 2008; 190 (4): 1390-1400). In some embodiments, the CRISPR-Cas effector protein may be a Cas9 derived from Streptococcus mutans and/or may recognize the PAM sequence motif NGG and/or NAAR (R=A or G) (See, e.g., Deveau et al, J BACTERIOL 2008; 190 (4): 1390-1400). In some embodiments, the CRISPR-Cas effector protein may be a Cas9 derived from Streptococcus aureus and/or may recognize the PAM sequence motif NNGRR (R=A or G). In some embodiments, the CRISPR-Cas effector protein may be a Cas9 derived from S. aureus and/or may recognize the PAM sequence motif N GRRT (R=A or G). In some embodiments, the CRISPR-Cas effector protein may be a Cas9 derived from S. aureus and/or may recognize the PAM sequence motif N GRRV (R=A or G). In some embodiments, the CRISPR-Cas effector protein may be a Cas9 that is derived from Neisseria meningitidis and/or may recognize the PAM sequence motif N GATT or N GCTT (R=A or G, V=A, G or C) (See, e.g., Hou et ah, PNAS 2013, 1-6). In the aforementioned embodiments in this paragraph, N in the PAM sequence motif can be any nucleotide residue, e.g., any of A, G, C or T. In some embodiments, the CRISPR-Cas effector protein may be a Cas13a derived from Leptotrichia shahii and/or may recognize a protospacer flanking sequence (PFS) (or RNA PAM (rPAM)) sequence motif of a single 3′ A, U, or C, which may be located within the target nucleic acid.

A Type V CRISPR-Cas effector protein useful with embodiments of the invention may be any Type V CRISPR-Cas nuclease. Exemplary Type V CRISPR-Cas effector proteins include, but are not limited, to Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c nuclease. In some embodiments, a Type V CRISPR-Cas effector protein may be a Cas12a. In some embodiments, a Type V CRISPR-Cas effector protein may be a nickase, optionally, a Cas12a nickase.

In some embodiments, the CRISPR-Cas effector protein may be a Type V Clustered Regularly Interspaced Short Palindromic Repeats (CRISPR)-Cas nuclease. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein useful with this invention may be any known or later identified Cas12a (previously known as Cpf1) (see, e.g., U.S. Pat. No. 9,790,490, which is incorporated by reference for its disclosures of Cpf1 (Cas12a) sequences). The term “Cas12a” refers to an RNA-guided protein that can have nuclease activity, the protein comprising a guide nucleic acid binding domain and an active, inactive, or partially active DNA cleavage domain, thereby the RNA-guided nuclease activity of the Cas12a may be active, inactive or partially active, respectively. In some embodiments, a Cas12a useful with the invention may comprise a mutation in the nuclease active site (e.g., a RuvC site of the Cas12a domain). A Cas12a having a mutation in its nuclease domain and/or nuclease active site, and therefore, no longer comprising nuclease activity, is commonly referred to as deadCas 12a (e.g., dCas12a). In some embodiments, a Cas12a having a mutation in its nuclease domain and/or nuclease active site may have impaired activity, e.g., may have reduced nickase activity. In some embodiments, a Cas12a may have an amino acid sequence of any one of SEQ ID NOs: 39-65, 298, and 850-852.

In some embodiments, a CRISPR-Cas effector protein may be optimized for expression in an organism, for example, in an animal (e.g., a mammal such as a human), a plant, a fungus, an archaeon, or a bacterium. In some embodiments, a CRISPR-Cas effector protein (e.g., Cas12a polypeptide/domain or a Cas9 polypeptide/domain) may be optimized for expression in a plant.

A polypeptide of the present invention may be used in combination with a guide nucleic acid (e.g., guide RNA (gRNA), CRISPR array, CRISPR RNA, crRNA, or extended guide nucleic acid) that is designed to function with a CRISPR-Cas effector protein to modify a target nucleic acid. A guide nucleic acid useful with this invention may comprise at least one spacer sequence and at least one repeat sequence. The guide nucleic acid may be capable of forming a complex with a CRISPR-Cas effector protein (e.g., with a nuclease domain of the protein) and/or a polypeptide of the present invention and the spacer sequence is capable of hybridizing to a target nucleic acid, thereby guiding the complex to the target nucleic acid, wherein the target nucleic acid may be modified (e.g., cleaved or edited) and/or modulated (e.g., modulating transcription) by a polypeptide of the present invention, optionally present in and/or recruited to the complex).

In some embodiments, a CRISPR-Cas effector protein comprising a Cas9 domain (or a nucleic acid construct encoding the same) may be used in combination with a Cas9 guide nucleic acid to modify a target nucleic acid, and may be in or may form a complex.

Likewise, a CRISPR-Cas effector protein may comprise a Cas12a domain (or other selected CRISPR-Cas nuclease, e.g., C2c1, C2c3, Cas12b, Cas12c, Cas12d, Cas12f, Cas12i, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5), which may be used in combination with a Cas12a guide nucleic acid (or the guide nucleic acid for the other selected CRISPR-Cas nuclease) to modify a target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target nucleic acid (e.g., a target DNA and/or a protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas12f, Cas12i, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. In some embodiments, the guide nucleic acid comprises DNA. In some embodiments, the guide nucleic acid comprises RNA (e.g., is a guide RNA). The design of a gRNA of this invention may be based on a Type I, Type II, Type III, Type IV, Type V, or Type VI CRISPR-Cas system.

In some embodiments, a spacer sequence of the present invention comprises a polynucleotide having at least 70%, 75%, 80%, 85%, 90%, 95%, 96%, 97%, 98%, 99%, or 100% sequence identity to one or more of any one of SEQ ID NOs: 275-277, 799-802, 804-808, 865-866, 873-877, and 889-917. In some embodiments, a spacer sequence of the present invention may be comprised in a vector. In some embodiments, a vector comprising a spacer sequence of the present invention comprises a polynucleotide having at least 70%, 75%, 80%, 85%, 90%, 95%, 96%, 97%, 98%, 99%, or 100% sequence identity to one or more of any one of SEQ ID NOs: 278-280, 809-816, 867-868, 878-882, and 918-946.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas 12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35 (Web Server issue): W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild-type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild-type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). In some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length. The numbering and/or positioning of nucleotides in a spacer sequence starts at the first nucleotide at the 5′ end of the spacer sequence (i.e., nucleotide 1) and is consecutively numbered to the last nucleotide at the 3′ end of the spacer sequence. Thus, a spacer having a length of 30 nucleotides has nucleotide 1 up to nucleotide 30 in the 5′ to 3′ direction.

In some embodiments, the 5′ region of a spacer sequence of a guide nucleic acid may be fully complementary to a target nucleic acid, while the 3′ region of the spacer may be substantially complementary to the target nucleic acid (such as for a spacer in a Type V CRISPR-Cas system), or the 3′ region of a spacer sequence of a guide nucleic acid may be fully complementary to a target nucleic acid, while the 5′ region of the spacer may be substantially complementary to the target nucleic acid (such as for a spacer in a Type II CRISPR-Cas system), and therefore, the overall complementarity of the spacer sequence to the target nucleic acid may be less than 100%. Thus, for example, in a guide nucleic acid for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target nucleic acid, while the remaining nucleotides in the 3′ region of the spacer sequence are substantially complementary (e.g., at least about 70% complementary) to the target nucleic acid. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target nucleic acid, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleic acid.

As a further example, in a guide nucleic acid for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target nucleic acid, while the remaining nucleotides in the 5′ region of the spacer sequence are substantially complementary (e.g., at least about 70% complementary) to the target nucleic acid. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target nucleic acid, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target nucleic acid. A recruiting guide RNA further comprises one or more recruiting motifs as described herein, which may be linked to the 5′ end of the guide or the 3′ end or it may be inserted into the recruiting guide nucleic acid (e.g., within the hairpin loop).

In some embodiments, a seed region of a spacer may be about 8 to about 10 nucleotides in length, about 5 to about 6 nucleotides in length, or about 6 nucleotides in length.

In some embodiments, a guide nucleic acid further comprises a reverse transcriptase template and may be referred to as an extended guide nucleic acid.

A guide nucleic acid and/or an extended guide nucleic acid may comprise one or more recruiting motifs as described herein, which may be linked to the 5′ end and/or the 3′ end of the guide nucleic acid and/or it may be inserted into the guide nucleic acid (e.g., within a hairpin loop of the guide nucleic acid).

A “target nucleic acid”, “target DNA,” “target nucleotide sequence,” “target region,” and “target region in the genome” are used interchangeably herein and refer to a region of an organism's (e.g., a plant's) genome that comprises a sequence that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid as defined herein. A target nucleic acid is targeted by an editing system (or a component thereof) as described herein. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome or mammalian (e.g., human) genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” or “protospacer” as used herein refer to a sequence that is fully or substantially complementary to (and can hybridize to) a spacer sequence of a guide nucleic acid. In some embodiments, the protospacer is all or a portion of a target nucleic acid as defined herein that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type V CRISPR-Cas systems, the PAM is located at the 5′ end on the non-target strand and at the 3′ end of the target strand (see below, as an example).

```
    5′-NNNNNNNNNNNNNNNNNNN-3′ RNA Spacer
            ||||||||||||||||||
3′AAANNNNNNNNNNNNNNNNNNN-5′ Target strand
    ||||
5′TTTNNNNNNNNNNNNNNNNNNN-3′ Non-target strand
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient. In some embodiments, non-canonical PAM sequences may include, but are not limited to, 5′-CCCC-3′ or 5′-TCCG-3′.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encodes a polypeptide of the present invention and/or a CRISPR-Cas effector protein, and each may be comprised on the same or on a separate expression cassette or vector from that comprising the one or more guide nucleic acids. When the nucleic acid construct encoding a polypeptide of the present invention or the components of an editing system is/are comprised on separate expression cassette(s) or vector(s) from that comprising the guide nucleic acid, a target nucleic acid may be contacted with (e.g., provided with) the expression cassette(s) or vector(s) encoding the polypeptide of the present invention or components of an editing system in any order from one another and the guide nucleic acid, e.g., prior to, concurrently with, or after the expression cassette comprising the guide nucleic acid is provided (e.g., contacted with the target nucleic acid).

Methods of recruiting one or more components of an editing system to each other and/or to a target nucleic acid are known in the art and may include the use of a peptide tag or an affinity polypeptide that interacts with the peptide tag. In some embodiments, a guide nucleic acid may be linked to an RNA recruiting motif and a polypeptide of the present invention may be linked to an affinity polypeptide capable of interacting with the RNA recruiting motif, thereby recruiting the polypeptide of the invention to the target nucleic acid. Alternatively, chemical interactions may be used to recruit a polypeptide (e.g., a polypeptide of the invention) to a target nucleic acid.

A “recruiting motif” as used herein refers to one half of a binding pair that may be used to recruit a compound to which the recruiting motif is bound to another compound that includes the other half of the binding pair (i.e., a “corresponding motif”). The recruiting motif and corresponding motif may bind noncovalently. In some embodiments, a recruiting motif is an RNA recruiting motif (e.g., an RNA recruiting motif that is capable of binding and/or configured to bind to an affinity polypeptide), an affinity polypeptide (e.g., an affinity polypeptide that is capable of binding and/or configured to bind an RNA recruiting motif and/or a peptide tag), or a peptide tag (e.g., a peptide tag that is capable of binding and/or configured to bind an affinity polypeptide). For example, when a recruiting motif is an RNA recruiting motif, the corresponding motif for the RNA recruiting motif may be an affinity polypeptide that binds the RNA recruiting motif. A further example is that when a recruiting motif is a peptide tag, the corresponding motif for the peptide tag may be an affinity polypeptide that binds the peptide tag. Thus, a compound comprising a recruiting motif (e.g., an affinity polypeptide) may be recruited to another compound (e.g., a guide nucleic acid) comprising a corresponding motif for the recruiting motif (e.g., an RNA recruiting motif).

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26 (5): 910-924 (2017)); Gilbreth (Curr Opin Struc Biol 22 (4): 413-420 (2013)), U.S. Pat. No. 9,982,053, each of which are incorporated by reference in their entireties for the teachings relevant to affibodies, anticalins, monobodies and/or DARPins.

In some embodiments, a guide nucleic acid may be linked to an RNA recruiting motif, and a polypeptide to be recruited (e.g., a polypeptide of the present invention) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., a polypeptide of the present invention). In some embodiments, two or more polypeptides may be recruited to a guide nucleic acid, thereby contacting the target nucleic acid with two or more polypeptides (e.g., one or more polypeptide(s) of the present invention).

In some embodiments of the invention, a guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF). Exemplary RNA recruiting motifs and corresponding affinity polypeptides that may be useful with this invention can include, but are not limited to, SEQ ID NOs: 220-230.

In some embodiments, the components for recruiting polypeptides and nucleic acids may include those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., chemically induced dimerization).

As described herein, a “peptide tag” may be employed to recruit one or more polypeptides. A peptide tag may be any polypeptide that is capable of being bound by a corresponding motif such as an affinity polypeptide. A peptide tag may also be referred to as an “epitope” and when provided in multiple copies, a “multimerized epitope.” Example peptide tags can include, but are not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. In some embodiments, a peptide tag may also include phosphorylated tyrosines in specific sequence contexts recognized by SH2 domains, characteristic consensus sequences containing phosphoserines recognized by 14-3-3 proteins, proline rich peptide motifs recognized by SH3 domains, PDZ protein interaction domains or the PDZ signal sequences, and an AGO hook motif from plants. Peptide tags are disclosed in WO2018/136783 and U.S. Patent Application Publication No. 2017/0219596, which are incorporated by reference for their disclosures of peptide tags. Peptide tags that may be useful with this invention can include, but are not limited to, SEQ ID NO:231 and SEQ ID NO:232. An affinity polypeptide useful with peptide tags includes, but is not limited to, SEQ ID NO:233.

A peptide tag may comprise or be present in one copy or in 2 or more copies of the peptide tag (e.g., multimerized peptide tag or multimerized epitope) (e.g., about 2, 3, 4, 5, 6, 7, 8, 9, 10, 11, 12, 13, 14, 15, 16, 17, 18, 9, 20, 21, 22, 23, 24, or 25 or more peptide tags). When multimerized, the peptide tags may be fused directly to one another or they may be linked to one another via one or more amino acids (e.g., 1, 2, 3, 4, 5, 6, 7, 8, 9, 10, 11, 12, 13, 14, 15, 16, 17, 18, 19, 20 or more amino acids, optionally about 3 to about 10, about 4 to about 10, about 5 to about 10, about 5 to about 15, or about 5 to about 20 amino acids, and the like, and any value or range therein. Thus, in some embodiments, a CRISPR-Cas effector protein and/or polypeptide of the invention may be fused to one peptide tag or to two or more peptide tags, optionally wherein the two or more peptide tags are fused to one another via one or more amino acid residues. In some embodiments, a peptide tag useful with the invention may be a single copy of a GCN4 peptide tag or epitope or may be a multimerized GCN4 epitope comprising about 2 to about 25 or more copies of the peptide tag (e.g., about 2, 3, 4, 5, 6, 7, 8, 9, 10, 11, 12, 13, 14, 15, 16, 17, 18, 19, 20, 21, 22, 23, 24, 25 or more copies of a GCN4 epitope or any range therein).

In some embodiments, a peptide tag may be fused to a CRISPR-Cas polypeptide or domain. In some embodiments, a peptide tag may be fused or linked to the C-terminus of a CRISPR-Cas effector protein to form a CRISPR-Cas fusion protein. In some embodiments, a peptide tag may be fused or linked to the N-terminus of a CRISPR-Cas effector protein to form a CRISPR-Cas fusion protein. In some embodiments, a peptide tag may be fused within a CRISPR-Cas effector protein (e.g., a peptide tag may be in a loop region of a CRISPR-Cas effector protein). In some embodiments, peptide tag may be fused to or a polypeptide of the present invention.

An “affinity polypeptide” (e.g., “recruiting polypeptide”) refers to any polypeptide that is capable of binding to its corresponding peptide tag, peptide tag, or RNA recruiting motif. An affinity polypeptide for a peptide tag may be, for example, an antibody and/or a single chain antibody that specifically binds the peptide tag, respectively. In some embodiments, an antibody for a peptide tag may be, but is not limited to, an scFv antibody. In some embodiments, an affinity polypeptide may be fused or linked to the N-terminus of a polypeptide 25 of the present invention. In some embodiments, the affinity polypeptide is stable under the reducing conditions of a cell or cellular extract.

The nucleic acid constructs of the invention and/or guide nucleic acids may be comprised in one or more expression cassettes as described herein. In some embodiments, a nucleic acid construct of the invention may be comprised in the same or in a separate expression cassette or vector from that comprising a guide nucleic acid and/or an extended guide nucleic acid.

In some embodiments, a nucleic acid construct, expression cassette, or vector of the invention that is optimized for expression in an organism (e.g., a human or plant) may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to a nucleic acid construct, expression cassette or vector comprising the same polynucleotide(s) but which have not been codon optimized for expression in the organism.

When used in combination with a guide nucleic acid, a nucleic acid construct of the invention (and expression cassette and/or vector comprising the same) may be used to modify a target nucleic acid and/or its expression. A target nucleic acid may be contacted with a nucleic acid construct of the invention and/or expression cassettes and/or vectors comprising the same prior to, concurrently with or after contacting the target nucleic acid with the guide nucleic acid/recruiting guide nucleic acid (and/or expression cassettes and vectors comprising the same.

According to embodiments of the present invention, provided herein are polypeptides comprising a circular permutant. A circular permutant of the present invention is a circular permutant of a Cas 12a or an engineered protein that comprises a Cas 12a polypeptide. “Circular permutant” as used herein refers to an altered polypeptide compared to an original polypeptide (e.g., an initial polypeptide, reference polypeptide, and/or control polypeptide), wherein the order of the amino acid residues in the altered polypeptide is different than the order of the amino acid residues in the original polypeptide and the altered polypeptide has the N-terminal amino acid residue and the C-terminal amino acid residue of the original polypeptide covalently linked together (directly or indirectly). The altered polypeptide can thus have a new N-terminal end and a new C-terminal end compared to the N-terminal end and C-terminal end, respectively, of the original polypeptide. Accordingly, the circular permutant has a different sequence than the original polypeptide. An “altered polypeptide” as used herein refers to a polypeptide that comprises a non-natural mutation compared to another polypeptide (e.g., an original polypeptide). For example, an altered polypeptide may be a circular permutant and the non-natural mutation is the covalent linkage of the N- and C-termini of an original polypeptide and/or the formation of new N- and C-termini compared to the N- and C-termini, respectively, of an original polypeptide. “N-terminus” and “N-terminal amino acid residue” as used herein refer to the amino acid residue at the start of a polypeptide (i.e., amino acid residue 1) that has a free amino group (e.g., an amine or ammonium cation). “C-terminus” and “C-terminal amino acid residue” as used herein refer to the amino acid residue at the end of a polypeptide that has a free carboxylate group. “N-terminal end” as used herein refers to all or a portion of the amino acid residues starting at and including the N-terminus up to the middle of the polypeptide. “C-terminal end” as used herein refers to all or a portion of the amino acid residues starting at and including the C-terminus up to the middle of the polypeptide. Thus, for a polypeptide including 500 amino acid residues, the N-terminal end can include all or a portion of the sequence that starts at amino acid residue 1 (the N-terminus) to amino acid residue 250 and the C-terminal end can include all or a portion of the sequence that starts at amino acid residue 251 to amino acid residue 500 (the C-terminus). As another example, for a polypeptide including 223 amino acid residues, the N-terminal end can include all or a portion of the sequence that starts at amino acid residue 1 (the N-terminus) to amino acid residue 111 and the C-terminal end can include all or a portion of the sequence that starts at amino acid residue 113 to amino acid residue 223 (the C-terminus).

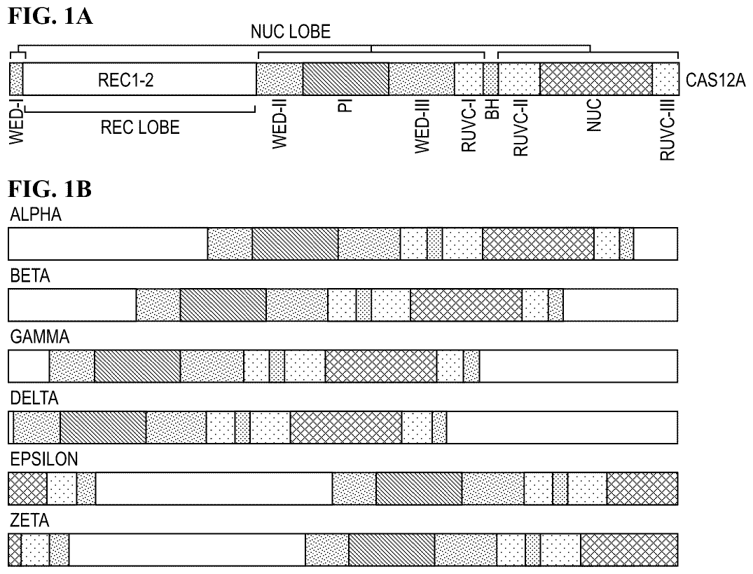

In general, a circular permutant may be conceptualized as having the N- and C-termini of a polypeptide (e.g., an original polypeptide) linked together and new N- and C-terminal ends that result from breaking a peptide bond in the polypeptide. For example, a circular permutant of the present invention may comprise fused N- and C-termini of a Cas12a or engineered protein that comprises a Cas 12a polypeptide with the circular permutant having new sequences at N- and C-terminal ends that are different than the sequences at the N- and C-terminal ends of the Cas12a or engineered protein and/or having a new N-terminal residue and/or new C-terminal residue compared to the N-terminal residue and/or new C-terminal residue, respectively, of the Cas12a or engineered protein as shown, for example, in FIG. 1A and FIG. 1B. A circular permutant of the present invention is derived and/or obtained from a Cas12a or is derived and/or obtained from an engineered protein that comprises a Cas12a polypeptide and/or may be a variant of a Cas12a or an engineered protein that comprises a Cas12a polypeptide. “Variant” as used herein in reference to a circular permutant of the present invention being a variant of a referenced polypeptide means that the circular permutant has the N- and C-termini of the referenced polypeptide (e.g., an original polypeptide such as a Cas12a or an engineered protein that comprises a Cas12a polypeptide) covalently linked (e.g., directly or indirectly) and the N- and C-termini of the circular permutant can be provided by breaking a peptide bond in the sequence of the referenced polypeptide. In some embodiments, a circular permutant of the present invention is a variant of a Cas12a or an engineered protein that comprises a Cas 12a polypeptide (e.g., a variant of at least one of SEQ ID NOs: 39-65, 69-132, 159-182, 281-284, 298, 400-401, 741, 761, and 850-861) and the circular permutant has one or more (e.g., 1, 2, 3, 4, 5, or more) mutation(s) compared to the Cas12a or the engineered protein of which it is a variant. For example, a circular permutant of the present invention may be a variant of a Cas12a and the circular permutant may include a mutation in the nuclease active site (e.g., in a RuvC site) that is not present in the Cas12a. In some embodiments, a circular permutant of the present may have a sequence that, when reordered to have the same N- and C-termini as a referenced polypeptide of which the circular permutant is a variant, has at least 90%, 91%, 92%, 93%, 94%, 95%, 96%, 97%, 98%, 99%, or 100% sequence identity to the sequence of the referenced polypeptide.

An “engineered protein that comprises a Cas12a polypeptide” as used herein refers to a polypeptide that comprises (i) a Cas 12a polypeptide and/or domain and (ii) a polypeptide that is heterologous to the Cas12a polypeptide and/or domain. The Cas12a polypeptide and/or domain may be a portion of a Cas12a (e.g., a wild-type Cas12a or inactivated Cas12a). For example, an engineered protein that comprises a Cas12a polypeptide may comprise a portion of any one of SEQ ID NOs: 39-65, 298, and 850-852 and another polypeptide that is heterologous to the Cas12a polypeptide. An engineered protein that comprises a Cas12a polypeptide may comprise at least one non-natural mutation compared to a Cas12a (e.g., a wild-type Cas12a or inactivated Cas12a). In some embodiments, an engineered protein that comprises a Cas12a polypeptide has a similar structure and/or function compared to a Cas12a and/or comprises all or a portion of a Cas12a (e.g., a wild-type Cas12a or inactivated Cas12a). An engineered protein that comprises a Cas12a polypeptide is not found in nature (e.g., comprises a non-natural mutation) and may be altered (e.g., mutated) compared to a wild-type Cas12a or inactivated Cas12a. In some embodiments, an engineered protein that comprises a Cas12a polypeptide is devoid of a portion of a Cas12a.

In some embodiments, an engineered protein that comprises a Cas12a polypeptide comprises a domain that is not present in a Cas12a. In some embodiments, an engineered protein that comprises a Cas12a polypeptide comprises all or a portion of a wedge domain, a Rec1 domain, a Rec2 domain, a PAM-interacting domain, a RuvC domain, a bridge helix, and/or a Nuc domain each of which may be from a Cas12a and/or a protein having a sequence of any one of SEQ ID NOs: 39-65, 298, and 850-852. In some embodiments, an engineered protein that comprises a Cas12a polypeptide comprises all or a portion of a Cas12a domain having a structure as described in Yamano, Takashi, et al., Mol Cell 67:633-645 (2017). In some embodiments, an engineered protein that comprises a Cas 12a polypeptide comprises all or a portion of an alpha-helical recognition (REC) lobe, optionally wherein the domains of all or a portion of the REC lobe in the engineered protein may be in a different order and/or structure than in a Cas12a. A REC lobe may contain a Rec1 domain and Rec2 domain. A Rec1 domain may comprise 13 alpha helices and/or a Rec2 domain may comprise 10 alpha helices and two beta strands that may form a small antiparallel sheet. In some embodiments, an engineered protein that comprises a Cas12a polypeptide may comprise an HNH domain (e.g., an HNH domain of a Cas9, optionally Cas9 having a sequence of any one of SEQ ID NOs: 133-140 and 156-157) between a first polypeptide comprising all or a portion of a Rec1 domain and a second polypeptide comprising all or a portion of a Rec2 domain, wherein first and second polypeptides may each independently be from a Cas12a and/or a protein having a sequence of any one of SEQ ID NOs: 39-65, 298, and 850-852. In some embodiments, an engineered protein that comprises a Cas12a polypeptide comprises all or a portion of a RuvC domain.

In some embodiments, an engineered protein that comprises a Cas12a polypeptide may comprise an HNH domain (e.g., an HNH domain of a Cas9, optionally Cas9 having a sequence of any one of SEQ ID NOs: 133-140 and 156-157) between a first polypeptide comprising all or a portion of a Rec1 domain and a second polypeptide comprising all or a portion of a Rec2 domain, wherein first and second polypeptides may each independently be from a Cas12a and/or a protein having a sequence of any one of SEQ ID NOs: 39-65, 298, and 850-852, and the engineered protein may comprise all or a portion of a RuvC domain. In some embodiments, an engineered protein that comprises a Cas12a polypeptide may be an engineered protein as described in U.S. Patent Application Publication No. 2002/0112473 and/or PCT/US2023/063398. As one of skill in the art would understand, some domains (e.g., the wedge and RuvC domains of Cas12a) are not continuous in sequence and may be split into two or more (e.g., 2, 3, 4, or more)non-continuous sequences. In some embodiments, an engineered protein that comprises a Cas12a polypeptide comprises a Cas12a domain.

In some embodiments, an engineered protein that comprises a Cas12a polypeptide comprises, optionally in the N- to C-terminus direction, all or a portion of one or more of the following: first portion of the wedge domain (WED-1), Rec1 domain, Rec2 domain, second portion of the wedge domain (WED-2), PAM-interacting domain (PI), third portion of the wedge domain (WED-3), first portion of the RuvC domain (RuvC-1), bridge helix, second portion of the RuvC domain (RuvC-2), Nuc domain, and/or third portion of the RuvC domain (RuvC-3). For example, based on SEQ ID NO:246, an engineered protein that comprises a Cas12a polypeptide may be encoded by all or a portion of one or more of the following: nucleotides 1-69 making up WED-1, nucleotides 70-1,560 making up the REC lobe, nucleotides 1,561-1,755 making up WED-2, nucleotides 1,756-2,031 making up PI, nucleotides 2,032-2,421 WED-3, nucleotides 2,422-2,613 making up RuvC-1, nucleotides 2,614-2,667 making up the bridge helix, nucleotides 2,668-2,988 making up RuvC-2, nucleotides 2,989-3,534 making up Nuc domain, and/or nucleotides 3,535-3,681 making up RuvC-3. As another example, based on SEQ ID NO:56, an engineered protein that comprises a Cas12a polypeptide may comprise, optionally in the N- to C-terminus direction, all or a portion of one or more of the following: amino acid residues 1-23 making up WED-1, amino acid residues 24-520 making up the REC lobe, amino acid residues 521-585 making up WED-2, amino acid residues 586-677 making up PI, amino acid residues 678-807 WED-3, amino acid residues 808-871 making up RuvC-1, amino acid residues 872-889 making up the bridge helix, amino acid residues 890-996 making up RuvC-2, amino acid residues 997-1,178 making up Nuc domain, and/or amino acid residues 1,179-1,227 making up RuvC-3. In some embodiments, an engineered protein that comprises a Cas12a polypeptide comprises all or a portion of an active RuvC domain. In some embodiments, an engineered protein that comprises a Cas 12a polypeptide comprises all or a portion of an inactivated RuvC domain, optionally all or a portion of an inactivated RuvC domain that has a D10A mutation. In some embodiments, an engineered protein that comprises a Cas12a polypeptide comprises all or a portion of an inactivated RuvC domain and has an alanine at a position corresponding to amino acid residue 831 of SEQ ID NO:59 when the engineered protein is optimally aligned to SEQ ID NO:59, optionally wherein the mutation is referred to as a D10A and/or D832A mutation. In some embodiments, an engineered protein that comprises a Cas12a polypeptide comprises a polypeptide that comprises all or a portion of an inactivated RuvC domain and has a mutation of: D832A and/or E925A with reference to position numbering of SEQ ID NO:44, 45, 55, 56, and/or 59 (LbCas12a), D908A and/or E993A with reference to position numbering of SEQ ID NO: 39 (AsCas12a), D917A and/or E1006A with reference to position numbering of SEQ ID NO: 43 and/or 58 (FnCas12a), or a mutation corresponding thereto. In some embodiments, an engineered protein that comprises a Cas 12a polypeptide has an amino acid sequence having at least 70%, 75%, 80%, 85%, 90%, 95%, 96%, 97%, 98%, 99%, or more sequence identity to one or more of SEQ ID NOs: 69-132, 159-182, 281-284, 400-401, 761, and 853-861. In some embodiments, an engineered protein that comprises a Cas 12a polypeptide is a target strand nickase and/or a non-target strand nickase. In some embodiments, an engineered protein that comprises a Cas12a polypeptide is a non-target strand nickase.

A circular permutant of the present invention has an amino acid sequence in a different order compared to the amino acid sequence of a Cas12a or compared to the amino acid sequence of an engineered protein that comprises a Cas 12a polypeptide. For example, referring now to FIG. 1A and FIG. 1B, a Cas 12a may have a sequence in which its domains are ordered as shown in FIG. 1A, and a circular permutant of the present invention may have one or more domain(s) in a different location as shown in FIG. 1B compared to the location in Cas12a as shown in FIG. 1A. Alpha, Beta, Gamma, Delta, Epsilon, and Zeta as shown in FIG. 1B are each examples of circular permutants of the present invention. In some embodiments, in a circular permutant of the present invention, the WED-1 domain of a Cas12a or an engineered protein that comprises a Cas12a polypeptide may not be located at the N-terminus and/or N-terminal end. In some embodiments, in a circular permutant of the present invention, all or a portion of the WED-1 domain of a Cas12a or an engineered protein that comprises a Cas12a polypeptide may be located at the C-terminal end of the circular permutant. In some embodiments, in a circular permutant of the present invention, all or a portion of the REC lobe of a Cas12a or an engineered protein that comprises a Cas12a polypeptide may be located at the C-terminal end of the circular permutant. In some embodiments, in a circular permutant of the present invention, all or a portion of the Rec1 domain and/or all or a portion of the Rec2 domain of a Cas12a or an engineered protein that comprises a Cas12a polypeptide may be located at the C-terminal end of the circular permutant. In some embodiments, a circular permutant of the present invention may have all or a portion of a domain corresponding to a WED-1 domain, REC lobe, Rec1 domain, and/or Rec2 domain at its C-terminal end. In some embodiments, in a circular permutant of the present invention, all or a portion of the RuvC-3 domain of a Cas12a or an engineered protein that comprises a Cas12a polypeptide may be located at the N-terminal end of the circular permutant. In some embodiments, in a circular permutant of the present invention, all or a portion of the Nuc domain of a Cas12a or an engineered protein that comprises a Cas12a polypeptide may be located at the N-terminal end of the circular permutant. In some embodiments, a circular permutant of the present invention may have all or a portion of a domain corresponding to a RuvC-3 domain and/or a Nuc domain at its C-terminal end. In some embodiments, the N-terminus of a circular permutant of the present invention may be an amino acid residue that is in the REC lobe (e.g., in the Rec1 domain or Rec2 domain) of a Cas12a or an engineered protein that comprises a Cas12a polypeptide. For example, the N-terminus of a circular permutant of the present invention may be one of amino acid residues 24-520 of Cas12a having a sequence of SEQ ID NO:56 or a sequence optimally aligned thereto. In some embodiments, the N-terminus of a circular permutant of the present invention may be an amino acid residue that is in the Nuc domain of a Cas12a or an engineered protein that comprises a Cas12a polypeptide. For example, the N-terminus of a circular permutant of the present invention may be one of amino acid residues 997-1,178 of Cas12a having a sequence of SEQ ID NO:56 or a sequence optimally aligned thereto. In some embodiments, the N-terminus of a circular permutant of the present invention may be one of amino acid residues 50-100, 70-100, 80-90, 100-200, 110-140, 120-130, 700-800, 710-740, 720-730, 780-810, 790-800, 900-1000, 940-970, 1100-1200, 1140-1170, or 1150-1160 of Cas12a having a sequence of SEQ ID NO:56 or a sequence optimally aligned thereto. For example, the N-terminus of a circular permutant of the present invention may be amino acid residue 85, 292, 443, 517, 1118, or 1156 of Cas12a having a sequence of SEQ ID NO: 56 or a sequence optimally aligned thereto. In some embodiments, the N-terminus of a circular permutant of the present invention may be one of amino acid residues 50-1300 of Cas12a having a sequence of SEQ ID NO:298 or a sequence optimally aligned thereto. In some embodiments, the N-terminus of a circular permutant of the present invention may be one of amino acid residues 50-100, 70-100, 80-90, 85-95, 300-400, 315-325, 320-330, 400-500, 440-460, 455-465, 500-600, 520-530, 525-535, 1150-1250, 1205-1215, 1200-1300, or 1240-1250 of Cas12a having a sequence of SEQ ID NO:298 or a sequence optimally aligned thereto. For example, the N-terminus of a circular permutant of the present invention may be amino acid residue 88, 322, 457, 529, 1208, or 1245 of Cas 12a having a sequence of SEQ ID NO: 298 or a sequence optimally aligned thereto. In some embodiments, a circular permutant of the present invention is a circular permutant of any one of SEQ ID NOs: 39-65, 69-132, 159-182, 281-284, 298, 400-401, 741, 761, and 850-861 or is a circular permutant of a sequence have at least 70%, 75%, 80%, 85%, 90%, 95%, 96%, 97%, 98%, 99%, or more sequence identity to one of SEQ ID NOs: 39-65, 69-132, 159-182, 281-284, 298, 400-401, 741, 761, and 850-861.

In some embodiments, a circular permutant of the present invention is a protein or polypeptide that cleaves, cuts, or nicks a nucleic acid; binds a nucleic acid (e.g., a target nucleic acid and/or a guide nucleic acid); and/or that identifies, recognizes, or binds a guide nucleic acid as defined herein. In some embodiments, a circular permutant of the present invention may be an enzyme (e.g., a nuclease, endonuclease, nickase, etc.) and/or may function as an enzyme. In some embodiments, a circular permutant of the present invention is a nuclease and/or has at least one function and/or activity similar to a CRISPR-Cas nuclease (e.g., a Cas12a nuclease). In some embodiments, a circular permutant of the present invention comprises nuclease activity and/or nickase activity, comprises a nuclease domain whose nuclease activity and/or nickase activity has been reduced or eliminated, comprises single stranded DNA cleavage activity (ss DNase activity) or which has ss DNase activity that has been reduced or eliminated, and/or comprises self-processing RNase activity or which has self-processing RNase activity that has been reduced or eliminated. A circular permutant of the present invention may bind to a target nucleic acid. In some embodiments, a circular permutant of the present invention is a target strand nickase and/or a non-target strand nickase. In some embodiments, a circular permutant of the present invention is a non-target strand nickase.

In some embodiments, a circular permutant of the present invention is a circular permutant of a Cas12a (e.g., a Cas12a having at least 70%, 75%, 80%, 85%, 90%, 95%, 96%, 97%, 98%, 99%, or 100% sequence identity to one or more of SEQ ID NOs: 39-65, 298, and 850-852), wherein the Cas12a is a non-target strand nickase that may modify the non-target strand of a target nucleic acid, and the circular permutant of the Cas12a is a non-target strand nickase and/or modifies the non-target strand of a target nucleic acid. In some embodiments, a circular permutant of the present invention is a circular permutant of a Cas12a (e.g., a Cas12a having at least 70%, 75%, 80%, 85%, 90%, 95%, 96%, 97%, 98%, 99%, or 100% sequence identity to one or more of SEQ ID NOs: 39-65, 298, and 850-852), wherein the Cas12a is a non-target strand nickase that may modify the non-target strand of a target nucleic acid, and the circular permutant of the Cas 12a is a target strand nickase and/or modifies the target strand of a target nucleic acid and optionally the circular permutant of the Cas 12a may be a non-target strand nickase and/or may modify the non-target strand of a target nucleic acid. In some embodiments, a circular permutant of the present invention is a circular permutant of a Cas12a (e.g., a Cas12a having at least 70%, 75%, 80%, 85%, 90%, 95%, 96%, 97%, 98%, 99%, or 100% sequence identity to one or more of SEQ ID NOs: 39-65, 298, and 850-852), wherein the Cas12a is a non-target strand nickase that may modify the non-target strand of a target nucleic acid, and the circular permutant of the Cas12a modifies the target strand of a target nucleic acid and the circular permutant of the Cas 12a is a non-target strand nickase. In some embodiments, a circular permutant of the present invention is a circular permutant of a Cas12a (e.g., a Cas12a having at least 70%, 75%, 80%, 85%, 90%, 95%, 96%, 97%, 98%, 99%, or 100% sequence identity to one or more of SEQ ID NOs: 39-65, 298, and 850-852), wherein the Cas12a is a deactivated non-target strand nickase that may modify the non-target strand of a target nucleic acid, and the circular permutant of the deactivated Cas12a is a deactivated non-target strand nickase and/or modifies the non-target strand of a target nucleic acid. In some embodiments, a circular permutant of the present invention is a circular permutant of a Cas12a (e.g., a Cas12a having at least 70%, 75%, 80%, 85%, 90%, 95%, 96%, 97%, 98%, 99%, or 100% sequence identity to one or more of SEQ ID NOs: 39-65, 298, and 850-852), wherein the Cas12a is a deactivated non-target strand nickase that may modify the non-target strand of a target nucleic acid, and the circular permutant of the deactivated Cas12a is a deactivated target strand nickase and/or modifies the target strand of a target nucleic acid and optionally the circular permutant of the deactivated Cas12a may also be a deactivated non-target strand nickase and/or may modify the non-target strand of a target nucleic acid. In some embodiments, a circular permutant of the present invention is a circular permutant of a dead Cas12a, and the circular permutant of the dead Cas12a has no nuclease activity. In some embodiments, a circular permutant of the present invention is a circular permutant of a polypeptide (e.g., a polypeptide having at least 70%, 75%, 80%, 85%, 90%, 95%, 96%, 97%, 98%, 99%, or 100% sequence identity to one or more of SEQ ID NOs: 69-132, 159-182, 281-284, 400-401, 761, and 853-861), wherein the polypeptide is a target strand nickase and/or a non-target strand nickase that may modify the non-target strand and/or target strand of a target nucleic acid, and the circular permutant of the polypeptide is a target strand nickase and/or a non-target strand nickase that may modify the target and/or non-target strand of a target nucleic acid.

In some embodiments, a circular permutant of the present invention may comprise a mutation in its nuclease active site and/or nuclease domain (e.g., a RuvC, HNH, e.g., a RuvC site of a Cas12a nuclease domain; e.g., a RuvC site and/or HNH site of a Cas9 nuclease domain). A circular permutant of the present invention having a mutation in its nuclease active site and/or nuclease domain that causes the circular permutant to not have nuclease activity, is commonly referred to as “inactive” or “dead” circular permutant. In some embodiments, a circular permutant of the present invention having a mutation in its nuclease active site and/or nuclease domain may have impaired activity or reduced activity (e.g., nickase activity) as compared to the same circular permutant without the mutation and/or compared to a CRISPR-Cas effector protein without the mutation.

A circular permutant of the present invention may bind to a nucleic acid (e.g., may bind to a target nucleic acid). In some embodiments, a circular permutant of the present invention may bind a target nucleic acid and/or a guide nucleic acid. In some embodiments, a circular permutant of the present invention may be used to target a desired nucleic acid (e.g., a target nucleic acid) and/or to bring another component to a desired nucleic acid. In some embodiments, a circular permutant of the present invention has nuclease activity. Nuclease activity may be measured and/or tested using methods known in the art. In some embodiments, nuclease activity may be measured and/or tested by measuring the ability of a polypeptide (e.g., a circular permutant and/or enzyme) to make an insertion and/or deletion of one or more base(s) (e.g., INDELs) at one or more target nucleic acids (e.g., at about 3 to about 5 different target nucleic acids) in a cell such as HEK293T cells. In some embodiments, a polypeptide having a measurable insertion and/or deletion activity (e.g., INDEL activity) indicates that the polypeptide is expressed, folded, and/or performing one or more function(s) such as crRNA processing, nucleic acid binding (e.g., crRNA binding and/or DNA binding), and/or nuclease activity.

In some embodiments, a circular permutant of the present invention comprises a linker (e.g., a peptide linker) between two amino acid residues such as between two consecutive amino acid residues. When compared relative to the amino acid sequence of an original polypeptide (e.g., a Cas12a or an engineered protein that comprises a Cas 12a polypeptide) upon which the circular permutant is based, the linker may be between two non-consecutive amino acid residues of the original polypeptide. In some embodiments, for a linker that is between two amino acid residues of a circular permutant of the present invention, one of the two amino acid residues is at the N-terminal end of the original polypeptide and the other of the two amino acids is at the C-terminal end of the original polypeptide. In some embodiments, for a linker that is between two amino acid residues of a circular permutant of the present invention, one of the two amino acid residues is the N-terminal amino acid residue of the original polypeptide and the other of the two amino acids is the C-terminal amino acid residue of the original polypeptide. A linker present in a circular permutant of the present invention may have a length of about 1, 2, 3, 4, 5, 6, 7, 8, 9, 10, 11, 12, 13, 14, 15, 15, 16, 17, 18, 19, or 20 amino acid(s). In some embodiments, a linker present in a circular permutant of the present invention may have a length of about 1, 2, 3, 4, 5, 6, 7, 8, 9, or 10 amino acid(s) to about 11, 12, 13, 14, 15, 15, 16, 17, 18, 19, or 20 amino acid(s). In some embodiments, a linker present in a circular permutant of the present invention has a length of 10 amino acids or 16 amino acids. In some embodiments, a linker present in a circular permutant of the present invention is an amino acid linker that comprises glycine and/or serine. In some embodiments, a linker present in a circular permutant of the present invention has one of the amino acid sequences of SEQ ID NOs: 1-36, 313-314, 869-870, 883 and/or 885. In some embodiments, the peptide linker is encoded by a sequence of SEQ ID NO:311 or 312. In some embodiments, a linker present in a circular permutant of the present invention has a sequence of (GSS)ₙG (SEQ ID NO:36), (SGS)ₙ (SEQ ID NO: 869), or (SGGS)ₙ (SEQ ID NO:870) wherein n is an integer of 1-20 (e.g., 1, 2, 3, 4, 5, 6, 7, 8, 9, 10, 11, 12, 13, 14, 15, 16, 17, 18, 19, or 20). In some embodiments, a linker present in a circular permutant of the present invention has a sequence of (GSS)ₙG (SEQ ID NO:36), (SGS)ₙ (SEQ ID NO:869), or (SGGS)ₙ (SEQ ID NO:870) wherein n is 2, 3, 4, or 5. In some embodiments, a linker present in a circular permutant of the present invention has a sequence of STSQSDGSSVPADIDQSSDSDQSSSQGQPG (SEQ ID NO:5), SGSETPGTSESATPES (SEQ ID NO:29), SGGSSGSETPGTSESATPESSGGS (SEQ ID NO:883), SGGSSGGSSGSETPGTSESATPESSGGSSGGS (SEQ ID NO:30), SGGSSGSETPGTSESATPESSGGS (SEQ ID NO:883), or GSPKKKRKVSGGS (SEQ ID NO: 885). In some embodiments, a circular permutant of the present invention is devoid of a linker. In some embodiments, a circular permutant of the present invention is devoid of a linker between a first portion of an original polypeptide that comprises the C-terminal end of the original polypeptide and a second portion of the original polypeptide that comprises the N-terminal end of the original polypeptide.

In some embodiments, a circular permutant of the present invention may be fused to a polypeptide of interest (e.g., a deaminase, a reverse transcriptase, etc.) with a linker in between the circular permutant and the polypeptide of interest. The linker may have a length of 4, 5, 6, 7, 8, 9, 10, 11, 12, 13, 14, 15, 15, or 16 amino acids. In some embodiments, the linker has a length of 4 amino acids. In some embodiments, the linker has a length of 6 amino acids. In some embodiments, the linker has a length of 8 amino acids.

A circular permutant of the present invention and/or a polypeptide of the present invention comprising a circular permutant of the present invention may have a different editing window than that for a Cas12a or an engineered protein that comprises a Cas12a polypeptide, optionally measured and/or tested under the same conditions. In some embodiments, a circular permutant of the present invention and/or a polypeptide of the present invention comprising a circular permutant of the present invention has a different editing window than the original polypeptide upon which the circular permutant is based and/or for which the circular permutant is a variant, optionally measured and/or tested under the same conditions. In some embodiments, a circular permutant of the present invention and/or a polypeptide of the present invention comprising a circular permutant of the present invention has an editing window that is increased or decreased in length compared to the length of the editing window for a Cas12a or an engineered protein that comprises a Cas12a polypeptide, optionally measured and/or tested under the same conditions. In some embodiments, a circular permutant of the present invention and/or a polypeptide of the present invention comprising a circular permutant of the present invention has an editing window that is increased or decreased in length compared to the length of the editing window for the original polypeptide upon which the circular permutant is based and/or for which the circular permutant is a variant, optionally measured and/or tested under the same conditions. The polypeptide and/or circular permutant may have an altered (e.g., increased or decreased) editing window for base editing and/or REDRAW editing. The editing window for an editing system (e.g., for a base editing system and/or REDRAW system) may be measured and/or determined using methods known in the art. In some embodiments, an editing window for an editing system may be measured and/or determined by measuring editing efficiency of C's, A's, and/or precise edits within a spacer using sequencing (e.g., Next Generation sequencing (NGS)). In some embodiments, an editing window for an editing system may be measured and/or determined by an experiment in which a plasmid comprising a CRISPR-Cas effector protein and/or a circular permutant of the present invention is transiently co-transfected into a cell (e.g., an HEK293T cell) along with a plasmid comprising a guide or stagRNA, and the ability of the CRISPR-Cas effector protein and/or a circular permutant to make an edit (e.g., an edit within the spacer of the guide or stagRNA) at one or more target nucleic acids (e.g., at about 1 to about 3 or 5 different target nucleic acids) in the cell is detected and/or measured.

In some embodiments, a circular permutant of the present invention has an editing window in a region of a target nucleic acid corresponding to nucleotide 9 to nucleotide 23 (or any range or nucleotide therein) of the spacer sequence that has complementarity to the target nucleic acid (i.e., the region is the corresponding nucleotides of the target nucleic acid that are opposite (e.g., matched or mismatched) to nucleotide 9 to nucleotide 23 of the spacer sequence). In some embodiments, a circular permutant of the present invention that is fused to a polypeptide of interest (e.g., a deaminase, a reverse transcriptase, etc.) with a linker (e.g., a linker having a length of 4, 5, 6, 7, or 8 amino acids) in between the circular permutant and the polypeptide of interest has an editing window in a region of a target nucleic acid corresponding to nucleotide 9 to nucleotide 23 (or any range or nucleotide therein) of the spacer sequence that has complementarity to the target nucleic acid. In some embodiments, a protein of the present invention comprises an amino acid sequence having at least 70%, 75%, 80%, 85%, 90%, 95%, 96%, 97%, 98%, 99%, or 100% sequence identity to one or more of SEQ ID NOs: 453, 459, and 629-639 and has an editing window in a region of a target nucleic acid corresponding to nucleotide 9 to nucleotide 23 (or any range or nucleotide therein) of the spacer sequence that has complementarity to the target nucleic acid.

In some embodiments, a protein of the present invention has an editing window in a region of a target nucleic acid corresponding to nucleotide 9 to nucleotide 33 (or any range or nucleotide therein) of the spacer sequence that has complementarity to the target nucleic acid. In some embodiments, a protein of the present invention has an editing window in a region of a target nucleic acid corresponding to nucleotide 24 to nucleotide 33 (or any range or nucleotide therein) of the spacer sequence that has complementarity to the target nucleic acid. In some embodiments, a protein of the present invention comprises an amino acid sequence having at least 70%, 75%, 80%, 85%, 90%, 95%, 96%, 97%, 98%, 99%, or 100% sequence identity to one or more of SEQ ID NOs: 453, 459, and 629-639 and has an editing window in a region of a target nucleic acid corresponding to nucleotide 9 to nucleotide 33 (or any range or nucleotide therein) of the spacer sequence that has complementarity to the target nucleic acid. In some embodiments, a protein of the present invention comprises an amino acid sequence having at least 70%, 75%, 80%, 85%, 90%, 95%, 96%, 97%, 98%, 99%, or 100% sequence identity to one or more of SEQ ID NOs: 453, 459, and 629-639 and has an editing window in a region of a target nucleic acid corresponding to nucleotide 24 to nucleotide 33 (or any range or nucleotide therein) of the spacer sequence that has complementarity to the target nucleic acid.

In some embodiments, a circular permutant and/or protein of the present invention deletes one or more nucleotide(s) (e.g., 1, 2, 5, 10, 20, 30, 40, or 50 nucleotide(s) or more) from a target nucleic acid, optionally wherein the deletion is longer than the spacer sequence for the target nucleic acid and/or the deletion is in a region that extends outside the spacer sequence. In some embodiments, a protein of the present invention comprises an amino acid sequence having at least 70%, 75%, 80%, 85%, 90%, 95%, 96%, 97%, 98%, 99%, or 100% sequence identity to one or more of SEQ ID NOs: 234-245, 299-310, 390-399, 537-560, 702-704, 735-740, and 757-760 and deletes one or more nucleotide(s) (e.g., 1, 2, 5, 10, 20, 30, 40, or 50 nucleotide(s) or more) from a target nucleic acid, optionally wherein the deletion is longer than the spacer sequence for the target nucleic acid and/or the deletion is in a region that extends outside the spacer sequence.

In some embodiments, a circular permutant of the present invention has an editing window in a region of a target nucleic acid corresponding to nucleotide 6 to nucleotide 23 (or any range or nucleotide therein) of the spacer sequence that has complementarity to the target nucleic acid (i.e., the region is the corresponding nucleotides of the target nucleic acid that are opposite (e.g., matched or mismatched) to nucleotide 6 to nucleotide 23 of the spacer sequence). In some embodiments, a circular permutant of the present invention that is fused to a polypeptide of interest (e.g., a deaminase, a reverse transcriptase, etc.) with a linker (e.g., a linker having a length of 4, 5, 6, 7, or 8 amino acids) in between the circular permutant and the polypeptide of interest has an editing window in a region of the target nucleic acid corresponding to nucleotide 6 to nucleotide 23 (or any range or nucleotide therein) of the spacer sequence that has complementarity to the target nucleic acid. In some embodiments, a protein of the present invention comprises an amino acid sequence having at least 70%, 75%, 80%, 85%, 90%, 95%, 96%, 97%, 98%, 99%, or 100% sequence identity to one or more of SEQ ID NOs: 450-452, 454-458, 560-461, and 624-628 and has an editing window in a region of the target nucleic acid corresponding to nucleotide 6 to nucleotide 23 (or any range or nucleotide therein) of the spacer sequence that has complementarity to the target nucleic acid.

In some embodiments, a protein of the present invention has an editing window in a region of a target nucleic acid corresponding to nucleotide 6 to nucleotide 30 (or any range or nucleotide therein) of the spacer sequence that has complementarity to the target nucleic acid (i.e., the region is the corresponding nucleotides of the target nucleic acid that are opposite (e.g., matched or mismatched) to nucleotide 6 to nucleotide 30 of the spacer sequence). In some embodiments, a protein of the present invention has an editing window in a region of the target nucleic acid corresponding to nucleotide 24 to nucleotide 30 (or any range or nucleotide therein) of the spacer sequence that has complementarity to the target nucleic acid. In some embodiments, a protein of the present invention comprises an amino acid sequence having at least 70%, 75%, 80%, 85%, 90%, 95%, 96%, 97%, 98%, 99%, or 100% sequence identity to one or more of SEQ ID NOs: 450-452, 454-458, 560-461, and 624-628 and has an editing window in a region of a target nucleic acid corresponding to nucleotide 6 to nucleotide 30 (or any range or nucleotide therein) of the spacer sequence that has complementarity to the target nucleic acid. In some embodiments, a protein of the present invention comprises an amino acid sequence having at least 70%, 75%, 80%, 85%, 90%, 95%, 96%, 97%, 98%, 99%, or 100% sequence identity to one or more of SEQ ID NOs: 450-452, 454-458, 560-461, and 624-628 and has an editing window in a region of the target nucleic acid corresponding to nucleotide 24 to nucleotide 30 (or any range or nucleotide therein) of the spacer sequence that has complementarity to the target nucleic acid.

In some embodiments, a circular permutant of the present invention has an editing window in a region of a target nucleic acid corresponding to nucleotide 8 to nucleotide 21 (or any range or nucleotide therein) of the spacer sequence that has complementarity to the target nucleic acid (i.e., the region is the corresponding nucleotides of the target nucleic acid that are opposite (e.g., matched or mismatched) to nucleotide 8 to nucleotide 21 of the spacer sequence). In some embodiments, a circular permutant of the present invention that is fused to a polypeptide of interest (e.g., a deaminase, a reverse transcriptase, etc.) with a linker (e.g., a linker having a length of 4, 5, 6, 7, or 8 amino acids) in between the circular permutant and the polypeptide of interest has an editing window in a region of the target nucleic acid corresponding to nucleotide 8 to nucleotide 21 (or any range or nucleotide therein) of the spacer sequence that has complementarity to the target nucleic acid. In some embodiments, a protein of the present invention comprises an amino acid sequence having at least 70%, 75%, 80%, 85%, 90%, 95%, 96%, 97%, 98%, 99%, or 100% sequence identity to one or more of SEQ ID NOs: 486-497 and 609-623 and has an editing window in a region of the target nucleic acid corresponding to nucleotide 8 to nucleotide 21 (or any range or nucleotide therein) of the spacer sequence that has complementarity to the target nucleic acid.

A circular permutant of the present invention and/or a polypeptide of the present invention comprising a circular permutant of the present invention may have increased nuclease activity compared to the nuclease activity of a Cas12a or an engineered protein that comprises a Cas 12a polypeptide, optionally measured and/or tested under the same conditions. In some embodiments, a circular permutant of the present invention and/or a polypeptide of the present invention comprising a circular permutant of the present invention has increased nuclease activity compared to the original polypeptide upon which the circular permutant is based and/or for which the circular permutant is a variant, optionally measured and/or tested under the same conditions.

In some embodiments, the N-terminus and/or C-terminus of a circular permutant of the present invention are closer (e.g., in distance such as Angstroms) to a target nucleic acid (e.g., a bound target nucleic acid) than the N-terminus and/or C-terminus of a Cas12a or an engineered protein that comprises a Cas12a polypeptide, optionally measured and/or tested under the same conditions. In some embodiments, the N-terminus and/or C-terminus of a polypeptide of the present invention comprising a circular permutant of the present invention are closer (e.g., in distance such as Angstroms) to a target nucleic acid (e.g., a bound target nucleic acid) than the N-terminus and/or C-terminus of a Cas 12a or an engineered protein that comprises a Cas 12a polypeptide, optionally measured and/or tested under the same conditions. In some embodiments, the N-terminus and/or C-terminus of a circular permutant of the present invention are closer to the non-target strand of a target nucleic acid than the N-terminus and/or C-terminus of a Cas12a or an engineered protein that comprises a Cas12a polypeptide, optionally measured and/or tested under the same conditions. In some embodiments, the N-terminus and/or C-terminus of a polypeptide of the present invention comprising a circular permutant of the present invention are closer to the non-target strand of a target nucleic acid than the N-terminus and/or C-terminus of a Cas12a or an engineered protein that comprises a Cas12a polypeptide, optionally measured and/or tested under the same conditions. In some embodiments, the N-terminus and/or C-terminus of a circular permutant of the present invention are closer (e.g., in distance such as Angstroms) to a target nucleic acid (e.g., a bound target nucleic acid) than the N-terminus and/or C-terminus of the original polypeptide upon which the circular permutant is based and/or for which the circular permutant is a variant, optionally measured and/or tested under the same conditions. In some embodiments, the N-terminus and/or C-terminus of a circular permutant of the present invention are closer to the non-target strand of a target nucleic acid than the N-terminus and/or C-terminus of the original polypeptide upon which the circular permutant is based and/or for which the circular permutant is a variant, optionally measured and/or tested under the same conditions. In some embodiments, a nucleic acid (e.g., DNA and/or RNA) binding domain of a circular permutant of the present invention is closer (e.g., in distance such as Angstroms) to a target nucleic acid (e.g., a bound target nucleic acid) than the nucleic acid binding domain of a Cas12a or an engineered protein that comprises a Cas12a polypeptide, optionally measured and/or tested under the same conditions. In some embodiments, a nucleic acid (e.g., DNA and/or RNA) binding domain of a polypeptide of the present invention comprising a circular permutant of the present invention is closer (e.g., in distance such as Angstroms) to a target nucleic acid (e.g., a bound target nucleic acid) than the nucleic acid binding domain of a Cas12a or an engineered protein that comprises a Cas12a polypeptide, optionally measured and/or tested under the same conditions. Access and/or distance may be determined using methods known in the art. In some embodiments, access (e.g., a N- or C termini's access to a nucleic acid or a binding domain's access to a nucleic acid) and/or distance (e.g., a distance from a N- or C-termini to a nucleic acid or a distance from a N- or C-termini to a nucleic acid) may be determined and/or measured based on a structural analysis of the polypeptide and/or nucleic acid (e.g., a structure determined via modeling, nuclear magnetic resonance, and/or crystallography).

In some embodiments, a circular permutant of the present invention and/or a polypeptide of the present invention comprising a circular permutant of the present invention has improved (e.g., better) access to a nucleic acid (e.g., a target nucleic acid, a ss-RNA) compared to the access of a Cas12a or an engineered protein that comprises a Cas12a polypeptide, optionally measured and/or tested under the same conditions. In some embodiments, a circular permutant of the present invention and/or a polypeptide of the present invention comprising a circular permutant of the present invention has improved (e.g., better) access to a nucleic acid (e.g., a target nucleic acid, a ss-RNA) compared to the access of the original polypeptide upon which the circular permutant is based and/or for which the circular permutant is a variant, optionally measured and/or tested under the same conditions. In some embodiments, a polypeptide of the present invention comprising a circular permutant of the present invention and a second polypeptide (e.g., a deaminase and/or reverse transcriptase) provides the second polypeptide with improved (e.g., better) access to a nucleic acid (e.g., a target nucleic acid, a ss-RNA) compared to the access of a Cas12a or an engineered protein that comprises a Cas12a polypeptide, optionally measured and/or tested under the same conditions. In some embodiments, a polypeptide of the present invention comprising a circular permutant of the present invention and a second polypeptide (e.g., a deaminase and/or reverse transcriptase) provides the second polypeptide with improved (e.g., better) access to a nucleic acid (e.g., a target nucleic acid, a ss-RNA) compared to the access of the original polypeptide upon which the circular permutant is based and/or for which the circular permutant is a variant, optionally measured and/or tested under the same conditions. In some embodiments, improved access to a nucleic acid (e.g., a target nucleic acid) may result in and/or provide an increased editing efficiency and/or modified (e.g., increased or decreased) editing window, which may indicate improved access to the nucleic acid.

A circular permutant of the present invention and/or a polypeptide of the present invention comprising a circular permutant of the present invention may bind to a target nucleic acid. In some embodiments, the polypeptide and/or circular permutant binds the target nucleic acid in an improved (e.g., more accessible) orientation compared to a Cas12a or an engineered protein that comprises a Cas 12a polypeptide and/or to the original polypeptide upon which the circular permutant is based and/or for which the circular permutant is a variant.

In some embodiments, a circular permutant of the present invention and/or a polypeptide of the present invention comprising a circular permutant of the present invention may have increased RNase activity compared to a Cas12a or an engineered protein that comprises a Cas12a polypeptide and/or to the original polypeptide upon which the circular permutant is based and/or for which the circular permutant is a variant. A circular permutant of the present invention and/or a polypeptide of the present invention comprising a circular permutant of the present invention may be able to self-process a guide nucleic acid. RNase activity and/or ability to self-process a guide nucleic acid may be measured and/or determined using methods known in the art. In some embodiments, RNase activity and/or ability to self-process a guide nucleic acid may be measured and/or determined by testing the length of an array a polypeptide (e.g., a circular permutant of the present invention) can process. For example, a crRNA array may be generated with guide nucleic acids directed to one or more different target nucleic acids (e.g., at the beginning, middle, and end of the array) in a cell (e.g., an HEK293T cell), then the INDEL efficiency at each of the target nucleic acids may be determined using sequencing (e.g., NGS).

A circular permutant of the present invention and/or a polypeptide of the present invention comprising a circular permutant of the present invention may have modified (e.g., increased) crRNA binding compared a Cas12a or an engineered protein that comprises a Cas12a polypeptide and/or to the original polypeptide upon which the circular permutant is based and/or for which the circular permutant is a variant. In some embodiments, a circular permutant of the present invention and/or a polypeptide of the present invention comprising a circular permutant of the present invention may have modified (e.g., increased) PAM access compared to a Cas12a or an engineered protein that comprises a Cas 12a polypeptide and/or to the original polypeptide upon which the circular permutant is based and/or for which the circular permutant is a variant. PAM access and/or crRNA binding may be measured and/or determined using methods known in the art. In some embodiments, PAM access may be measured and/or determined by performing a bacterial, in vivo PAM binding assay (PAM-SCANR) on a polypeptide (e.g., a circular permutant of the present invention) and sequencing the assay result (e.g., NGS).

A circular permutant of the present invention may include a non-natural PAM recognition site and/or sequence. For example, a circular permutant of the present invention may be a nuclease that comprises non-natural PAM recognition specificity (e.g., altered binding affinity) in addition to or instead of the natural PAM recognition specificity (e.g., wild-type binding affinity) for a Cas12a. As used herein in reference to a circular permutant, modified protein, engineered protein, and/or nuclease, “altered PAM specificity” means that the PAM specificity of the circular permutant, modified protein, engineered protein, and/or nuclease is altered from that of the wild-type nuclease (e.g., non-native PAM sequences are recognized by the circular permutant, modified protein, engineered protein, and/or nuclease in addition to and/or instead of the native PAM sequence that are recognized by the wild-type nuclease). For example, a modified Cas12a nuclease (e.g., a modified protein) or a circular permutant would be altered in its PAM specificity if it recognizes a PAM sequence other than and/or in addition to the native Cas12a PAM sequence of TTTV, wherein V is A, C or G. In some embodiments, a circular permutant of the present invention comprises a polypeptide that is a portion of Cas12a and the circular permutant has altered PAM specificity in that it recognizes a PAM sequence other than and/or in addition to the native Cas 12a PAM sequence of TTTV, wherein V is A, C or G. In some embodiments, a circular permutant of the present invention recognizes a native PAM sequence (e.g., a PAM sequence of TTTV, wherein V is A, C or G) and/or recognizes a non-native PAM sequence (e.g., a PAM sequence of CCCC, TCCA, TCCC, TCCG, TTCA, TTCC, TATA, TATC, and TATG, and/or TTCG).

In some embodiments, a circular permutant of the present invention may comprise an altered protospacer adjacent motif (PAM) specificity as compared to wild type LbCas 12a (e.g., SEQ ID NO:56) and/or a modified Cas12a having a sequence of one of SEQ ID NOs: 850-852. A circular permutant of the present invention may have an altered PAM specificity, wherein the altered PAM specificity includes, but is not limited to, NNNG, NNNT, NNNA, NNNC, NNG, NNT, NNC, NNA, NG, NT, NC, NA, NN, NNN, NNNN, wherein each N of each sequence is independently selected from any of T, C, G, or A. In some embodiments, the altered PAM specificity may include, but is not limited to, TTTA, TTTC, TTTG, TTTT, TTCA, TTCC, TTCG, TATA, TATC, TCCG, TCCC, TCCA, and/or TATG. In some embodiments, the altered PAM specificity may be NNNN, wherein each N of each sequence is independently selected from any of T, C, G, or A.

In addition to having an altered PAM recognition specificity a circular permutant of the present invention may further comprise a mutation in the nuclease active site (e.g., RuvC domain) (e.g., deadLbCas12a, dLbCas12a). Such modifications may result in the circular permutant having reduced nuclease activity (e.g., nickase activity) or no nuclease activity.

In some embodiments, a circular permutant of the present invention and/or a polypeptide of the present invention comprising a circular permutant of the present invention may have an increased stability (e.g., increased thermal stability, expression stability, and/or purified stability) compared to a Cas12a or an engineered protein that comprises a Cas12a polypeptide and/or to the original polypeptide upon which the circular permutant is based and/or for which the circular permutant is a variant. Stability may be measured and/or determined using methods known in the art. In some embodiments, stability (e.g., expression and/or purification stability) may be measured and/or determined by quantifying the protein expression yield in bacteria (e.g., E. coli), optionally as compared to the protein expression yield for a control (e.g., a Cas12a such as LbCas12a and/or an engineered protein that comprises a Cas 12a polypeptide). In some embodiments, thermal stability may be measured in vitro by performing a cutting assay at two or more different temperatures, or in vivo by measuring cutting efficiency of a polypeptide (e.g., a circular permutant of the present invention) compared to a Cas 12a or an engineered protein that comprises a Cas 12a polypeptide in a cell (e.g., an HEK293T cell or plant cell) or in a plant. In some embodiments, thermal stability may be measured using differential scanning calorimetry (DSC).

A circular permutant of the present invention and/or a polypeptide of the present invention comprising a circular permutant of the present invention may have at least one improvement (e.g., modified editing window, increased nuclease activity, increased stability, increased binding to a nucleic acid, etc.) compared to a polypeptide (e.g., a Cas12a and/or an engineered protein that comprises a Cas 12a polypeptide) having an amino acid sequence of any one of SEQ ID NOs: 39-65, 69-132, 159-182, 281-284, 298, 400-401, 741, 761, and 850-861.

The N-terminal amino acid residue and/or the C-terminal amino acid residue of a circular permutant of the present invention may be an amino acid residue that is present in a loop of the original polypeptide (e.g., a Cas12a or an engineered protein that comprises a Cas12a polypeptide) upon which the circular permutant is based and/or for which the circular permutant is a variant or may be an acid residue that is present immediately before or immediately after a loop of the original polypeptide upon which the circular permutant is based and/or for which the circular permutant is a variant. The loop may be a surface exposed loop. In some embodiments, the N-terminal amino acid residue and the C-terminal amino acid residue of the circular permutant are each amino acid residues that are present in a loop (e.g., a surface exposed loop) of the original polypeptide. In some embodiments, one of the N-terminal amino acid residue and the C-terminal amino acid residue of the circular permutant is an amino acid residue that is present in a loop (e.g., a surface exposed loop) of the original polypeptide and the other of the N-terminal amino acid residue and the C-terminal amino acid residue of the circular permutant is an amino acid residue that is immediately before or immediately after a loop of the original polypeptide. A Cas12a and/or an engineered protein that comprises a Cas12a polypeptide may have 1, 2, 3, 4, 5, 6, 7, 8, 9, 10, 11, 12, 13, 14, or more loop(s). In some embodiments, the N-terminal amino acid residue and/or the C-terminal amino acid residue of a circular permutant of the present invention may be an amino acid residue that is present in, optionally from the N- to C-terminus direction, the first, second, third, fourth, fifth, sixth, seventh, eighth, ninth, tenth, eleventh, twelfth, thirteenth, or fourteenth loop, etc., (e.g., Loop 1, Loop 2, . . . . Loop 14, etc., respectively) of the original polypeptide upon which the circular permutant is based and/or for which the circular permutant is a variant or may be an amino acid residue that is immediately before or immediately after, optionally from the N- to C-terminus direction, the first, second, third, fourth, fifth, sixth, seventh, eighth, ninth, tenth, eleventh, twelfth, thirteenth, or fourteenth loop, etc. of the original polypeptide. The N-terminal amino acid residue and/or the C-terminal amino acid residue of the circular permutant may be present in a loop of a Cas 12a having a sequence of SEQ ID NO:55 or 56 and/or present in a corresponding region (e.g., loop) that is optimally aligned to SEQ ID NO:55 or 56, or may be an amino acid residue that is immediately before or immediately after a loop of a Cas12a having a sequence of SEQ ID NO:55 or 56 and/or present in a corresponding region (e.g., loop) that is optimally aligned to SEQ ID NO:55 or 56. In some embodiments, a circular permutant of the present invention is a based on and/or is a variant of SEQ ID NO:55 or a Cas12a or an engineered protein that comprises a Cas12a polypeptide that is optimally aligned to SEQ ID NO:55, wherein in SEQ ID NO:55 Loop 1 is amino acid residues 1141-1145, Loop 2 is amino acid residues 997-1019, Loop 3 is amino acid residues 84-86, Loop 4 is amino acid residues 268-295, Loop 5 is amino acid residues 1156-1160, Loop 6 is amino acid residues 1138-1141, Loop 7 is amino acid residues 1170-1177, Loop 8 is amino acid residues 1101-1110, Loop 9 is amino acid residues 513-520, Loop 10 is amino acid residues 438-448, Loop 11 is amino acid residues 476-482, Loop 12 is amino acid residues 405-408, Loop 13 is amino acid residues 332-336, and Loop 14 is amino acid residues 1118-1119, and wherein the N-terminal amino acid residue and/or the C-terminal amino acid residue of the circular permutant is an amino acid residue that is present in a loop of SEQ ID NO:55 or a sequence optimally aligned thereto or is an acid residue that is present immediately before or immediately after a loop of SEQ ID NO:55 or a sequence optimally aligned thereto. The N-terminal amino acid residue and/or the C-terminal amino acid residue of the circular permutant may be present in a loop of a Cas12a having a sequence of SEQ ID NO:298 and/or present in a corresponding region (e.g., loop) that is optimally aligned to SEQ ID NO:298, or may be an amino acid residue that is immediately before or immediately after a loop of a Cas 12a having a sequence of SEQ ID NO:298 and/or present in a corresponding region (e.g., loop) that is optimally aligned to SEQ ID NO:298. In some embodiments, the N-terminal amino acid residue and/or the C-terminal amino acid residue of a circular permutant of the present invention is in a Nuc domain of the original polypeptide (e.g., a Cas12a or an engineered protein that comprises a Cas12a polypeptide), optionally in a loop in the Nuc domain of the original polypeptide or is an amino acid residue that is immediately before or immediately after a loop in the Nuc domain of the original polypeptide. In some embodiments, the N-terminal amino acid residue and/or the C-terminal amino acid residue of a circular permutant of the present invention is in a Rec domain (e.g., Rec1 domain and/or Rec2 domain) of the original polypeptide (e.g., a Cas 12a or an engineered protein that comprises a Cas12a polypeptide), optionally in a loop in the Rec domain (e.g., Rec1 domain and/or Rec2 domain) of the original polypeptide or is an amino acid residue that is immediately before or immediately after a loop in the Rec domain (e.g., Rec1 domain and/or Rec2 domain) of the original polypeptide.

In some embodiments, a circular permutant of the present invention is a based on and/or is a variant of SEQ ID NO:55 or a Cas12a or an engineered protein that comprises a Cas12a polypeptide that is optimally aligned to SEQ ID NO:55, wherein the N-terminal amino acid residue of the circular permutant is amino acid residue 80, 81, 82, 83, 84, 85, 86, 87, 88, or 89 of SEQ ID NO:55 or a sequence optimally aligned thereto, and the C-terminal amino acid residue of the circular permutant is the acid residue of SEQ ID NO:55 or the sequence optimally aligned thereto that is present immediately before that corresponding to the N-terminal amino acid of the circular permutant. For example, if the N-terminal amino acid residue of a circular permutant of the present invention is amino acid residue 85 of SEQ ID NO: 55, then the C-terminal amino acid residue of the circular permutant is acid residue 84 of SEQ ID NO:55. In some embodiments, a circular permutant of the present invention is a based on and/or is a variant of SEQ ID NO:55 or a Cas12a or an engineered protein that comprises a Cas 12a polypeptide that is optimally aligned to SEQ ID NO:55, wherein the N-terminal amino acid residue of the circular permutant is amino acid residue 264, 265, 266, 267, 276, 277, 278, 279, 290, 291, 292, 293, 294, 295, 296, 297, or 298 of SEQ ID NO:55 or a sequence optimally aligned thereto, and the C-terminal amino acid residue of the circular permutant is the acid residue of SEQ ID NO:55 or the sequence optimally aligned thereto that is present immediately before that corresponding to the N-terminal amino acid of the circular permutant. In some embodiments, a circular permutant of the present invention is a based on and/or is a variant of SEQ ID NO:55 or a Cas12a or an engineered protein that comprises a Cas12a polypeptide that is optimally aligned to SEQ ID NO:55, wherein the N-terminal amino acid residue of the circular permutant is amino acid residue 434, 435, 436, 437, 438, 439, 440, 441, 442, 443, 444, 445, 446, 447, 448, 449, or 450 of SEQ ID NO:55 or a sequence optimally aligned thereto, and the C-terminal amino acid residue of the circular permutant is the acid residue of SEQ ID NO:55 or the sequence optimally aligned thereto that is present immediately before that corresponding to the N-terminal amino acid of the circular permutant. In some embodiments, a circular permutant of the present invention is a based on and/or is a variant of SEQ ID NO:55 or a Cas12a or an engineered protein that comprises a Cas12a polypeptide that is optimally aligned to SEQ ID NO:55, wherein the N-terminal amino acid residue of the circular permutant is amino acid residue 510, 511, 512, 513, 514, 515, 516, 517, 518, 519, 520, 521, 522, 523, 524, or 525 of SEQ ID NO:55 or a sequence optimally aligned thereto, and the C-terminal amino acid residue of the circular permutant is the acid residue of SEQ ID NO:55 or the sequence optimally aligned thereto that is present immediately before that corresponding to the N-terminal amino acid of the circular permutant. In some embodiments, a circular permutant of the present invention is a based on and/or is a variant of SEQ ID NO:55 or a Cas12a or an engineered protein that comprises a Cas 12a polypeptide that is optimally aligned to SEQ ID NO:55, wherein the N-terminal amino acid residue of the circular permutant is amino acid residue 1110, 1111, 1112, 1113, 1114, 1115, 1116, 1117, 1118, 1119, 1120, 1121, 1122, 1123, 1124, or 1125 of SEQ ID NO:55 or a sequence optimally aligned thereto, and the C-terminal amino acid residue of the circular permutant is the acid residue of SEQ ID NO:55 or the sequence optimally aligned thereto that is present immediately before that corresponding to the N-terminal amino acid of the circular permutant. In some embodiments, a circular permutant of the present invention is a based on and/or is a variant of SEQ ID NO:55 or a Cas12a or an engineered protein that comprises a Cas12a polypeptide that is optimally aligned to SEQ ID NO:55, wherein the N-terminal amino acid residue of the circular permutant is amino acid residue 1150, 1151, 1152, 1153, 1154, 1155, 1156, 1157, 1158, 1159, 1160, 1161, 1162, 1163, 1164, or 1165 of SEQ ID NO:55 or a sequence optimally aligned thereto, and the C-terminal amino acid residue of the circular permutant is the acid residue of SEQ ID NO:55 or the sequence optimally aligned thereto that is present immediately before that corresponding to the N-terminal amino acid of the circular permutant.

In some embodiments, a circular permutant of the present invention comprises a first amino acid sequence having at least 70%, 75%, 80%, 85%, 90%, 95%, 96%, 97%, 98%, 99%, or 100% sequence identity to a first portion of one of SEQ ID NOs: 39-65, 69-132, 159-182, 281-284, 298, 400-401, 741, 761, and 850-861, wherein the first portion includes the N-terminus and/or all or a portion of the N-terminal end of the one of SEQ ID NOs: 39-65, 69-132, 159-182, 281-284, 298, 400-401, 741, 761, and 850-861 and the first portion is less than the entire amino acid sequence of SEQ ID NOs: 39-65, 69-132, 159-182, 281-284, 298, 400-401, 741, 761, and 850-861. In some embodiments, the N-terminus of the one of SEQ ID NOs: 39-65, 69-132, 159-182, 281-284, 298, 400-401, 741, 761, and 850-861 is at or after amino acid residue 50, 70, 100, 200, 300, 400, 500, 600, 700, 800, 900, 1000, 1100, 1200, or 1300 of the circular permutant. In some embodiments, the N-terminus of the one of SEQ ID NOs: 39-65, 69-132, 159-182, 281-284, 298, 400-401, 741, 761, and 850-861 is between amino acid residues 50 and 100, 70 and 100, 80 and 90, 100 and 200, 110 and 140, 120 and 130, 700 and 800, 710 and 740, 720 and 730, 780 and 810, 790 and 800, 900 and 1000, 940 and 970, 1100 and 1200, 1140 and 1170, or 1150 and 1160 of the circular permutant. In some embodiments, the first portion of the one of SEQ ID NOs: 39-65, 69-132, 159-182, 281-284, 298, 400-401, 741, 761, and 850-861 includes a portion of the C-terminal end of the one of SEQ ID NOs: 39-65, 69-132, 159-182, 281-284, 298, 400-401, 741, 761, and 850-861. In some embodiments, the first portion of the one of SEQ ID NOs: 39-65, 69-132, 159-182, 281-284, 298, 400-401, 741, 761, and 850-861 includes the C-terminus of the circular permutant and/or the first amino acid sequence of the circular permutant includes the C-terminus. In some embodiments, the circular permutant comprises a second amino acid sequence having at least 70%, 75%, 80%, 85%, 90%, 95%, 96%, 97%, 98%, 99%, or 100% sequence identity to a second portion of the one of SEQ ID NOs: 39-65, 69-132, 159-182, 281-284, 298, 400-401, 741, 761, and 850-861, wherein the second portion includes the C-terminus and/or all or a portion of the C-terminal end of the one of SEQ ID NOs: 39-65, 69-132, 159-182, 281-284, 298, 400-401, 741, 761, and 850-861 and the second portion is less than the entire amino acid sequence of SEQ ID NOs: 39-65, 69-132, 159-182, 281-284, 298, 400-401, 741, 761, and 850-861. In some embodiments, the second portion of the one of SEQ ID NOs: 39-65, 69-132, 159-182, 281-284, 298, 400-401, 741, 761, and 850-861 includes the N-terminus of the circular permutant and/or the second amino acid sequence of the circular permutant includes the N-terminus. In some embodiments, the circular permutant comprises, in the N- to C-terminus direction, the second portion of the one of SEQ ID NOs: 39-65, 69-132, 159-182, 281-284, 298, 400-401, 741, 761, and 850-861 and the first portion of the one of SEQ ID NOs: 39-65, 69-132, 159-182, 281-284, 298, 400-401, 741, 761, and 850-861, wherein the first and second portions are covalently linked optionally via a linker (e.g., a peptide linker).

A circular permutant of the present invention may have an amino acid sequence comprising at least 70%, 75%, 80%, 85%, 90%, 95%, 96%, 97%, 98%, 99%, or 100% sequence identity to one or more of SEQ ID NOs: 234-245, 299-310, 390-399, 537-560, 702-704, 735-740, and 757-760. In some embodiments, a circular permutant of the present invention may be encoded by a nucleic acid comprising at least 70%, 75%, 80%, 85%, 90%, 95%, 96%, 97%, 98%, 99%, or 100% sequence identity to one or more of SEQ ID NOs: 247-258, 286-297, 327-338, 402-411, 561-584, 706-708, 742-747, and 763-766.

In some embodiments, a circular permutant of the present invention is a circular permutant of an engineered polypeptide, wherein the engineered polypeptide, optionally when optimally aligned to the amino acid sequence of SEQ ID NO:56, comprises one or more amino acid mutation(s) at a position selected from the group consisting of: D155, G531, K594, D831, R1137, and any combination thereof with reference to the position numbering of SEQ ID NO: 56. In some embodiments, a circular permutant of the present invention is a circular permutant of an engineered polypeptide that, optionally when optimally aligned to the amino acid sequence of SEQ ID NO:56, comprises one or more amino acid mutations selected from the group consisting of: D155R, G531R, K594R, D831A, R1137A, and any combination thereof with reference to the position numbering of SEQ ID NO:56. In some embodiments, the engineered polypeptide, optionally when optimally aligned to the amino acid sequence of SEQ ID NO:56, comprises G531R and K594R mutations, with reference to the position numbering of SEQ ID NO:56.

In some embodiments, a circular permutant of the present invention is a circular permutant of an engineered polypeptide, wherein the engineered polypeptide, optionally when optimally aligned to the amino acid sequence of SEQ ID NO:55, comprises one or more amino acid mutation(s) at a position selected from the group consisting of: D156, G532, K595, D832, R1138, and any combination thereof with reference to the position numbering of SEQ ID NO: 55. In some embodiments, a circular permutant of the present invention is a circular permutant of an engineered polypeptide that, optionally when optimally aligned to the amino acid sequence of SEQ ID NO:55, comprises one or more amino acid mutations selected from the group consisting of: D156R, G532R, K595R, D832A, R1138A, and any combination thereof with reference to the position numbering of SEQ ID NO:55. In some embodiments, the engineered polypeptide, optionally when optimally aligned to the amino acid sequence of SEQ ID NO:55, comprises G532R and K595R mutations, with reference to the position numbering of SEQ ID NO:55.

In some embodiments, a circular permutant of the present invention and/or a polypeptide of the present invention comprising a circular permutant of the present invention, optionally when optimally aligned to the amino acid sequence of SEQ ID NO:235, comprises one or more amino acid mutation(s) at a position selected from the group consisting of: D71, G447, K510, D747, R1053, and any combination thereof with reference to the position numbering of SEQ ID NO:235. In some embodiments, a circular permutant of the present invention and/or a polypeptide of the present invention comprising a circular permutant of the present invention, optionally when optimally aligned to the amino acid sequence of SEQ ID NO: 235, comprises one or more amino acid mutations selected from the group consisting of: D71R, G447R, K510R, D747A, R1053A, and any combination thereof with reference to the position numbering of SEQ ID NO:235. In some embodiments, a circular permutant of the present invention and/or a polypeptide of the present invention comprising a circular permutant of the present invention, optionally when optimally aligned to the amino acid sequence of SEQ ID NO: 235, comprises G447R and K510R mutations, with reference to the position numbering of SEQ ID NO:235.

In some embodiments, a circular permutant of the present invention and/or a polypeptide of the present invention comprising a circular permutant of the present invention, optionally when optimally aligned to the amino acid sequence of SEQ ID NO:237, may comprise one or more amino acid mutation(s) at a position selected from the group consisting of: D1107, D540, R846, and any combination thereof with reference to the position numbering of SEQ ID NO:237. In some embodiments, a circular permutant of the present invention and/or a polypeptide of the present invention comprising a circular permutant of the present invention, optionally when optimally aligned to the amino acid sequence of SEQ ID NO:237, comprises one or more amino acid mutations selected from the group consisting of: D1107R, D540A, R846A, and any combination thereof with reference to the position numbering of SEQ ID NO: 237.

In some embodiments, a circular permutant of the present invention and/or a polypeptide of the present invention comprising a circular permutant of the present invention, optionally when optimally aligned to the amino acid sequence of SEQ ID NO:239, comprises one or more amino acid mutation(s) at a position selected from the group consisting of: D243, G619, K682, D919, R1225, and any combination thereof with reference to the position numbering of SEQ ID NO:239. In some embodiments, a circular permutant of the present invention and/or a polypeptide of the present invention comprising a circular permutant of the present invention, optionally when optimally aligned to the amino acid sequence of SEQ ID NO: 239, comprises one or more amino acid mutations selected from the group consisting of: D243R, G619R, K682R, D919A, R1225A, and any combination thereof with reference to the position numbering of SEQ ID NO:239. In some embodiments, a circular permutant of the present invention and/or a polypeptide of the present invention comprising a circular permutant of the present invention, optionally when optimally aligned to the amino acid sequence of SEQ ID NO: 239, comprises G619R and K682R mutations, with reference to the position numbering of SEQ ID NO:239.

In some embodiments, a circular permutant of the present invention and/or a polypeptide of the present invention comprising a circular permutant of the present invention, optionally when optimally aligned to the amino acid sequence of SEQ ID NO:241, comprises one or more amino acid mutation(s) at a position selected from the group consisting of: D882, G15, K78, D315, R621, and any combination thereof with reference to the position numbering of SEQ ID NO:241. In some embodiments, a circular permutant of the present invention and/or a polypeptide of the present invention comprising a circular permutant of the present invention, optionally when optimally aligned to the amino acid sequence of SEQ ID NO:241, comprises one or more amino acid mutations selected from the group consisting of: D882R, G15R, K78R, D315A, R621A, and any combination thereof with reference to the position numbering of SEQ ID NO:241. In some embodiments, a circular permutant of the present invention and/or a polypeptide of the present invention comprising a circular permutant of the present invention, optionally when optimally aligned to the amino acid sequence of SEQ ID NO: 241, comprises G15R and K78R mutations, with reference to the position numbering of SEQ ID NO:241.

In some embodiments, a circular permutant of the present invention and/or a polypeptide of the present invention comprising a circular permutant of the present invention, optionally when optimally aligned to the amino acid sequence of SEQ ID NO:243, comprises one or more amino acid mutation(s) at a position selected from the group consisting of: D956, D389, R695, and any combination thereof with reference to the position numbering of SEQ ID NO: 243. In some embodiments, a circular permutant of the present invention and/or a polypeptide of the present invention comprising a circular permutant of the present invention, optionally when optimally aligned to the amino acid sequence of SEQ ID NO:243, comprises one or more amino acid mutations selected from the group consisting of: D956R, D389A, R695A, and any combination thereof with reference to the position numbering of SEQ ID NO: 243.

In some embodiments, a circular permutant of the present invention and/or a polypeptide of the present invention comprising a circular permutant of the present invention, optionally when optimally aligned to the amino acid sequence of SEQ ID NO:245, comprises one or more amino acid mutation(s) at a position selected from the group consisting of: D281, R20, D957, and any combination thereof with reference to the position numbering of SEQ ID NO: 245. In some embodiments, a circular permutant of the present invention and/or a polypeptide of the present invention comprising a circular permutant of the present invention, optionally when optimally aligned to the amino acid sequence of SEQ ID NO:245, comprises one or more amino acid mutations selected from the group consisting of: D281R, R20A, D957A, and any combination thereof with reference to the position numbering of SEQ ID NO: 245.

In some embodiments, a circular permutant of the present invention is a circular permutant of an engineered polypeptide, wherein the engineered polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more identity to the amino acid sequence of SEQ ID NO:55 and, relative to the amino acid sequence of SEQ ID NO:55 (e.g., optimally aligned to SEQ ID NO:55), the engineered polypeptide comprises a mutation at one or more of the following positions selected from the group consisting of: N100, K116, K120, K121, D122, E125, T148, T149, T152, D156, E159, N211, N263, T296, E330, K387, A404, D405, D423, E484, L498, N527, Q529, G532, D535, K538, E539, D541, Y542, Y553, Y554, D572, L585, K591, M592, K595, V596, S599, K600, K601, Y616, Y646, W649, and any combination thereof with reference to position numbering of SEQ ID NO:55.

In some embodiments, a circular permutant of the present invention is a circular permutant of an engineered polypeptide, wherein the engineered polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more identity to the amino acid sequence of SEQ ID NO:55 and, relative to the amino acid sequence of SEQ ID NO:55 (e.g., optimally aligned to SEQ ID NO:55), the engineered polypeptide comprises one or more amino acid mutation(s) selected from the group consisting of: N100S, K116D, K116R, K116N, K120R, K120H, K120N, K120T, K120Y, K120Q, K121S, K121T, K121H, K121R, K121G, K121D, K121Q, D122R, D122K, D122H, D122E, D122N, E125G, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D156I, D156V, D156L, D156E, E159K, E159R, E159H, E159Y, E159Q, N211S, N263I, T296I, E330V, K387E, A404V, D405G, D423V, E484D, L498M, N527S, Q529N, Q529T, Q529H, Q529A, Q529F, Q529G, Q529S, Q529P, Q529W, Q529D, G532D, G532N, G532S, G532H, G532F, G532K, G532R, G532Q, G532A, G532L, G532C, D535N, D535H, D535V, D535T, D535S, D535A, D535W, D535K, K538R, K538V, K538Q, K538W, K538Y, K538F, K538H, K538L, K538M, K538C, K538G, K538A, K538P, E539V, D541N, D541H, D541R, D541K, D541Y, D541I, D541A, D541S, D541E, Y542R, Y542K, Y542H, Y542Q, Y542F, Y542L, Y542M, Y542P, Y542V, Y542N, Y542T, Y553H, Y554N, D572G, L585Q, L585G, L585H, L585F, K591W, K591F, K591Y, K591H, K591R, K591S, K591A, K591G, K591P, M592R, M592K, M592Q, M592E, M592A, K595R, K595Q, K595Y, K595L, K595W, K595H, K595E, K595S, K595D, K595M, V596T, V596H, V596G, V596A, S599G, S599H, S599N, S599D, K600R, K600H, K600G, K601R, K601H, K601Q, K601T, Y616K, Y616R, Y616E, Y616F, Y616H, Y646R, Y646E, Y646K, Y646H, Y646Q, Y646W, Y646N, W649H, W649K, W649Y, W649R, W649E, W649S, W649V, W649T, and any combination thereof with reference to the position numbering of SEQ ID NO:55. In some embodiments, a circular permutant of the present invention is a circular permutant of an engineered polypeptide that, optionally when optimally aligned to the amino acid sequence of SEQ ID NO:55, comprises one or more amino acid mutations selected from the group consisting of: N100S, K116D, E125G, T152R, D156E, N211S, N263I, T296I, E330V, K387E, A404V, D405G, D423V, E484D, L498M, N527S, G532R, K538V, E539V, Y542R, Y553H, Y554N, D572G, L585Q, K595R, K595Y, and any combination thereof with reference to the position numbering of SEQ ID NO:55.

In some embodiments, a circular permutant of the present invention is a circular permutant of an engineered polypeptide, wherein the engineered polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more identity to the amino acid sequence of SEQ ID NO:951 and, relative to the amino acid sequence of SEQ ID NO:951 (e.g., optimally aligned to SEQ ID NO:951), the engineered polypeptide comprises a mutation at one or more of the following positions selected from the group consisting of: N100, K116, K120, K121, D122, E125, T148, T149, T152, D156, E159, N211, N263, T443, E478, K535, A552, D553, D571, E632, L646, N675, Q677, G680, D683, K686, E687, D689, Y690, Y701, Y702, D720, L733, K739, M740, K743, V744, S747, K748, K749, Y764, Y794, W797, and any combination thereof with reference to position numbering of SEQ ID NO:951.

In some embodiments, a circular permutant of the present invention is a circular permutant of an engineered polypeptide, wherein the engineered polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more identity to the amino acid sequence of SEQ ID NO:951 and, relative to the amino acid sequence of SEQ ID NO:951 (e.g., optimally aligned to SEQ ID NO:951), the engineered polypeptide comprises one or more amino acid mutation(s) selected from the group consisting of: N100S, K116D, K116R, K116N, K120R, K120H, K120N, K120T, K120Y, K120Q, K121S, K121T, K121H, K121R, K121G, K121D, K121Q, D122R, D122K, D122H, D122E, D122N, E125G, E125R, E125K, E125Q, E125Y, T148H, T148S, T148A, T148C, T149A, T149C, T149S, T149G, T149H, T149P, T149F, T149N, T149D, T149V, T152R, T152K, T152W, T152Y, T152H, T152Q, T152E, T152L, T152F, D156R, D156K, D156Y, D156W, D156Q, D156H, D156I, D156V, D156L, D156E, E159K, E159R, E159H, E159Y, E159Q, N211S, N263I, T443I, E478V, K535E, A552V, D553G, D571V, E632D, L646M, N675S, Q677N, Q677T, Q677H, Q677A, Q677F, Q677G, Q677S, Q677P, Q677W, Q677D, G680D, G680N, G680S, G680H, G680F, G680K, G680R, G680Q, G680A, G680L, G680C, D683N, D683H, D683V, D683T, D683S, D683A, D683W, D683K, K686R, K686V, K686Q, K686W, K686Y, K686F, K686H, K686L, K686M, K686C, K686G, K686A, K686P, E687V, D689N, D689H, D689R, D689K, D689Y, D689I, D689A, D689S, D689E, Y690R, Y690K, Y690H, Y690Q, Y690F, Y690L, Y690M, Y690P, Y690V, Y690N, Y690T, Y701H, Y702N, D720G, L733Q, L733G, L733H, L733F, K739W, K739F, K739Y, K739H, K739R, K739S, K739A, K739G, K739P, M740R, M740K, M740Q, M740E, M740A, K743R, K743Q, K743Y, K743L, K743W, K743H, K743E, K743S, K743D, K743M, V744T, V744H, V744G, V744A, S747G, S747H, S747N, S747D, K748R, K748H, K748G, K749R, K749H, K749Q, K749T, Y764K, Y764R, Y764E, Y764F, Y764H, Y794R, Y794E, Y794K, Y794H, Y794Q, Y794W, Y794N, W797H, W797K, W797Y, W797R, W797E, W797S, W797V, W797T, and any combination thereof with reference to the position numbering of SEQ ID NO:951.

A circular permutant of the present invention may be fused (e.g., directly or indirectly such as via a linker) to one or more polypeptide(s) that are the same or different from each other. In some embodiments, a circular permutant of the present invention is fused (e.g., directly or indirectly such as via a linker) to a second polypeptide. In some embodiments, the second polypeptide is fused to the N-terminus or the C-terminus of the circular permutant via a peptide bond between an amino acid residue of the circular permutant and an amino acid residue of the second polypeptide. In some embodiments, the second polypeptide is fused to the N-terminus or the C-terminus of the circular permutant via a linker (e.g., a peptide linker). The second polypeptide may be a polypeptide of interest. In some embodiments, a circular permutant of the present invention is fused to one or more (e.g., 2, 3, 4, 5, or more) polypeptide(s) of interest. In some embodiments, a circular permutant of the present invention is fused to a cytosine deaminase (e.g., all or a portion of a cytosine deaminase having at least 70%, 75%, 80%, 85%, 90%, 95%, 96%, 97%, 98%, 99%, or 100% sequence identity to one or more of SEQ ID NOs: 183-193 and 798), optionally wherein the cytosine deaminase is fused (directly or indirectly) to the N- or C-terminus of the circular permutant. In some embodiments, a circular permutant of the present invention is fused to a glycosylase inhibitor, optionally wherein the glycosylase inhibitor is a uracil glycosylase inhibitor (UGI), e.g., all or a portion of a UGI having an amino acid sequence having at least 70%, 75%, 80%, 85%, 90%, 95%, 96%, 97%, 98%, 99%, or 100% sequence identity to SEQ ID NO:204. In some embodiments, a circular permutant of the present invention is fused (directly or indirectly) to a cytosine deaminase and a glycosylase inhibitor in any order. In some embodiments, a circular permutant of the present invention is fused to an adenine deaminase (e.g., all or a portion of an adenine deaminase having at least 70%, 75%, 80%, 85%, 90%, 95%, 96%, 97%, 98%, 99%, or 100% sequence identity to one or more of SEQ ID NOs: 194-203 and 884), optionally wherein the adenine deaminase is fused (directly or indirectly) to the N- or C-terminus of the circular permutant. In some embodiments, a circular permutant of the present invention is fused (directly or indirectly) to an adenine deaminase and a cytosine deaminase in any order, optionally wherein the adenine deaminase is fused (directly or indirectly) to one of the N- or C-terminus of the circular permutant and the cytosine deaminase is fused (directly or indirectly) to the other of the N- or C-terminus of the circular permutant. In some embodiments, a nucleotide sequence encoding a deaminase (e.g., a cytosine deaminase or adenine deaminase) comprises an intronic nucleotide sequence. In some embodiments, the intronic nucleotide sequence has at least about 70%, 75%, 80%, 85%, 90%, 95%, 96%, 97%, 98%, 99%, or more sequence identity to SEQ ID NO:950. In some embodiments, an intron (e.g., having at least about 70%, 75%, 80%, 85%, 90%, 95%, 96%, 97%, 98%, 99%, or more sequence identity to SEQ ID NO:950) may be between a first nucleotide sequence encoding a polypeptide of interest (e.g., a cytosine deaminase, adenine deaminase, reverse transcriptase, etc.) and a second nucleotide sequence encoding a circular permutant of the present invention.

In some embodiments, a circular permutant of the present invention is fused to a reverse transcriptase, optionally wherein the reverse transcriptase is fused (directly or indirectly) to the N- or C-terminus of the circular permutant. In some embodiments, a reverse transcriptase may have a sequence that has at least about 70%, 75%, 80%, 85%, 90%, 95%, 96%, 97%, 98%, 99%, or more sequence identity to one or more of SEQ ID NOs: 205-216. In some embodiments, a circular permutant of the present invention is fused to a nuclear localization signal. In some embodiments, a nuclear localization signal has at least 70%, 75%, 80%, 85%, 90%, 95%, 96%, 97%, 98%, 99%, or 100% sequence identity to one or more of SEQ ID NOs: 217-219 and 842-844. In some embodiments, a nuclear localization signal (NLS) of the present invention is encoded by a polynucleotide having at least 70%, 75%, 80%, 85%, 90%, 95%, 96%, 97%, 98%, 99%, or 100% sequence identity to one or more of any one of SEQ ID NOs: 845-849. In some embodiments, a circular permutant of the present invention comprises a NLS that is fused (directly or indirectly) to the N- or C-terminus of the circular permutant. In some embodiments, a NLS is present at the N-terminus and/or C-terminus of a circular permutant of the present invention.

In some embodiments, a polypeptide of the present invention comprises a circular permutant of the present invention and has polymerase activity. Polymerase activity may be measured using methods known in the art. Exemplary assays for measuring and/or determining polymerase activity include, but are not limited to, measuring incorporation of a labeled nucleotide, e.g., a radiolabeled and/or colorimetrically (e.g., fluorescently) labeled nucleotide. In some embodiments, radiolabeling is used to measure and/or determine polymerase activity (e.g., measuring the quantity of a radiolabeled nucleotide included in a polymerized nucleic acid). In some embodiments, a primer extension assay is used to measure and/or determine polymerase activity, optionally wherein the primer extension assay uses a labeled primer (e.g., a fluorescently labeled primer such as a Cy3 or Cy5 labeled primer) to determine the rate of synthesis. In some embodiments, a cleavage assay is used to measure and/or determine polymerase activity (e.g., RNase activity).

In some embodiments, the activity of a polypeptide of the present invention is measured by the number of nucleotides generated (e.g., polymerized) during one cell division and/or in about 20 minutes. Cell division and/or the time period for one cell division can be readily determined by one of skill in the art. In some embodiments, the cell division is a cell division for a bacterial cell, a human cell, or a plant cell. In some embodiments, the time of one cell division is about 18 to about 22 minutes, about 19 to about 21 minutes, or about 20 minutes. In some embodiments, a polypeptide of the present invention generates (e.g., polymerizes) at least 18, 19, 20, 21, 22, 23, 24, 25 or more nucleotides during one cell division and/or in about 20 minutes, optionally at a temperature of about 10° C., 15° C., 20° C., 25° C., 30° C., 35° C., 40° C., 45° C., 50° C., 55° C., 60° C., 65° C., 70° C., 75° C., or 80° C. Polypeptide activity may be measured by the rate of nucleotides generated (e.g., polymerized) in a period of time. In some embodiments, a polypeptide of the present invention polymerizes at least 18, 19, 20, 21, 22, 23, 24, or 25 nucleotides in 20 minutes. In some embodiments, a polypeptide of the present invention polymerizes at least 0.9, 0.95, 1.0, 1.05, 1.1, 1.15, 1.20, 1.25 or more nucleotides per minute at a physiologically relevant temperature. In some embodiments, a polypeptide of the present invention generates nucleotides at a rate of at least 0.9, 0.95, 1.0, 1.05, 1.1, 1.15, 1.20, or 1.25 nucleotides per minute at a temperature of about 10° C., 15° C., 20° C., 25° C., 30° C., 35° C., 40° C., 45° C., 50° C., 55° C., 60° C., 65° C., 70° C., 75° C., or 80° C. In some embodiments, a polypeptide of the present invention generates (e.g., polymerizes) at least 23 nucleotides during one cell division and/or in about 20 minutes, optionally at a temperature of about 10° C., 15° C., 20° C., 25° C., 30° C., 35° C., 40° C., 45° C., 50° C., 55° C., 60° C., 65° C., 70° C., 75° C., or 80° C.

In some embodiments, a polypeptide of the present invention generates DNA from RNA at a temperature in a range from about 10° C., 15° C., 20° C., 25° C., 30° C., 35° C., 40° C., 45° C. or 50° C. to about 55° C., 60° C., 65° C., 70° C., 75° C., or 80° C. In some embodiments, a polypeptide of the present invention generates DNA from RNA at a temperature of about 50° C. or less such as at a temperature of about 10° C., 15° C., 20° C., 25° C., 30° C., 35° C., 40° C., or 45° C. In some embodiments, a polypeptide of the present invention generates DNA from RNA at a temperature in a range of about 10° C. to about 40° C., about 15° C. to about 35° C., about 15° C. to about 40° C., about 15° C. to about 50° C., about 18° C. to about 30° C., about 18° C. to about 25° C., about 20° C. to about 25° C., about 20° C. to about 80° C., about 20° C. to about 50° C., about 20° C. to about 45° C., about 30° C. to about 50° C., about 30° C. to about 45° C., about 30° C. to about 40° C., about 20° C. to about 22° C., about 18° C., 19° C., 20° C., 21° C., 22° C., 23° C., 24° C., or 25° C., or about room temperature.

As used herein, “processivity” refers to the number of nucleotides generated (e.g., synthesized) in a single binding event of a polypeptide of the present invention. Processivity may be measured in vitro and/or in vivo using methods known in the art. In some embodiments, processivity may be measured by the number of bases generated over a period of time per unit of enzyme (e.g., polypeptide of the present invention), wherein 1 unit of enzyme is the amount of enzyme that will incorporate 1 nmol of dTTP into acid-insoluble material in a total reaction volume of 50 μl in 10 minutes at 37° C. using poly(rA)·oligo(dT) as the template primer with 50 mM Tris-HCl (pH 8.3), 6 mM MgCl2, 10 mM dithiothreitol, 0.5 mM [3H]-dTTP and 0.4 mM poly(rA)·oligo(dT) 12-18. In some embodiments, a polypeptide of the present invention has a processivity of at least about 100, 250, 500, 1000, 1500, or 2000 nucleotides or more, e.g. about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2500, 3000, 3500, 4000, 4500, or 5000 nucleotides or more. In some embodiments, a polypeptide of the present invention has a processivity of about 100 to about 600 nucleotides, about 200 to about 500 nucleotides, or at least about 300 nucleotides.

A polypeptide of the present invention may have a processivity that is increased compared to the processivity of a control reverse transcriptase. A “control reverse transcriptase” or “control RT” as used herein refers to a naturally occurring reverse transcriptase or commercially available reverse transcriptase. Exemplary control RTs include, but are not limited to, Moloney Murine Leukemia Virus Reverse Transcriptase (MMLV-RT or M-MuLV-RT), mutated MMLV (e.g., 5M-MMLV), Avian Myeloblastosis Virus Reverse Transcriptase (AMV-RT), Human Immunodeficiency Virus Reverse Transcriptase (HIV-RT). In some embodiments, the control RT may be MMLV and/or 5M-MMLV. In some embodiments, a control RT may have a sequence of one of SEQ ID NOs: 205-216. Processivity for a polypeptide of the present invention and for control RT may be measured by using one or more sequences under the same reaction conditions (e.g., same time, temperature, concentration, sequence identity, modification, etc.). In some embodiments, a polypeptide of the present invention may have a processivity that is reduced compared to (e.g., lower than) the processivity of a control RT and/or may have a processivity that is faster than a DNA repair enzyme's ability to correct a modification such that the polypeptide can out-run the DNA repair enzyme. In some embodiments, a polypeptide of the present invention has a processivity at a first temperature that is equal to or better than the processivity of a control reverse transcriptase at a second temperature, wherein the first temperature is lower than the second temperature. For example, a polypeptide of the present invention may have a processivity at room temperature that is equal to or better than the processivity of a control reverse transcriptase such as 5M-MMLV at a temperature of greater than room temperature, for example, about 42° C. or about 55° C. In some embodiments, the processivity of a polypeptide of the present invention is within ±about 5%, about 10%, about 15%, about 20%, or about 25% of the processivity of the DNA polymerase in the cell. In some embodiments, the processivity of the polypeptide of the present invention is within about 5%, about 10%, about 15%, about 20% or about 25% of the rate of one or more replicative polymerases of the cell, one or more repair polymerases of the cell, or a combination thereof.

In some embodiments, a polypeptide of the present invention is devoid of at least a portion of an RNaseH domain. RNaseH domains are conserved across viral RTs and are structurally similar to RNaseH domains in Escherichia coli, Bacillus halodurans and human RNase H1. See, e.g., Champoux et al., FEBS Journal, 276:6, 1506-1516 (2009). Enzymatic activity is conferred by the DEDD sequence motif, a conserved sequence composed of aspartate and glutamate residues, which, in HIV-1 RNase H, are Asp443, Glu478, Asp498 and Asp549, with the corresponding active site amino acids in the M-MLV enzyme of Asp524, Glu562, Asp583 and Asp653.

In some embodiments, a polypeptide of the present invention has reduced (e.g., about 90%, 80%, 70%, 60%, 50%, 40%, 30%, 20%, 15%, 10%, 5%, or less) or no ribonuclease (RNase) activity. In some embodiments, a polypeptide of the present invention has reduced (e.g., about 90%, 80%, 70%, 60%, 50%, 40%, 30%, 20%, 15%, 10%, 5%, or less) or no ribonuclease (RNase) activity compared to a control RT. In some embodiments, the control reverse transcriptase may be an RT with high RNase activity, for example, avian myeloblastosis virus (AMV) or medium RNasH activity, for example, MMLV-RT.

In some embodiments, a polypeptide of the present invention generates and/or is capable of generating DNA from RNA in two or more different species (e.g., 3, 4, 5, 6, 7, 8, 9, or 10, or more different species). In some embodiments, a polypeptide of the present invention generates DNA from RNA in two or more different species and has a processivity of at least about 100, 200, 300, 400, 500, 600, 700, 800, 900, 1,000, 1100, 1200, 1300, 1400, 1500, 1600, 1700, 1800, 1900, or 2,000 nucleotides or more, optionally at a temperature in a range of about 25° C., 30° C., 35° C., 40° C., 45° C. or 50° C. to about 55° C., 60° C., 65° C., 70° C., 75° C., or 80° C. In some embodiments, a polypeptide of the present invention generates DNA from RNA in two or more different species at a temperature in a range of about 20° C., 25° C., 30° C., 35° C., 40° C., 45° C. or 50° C. to about 55° C., 60° C., 65° C., 70° C., 75° C., or 80° C. for each of the two or more different species. In some embodiments, a polypeptide of the present invention generates DNA from RNA in two or more different species and has a processivity of at least about 100, 200, 300, 400, 500, 600, 700, 800, 900, 1,000, 1100, 1200, 1300, 1400, 1500, 1600, 1700, 1800, 1900, or 2,000 nucleotides or more, optionally at a temperature of less than 50° C. (e.g., about 20° C., 25° C., 30° C., 35° C., 40° C., or 45° C.) or at a temperature in a range of about 20° C., 25° C., or 30° C. to about 35° C., 40° C., or 45° C.

A polypeptide of the present invention may generate DNA from RNA in a prokaryote or a eukaryote. In some embodiments, a polypeptide of the present invention generates DNA from RNA in a plant and/or an animal. In some embodiments, a polypeptide of the present invention generates DNA from RNA in corn, soy, canola, wheat, rice, cotton, sugarcane, sugar beet, barley, oats, alfalfa, sunflower, safflower, oil palm, sesame, coconut, tobacco, potato, sweet potato, cassava, coffee, apple, plum, apricot, peach, cherry, pear, fig, banana, citrus, cocoa, avocado, olive, almond, walnut, strawberry, watermelon, pepper, grape, tomato, cucumber, blackberry, raspberry, black raspberry, and/or a Brassica spp.

A polypeptide of the present invention may be a fusion protein. In some embodiments, a polypeptide of the present invention is fused (e.g., directly or indirectly such as via a linker) to a second polypeptide (e.g., a deaminase and/or reverse transcriptase).

A polypeptide of the present invention (optionally in a complex of the present invention) may have an editing efficiency (e.g., indel percentage) that is the same as or greater than (e.g., increased compared to) a Cas12a (optionally in a complex with comparable components to those in the complex with the polypeptide of the present invention). In some embodiments, a polypeptide of the present invention has an editing efficiency that is the same as or greater than a Cas12a when performed at a temperature of less than 50° C. such as about 45° C., about 42° C., about 37° C., about 30° C., about 25° C., or about room temperature.

According to some embodiments of the present invention, a complex is provided that comprises a circular permutant of the present invention and a guide nucleic acid. In some embodiments, the guide nucleic acid is an extended guide nucleic acid and/or the complex further comprises an extended guide nucleic acid. In some embodiments, the circular permutant is fused to a second polypeptide (e.g., a deaminase and/or reverse transcriptase). In some embodiments, the circular permutant may be fused (e.g., directly or indirectly) to a peptide tag, optionally a peptide tag that is capable of binding an affinity polypeptide. In some embodiments, the circular permutant may be fused (e.g., directly or indirectly) an affinity polypeptide, optionally an affinity polypeptide that is capable of binding a peptide tag. In some embodiments, the circular permutant may be fused (e.g., directly or indirectly) to an affinity polypeptide that is capable of binding an RNA recruiting motif. In some embodiments, a system and/or composition of the present invention comprises a circular permutant of the present invention that comprises a peptide tag and a deaminase and/or reverse transcriptase comprising an affinity polypeptide that is capable of binding the peptide tag. In some embodiments, a system and/or composition of the present invention comprises a circular permutant of the present invention that comprises an affinity polypeptide and a deaminase and/or reverse transcriptase comprising a peptide tag that is capable of binding the affinity polypeptide. In some embodiments, a polypeptide of the present invention may be fused to an affinity polypeptide that is capable of binding an RNA recruiting polypeptide. A complex of the present invention may further comprise a guide nucleic acid that is optionally devoid of a reverse transcriptase template. In some embodiments, the guide nucleic acid is directed to a different target nucleic acid than the extended guide nucleic acid.

A nucleic acid molecule may encode a polypeptide of the present invention, which may be present in an expression cassette and/or vector. A polynucleotide and/or recombinant nucleic acid construct of this invention can be codon optimized for expression. In some embodiments, a polynucleotide, nucleic acid construct, expression cassette, and/or vector of the present invention (e.g., that comprises/encodes a polypeptide of the present invention, a nucleic acid binding polypeptide (e.g., a DNA binding domain such as a sequence-specific DNA binding domain from a polynucleotide-guided endonuclease such as a CRISPR-Cas effector protein), and/or an extended guide nucleic acid) may be codon optimized for expression in an organism (e.g., an animal, a plant, a fungus, an archaeon, or a bacterium). In some embodiments, a polynucleotide of the present invention comprises an intron, optionally an intron having at least about 70%, 75%, 80%, 85%, 90%, 95%, 96%, 97%, 98%, 99%, or more sequence identity to SEQ ID NO:950. In some embodiments, an intron (e.g., having at least about 70%, 75%, 80%, 85%, 90%, 95%, 96%, 97%, 98%, 99%, or more sequence identity to SEQ ID NO:950) may be between a first nucleotide sequence encoding a polypeptide of interest (e.g., a cytosine deaminase, adenine deaminase, reverse transcriptase, etc.) and a second nucleotide sequence encoding a circular permutant of the present invention.

In some embodiments, an expression cassette and/or vector of the present invention comprises a polynucleotide that has at least about 70%, 75%, 80%, 85%, 90%, 95%, 96%, 97%, 98%, 99%, or more sequence identity to one or more of SEQ ID NOs: 247-258, 260-271, 286-297, 327-338, 364-375, 377-388, 402-411, 414-423, 462-485, 503-514, 520-531, 561-608, 640-701, 706-708, 710-712, 728-733, 742-747, 749-754, 763-766, 768-772, 782, 784, 787, and 792-797. In some embodiments, an expression cassette and/or vector of the present invention comprises a polynucleotide that encodes a promoter sequence and a polynucleotide that encodes a polypeptide that has at least about 70%, 75%, 80%, 85%, 90%, 95%, 96%, 97%, 98%, 99%, or more sequence identity to one or more of SEQ ID NOs: 234-245, 299-310, 351-362, 390-399, 450-461, 486-497, 537-560, 609-639, 702-704, 735-740, and 757-760. In some embodiments, the vector comprising the polynucleotide that encodes a polypeptide has at least about 70%, 75%, 80%, 85%, 90%, 95%, 96%, 97%, 98%, 99%, or more sequence identity to one or more of SEQ ID NOs: 260-271, 286-297, 377-388, 414-423, 474-485, 520-531, 585-608, 671-701, 710-712, 728-733, 749-754, 768-772, and 792-797. The polynucleotide encoding the polypeptide of the present invention may be codon-optimized for expression in a particular organism (e.g., a human or plant). In some embodiments, the organism is an animal (e.g., a human), a plant, a fungus, an archaeon, or a bacterium.

Methods of modifying a target nucleic acid in a cell are provided, and may comprise introducing an expression cassette and/or vector of the present invention into the cell to provide a modified target nucleic acid. In some embodiments, the cell is a mammalian cell (e.g., a human cell). In some embodiments, the cell is a plant cell and the method further comprises regenerating the plant cell comprising the modified target nucleic acid to produce a plant comprising the modified target nucleic acid. In some embodiments, the introducing of the expression cassette is carried out at a temperature of about 20° C. to about 42° C.

Methods for producing a polypeptide of the present invention are provided herein and may comprise culturing a cell or a plurality of cells that have been transformed with a nucleic acid encoding a polypeptide of the present invention; and isolating the polypeptide of the present invention to thereby produce the polypeptide. In some embodiments, the step of isolating is performed by dialysis, centrifugation, column purification, and/or the like.

Methods for performing reverse transcription are provided herein and may comprise contacting a target nucleic acid with a polypeptide of the present invention. In some embodiments, all or a portion of the polypeptide has at least about 70%, 75%, 80%, 85%, 90%, 95%, 96%, 97%, 98%, 99%, or more sequence identity to one or more of SEQ ID NOs: 234-245, 299-310, 390-399, 537-560, 702-704, 735-740, and 757-760. A polypeptide of the present invention may reverse transcribe the target nucleic acid to provide a DNA (e.g., a cDNA). In some embodiments, the target nucleic acid is in a plant and/or in a plant cell that includes a cell wall. In some embodiments, the target nucleic acid is in a mammal and/or is in a mammalian cell.

Methods of modifying a target nucleic acid are provided herein and may comprise contacting the target nucleic acid with a polypeptide of the present invention and a guide nucleic acid. In some embodiments, all or a portion of the polypeptide has at least about 70%, 75%, 80%, 85%, 90%, 95%, 96%, 97%, 98%, 99%, or more sequence identity to one or more of SEQ ID NOs: 234-245, 299-310, 351-362, 390-399, 450-461, 486-497, 537-560, 609-639, 702-704, 735-740, and 757-760. In some embodiments, the target nucleic acid is in a plant and/or in a plant cell that includes a cell wall. In some embodiments, the method of modifying the target nucleic acid is a templated editing method.

A method of the present invention may comprise contacting the target nucleic acid with an extended guide nucleic acid. In some embodiments, the extended guide nucleic acid comprises a primer binding site and the target nucleic acid is double stranded and comprises a first strand and a second strand. In some embodiments, the primer binding site of the extended guide nucleic acid binds to the first strand or to the second strand of the target nucleic acid. In some embodiments, the second strand is the non-target strand of the target nucleic acid. In some embodiments, the target nucleic acid is double stranded and comprises a first strand and a second strand and the primer binding site binds to the first strand of the target nucleic acid. The first strand may be the target strand of the target nucleic acid and/or a polypeptide of the present invention may be recruited to the first strand. In some embodiments, the target nucleic acid is double stranded and comprises a first strand and a second strand and the primer binding site of the extended guide nucleic acid binds to the second strand of the target nucleic acid. In some embodiments, the second strand is the non-target strand of the target nucleic acid and/or the polypeptide of the present invention is recruited to the second strand. In some embodiments, the polypeptide of the present invention is a double stranded nuclease that cuts the first strand and the second strand of the target nucleic acid resulting in a double stranded break. In some embodiments, the polypeptide of the present invention and the extended guide nucleic acid form a complex or are comprised in a complex.

A method of the present invention may comprise contacting a target nucleic acid with and/or introducing into a cell an extended guide nucleic acid that comprises (i) a CRISPR nucleic acid and/or a CRISPR nucleic acid and a tracr nucleic acid; and (ii) an extended portion comprising a primer binding site and a reverse transcriptase template (RT template). In some embodiments, the extended portion of the extended guide nucleic acid is fused to either the 5′ end or 3′ end of the CRISPR nucleic acid (e.g., 5′ to 3′: repeat-spacer-extended portion, or extended portion-repeat-spacer) and/or to the 5′ or 3′ end of the tracr nucleic acid. In some embodiments, the extended portion of the extended guide nucleic acid comprises, 5′ to 3′, an RT template and a primer binding site. In some embodiments, the extended portion of the extended guide nucleic acid is located 5′ of the crRNA.

In some embodiments, the primer binding site has a length of about one nucleotide to about 100 nucleotides. In some embodiments, the primer binding site is at least 45 nucleotides in length or about 45 nucleotides to about 100 nucleotides, e.g., 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the RT template has a length of about one to about 100 nucleotides, or the RT template can have a length of about 40 nucleotides or less, e.g., 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide.

In some embodiments, the extended portion of the extended guide nucleic acid is linked to the CRISPR nucleic acid and/or the tracrRNA via a linker. The linker may be about 1 to about 100 nucleotides in length, e.g., 1, 2, 3, 4, 5, 6, 7, 8, 9, 10, 11, 12, 13, 14, 15, 16, 17, 18, 19, 20, 21, 22, 23, 24, 25 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, in a method of the present invention, a polypeptide of the present invention is a fusion protein. In some embodiments, the polypeptide of the present invention is fused to one or more components that recruit a deaminase and/or reverse transcriptase to the polypeptide. In some embodiments, a circular permutant of the present invention is fused, e.g. linked, to a peptide tag, for example, an epitope or a multimerized epitope, and a reverse transcriptase is fused, e.g., linked, to an affinity polypeptide that binds to the peptide tag. In some embodiments, a circular permutant of the present invention is fused, e.g., linked, to a peptide tag, for example, an epitope or a multimerized epitope, and a reverse transcriptase is fused (linked) to an affinity polypeptide that binds to the peptide tag. In some embodiments, the target nucleic acid is contacted with two or more reverse transcriptases.

In some embodiments, an extended guide nucleic acid is linked to an RNA recruiting motif, and a reverse transcriptase is fused, e.g., linked, to an affinity polypeptide that binds to the RNA recruiting motif. In some embodiments, the target nucleic acid is contacted with two or more reverse transcriptases. In some embodiments, the extended guide nucleic acid, e.g. extended guide RNA, is linked to two or more RNA recruiting motifs, optionally wherein the two or more RNA recruiting motifs are the same RNA recruiting motif or different RNA recruiting motifs. In some embodiments, at least one of the two or more RNA recruiting motifs is located on the 3′ end of the extended portion of the extended guide nucleic acid or is embedded in the extended portion.

A method of the present invention may comprise contacting the target nucleic acid with a Dna2 polypeptide and/or a 5′ flap endonuclease (FEN). In some embodiments, the FEN and/or Dna2 polypeptide is overexpressed (e.g., overexpressed in the presence of the target nucleic acid). In some embodiments, the FEN is a fusion protein comprising an FEN domain fused to the CRISPR-Cas effector protein and/or wherein the Dna2 polypeptide is a fusion protein comprising an Dna2 domain fused to the CRISPR-Cas effector protein. In some embodiments, the CRISPR-Cas effector protein is a first CRISPR-Cas effector protein and the method includes a step of contacting the target nucleic acid with a second CRISPR-Cas effector protein. In some embodiments, the first CRISPR-Cas effector protein nicks or cuts a first site on the first strand of a double stranded target nucleic acid. In some embodiments, the nicking or cut site is located about 10 to about 125 base pairs upstream or downstream, e.g., 5′ or 3′, from a second site on the second strand of the target nucleic acid that has been nicked by a second CRISPR-Cas effector protein.

In some embodiments, a method of the present invention has increased efficiency in modifying a target nucleic acid compared to the efficiency of a control method (e.g., a method that uses a control Cas12a and is performed under the same conditions). A method of the present invention may generate increased indels and/or increased levels of modification (e.g., precise modifications) compared to a control method.

In some embodiments, a complex and/or method of the present invention may be a complex and/or method as described in U.S. Patent Application Publication No. 2021/0130835 and/or in U.S. Patent Application Publication No. 2022/0145334, the contents of each of which are incorporated herein by reference in their entirety.

In some embodiments, an editing system of the present invention is used in prime editing. “Prime editing” and grammatical variants thereof as used herein refer to a nucleic acid editing technology that uses a Cas9 nickase domain fused to a reverse transcriptase and modifies a target nucleic acid without a double strand break or a donor DNA template. In Prime editing, the Cas9 nickase domain cuts the non-complementary strand of DNA upstream of the PAM site, thereby providing a 3′ flap that is extended with the extension including a modification. Further details on Prime editing can be found in Anzalone et al. (2019) Nature 576, 149-157 and/or U.S. Patent Application Publication No. 2021/0147862, the contents of each of which are incorporated herein by reference in their entirety.

In some embodiments, an editing system of the present invention utilizes the Redraw editing system. Further details on the Redraw editing system can be found in U.S. Patent Application Publication No. 2021/0130835 and/or in U.S. Patent Application Publication No. 2022/0145334, the contents of each of which are incorporated herein by reference in their entirety.

As described herein, polypeptides of the present invention, nucleic acids, expression cassettes, and/or vectors of the present invention may be codon optimized for expression in an organism. An organism useful with this invention may be any organism or cell thereof for which nucleic acid modification may be useful. An organism can include, but is not limited to, any animal (e.g., a mammal), any plant, any fungus, any archaeon, or any bacterium. In some embodiments, the organism may be a plant or cell thereof. In some embodiments, the organism is an animal such as a mammal (e.g., a human). In some embodiments, an expression cassette and/or vector may be optimized for expression in an organism by including an organism specific promoter sequence, terminator sequence, nuclear localization sequence (NLS), and/or codon optimization. In some embodiments, an expression cassette and/or vector may be optimized for expression in a plant (e.g., soy).

The target nucleic acid may be a genomic sequence from any organism (e.g., eukaryote such as a mammal or a plant). In some embodiments, the target nucleic acid is a genomic sequence from a model organism such as, but not limited to, Escherichia coli, an immortalized human cell line (e.g., HEK293, HeLa, etc.), Caenorhabditis elegans, and/or Drosophila Melanogaster. In some embodiments, the target nucleic acid is a genomic sequence from a non-model organism. Exemplary non-model organisms include, but are not limited to crop plants (e.g., fruit crop plants, vegetable crop plants, and/or field crop plants) and/or animals such as humans, primates and/or mice. In some embodiments, the non-model organism is a crop plant such as corn, soybean, wheat, or canola. In some embodiments, the non-model organism is an animal for testing and/or use of a human therapeutic.

A target nucleic acid of any plant or plant part may be modified using the nucleic acid constructs of the invention. Any plant (or groupings of plants, for example, into a genus or higher order classification) may be modified using a polypeptide of the present invention including an angiosperm, a gymnosperm, a monocot, a dicot, a C3, C4, CAM plant, a bryophyte, a fern and/or fern ally, a microalgae, and/or a macroalgae. A plant and/or plant part useful with this invention may be a plant and/or plant part of any plant species/variety/cultivar. The term “plant part,” as used herein, includes but is not limited to, embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell can be in the form of an isolated single cell or can be a cultured cell or can be a part of a higher-organized unit such as, for example, a plant tissue or a plant organ.

Non-limiting examples of plants useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, Chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, sunflower, raspberry, blackberry, black raspberry and/or cherry.

In some embodiments, the invention provides cells (e.g., plant cells, animal cells, bacterial cells, archaeon cells, and the like) comprising the polypeptides, polynucleotides, nucleic acid constructs, expression cassettes or vectors of the invention.

The present invention further comprises a kit or kits to carry out the methods of this invention. A kit of this invention can comprise reagents, buffers, and apparatus for mixing, measuring, sorting, labeling, etc., as well as instructions and the like as would be appropriate for modifying a target nucleic acid.

In some embodiments, the invention provides a kit for comprising one or more polypeptides of the present invention (e.g., circular permutants), nucleic acid constructs of the present invention, and/or expression cassettes and/or vectors and/or cells comprising the same as described herein, with optional instructions for the use thereof. In some embodiments, a kit may further comprise a CRISPR-Cas guide nucleic acid (corresponding to a Cas12a protein as provided herein, which may be encoded by a polynucleotide) and/or expression cassettes and/or vectors and or cells comprising the same. In some embodiments, a guide nucleic acid may be provided on the same expression cassette and/or vector as one or more nucleic acid constructs of the invention. In some embodiments, the guide nucleic acid may be provided on a separate expression cassette or vector from that comprising the one or more nucleic acid constructs of the invention.

Accordingly, in some embodiments, kits are provided comprising a nucleic acid construct comprising (a) a polynucleotide(s) as provided herein and (b) a promoter that drives expression of the polynucleotide(s) of (a). In some embodiments, the kit may further comprise a nucleic acid construct encoding a guide nucleic acid, wherein the construct comprises a cloning site for cloning of a nucleic acid sequence identical or complementary to a target nucleic acid sequence into backbone of the guide nucleic acid.

In some embodiments, the nucleic acid construct of the invention may be an mRNA that may encode one or more introns within the encoded polynucleotide(s). In some embodiments, the nucleic acid constructs of the invention, and/or an expression cassettes and/or vectors comprising the same, may further encode one or more selectable markers useful for identifying transformants (e.g., a nucleic acid encoding an antibiotic resistance gene, herbicide resistance gene, and the like).

A polypeptide, polynucleotide, nucleic acid construct, expression cassette, vector, composition, kit, system and/or cell of the present invention may comprise all or a portion of a sequence of one or more of SEQ ID NOs: 1-951. In some embodiments, if not present in an amino acid sequence described herein and/or encoded in a nucleotide sequence described herein, the amino acid sequence and/or the nucleotide sequence may further include 1, 2, 3, 4, or 5 additional amino acid(s) or corresponding nucleotides such as a methionine at amino acid residue 1 of the amino acid sequence or the corresponding nucleotides and/or 1, 2, 3, or 4 additional amino acid(s) such as glycine or alanine or the corresponding nucleotides, which may aid in expression or enable the construction or cloning as needed for construction of a plasmid or construct or vector. In some embodiments, if present in an amino acid sequence described herein and/or encoded in a nucleotide sequence described herein, the amino acid sequence and/or the nucleotide sequence may be devoid of 1, 2, 3, 4, or 5 amino acid(s) present at amino acid residues 1-5 at the N-terminus of the amino acid sequence or corresponding nucleotides such as a methionine at amino acid residue 1 of the amino acid sequence or the corresponding nucleotides and/or 1, 2, 3, or 4 additional amino acid(s) such as glycine or alanine or the corresponding nucleotides, which may aid in expression or enable the construction or cloning as needed for construction of a plasmid or construct or vector. In some embodiments, a polypeptide, polynucleotide, nucleic acid construct, expression cassette, vector, composition, kit, system and/or cell of the present invention may comprise at least about 20%, 25%, 30%, 35%, 40%, 45%, 50%, 55%, 60%, 65%, 70%, 75%, 80%, 85%, 90%, 95%, 96%, 97%, 98%, 99%, or more consecutive amino acids or nucleotides of a sequence of one or more of SEQ ID NOs: 1-951.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

Circular permutants were prepared based on LbCas12a (SEQ ID NO:56). Table 1 describes the location at which the N-terminus for the circular permutant was selected to begin relative to LbCas12a.

TABLE 1
Circular permutation of LbCas12a designs

| | | Amino acid residue of |
|---|---|---|
| | | LbCas12a that corresponds |
| Loop Number of | | to the N-terminus of the |
| LbCas12a | Selected Cut Point | circular permutant |
| 3 | After amino acid residue 84 of LbCas12a | 85 |
| 4 | After amino acid residue 291 of LbCas12a | 292 |
| 5 | After amino acid residue 1155 of | 1156 |
| | LbCas12a | |
| 9 | After amino acid residue 516 of LbCas12a | 517 |
| 10 | After amino acid residue 442 of LbCas12a | 443 |
| 14 | After amino acid residue 1117 of | 1118 |
| | LbCas12a | |

A glycine-serine linker was used to connect the native N- and C-termini of LbCas12a. The glycine-serine linker had either 10 amino acids and a sequence of (GSS)ₙG (SEQ ID NO: 36), wherein n is 3, or 16 amino acids and a sequence of (GSS)ₙG (SEQ ID NO:36), wherein n is 5. The circular permutants are listed in Table 2.

TABLE 2
Circular Permutants

| | Vector | DNA | | Protein |
|---|---|---|---|---|
| | SEQ | SEQ | | SEQ |
| pWISE | ID NO: | ID NO: | Description | ID NO: |
| pWISE121 | 259 | 246 | WT-LbCas12a (control) | 56 |
| pWISE8345 | 260 | 247 | Loop 3; 10-aa GS linker | 234 |
| pWISE8346 | 261 | 248 | Loop 3; 16-aa GS linker | 235 |
| pWISE8347 | 262 | 249 | Loop 4; 10-aa GS linker | 236 |
| pWISE8348 | 263 | 250 | Loop 4; 16-aa GS linker | 237 |
| pWISE8349 | 264 | 251 | Loop 5; 10-aa GS linker | 238 |
| pWISE8350 | 265 | 252 | Loop 5; 16-aa GS linker | 239 |
| pWISE8351 | 266 | 253 | Loop 9; 10-aa GS linker | 240 |
| pWISE8352 | 267 | 254 | Loop 9; 16-aa GS linker | 241 |
| pWISE8353 | 268 | 255 | Loop 10; 10-aa GS linker | 242 |
| pWISE8354 | 269 | 256 | Loop 10; 16-aa GS linker | 243 |
| pWISE8355 | 270 | 257 | Loop 14; 10-aa GS linker | 244 |
| pWISE8356 | 271 | 258 | Loop 14; 16-aa GS linker | 245 |

### Example 2

The circular permutants of Example 1 were tested for their ability to generate INDELS at three different target nucleic acids (SEQ ID NOs: 272-274) in HEK293T cells. As a control, LbCas12a was also tested for its ability to generate INDELS at the three target nucleic acids (SEQ ID NOs: 272-274) in HEK293T cells (Table 3).

TABLE 3
Targets

| Target | Target | | Spacer | | Vector |
|---|---|---|---|---|---|
| Nucleic | SEQ | | SEQ | | SEQ |
| Acid | ID NO: | Spacer | ID NO: | pWISE | ID NO: |
| human DNMT1 | 272 | PWsp143 | 275 | pWISE264 | 278 |
| human FANCF | 273 | PWsp449 | 276 | pWISE878 | 279 |
| human RNF2 | 274 | PWsp453 | 277 | pWISE882 | 280 |

Edit characterization was performed using a bioinformatic workflow. First, low quality base-pairs and bases matching Illumina adapter sequences were trimmed using HtStream. High quality reads were then aligned to the amplicon references using bbmap. The CIGAR string, which represents the number of matches and INDELs, was parsed for each aligned read to generate an edit string. The edit string includes the starting location and length of each INDEL relative to the reference.

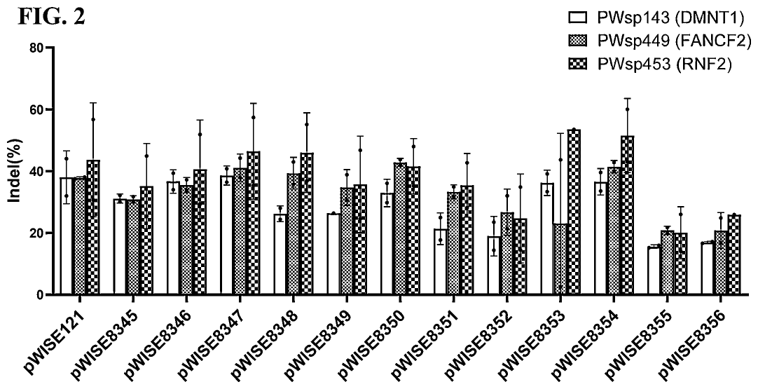

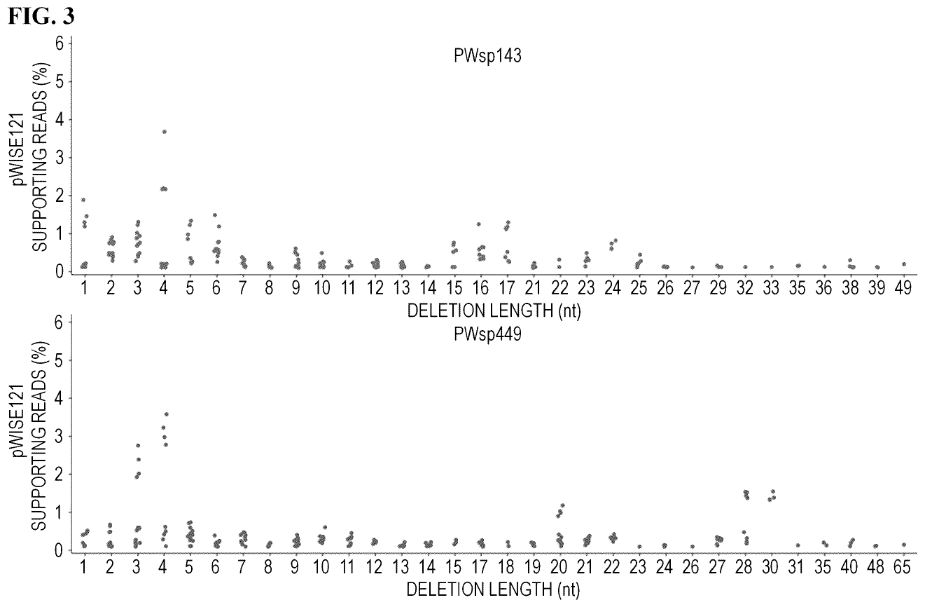

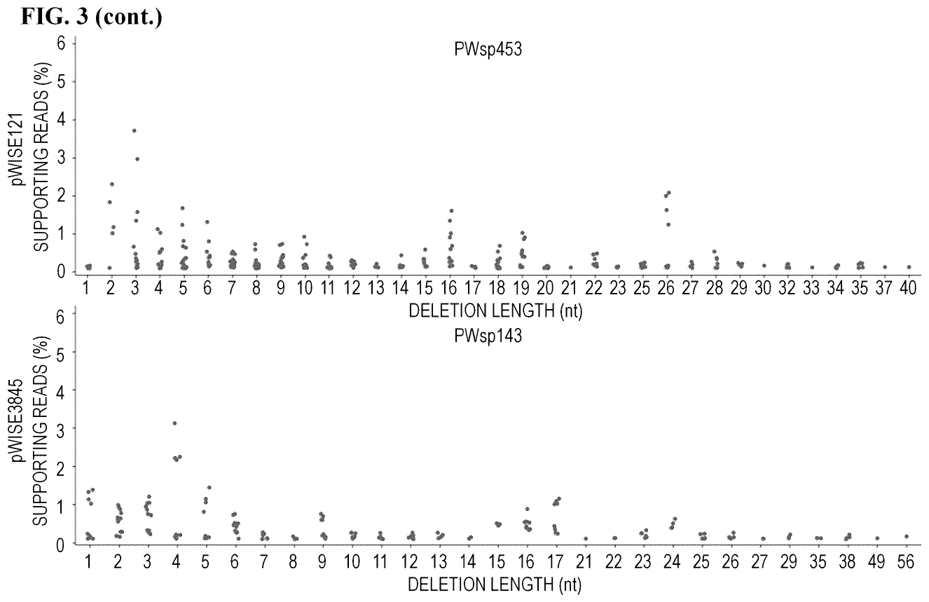

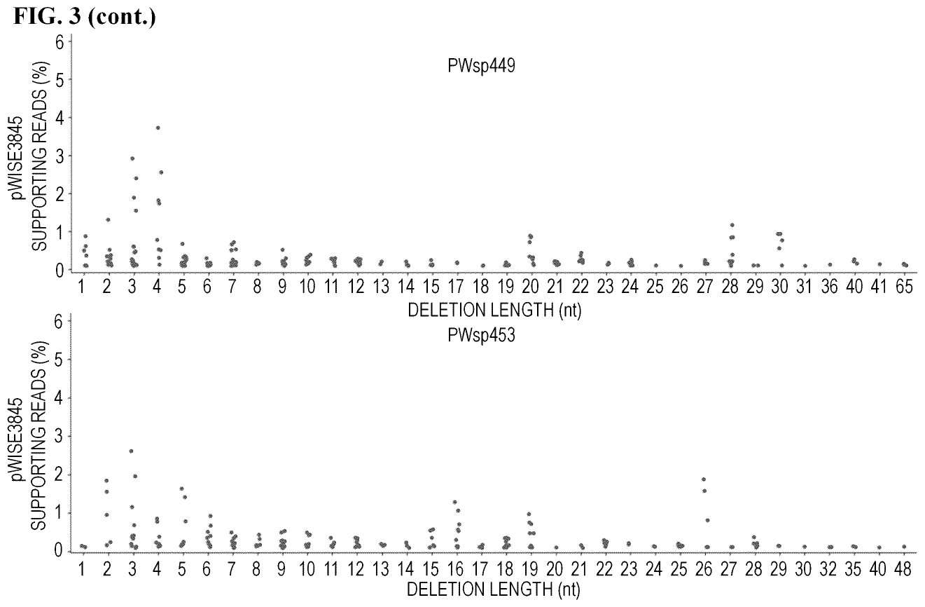

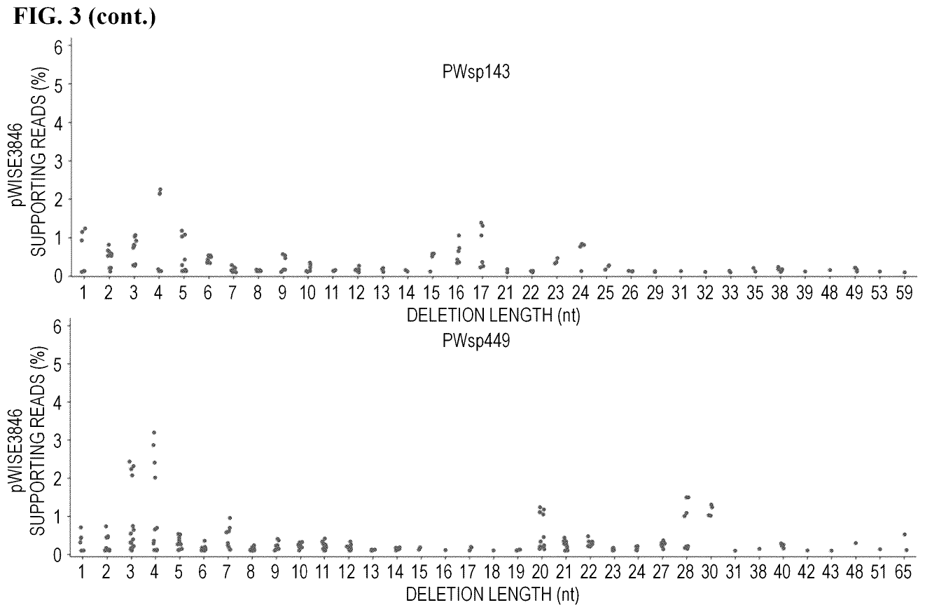

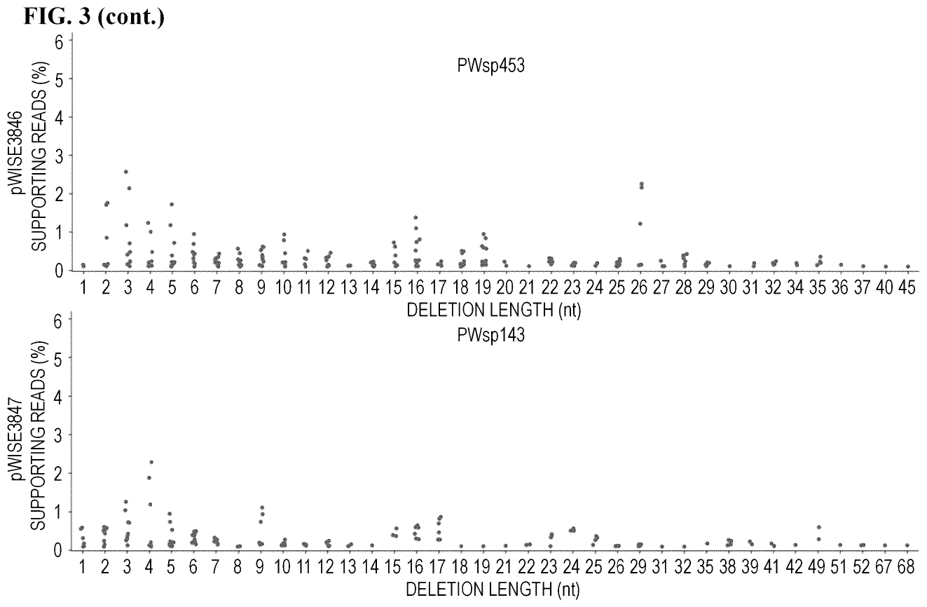

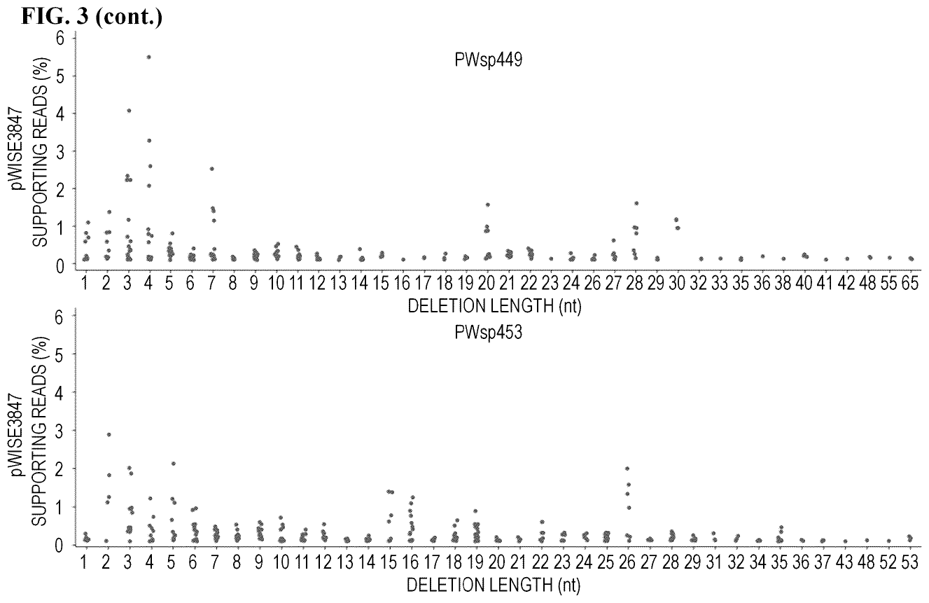

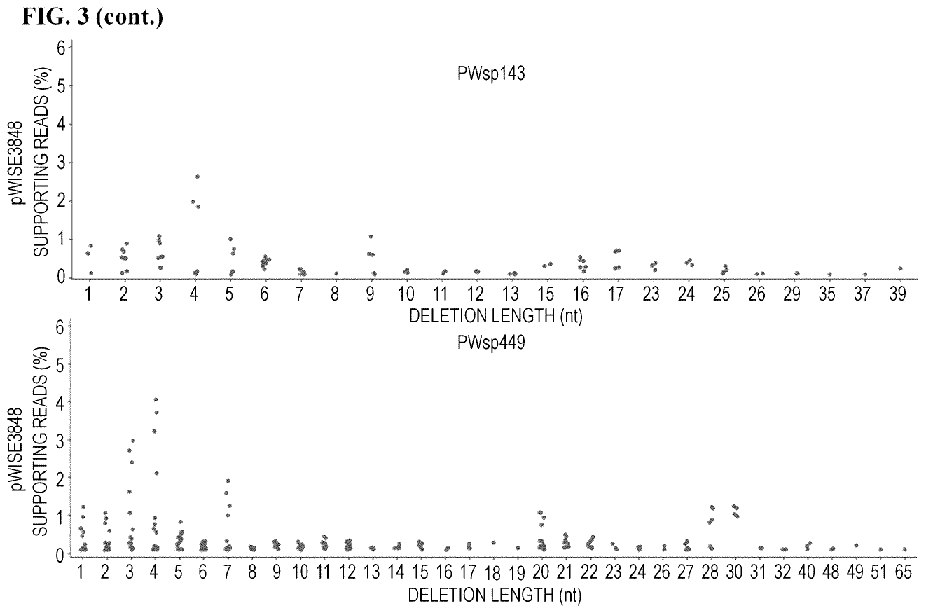

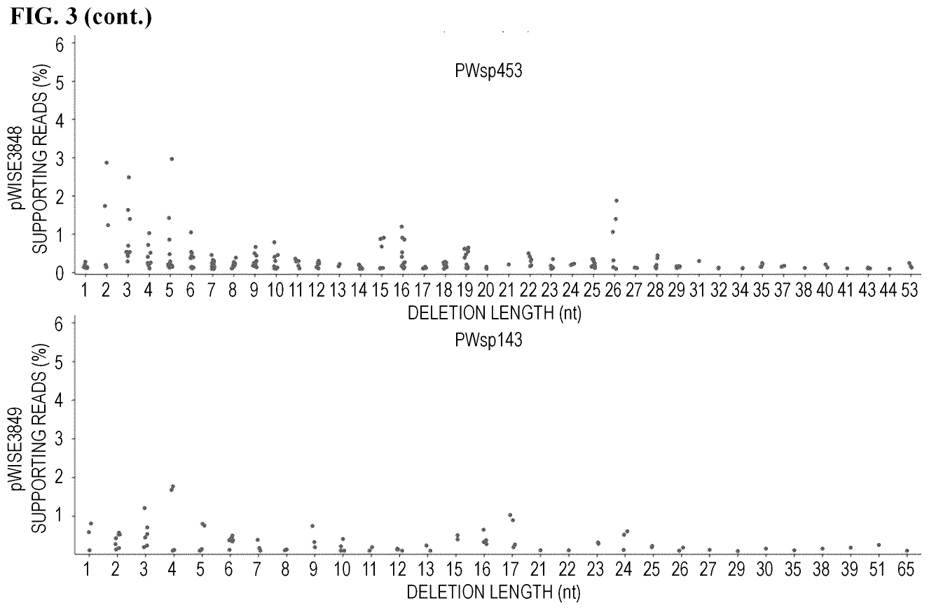

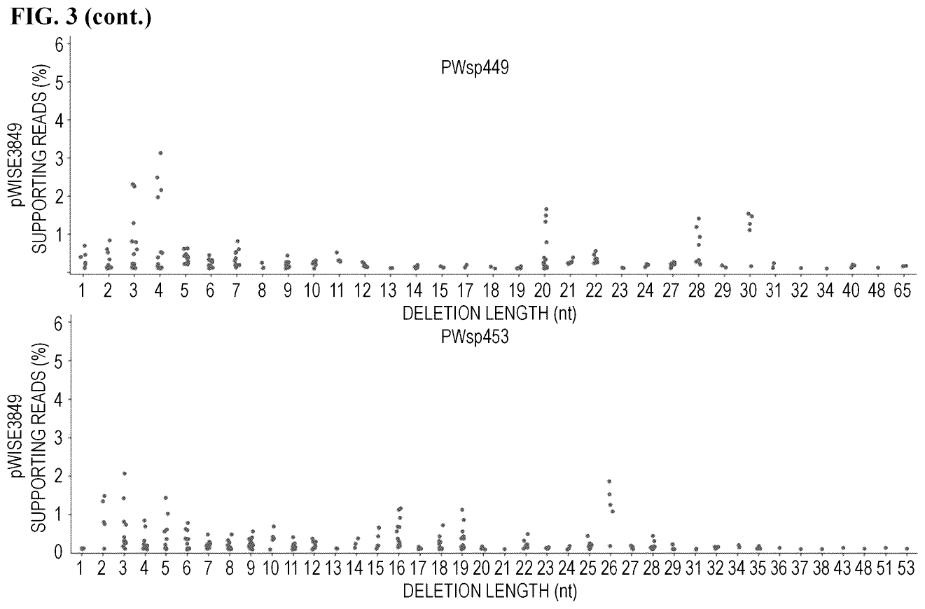

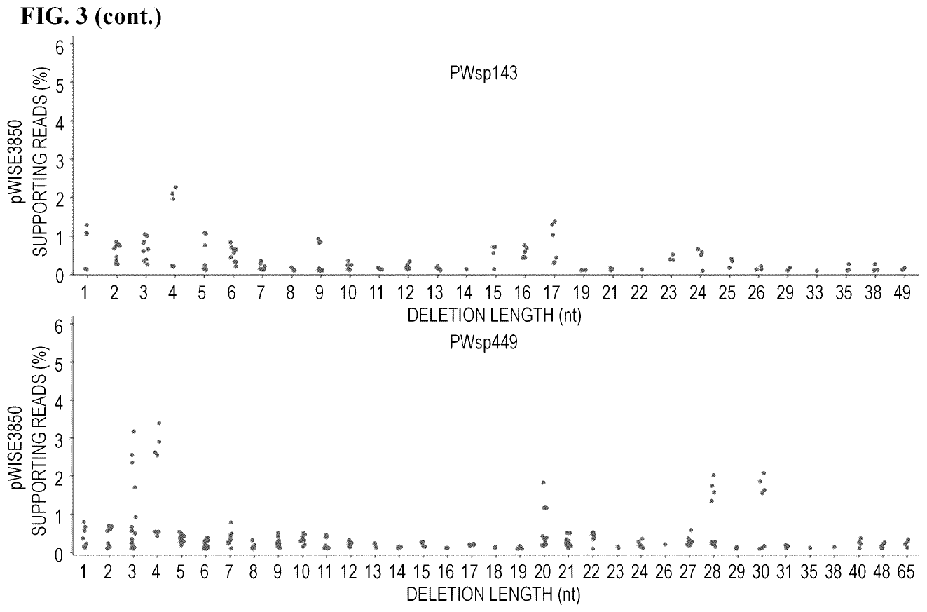

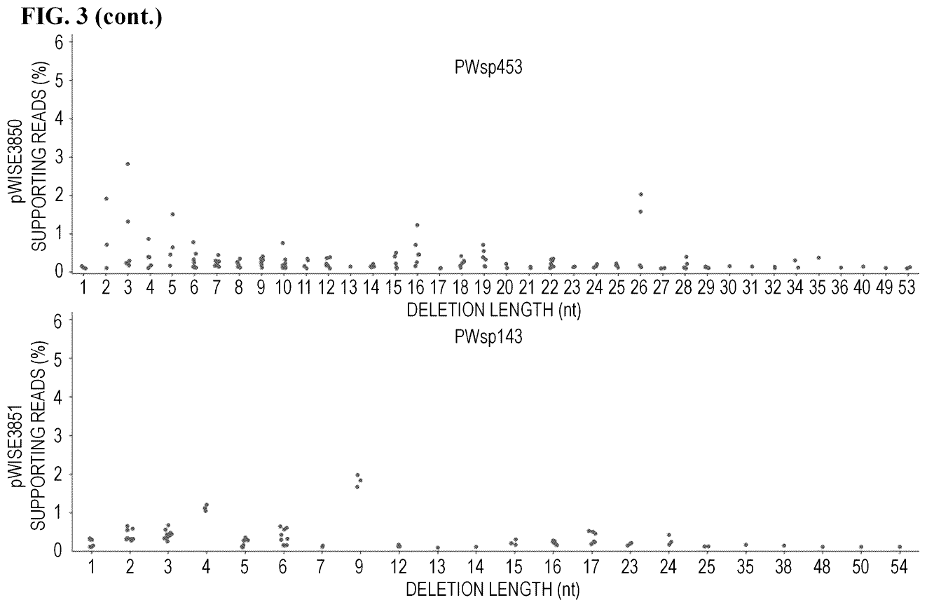

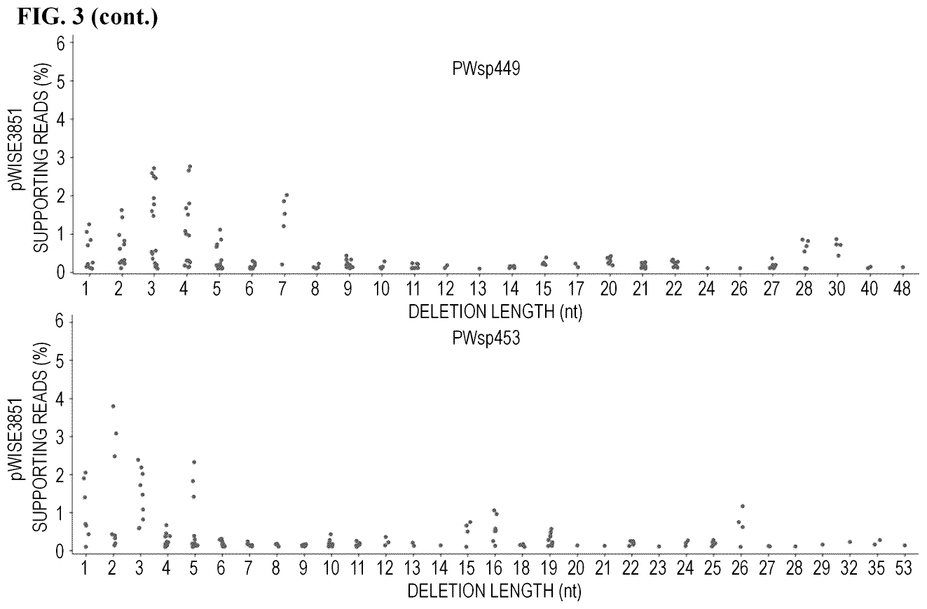

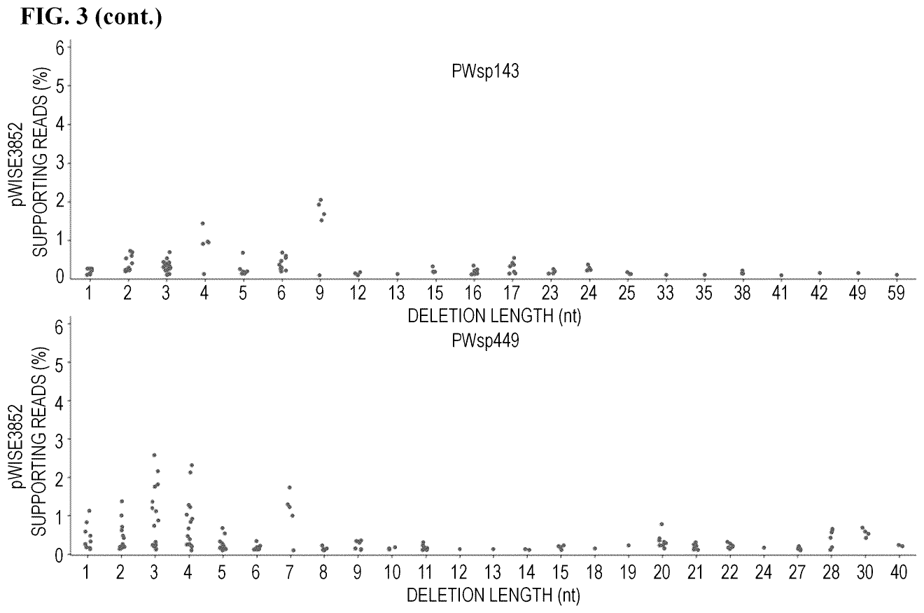

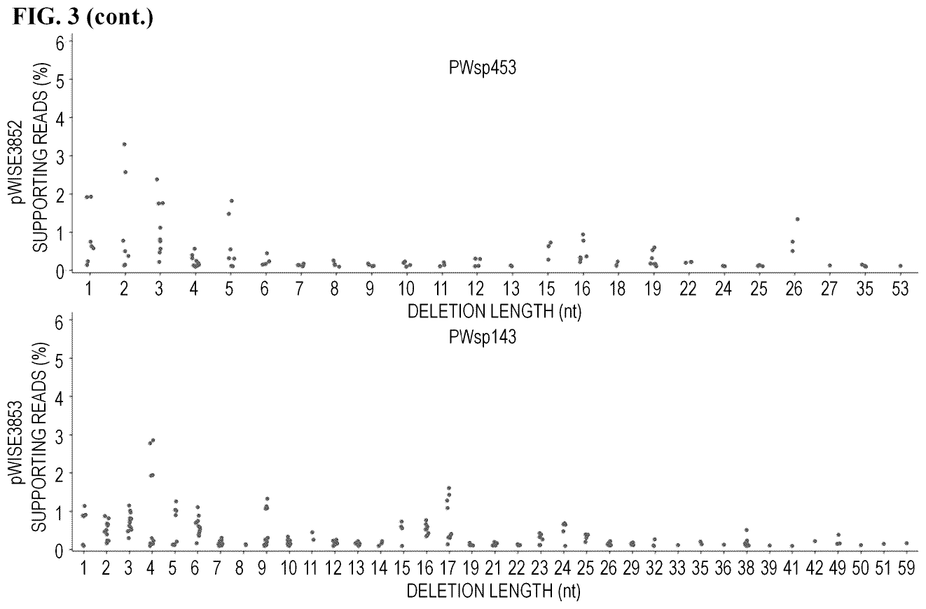

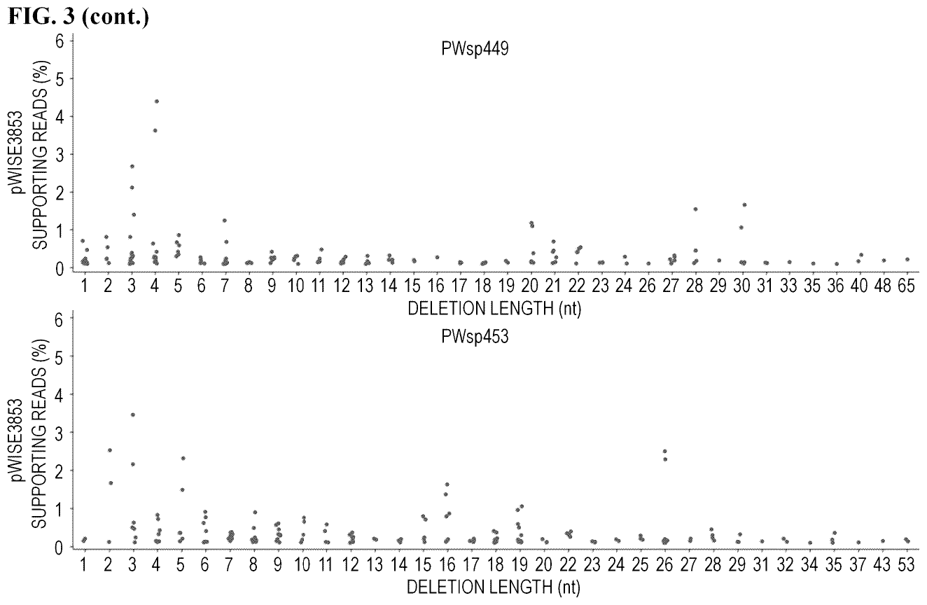

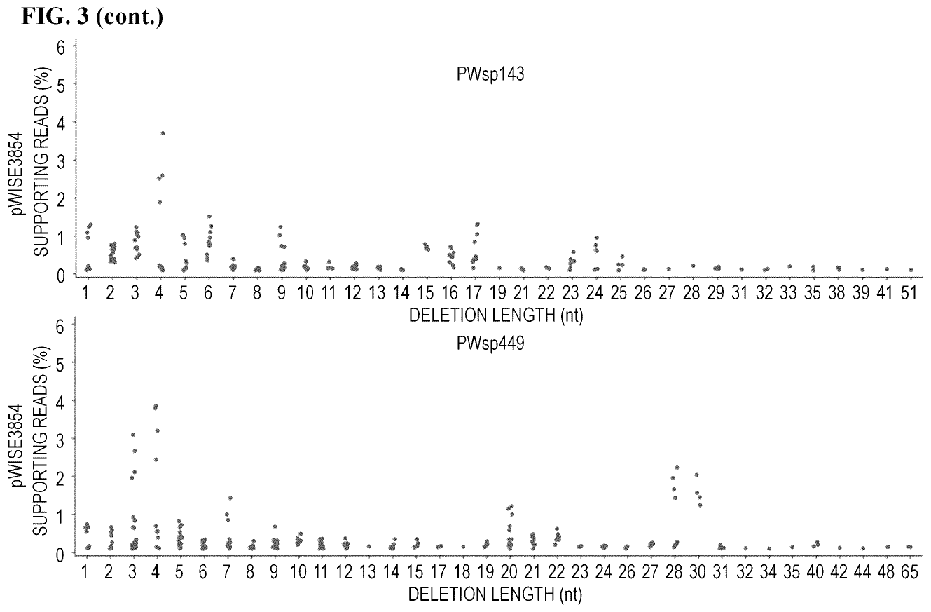

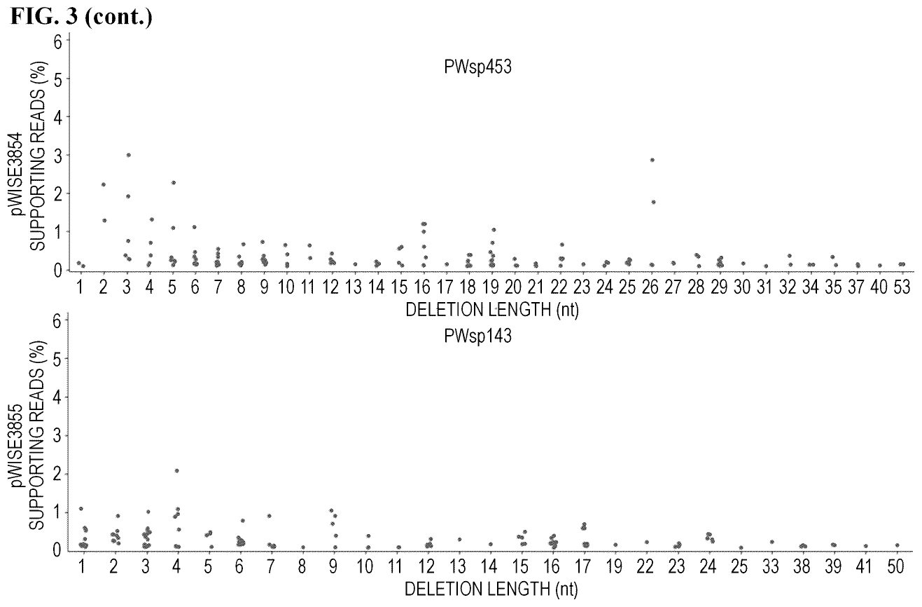

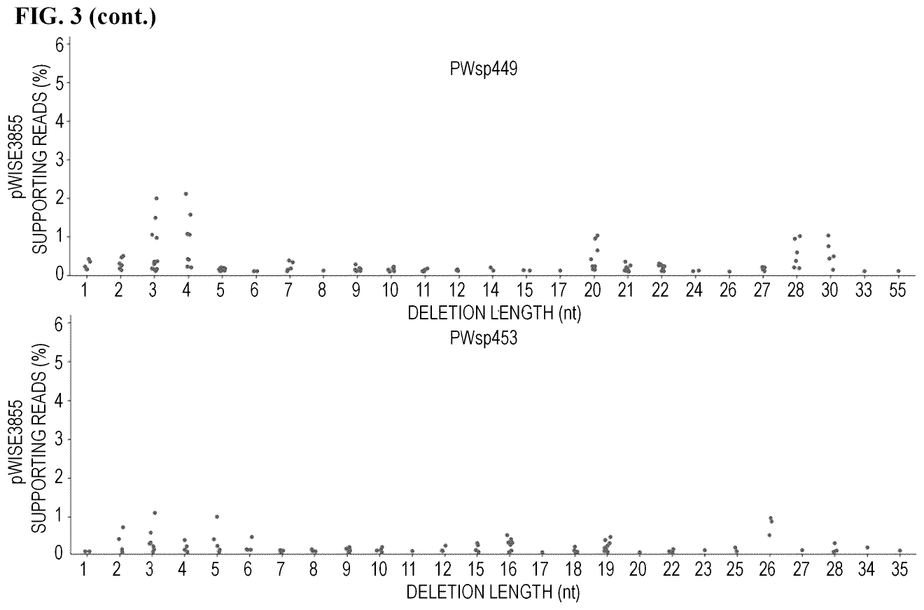

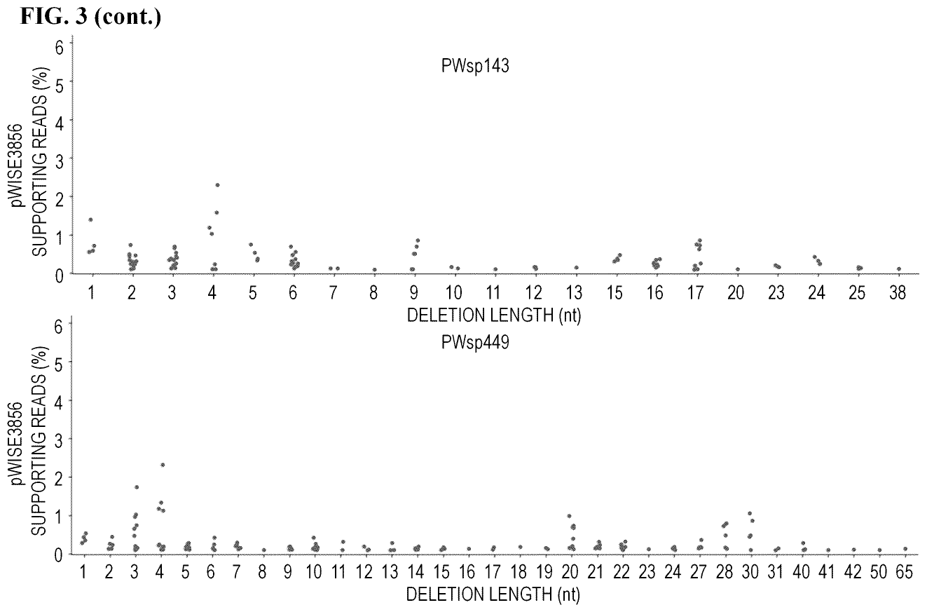

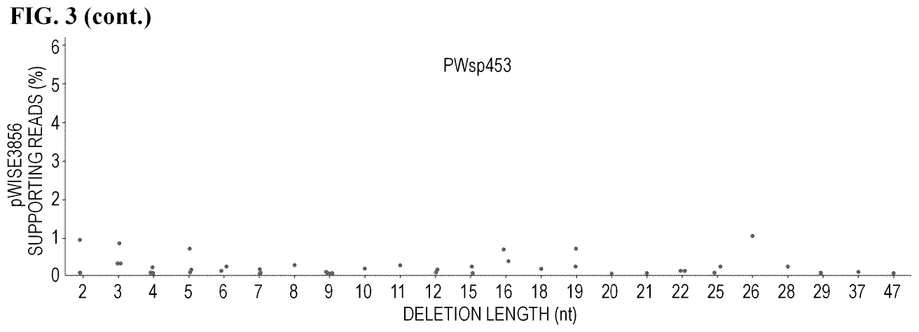

The results are shown in FIGS. 2-4 and demonstrate that all of the circular permutants are functional. As shown in FIG. 2, excellent INDELs were observed for control (WT-LbCas12a) at all three target sites and INDELs were generated at a high enough level for the circular permutants. For the control (pWISE121) and each circular permutant (pWISE8345-pWISE8356) the deletion lengths were parsed for each observed edit. As shown in FIG. 3, the deletion length distribution was examined at the three target sites (PWsp143, PWsp449, and PWsp453). The majority of deletions are between 1-5 base pair(s) (bp) long irrespective of the target. When looking at circular permutants compared to the controls, changes are observed in the deletion size distribution as shown in FIG. 3. For example, for pWISE8351 with PWsp143, it can be seen that 9 bp deletions were the most common, whereas, for the control (pWISE121) with PWsp143, 4 bp deletions were the most common. While at PWsp449 for pWISE8351, an increase in 1 and 2 bp deletions was seen compared to the control at PWsp449.

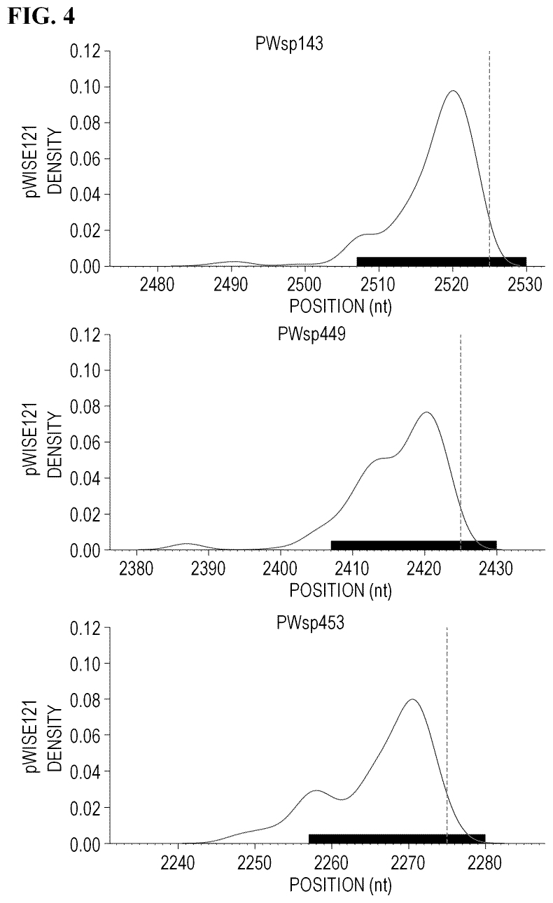

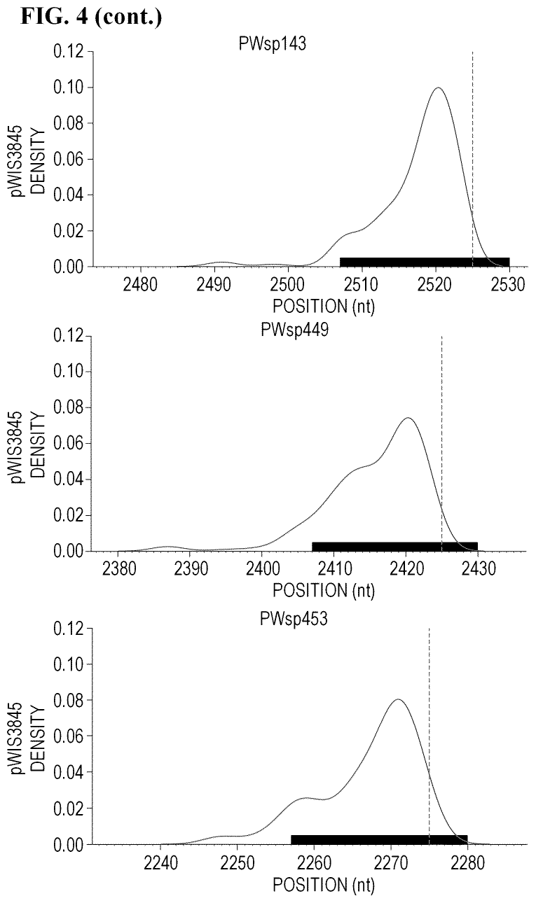

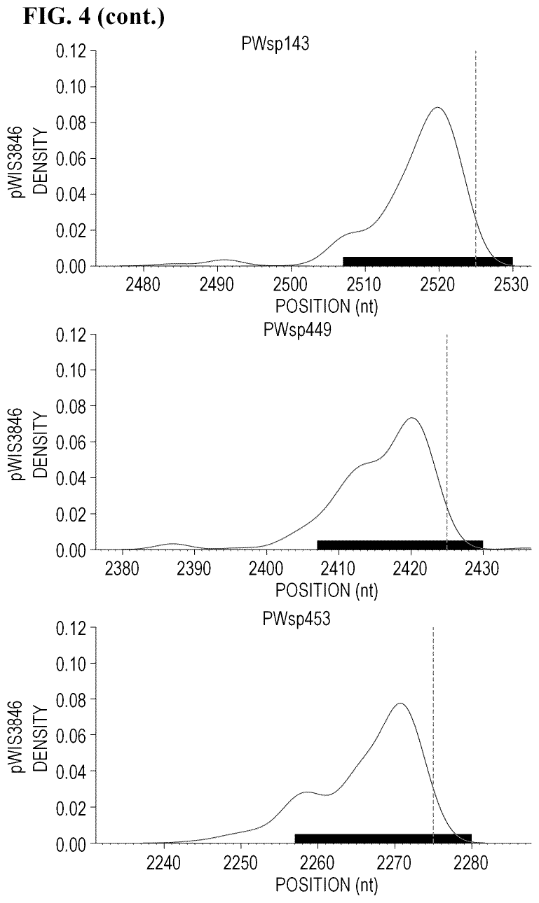

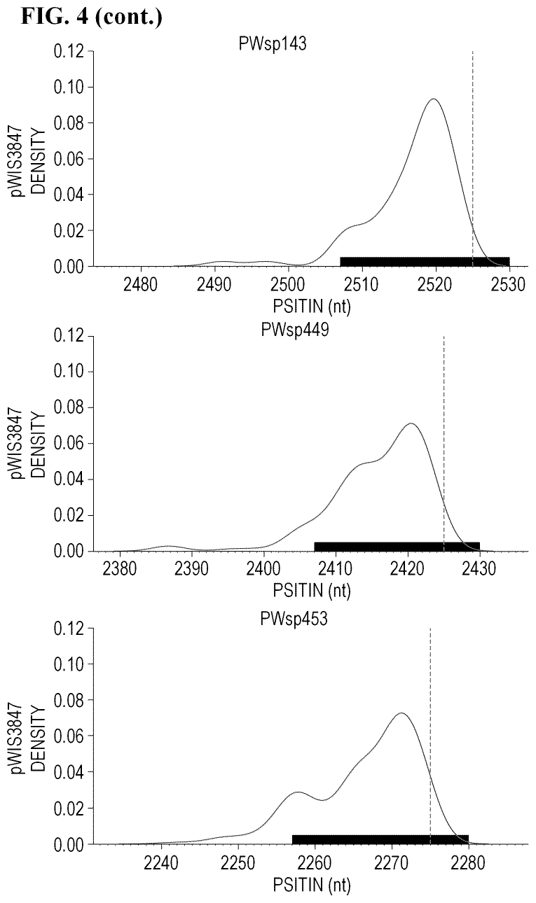

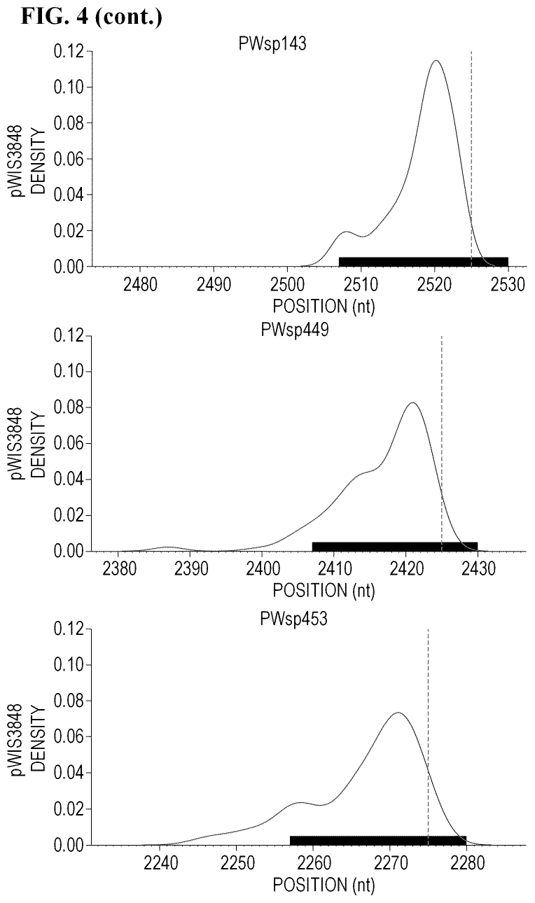

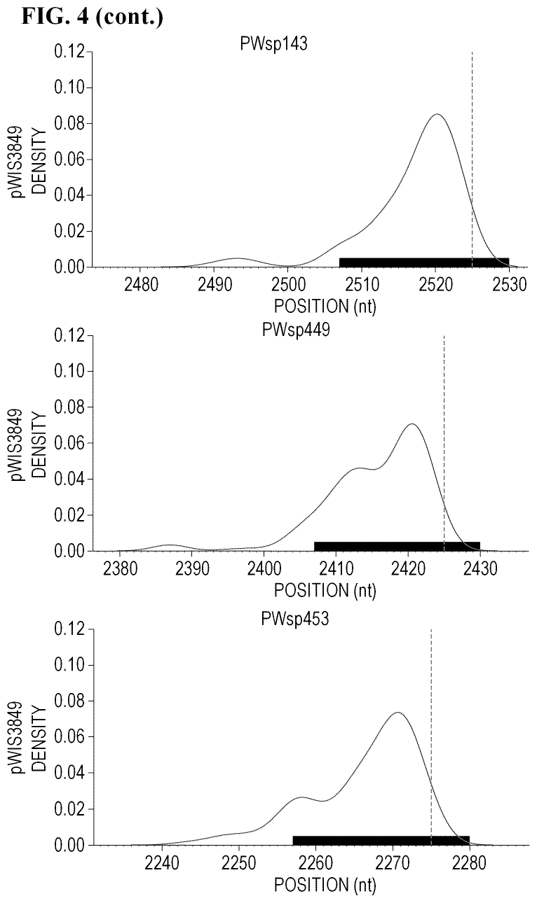

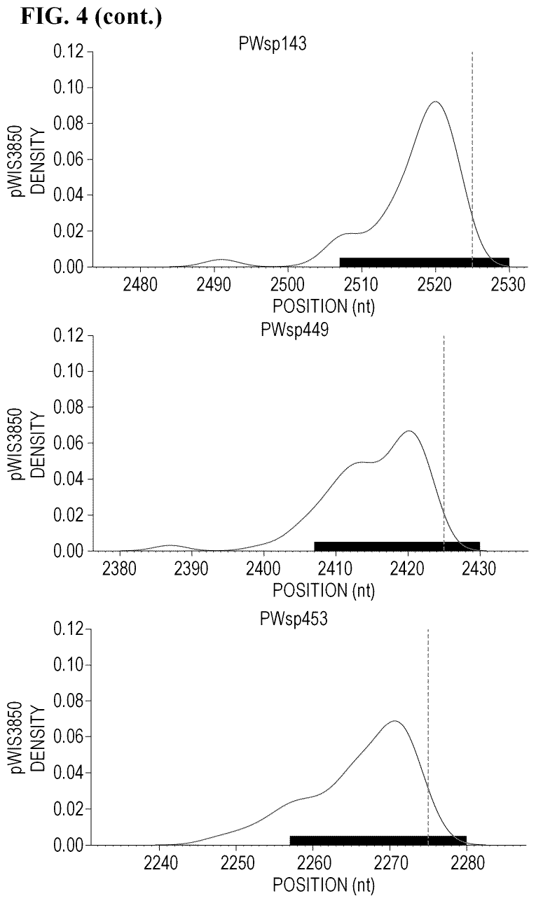

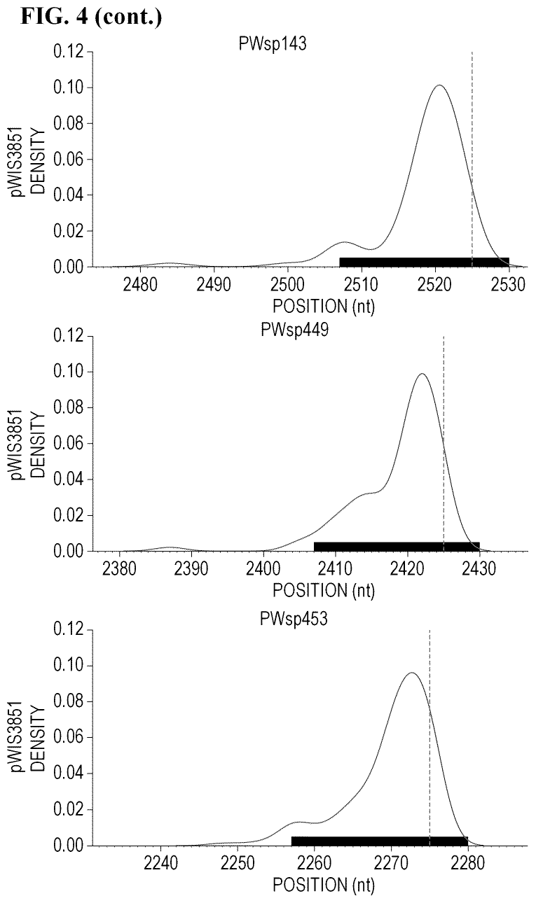

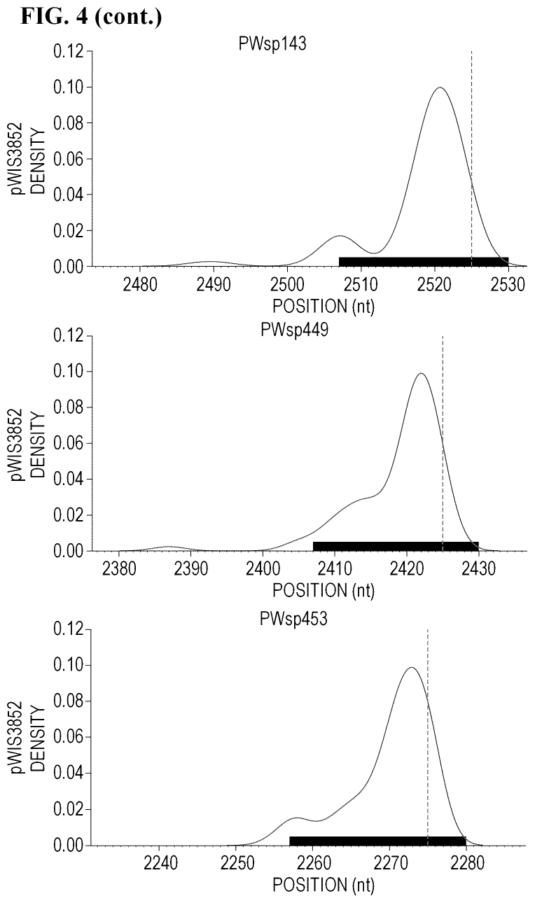

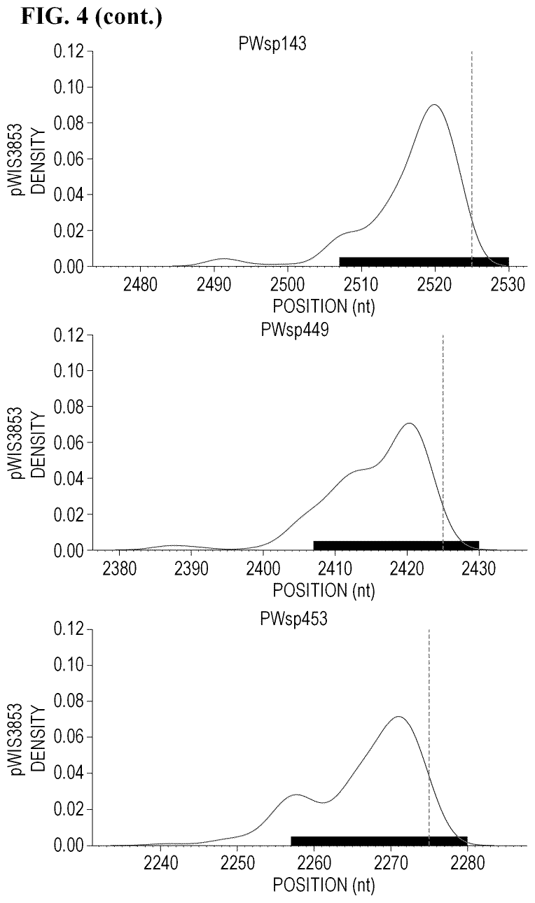

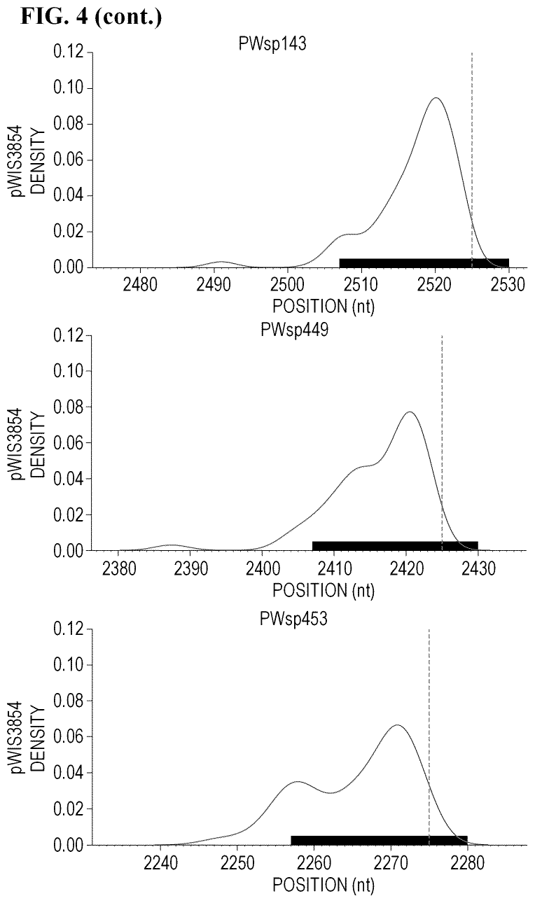

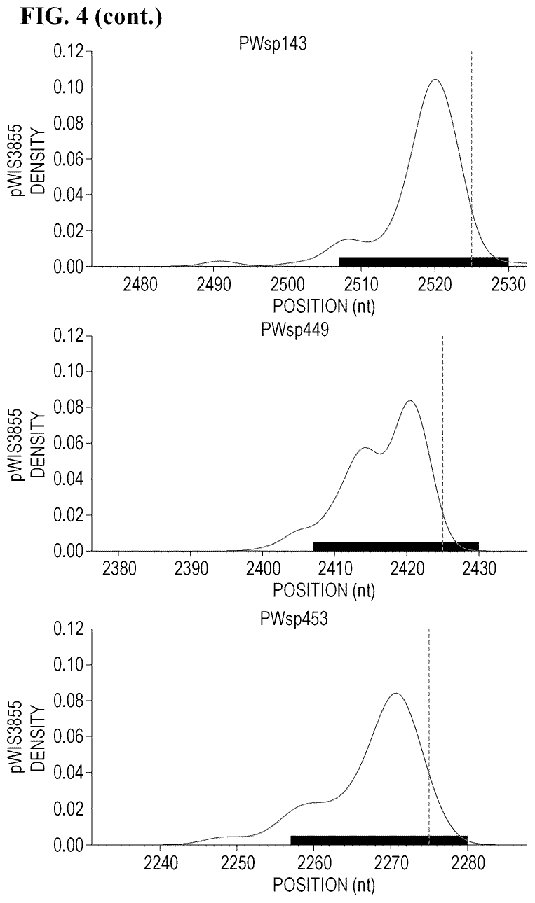

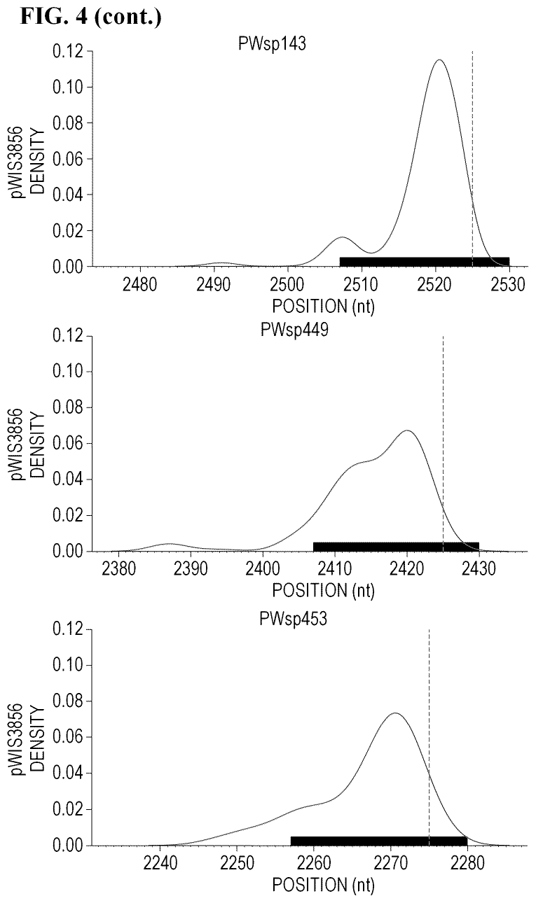

For the control (pWISE121) and each circular permutant (pWISE8345-pWISE8356) the deletion positions were parsed for each observed edit. As shown in FIG. 4, the deletion starting location distribution was examined at the three target sites (PWsp143, PWsp449, and PWsp453). Kenerl density estimate (kde) was used to fit a smoothed line to the observed distribution to aid in visualization. As can be seen in FIG. 4, there are clear differences in the location of the 1st bp of the deletion between circular permutants and the control. For example, as shown in FIG. 4, pWISE8352 shows a shift in the starting location towards the expected cut site on the target strand (dashed line) at all three sites. Conversely, as shown in FIG. 4, pWISE8354 shows a widening of the distribution with a shift away from the expected cut site at PWsp449 and PWsp453.

### Example 3

Circular permutants of LbCas12a according to some embodiments of the present invention were tested for REDRAW editing by fusing a mutated Moloney Murine Leukemia Virus Reverse Transcriptase (MMLV-RT (5M); SEQ ID NO:209) to either the N-terminus or C-terminus of the circular permutant to provide a fusion protein. These fusion proteins, along with the controls, are listed in Table 4.

TABLE 4
REDRAW constructs tested.

| | Vector SEQ | DNA SEQ | Protein SEQ |
|---|---|---|---|
| pWISE | ID NO: | ID NO: | ID NO: |
| pWISE121 | 259 | 246 | 56 |
| pWISE6099 | 886 | 948 | 158 |
| pWISE9459 | 377 | 364 | 351 |
| pWISE9460 | 378 | 365 | 352 |
| pWISE9461 | 379 | 366 | 353 |
| pWISE9462 | 380 | 367 | 354 |
| pWISE9463 | 381 | 368 | 355 |
| pWISE9464 | 382 | 369 | 356 |
| pWISE9465 | 383 | 370 | 357 |
| pWISE9466 | 384 | 371 | 358 |
| pWISE9467 | 385 | 372 | 359 |
| pWISE9468 | 386 | 373 | 360 |
| pWISE9469 | 387 | 374 | 361 |
| pWISE9470 | 388 | 375 | 362 |
| pWISE9471 | 389 | 376 | 363 |

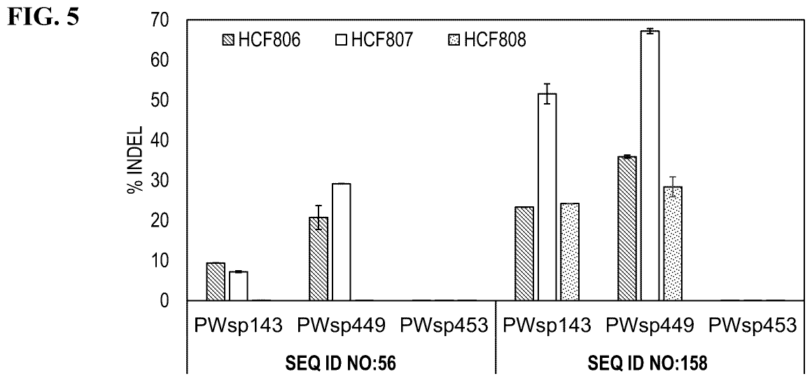

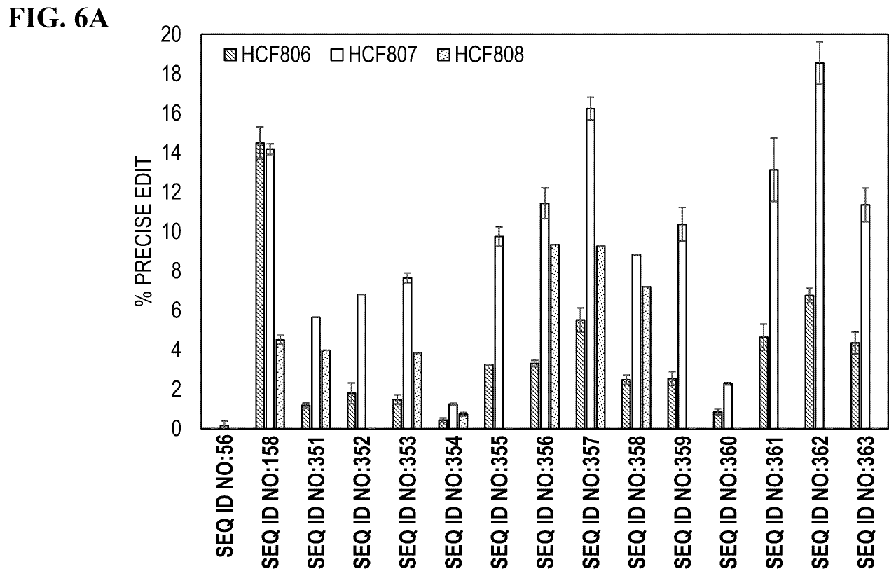

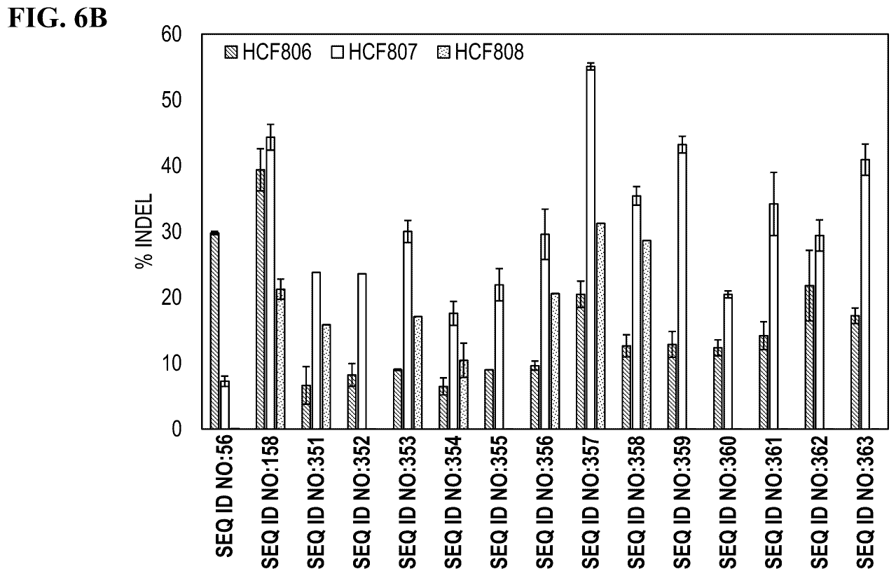

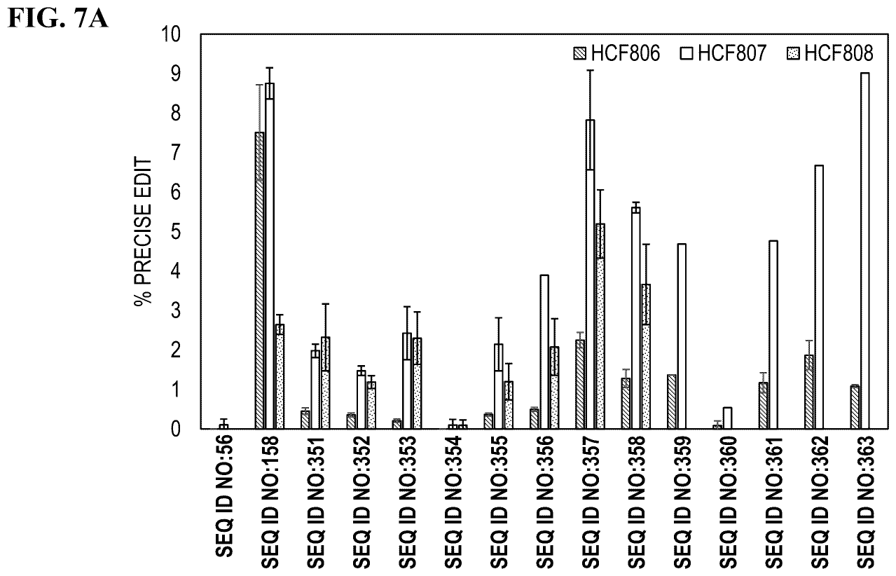

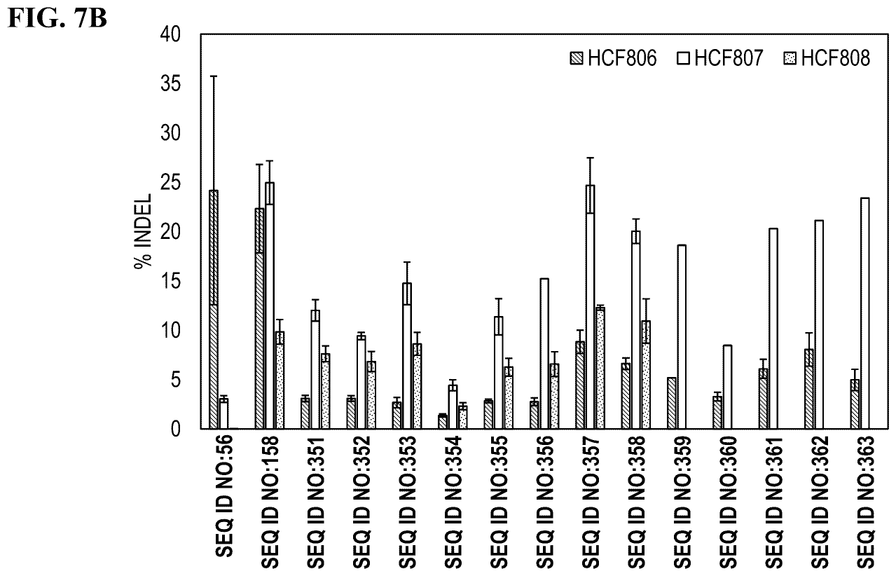

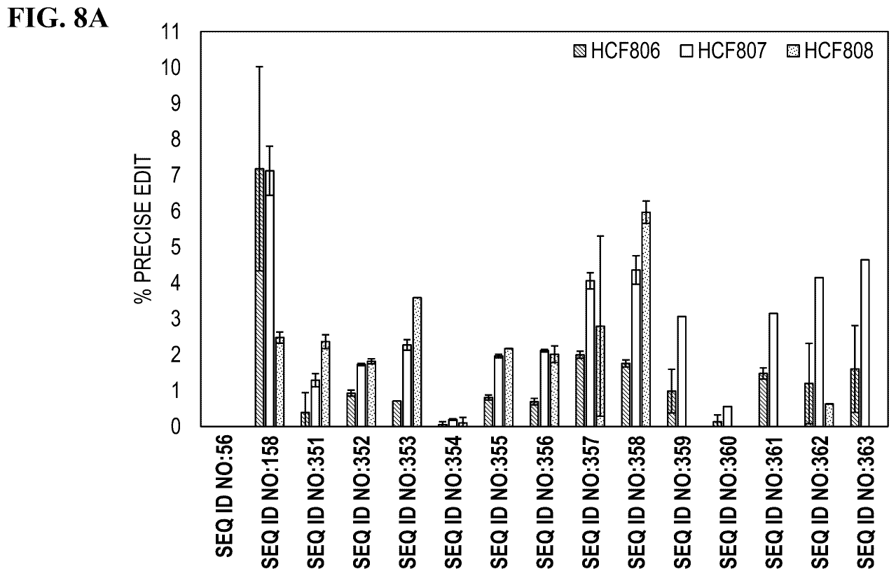

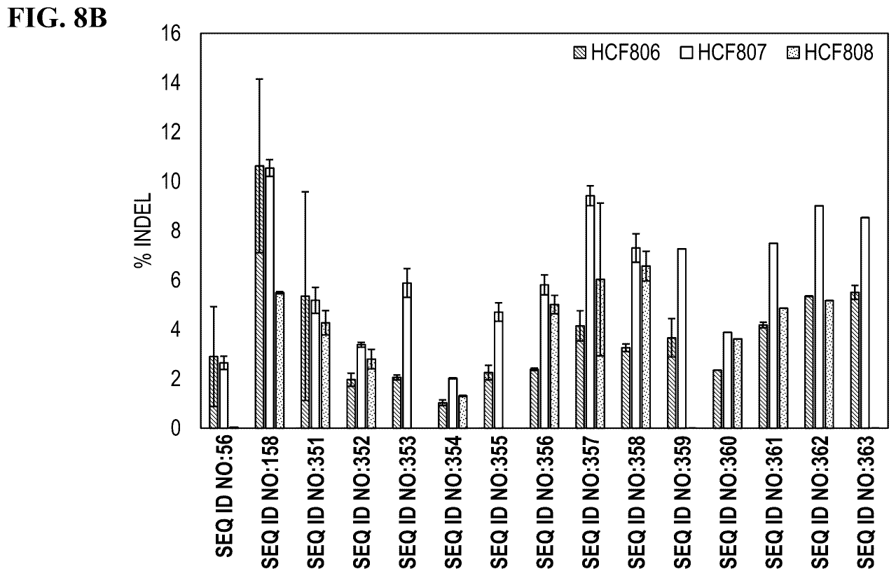

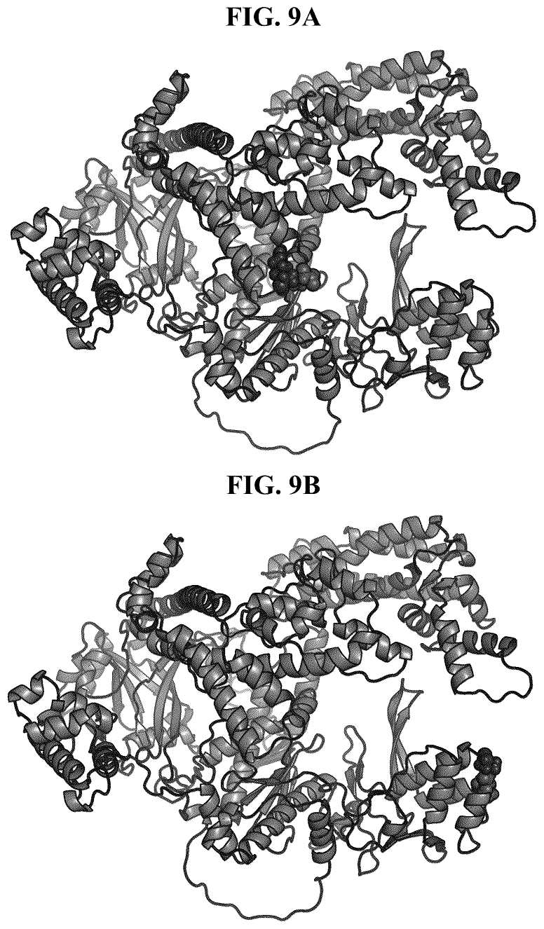

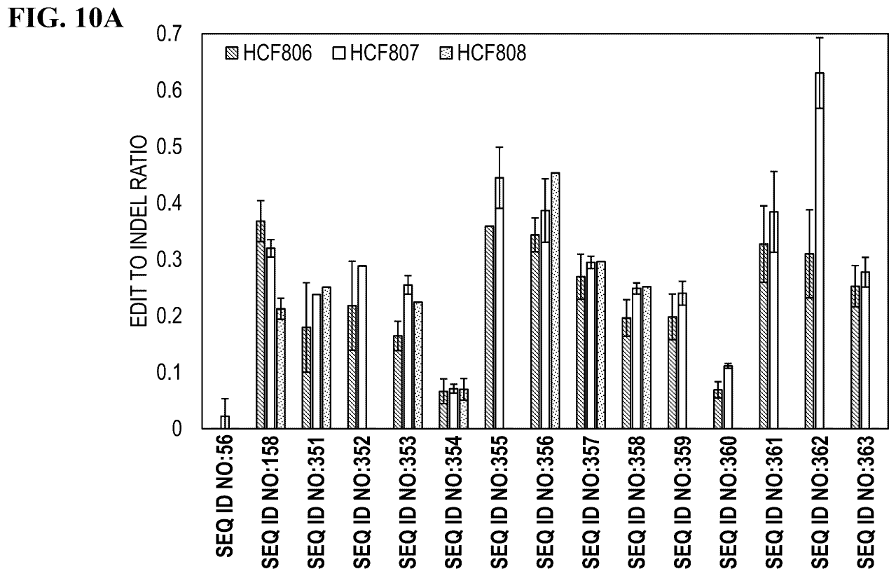

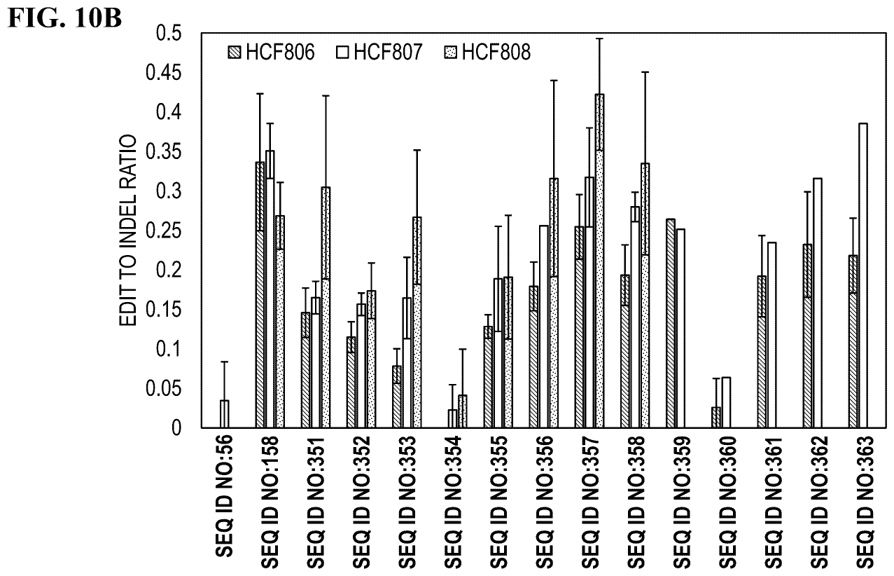

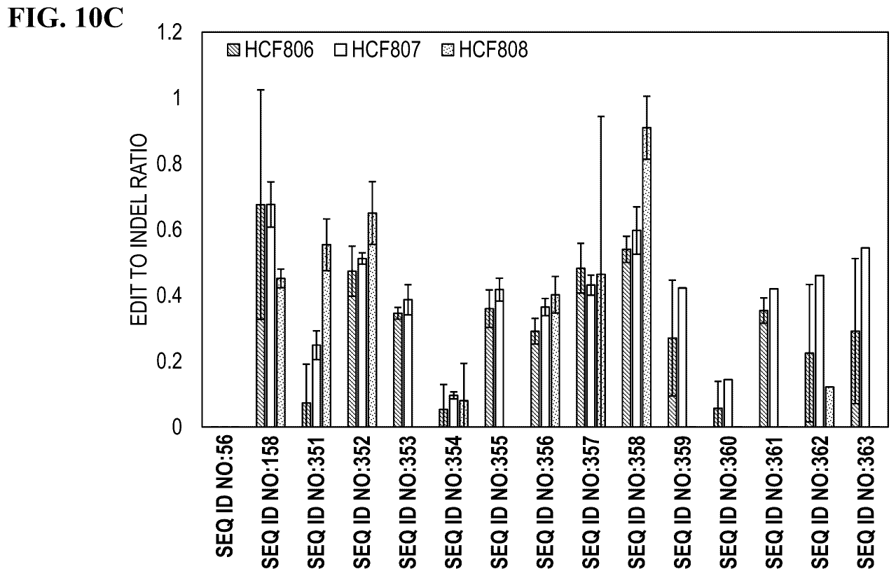

The fusion proteins were tested for their precise editing ability and ability to generate INDELS at three different target nucleic acids (SEQ ID NOs: 272-274) in HEK293T cells using three spacers (SEQ ID NOs: 275-277). Controls used in these experiments were LbCas12a (SEQ ID NO:56), RE2 (SEQ ID NO:158), and RE2 reverse (SEQ ID NO:363). Three biological replicates (HCF806, HCF807, and HCF808) and two technical replicates were tested. HEK293T cells were seeded into 48-well collagen-coated plates (Corning) in the absence of an antibiotic using DMEM media. At 70-80% confluency, cells were transfected with 1.5 μL of LTX (ThermoFisher Scientific) using 500 ng of the control or fusion protein plasmid and 500 ng of guide RNA plasmid according to manufacturer's protocol. After 3 days, the cells were lysed with a crude extraction method using TritonX buffer. Each control or fusion protein was scored based on the precise base pair editing and indel placement percentage in the DNMT1, FANCF, and RNF2 genes using the guide RNAs. Results are provided in FIGS. 6A, 6B, 7A, 7B, 8A, 8B, and 10A-10C and demonstrate that certain fusion proteins showed significant precise editing and indel frequency. In general, the fusion proteins with a C-terminal MMLV-RT (5M) showed slightly improved editing and INDEL efficiency in comparison to the fusion proteins with an N-terminal MMLV-RT (5M). Moreover, the fusion protein with the CP02 circular permutant and MMLV-RT (5M) at the C-terminus (SEQ ID NO: 357) and the fusion protein with the CP12 circular permutant and MMLV-RT (5M) at the C-terminus (SEQ ID NO:362) had the highest editing and INDEL efficiency. FIGS. 9A and 9B show that the newly placed termini of CP02 and CP12 are placed near the target strand DNA. The indel frequency of the tested guide RNAs, as crRNA, against the target DNMT1, FANCF, and RNF2 loci were tested as a control (FIG. 5).

### Example 4

Circular permutants of LbCas12a according to some embodiments of the present invention were tested for cytosine base editing by fusing a cytosine deaminase (SEQ ID NO: 798) to either the N-terminus or C-terminus of the circular permutant to provide a fusion protein. The tested constructs are provided in Table 5.

TABLE 5
Tested constructs

| | Vector SEQ | DNA SEQ | Protein SEQ |
|---|---|---|---|
| pWISE | ID NO: | ID NO: | ID NO: |
| pWISE121 | 259 | 246 | 56 |
| pWISE1921 | 444 | 436 | 428 |
| pWISE1925 | 445 | 437 | 429 |
| pWISE4332 | 446 | 438 | 430 |
| pWISE4339 | 447 | 439 | 431 |
| pWISE6852 | 448 | 440 | 432 |
| pWISE6853 | 449 | 441 | 433 |
| pWISE9117 | 474 | 462 | 450 |
| pWISE9118 | 475 | 463 | 451 |
| pWISE9119 | 476 | 464 | 452 |
| pWISE9120 | 477 | 465 | 453 |
| pWISE9121 | 478 | 466 | 454 |
| pWISE9122 | 479 | 467 | 455 |
| pWISE9123 | 480 | 468 | 456 |
| pWISE9124 | 481 | 469 | 457 |
| pWISE9125 | 482 | 470 | 458 |
| pWISE9126 | 483 | 471 | 459 |
| pWISE9127 | 484 | 472 | 460 |
| pWISE9128 | 485 | 473 | 461 |
| pWISE9134 | 442 | 434 | 426 |
| pWISE9135 | 443 | 435 | 427 |

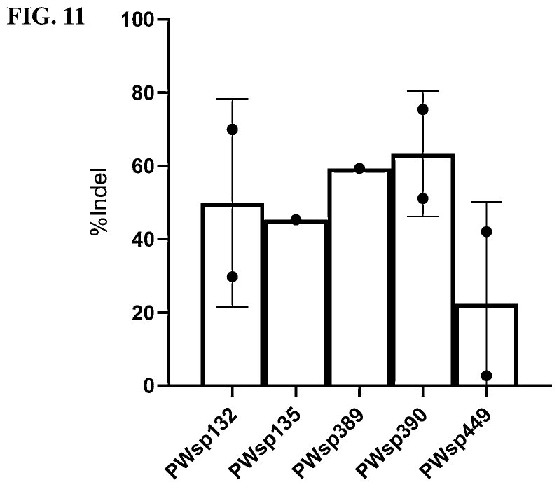

The fusion proteins and controls were tested for their ability to perform base editing at five different sites in three target nucleic acids (SEQ ID NOs: 273, 863, and 864) in HEK293T cells using five spacers (SEQ ID NOs: 276, and 799-802). A single biological replicate and two technical replicates were tested. Controls used in this study were: pWISE9134 which is a dead LbCas12a fused to a cytosine deaminase by a GS-XTEN-GS linker; pWISE9135 which is a dead LbCas12a fused a cytosine deaminase at the C-terminus of the dead LbCas12a via a GS-XTEN-GS linker; and pWISE121 which is wild-type LbCas12a. HEK293T cells were seeded into 48-well collagen-coated plates (Corning) in the absence of an antibiotic using DMEM media. At 70-80% confluency, cells were transfected with 1.5 μL of LTX (ThermoFisher Scientific) using 500 ng of the control or fusion protein plasmid and 500 ng of guide RNA plasmid according to manufacturer's protocol. After 3 days, the cells were lysed with a crude extraction method using TritonX buffer. Each control or fusion protein was scored based on the precise base pair editing in the FANCF, RUNX1, and AAVS1 genes using the guide RNAs (SEQ ID NOs: 276 and 799-802). These guide RNAs were also tested using control LbCas12a (SEQ ID NO:56) as shown in FIG. 11. The results are provided in Tables 6-25. As seen in Tables 6, 7, 10, 11, 14, 15, 18, 19, 22, and 23, fusion of a cytosine deaminase at the N-terminus of a circular permutant produced effective C to T editing on the non-target DNA strand in almost all genes. Surprisingly, the fusion protein with circular permutant CP08 (SEQ ID NO:453) demonstrated G to A editing on the non-target strand, corresponding to C to T changes on the target strand (Tables 8, 9, 12, 13, 16, 17, 20, 21, 24, and 25). The values in Tables 6-25 that are below 0.1% are considered to be in the noise of the instrument (below the limit of detection) and are not indicative of editing. Values that are between 0.1% and 0.5% indicate that editing is present in the experiment at the specified location, but the assay is not sensitive enough to accurately quantify the amount of base editing. This was unexpected as typical cytosine base editors (e.g., SEQ ID NOs: 426-429) are not capable of deaminating cytosines on the target strand within the spacer sequence.

TABLE 6

| | | Average C to T editing percent at the nucleotides numbered | | | | | | |
|---|---|---|---|---|---|---|---|---|
| | Protein | according to the position in the PWsp132 spacer sequence | | | | | | |
| | SEQ | on the non-target, genomic DNA strand | | | | | | |
| pWISE | ID NO: | C.-7 | C.-6 | C.-4 | C.-1 | C.2 | C.3 | C.11 |
| pWISE121 | 56 | 0.004% | 0.002% | 0.000% | 0.004% | 0.000% | 0.063% | 0.138% |
| pWISE1921 | 428 | 0.127% | 0.068% | 0.030% | 0.445% | 0.049% | 0.183% | 24.432% |
| pWISE1925 | 429 | 0.132% | 0.086% | 0.054% | 0.481% | 0.028% | 0.100% | 18.509% |
| pWISE4332 | 430 | 0.086% | 0.052% | 0.006% | 0.348% | 0.004% | 0.028% | 13.214% |
| pWISE4339 | 431 | 0.541% | 0.548% | 0.008% | 0.119% | 0.003% | 0.011% | 14.075% |
| pWISE6852 | 432 | 0.045% | 0.020% | 0.004% | 0.128% | 0.001% | 0.009% | 13.502% |
| pWISE6853 | 433 | 0.005% | 0.004% | 0.000% | 0.396% | 0.005% | 0.033% | 20.059% |
| pWISE9117 | 450 | 0.090% | 0.079% | 0.014% | 0.498% | 0.024% | 0.024% | 22.359% |
| pWISE9118 | 451 | 0.826% | 0.832% | 0.000% | 0.828% | 0.003% | 0.001% | 7.529% |
| pWISE9119 | 452 | 0.289% | 0.075% | 0.000% | 0.717% | 0.005% | 0.011% | 31.457% |
| pWISE9120 | 453 | 0.005% | 0.005% | 0.000% | 0.432% | 0.000% | 0.005% | 1.165% |
| pWISE9121 | 454 | 0.068% | 0.049% | 0.011% | 0.200% | 0.009% | 0.003% | 5.654% |
| pWISE9122 | 455 | 0.146% | 0.060% | 0.017% | 0.822% | 0.016% | 0.331% | 19.868% |
| pWISE9123 | 456 | 0.132% | 0.030% | 0.003% | 0.639% | 0.042% | 0.066% | 33.596% |
| pWISE9124 | 457 | 0.045% | 0.027% | 0.013% | 0.203% | 0.002% | 0.009% | 12.865% |
| pWISE9125 | 458 | 0.347% | 0.116% | 0.031% | 0.907% | 0.063% | 0.046% | 24.770% |
| pWISE9126 | 459 | 0.052% | 0.027% | 0.000% | 0.065% | 0.010% | 0.012% | 2.197% |
| pWISE9127 | 460 | 0.027% | 0.002% | 0.012% | 0.072% | 0.006% | 0.003% | 2.337% |
| pWISE9128 | 461 | 0.193% | 0.064% | 0.028% | 1.092% | 0.009% | 0.009% | 26.677% |
| pWISE9134 | 426 | No data | No data | No data | No data | No data | No data | No data |
| pWISE9135 | 427 | 0.495% | 0.225% | 0.071% | 1.249% | 0.065% | 0.106% | 30.645% |

| | | Average C to T editing percent at the nucleotides numbered | | | | | |
|---|---|---|---|---|---|---|---|
| | Protein | according to the position in the PWsp132 spacer sequence | | | | | |
| | SEQ | on the non-target, genomic DNA strand | | | | | |
| pWISE | ID NO: | C.14 | C.17 | C.19 | C.21 | C.24 | C.28 |
| pWISE121 | 56 | 0.277% | 0.166% | 0.468% | 0.077% | 0.141% | 0.100% |
| pWISE1921 | 428 | 14.118% | 0.888% | 12.688% | 12.201% | 0.077% | 0.000% |
| pWISE1925 | 429 | 9.889% | 0.804% | 10.183% | 7.735% | 0.065% | 0.000% |
| pWISE4332 | 430 | 10.446% | 0.655% | 7.603% | 4.914% | 0.088% | 0.004% |
| pWISE4339 | 431 | 10.742% | 0.229% | 3.610% | 1.972% | 0.030% | 0.006% |
| pWISE6852 | 432 | 9.480% | 0.205% | 7.076% | 4.012% | 0.000% | 0.001% |
| pWISE6853 | 433 | 14.378% | 0.274% | 7.868% | 3.897% | 0.056% | 0.005% |
| pWISE9117 | 450 | 15.118% | 1.821% | 13.456% | 7.506% | 0.068% | 0.017% |
| pWISE9118 | 451 | 4.418% | 1.974% | 7.519% | 10.685% | 0.001% | 0.001% |
| pWISE9119 | 452 | 29.350% | 5.438% | 25.789% | 29.548% | 1.016% | 0.075% |
| pWISE9120 | 453 | 0.742% | 0.005% | 0.785% | 2.237% | 0.371% | 0.000% |
| pWISE9121 | 454 | 3.083% | 0.809% | 2.868% | 4.221% | 0.030% | 0.006% |
| pWISE9122 | 455 | 22.961% | 1.819% | 18.625% | 18.934% | 0.530% | 0.015% |
| pWISE9123 | 456 | 30.464% | 3.149% | 25.577% | 18.123% | 0.084% | 0.021% |
| pWISE9124 | 457 | 10.678% | 1.617% | 13.999% | 10.984% | 0.088% | 0.013% |
| pWISE9125 | 458 | 13.647% | 3.699% | 17.469% | 5.738% | 0.065% | 0.029% |
| pWISE9126 | 459 | 0.858% | 0.193% | 1.201% | 1.043% | 0.072% | 0.002% |
| pWISE9127 | 460 | 0.791% | 0.040% | 0.819% | 0.713% | 0.013% | 0.002% |
| pWISE9128 | 461 | 16.895% | 0.743% | 9.214% | 10.269% | 0.101% | 0.000% |
| pWISE9134 | 426 | No data | No data | No data | No data | No data | No data |
| pWISE9135 | 427 | 17.210% | 4.584% | 20.697% | 7.073% | 0.079% | 0.010% |

TABLE 7

| | | Standard Deviation of the average editing percent at the nucleotides | | | | | | |
|---|---|---|---|---|---|---|---|---|
| | Protein | numbered according to the position in the PWsp132 spacer | | | | | | |
| | SEQ | sequence on the non-target, genomic DNA strand | | | | | | |
| pWISE | ID NO: | C.-7 | C.-6 | C.-4 | C.-1 | C.2 | C.3 | C.11 |
| pWISE121 | 56 | 0.000% | 0.003% | 0.000% | 0.005% | 0.000% | 0.089% | 0.149% |
| pWISE1921 | 428 | No data | No data | No data | No data | No data | No data | No data |
| pWISE1925 | 429 | 0.139% | 0.025% | 0.064% | 0.151% | 0.027% | 0.058% | 0.616% |
| pWISE4332 | 430 | No data | No data | No data | No data | No data | No data | No data |
| pWISE4339 | 431 | 0.708% | 0.688% | 0.011% | 0.168% | 0.004% | 0.006% | 2.500% |
| pWISE6852 | 432 | 0.064% | 0.020% | 0.005% | 0.173% | 0.001% | 0.012% | 2.875% |
| pWISE6853 | 433 | 0.007% | 0.004% | 0.000% | 0.335% | 0.007% | 0.047% | 0.156% |
| pWISE9117 | 450 | 0.091% | 0.065% | 0.020% | 0.114% | 0.015% | 0.012% | 0.380% |
| pWISE9118 | 451 | No data | No data | No data | No data | No data | No data | No data |
| pWISE9119 | 452 | No data | No data | No data | No data | No data | No data | No data |
| pWISE9120 | 453 | No data | No data | No data | No data | No data | No data | No data |
| pWISE9121 | 454 | 0.088% | 0.069% | 0.016% | 0.248% | 0.004% | 0.005% | 0.060% |
| pWISE9122 | 455 | 0.199% | 0.077% | 0.024% | 0.101% | 0.022% | 0.329% | 4.707% |
| pWISE9123 | 456 | No data | No data | No data | No data | No data | No data | No data |
| pWISE9124 | 457 | 0.063% | 0.038% | 0.001% | 0.067% | 0.002% | 0.012% | 1.371% |
| pWISE9125 | 458 | No data | No data | No data | No data | No data | No data | No data |
| pWISE9126 | 459 | 0.072% | 0.034% | 0.000% | 0.082% | 0.014% | 0.017% | 0.007% |
| pWISE9127 | 460 | 0.039% | 0.002% | 0.017% | 0.085% | 0.009% | 0.004% | 0.227% |
| pWISE9128 | 461 | No data | No data | No data | No data | No data | No data | No data |
| pWISE9134 | 426 | No data | No data | No data | No data | No data | No data | No data |
| pWISE9135 | 427 | No data | No data | No data | No data | No data | No data | No data |

| | | Standard Deviation of the average editing percent at the nucleotides | | | | | |
|---|---|---|---|---|---|---|---|
| | Protein | numbered according to the position in the PWsp132 spacer | | | | | |
| | SEQ | sequence on the non-target, genomic DNA strand | | | | | |
| pWISE | ID NO: | C.14 | C.17 | C.19 | C.21 | C.24 | C.28 |
| pWISE121 | 56 | 0.345% | 0.235% | 0.318% | 0.085% | 0.195% | 0.141% |
| pWISE1921 | 428 | No data | No data | No data | No data | No data | No data |
| pWISE1925 | 429 | 0.182% | 0.041% | 0.079% | 0.074% | 0.005% | 0.000% |
| pWISE4332 | 430 | No data | No data | No data | No data | No data | No data |
| pWISE4339 | 431 | 1.628% | 0.323% | 2.811% | 1.947% | 0.042% | 0.002% |
| pWISE6852 | 432 | 0.924% | 0.291% | 1.374% | 0.030% | 0.000% | 0.002% |
| pWISE6853 | 433 | 2.280% | 0.388% | 1.603% | 0.517% | 0.079% | 0.007% |
| pWISE9117 | 450 | 0.302% | 0.302% | 0.402% | 0.432% | 0.032% | 0.022% |
| pWISE9118 | 451 | No data | No data | No data | No data | No data | No data |
| pWISE9119 | 452 | No data | No data | No data | No data | No data | No data |
| pWISE9120 | 453 | No data | No data | No data | No data | No data | No data |
| pWISE9121 | 454 | 0.089% | 0.044% | 0.432% | 0.199% | 0.026% | 0.001% |
| pWISE9122 | 455 | 5.263% | 0.317% | 5.876% | 5.510% | 0.335% | 0.013% |
| pWISE9123 | 456 | No data | No data | No data | No data | No data | No data |
| pWISE9124 | 457 | 0.495% | 0.194% | 0.979% | 0.717% | 0.042% | 0.009% |
| pWISE9125 | 458 | No data | No data | No data | No data | No data | No data |
| pWISE9126 | 459 | 0.161% | 0.015% | 0.187% | 0.023% | 0.067% | 0.003% |
| pWISE9127 | 460 | 0.101% | 0.057% | 0.104% | 0.036% | 0.010% | 0.002% |
| pWISE9128 | 461 | No data | No data | No data | No data | No data | No data |
| pWISE9134 | 426 | No data | No data | No data | No data | No data | No data |
| pWISE9135 | 427 | No data | No data | No data | No data | No data | No data |

TABLE 8

| | | Average G to A editing percent at the nucleotides | | | | |
|---|---|---|---|---|---|---|
| | | numbered according to the position in the PWsp132 spacer | | | | |
| | Protein SEQ | sequence on the opposite, target genomic DNA strand | | | | |
| pWISE | ID NO: | G.−5 | G.6 | G.7 | G.9 | G.12 |
| pWISE121 | 56 | 0.000% | 0.002% | 0.000% | 0.152% | 0.000% |
| pWISE1921 | 428 | 0.019% | 0.005% | 0.035% | 0.056% | 0.089% |
| pWISE1925 | 429 | 0.016% | 0.007% | 0.002% | 0.115% | 0.026% |
| pWISE4332 | 430 | 0.017% | 0.009% | 0.010% | 0.033% | 0.038% |
| pWISE4339 | 431 | 0.021% | 0.001% | 0.001% | 0.038% | 0.014% |
| pWISE6852 | 432 | 0.011% | 0.005% | 0.004% | 0.008% | 0.003% |
| pWISE6853 | 433 | 0.001% | 0.003% | 0.006% | 0.023% | 0.012% |
| pWISE9117 | 450 | 0.006% | 0.005% | 0.003% | 0.025% | 0.003% |
| pWISE9118 | 451 | 0.008% | 0.002% | 0.001% | 0.001% | 1.265% |
| pWISE9119 | 452 | 0.037% | 0.037% | 0.075% | 0.257% | 0.059% |
| pWISE9120 | 453 | 0.000% | 0.000% | 0.000% | 1.560% | 8.974% |
| pWISE9121 | 454 | 0.005% | 0.136% | 0.216% | 0.122% | 0.391% |
| pWISE9122 | 455 | 0.002% | 0.025% | 0.051% | 0.118% | 0.059% |
| pWISE9123 | 456 | 0.021% | 0.012% | 0.009% | 0.264% | 0.054% |
| pWISE9124 | 457 | 0.026% | 0.002% | 0.013% | 0.191% | 1.569% |
| pWISE9125 | 458 | 0.007% | 0.007% | 0.055% | 0.118% | 0.082% |
| pWISE9126 | 459 | 0.011% | 0.009% | 0.004% | 2.134% | 3.260% |
| pWISE9127 | 460 | 0.009% | 0.015% | 0.014% | 0.002% | 0.025% |
| pWISE9128 | 461 | 0.037% | 0.000% | 0.009% | 0.101% | 0.101% |
| pWISE9134 | 426 | No data | No data | No data | No data | No data |
| pWISE9135 | 427 | 0.025% | 0.019% | 0.065% | 0.133% | 0.071% |

| | | Average G to A editing percent at the nucleotides | | | | |
|---|---|---|---|---|---|---|
| | | numbered according to the position in the PWsp132 spacer | | | | |
| | Protein SEQ | sequence on the opposite, target genomic DNA strand | | | | |
| pWISE | ID NO: | G.13 | G.15 | G.23 | G.25 | G.30 |
| pWISE121 | 56 | 0.004% | 0.000% | 0.666% | 0.008% | 0.016% |
| pWISE1921 | 428 | 0.045% | 0.391% | 0.087% | 0.023% | 2.625% |
| pWISE1925 | 429 | 0.030% | 0.244% | 0.020% | 0.045% | 1.279% |
| pWISE4332 | 430 | 0.018% | 0.170% | 0.119% | 0.119% | 1.136% |
| pWISE4339 | 431 | 0.010% | 0.102% | 0.029% | 0.079% | 0.342% |
| pWISE6852 | 432 | 0.011% | 0.081% | 0.012% | 0.037% | 0.349% |
| pWISE6853 | 433 | 0.009% | 0.183% | 0.025% | 0.059% | 0.391% |
| pWISE9117 | 450 | 0.001% | 0.417% | 0.321% | 0.825% | 3.152% |
| pWISE9118 | 451 | 0.589% | 2.668% | 1.004% | 0.653% | 1.403% |
| pWISE9119 | 452 | 0.128% | 0.717% | 2.743% | 1.962% | 10.667% |
| pWISE9120 | 453 | 9.416% | 10.981% | 2.763% | 0.470% | 3.007% |
| pWISE9121 | 454 | 0.204% | 0.868% | 0.213% | 0.208% | 0.793% |
| pWISE9122 | 455 | 0.253% | 0.674% | 0.552% | 0.835% | 5.303% |
| pWISE9123 | 456 | 0.039% | 0.874% | 1.039% | 2.152% | 7.802% |
| pWISE9124 | 457 | 1.563% | 7.892% | 3.097% | 1.319% | 2.874% |
| pWISE9125 | 458 | 0.109% | 0.702% | 0.188% | 0.492% | 6.920% |
| pWISE9126 | 459 | 3.110% | 6.625% | 0.798% | 0.193% | 0.538% |
| pWISE9127 | 460 | 0.000% | 0.235% | 0.140% | 0.116% | 0.222% |
| pWISE9128 | 461 | 0.046% | 0.587% | 0.422% | 1.037% | 5.249% |
| pWISE9134 | 426 | No data | No data | No data | No data | No data |
| pWISE9135 | 427 | 0.054% | 0.814% | 0.318% | 0.458% | 7.906% |

TABLE 9

| | | Standard deviation of the average G to A editing percent at the | | | | |
|---|---|---|---|---|---|---|
| | | nucleotides numbered according to the position in the PWsp132 | | | | |
| | Protein SEQ | spacer sequence on the opposite, target genomic DNA strand | | | | |
| pWISE | ID NO: | G.−5 | G.6 | G.7 | G.9 | G.12 |
| pWISE121 | 56 | 0.000% | 0.003% | 0.000% | 0.209% | 0.000% |
| pWISE1921 | 428 | No data | No data | No data | No data | No data |
| pWISE1925 | 429 | 0.011% | 0.003% | 0.003% | 0.078% | 0.036% |
| pWISE4332 | 430 | No data | No data | No data | No data | No data |
| pWISE4339 | 431 | 0.030% | 0.001% | 0.001% | 0.054% | 0.020% |
| pWISE6852 | 432 | 0.016% | 0.001% | 0.002% | 0.011% | 0.004% |
| pWISE6853 | 433 | 0.001% | 0.005% | 0.001% | 0.033% | 0.016% |
| pWISE9117 | 450 | 0.000% | 0.002% | 0.005% | 0.035% | 0.005% |
| pWISE9118 | 451 | No data | No data | No data | No data | No data |
| pWISE9119 | 452 | No data | No data | No data | No data | No data |
| pWISE9120 | 453 | No data | No data | No data | No data | No data |
| pWISE9121 | 454 | 0.007% | 0.137% | 0.189% | 0.172% | 0.236% |
| pWISE9122 | 455 | 0.003% | 0.035% | 0.072% | 0.167% | 0.084% |
| pWISE9123 | 456 | No data | No data | No data | No data | No data |
| pWISE9124 | 457 | 0.019% | 0.002% | 0.018% | 0.191% | 0.380% |
| pWISE9125 | 458 | No data | No data | No data | No data | No data |
| pWISE9126 | 459 | 0.013% | 0.004% | 0.000% | 0.230% | 0.285% |
| pWISE9127 | 460 | 0.004% | 0.021% | 0.019% | 0.002% | 0.035% |
| pWISE9128 | 461 | No data | No data | No data | No data | No data |
| pWISE9134 | 426 | No data | No data | No data | No data | No data |
| pWISE9135 | 427 | No data | No data | No data | No data | No data |
| | | Standard deviation of the average G to A editing percent at the | | | | |
| | | nucleotides numbered according to the position in the PWsp132 | | | | |
| | Protein SEQ | spacer sequence on the opposite, target genomic DNA strand | | | | |
| pWISE | ID NO: | G.13 | G.15 | G.23 | G.25 | G.30 |
| pWISE121 | 56 | 0.005% | 0.000% | 0.003% | 0.001% | 0.012% |
| pWISE1921 | 428 | No data | No data | No data | No data | No data |
| pWISE1925 | 429 | 0.018% | 0.040% | 0.005% | 0.057% | 0.327% |
| pWISE4332 | 430 | No data | No data | No data | No data | No data |
| pWISE4339 | 431 | 0.003% | 0.113% | 0.041% | 0.101% | 0.473% |
| pWISE6852 | 432 | 0.008% | 0.115% | 0.009% | 0.037% | 0.486% |
| pWISE6853 | 433 | 0.013% | 0.099% | 0.035% | 0.037% | 0.516% |
| pWISE9117 | 450 | 0.002% | 0.064% | 0.011% | 0.207% | 0.688% |
| pWISE9118 | 451 | No data | No data | No data | No data | No data |
| pWISE9119 | 452 | No data | No data | No data | No data | No data |
| pWISE9120 | 453 | No data | No data | No data | No data | No data |
| pWISE9121 | 454 | 0.041% | 0.254% | 0.037% | 0.079% | 0.038% |
| pWISE9122 | 455 | 0.266% | 0.321% | 0.781% | 0.635% | 1.675% |
| pWISE9123 | 456 | No data | No data | No data | No data | No data |
| pWISE9124 | 457 | 0.328% | 0.221% | 0.703% | 0.324% | 0.192% |
| pWISE9125 | 458 | No data | No data | No data | No data | No data |
| pWISE9126 | 459 | 0.072% | 0.011% | 0.118% | 0.083% | 0.110% |
| pWISE9127 | 460 | 0.000% | 0.211% | 0.127% | 0.152% | 0.010% |
| pWISE9128 | 461 | No data | No data | No data | No data | No data |
| pWISE9134 | 426 | No data | No data | No data | No data | No data |
| pWISE9135 | 427 | No data | No data | No data | No data | No data |

TABLE 10

| | | |
|---|---|---|
| | | Average C to T editing percent at the nucleotides numbered |
| | | according to the position in the PWsp449 spacer sequence on |
| Protein | Protein SEQ | the non-target, genomic DNA strand |

| Plasmid | ID NO: | C.−5 | C.−1 | C.2 | C.10 | C.11 |
|---|---|---|---|---|---|---|
| pWISE121 | 56 | 0.003% | 0.103% | 0.033% | 0.407% | 0.254% |
| pWISE1921 | 428 | 0.923% | 0.921% | 0.191% | 24.033% | 23.585% |
| pWISE1925 | 429 | 0.762% | 0.919% | 0.140% | 18.424% | 18.260% |
| pWISE4332 | 430 | 0.481% | 0.428% | 0.092% | 14.270% | 14.064% |
| pWISE4339 | 431 | 0.360% | 0.483% | 0.025% | 13.312% | 13.047% |
| pWISE6852 | 432 | 0.355% | 0.326% | 0.055% | 10.246% | 10.136% |
| pWISE6853 | 433 | 0.329% | 0.386% | 0.051% | 14.813% | 14.576% |
| pWISE9117 | 450 | No data | No data | No data | No data | No data |
| pWISE9118 | 451 | 0.690% | 0.795% | 0.294% | 8.872% | 9.034% |
| pWISE9119 | 452 | 1.769% | 1.903% | 0.245% | 34.072% | 32.966% |
| pWISE9120 | 453 | 0.256% | 0.294% | 0.063% | 4.899% | 4.716% |
| pWISE9121 | 454 | 0.672% | 0.580% | 0.195% | 7.651% | 7.707% |
| pWISE9122 | 455 | 1.646% | 2.007% | 0.210% | 25.512% | 25.766% |
| pWISE9123 | 456 | 1.416% | 1.711% | 0.234% | 28.032% | 27.623% |
| pWISE9124 | 457 | 0.539% | 0.718% | 0.112% | 23.185% | 21.996% |
| pWISE9125 | 458 | No data | No data | No data | No data | No data |
| pWISE9126 | 459 | 0.132% | 0.196% | 0.028% | 5.711% | 5.791% |
| pWISE9127 | 460 | 0.208% | 0.272% | 0.054% | 5.801% | 5.702% |
| pWISE9128 | 461 | 1.581% | 1.924% | 0.228% | 22.383% | 22.108% |
| pWISE9134 | 426 | 0.978% | 0.952% | 0.223% | 23.751% | 23.592% |
| pWISE9135 | 427 | 1.432% | 1.408% | 0.144% | 24.285% | 24.008% |

| | | Average C to T editing percent at the nucleotides numbered | | | | | |
|---|---|---|---|---|---|---|---|
| | | according to the position in the PWsp449 spacer sequence on the | | | | | |
| Protein | Protein SEQ | non-target, genomic DNA strand | | | | | |
| Plasmid | ID NO: | C.15 | C.20 | C.22 | C.30 | C.32 | C.33 |
| pWISE121 | 56 | 1.953% | 0.572% | 0.237% | 0.095% | 0.001% | 0.062% |
| pWISE1921 | 428 | 22.927% | 1.360% | 0.663% | 0.042% | 0.007% | 0.021% |
| pWISE1925 | 429 | 16.530% | 0.463% | 0.336% | 0.037% | 0.004% | 0.002% |
| pWISE4332 | 430 | 13.245% | 0.564% | 0.169% | 0.082% | 0.003% | 0.004% |
| pWISE4339 | 431 | 10.967% | 0.266% | 0.106% | 0.035% | 0.006% | 0.033% |
| pWISE6852 | 432 | 9.369% | 0.421% | 0.129% | 0.025% | 0.005% | 0.007% |
| pWISE6853 | 433 | 11.957% | 0.129% | 0.162% | 0.038% | 0.004% | 0.038% |
| pWISE9117 | 450 | No data | No data | No data | No data | No data | No data |
| pWISE9118 | 451 | 7.569% | 2.187% | 1.657% | 0.029% | 0.004% | 0.007% |
| pWISE9119 | 452 | 33.975% | 10.006% | 8.709% | 1.453% | 0.169% | 0.198% |
| pWISE9120 | 453 | 4.130% | 0.872% | 0.923% | 0.076% | 0.009% | 0.040% |
| pWISE9121 | 454 | 6.403% | 0.583% | 0.565% | 0.042% | 0.001% | 0.014% |
| pWISE9122 | 455 | 26.424% | 7.012% | 5.110% | 0.516% | 0.049% | 0.082% |
| pWISE9123 | 456 | 27.715% | 3.685% | 1.628% | 0.162% | 0.016% | 0.024% |
| pWISE9124 | 457 | 22.021% | 1.082% | 0.978% | 0.110% | 0.016% | 0.019% |
| pWISE9125 | 458 | No data | No data | No data | No data | No data | No data |
| pWISE9126 | 459 | 5.614% | 0.215% | 0.170% | 0.062% | 0.013% | 0.044% |
| pWISE9127 | 460 | 3.778% | 0.329% | 0.072% | 0.004% | 0.002% | 0.028% |
| pWISE9128 | 461 | 21.501% | 1.623% | 0.762% | 0.106% | 0.005% | 0.050% |
| pWISE9134 | 426 | 22.686% | 1.771% | 0.865% | 0.110% | 0.024% | 0.019% |
| pWISE9135 | 427 | 24.050% | 0.837% | 0.706% | 0.152% | 0.012% | 0.037% |

TABLE 11

| | | Standard Deviation of the average editing percent at the | | | | | |
|---|---|---|---|---|---|---|---|
| | | nucleotides numbered according to the position in the PWsp449 | | | | | |
| | Protein SEQ | spacer sequence on the non-target, genomic DNA strand | | | | | |
| pWISE | ID NO: | C.−5 | C.−1 | C.2 | C.10 | C.11 | C.15 |
| pWISE121 | 56 | 0.005% | 0.136% | 0.034% | 0.153% | 0.139% | 0.026% |
| pWISE1921 | 428 | 0.197% | 0.113% | 0.022% | 2.094% | 2.096% | 1.883% |
| pWISE1925 | 429 | 0.235% | 0.114% | 0.032% | 3.336% | 3.324% | 3.359% |
| pWISE4332 | 430 | 0.022% | 0.007% | 0.033% | 1.658% | 1.531% | 1.377% |
| pWISE4339 | 431 | 0.106% | 0.076% | 0.026% | 1.688% | 1.844% | 1.483% |
| pWISE6852 | 432 | 0.006% | 0.177% | 0.041% | 1.762% | 1.796% | 1.834% |
| pWISE6853 | 433 | 0.275% | 0.130% | 0.001% | 0.763% | 0.709% | 0.206% |
| pWISE9117 | 450 | No data | No data | No data | No data | No data | No data |
| pWISE9118 | 451 | 0.213% | 0.205% | 0.069% | 0.349% | 0.197% | 0.036% |
| pWISE9119 | 452 | 0.169% | 0.157% | 0.053% | 0.435% | 0.824% | 0.890% |
| pWISE9120 | 453 | 0.013% | 0.041% | 0.047% | 0.230% | 0.113% | 0.407% |
| pWISE9121 | 454 | 0.237% | 0.031% | 0.021% | 0.365% | 0.305% | 0.367% |
| pWISE9122 | 455 | 0.162% | 0.042% | 0.121% | 1.685% | 1.733% | 1.677% |
| pWISE9123 | 456 | 0.144% | 0.033% | 0.068% | 1.752% | 1.536% | 1.623% |
| pWISE9124 | 457 | 0.009% | 0.039% | 0.026% | 0.028% | 0.035% | 0.243% |
| pWISE9125 | 458 | No data | No data | No data | No data | No data | No data |
| pWISE9126 | 459 | 0.036% | 0.010% | 0.010% | 0.884% | 0.921% | 0.919% |
| pWISE9127 | 460 | 0.035% | 0.043% | 0.007% | 1.000% | 1.029% | 0.954% |
| pWISE9128 | 461 | 0.403% | 0.552% | 0.124% | 6.703% | 6.413% | 6.442% |
| pWISE9134 | 426 | 0.324% | 0.269% | 0.046% | 2.693% | 2.892% | 3.006% |
| pWISE9135 | 427 | 0.187% | 0.208% | 0.045% | 5.098% | 5.515% | 5.386% |

| | | Standard Deviation of the average editing percent at the | | | | |
|---|---|---|---|---|---|---|
| | | nucleotides numbered according to the position in the PWsp449 | | | | |
| | Protein SEQ | spacer sequence on the non-target, genomic DNA strand | | | | |
| pWISE | ID NO: | C.20 | C.22 | C.30 | C.32 | C.33 |
| pWISE121 | 56 | 0.259% | 0.093% | 0.060% | 0.002% | 0.085% |
| pWISE1921 | 428 | 0.071% | 0.083% | 0.029% | 0.003% | 0.023% |
| pWISE1925 | 429 | 0.119% | 0.107% | 0.049% | 0.003% | 0.002% |
| pWISE4332 | 430 | 0.366% | 0.021% | 0.045% | 0.002% | 0.006% |
| pWISE4339 | 431 | 0.032% | 0.063% | 0.001% | 0.006% | 0.041% |
| pWISE6852 | 432 | 0.022% | 0.010% | 0.034% | 0.002% | 0.000% |
| pWISE6853 | 433 | 0.046% | 0.220% | 0.048% | 0.006% | 0.044% |
| pWISE9117 | 450 | No data | No data | No data | No data | No data |
| pWISE9118 | 451 | 0.252% | 0.034% | 0.011% | 0.003% | 0.004% |
| pWISE9119 | 452 | 0.055% | 0.010% | 0.009% | 0.084% | 0.052% |
| pWISE9120 | 453 | 0.085% | 0.084% | 0.024% | 0.012% | 0.040% |
| pWISE9121 | 454 | 0.161% | 0.027% | 0.019% | 0.002% | 0.001% |
| pWISE9122 | 455 | 0.697% | 0.284% | 0.164% | 0.009% | 0.035% |
| pWISE9123 | 456 | 0.485% | 0.078% | 0.101% | 0.020% | 0.002% |
| pWISE9124 | 457 | 0.039% | 0.197% | 0.029% | 0.014% | 0.007% |
| pWISE9125 | 458 | No data | No data | No data | No data | No data |
| pWISE9126 | 459 | 0.029% | 0.059% | 0.001% | 0.015% | 0.011% |
| pWISE9127 | 460 | 0.005% | 0.053% | 0.000% | 0.003% | 0.009% |
| pWISE9128 | 461 | 0.501% | 0.191% | 0.023% | 0.004% | 0.054% |
| pWISE9134 | 426 | 0.442% | 0.164% | 0.075% | 0.010% | 0.022% |
| pWISE9135 | 427 | 0.145% | 0.109% | 0.071% | 0.014% | 0.004% |

TABLE 12

| | | Average G to A editing percent at the nucleotides numbered | | | | | |
|---|---|---|---|---|---|---|---|
| | | according to the position in the PWsp449 spacer sequence on the | | | | | |
| | Protein SEQ | opposite, target genomic DNA strand | | | | | |
| pWISE | ID NO: | G.−9 | G.1 | G.3 | G.4 | G.7 | G.17 |
| pWISE121 | 56 | 0.010% | 0.118% | 0.108% | 0.074% | 0.304% | 0.855% |
| pWISE1921 | 428 | 0.124% | 0.019% | 0.022% | 0.015% | 0.006% | 0.083% |
| pWISE1925 | 429 | 0.047% | 0.014% | 0.004% | 0.018% | 0.013% | 0.004% |
| pWISE4332 | 430 | 0.050% | 0.017% | 0.006% | 0.026% | 0.006% | 0.009% |
| pWISE4339 | 431 | 0.027% | 0.029% | 0.037% | 0.030% | 0.002% | 0.018% |
| pWISE6852 | 432 | 0.051% | 0.009% | 0.014% | 0.003% | 0.008% | 0.017% |
| pWISE6853 | 433 | 0.041% | 0.007% | 0.012% | 0.009% | 0.004% | 0.001% |
| pWISE9117 | 450 | No data | No data | No data | No data | No data | No data |
| pWISE9118 | 451 | 0.025% | 0.022% | 0.031% | 0.061% | 0.002% | 2.414% |
| pWISE9119 | 452 | 0.015% | 0.031% | 0.046% | 0.075% | 0.015% | 0.696% |
| pWISE9120 | 453 | 0.094% | 0.040% | 0.041% | 0.068% | 0.021% | 6.415% |
| pWISE9121 | 454 | 0.035% | 0.018% | 0.029% | 0.045% | 0.008% | 1.161% |
| pWISE9122 | 455 | 0.019% | 0.036% | 0.026% | 0.049% | 0.023% | 2.956% |
| pWISE9123 | 456 | 0.040% | 0.021% | 0.074% | 0.029% | 0.009% | 0.599% |
| pWISE9124 | 457 | 0.035% | 0.018% | 0.012% | 0.016% | 0.013% | 1.464% |
| pWISE9125 | 458 | No data | No data | No data | No data | No data | No data |
| pWISE9126 | 459 | 0.012% | 0.007% | 0.023% | 0.012% | 0.001% | 1.626% |
| pWISE9127 | 460 | 0.021% | 0.014% | 0.015% | 0.003% | 0.003% | 0.174% |
| pWISE9128 | 461 | 0.019% | 0.017% | 0.015% | 0.032% | 0.024% | 0.350% |
| pWISE9134 | 426 | 0.140% | 0.034% | 0.039% | 0.051% | 0.002% | 0.055% |
| pWISE9135 | 427 | 0.022% | 0.006% | 0.011% | 0.019% | 0.015% | 0.257% |

| | | Average G to A editing percent at the nucleotides numbered | | | | |
|---|---|---|---|---|---|---|
| | | according to the position in the PWsp449 spacer sequence on the | | | | |
| | Protein SEQ | opposite, target genomic DNA strand | | | | |
| pWISE | ID NO: | G.21 | G.24 | G.26 | G.28 | G.31 |
| pWISE121 | 56 | 0.260% | 0.324% | 0.064% | 0.047% | 0.012% |
| pWISE1921 | 428 | 0.191% | 1.681% | 2.012% | 0.030% | 0.188% |
| pWISE1925 | 429 | 0.063% | 0.573% | 0.879% | 0.002% | 0.044% |
| pWISE4332 | 430 | 0.181% | 0.768% | 0.820% | 0.040% | 0.076% |
| pWISE4339 | 431 | 0.099% | 0.481% | 0.411% | 0.075% | 0.050% |
| pWISE6852 | 432 | 0.111% | 0.557% | 0.376% | 0.014% | 0.062% |
| pWISE6853 | 433 | 0.035% | 0.395% | 0.253% | 0.092% | 0.030% |
| pWISE9117 | 450 | No data | No data | No data | No data | No data |
| pWISE9118 | 451 | 2.795% | 3.518% | 2.755% | 0.264% | 0.206% |
| pWISE9119 | 452 | 2.193% | 19.415% | 19.164% | 3.039% | 3.810% |
| pWISE9120 | 453 | 5.443% | 6.371% | 5.184% | 0.724% | 0.829% |
| pWISE9121 | 454 | 1.178% | 2.427% | 1.791% | 0.102% | 0.272% |
| pWISE9122 | 455 | 4.900% | 13.204% | 12.163% | 1.788% | 2.608% |
| pWISE9123 | 456 | 1.345% | 6.273% | 5.512% | 0.670% | 1.389% |
| pWISE9124 | 457 | 2.339% | 2.991% | 1.514% | 0.095% | 0.215% |
| pWISE9125 | 458 | No data | No data | No data | No data | No data |
| pWISE9126 | 459 | 1.027% | 1.063% | 0.567% | 0.029% | 0.053% |
| pWISE9127 | 460 | 0.155% | 0.483% | 0.338% | 0.016% | 0.062% |
| pWISE9128 | 461 | 0.569% | 3.365% | 3.377% | 0.240% | 0.656% |
| pWISE9134 | 426 | 0.201% | 2.538% | 3.027% | 0.041% | 0.207% |
| pWISE9135 | 427 | 0.424% | 4.395% | 4.906% | 0.315% | 1.174% |

TABLE 13

| | | Standard deviation of the average G to A editing percent at the | | | | | |
|---|---|---|---|---|---|---|---|
| | | nucleotides numbered according to the position in the PWsp449 | | | | | |
| | Protein SEQ | spacer sequence on the opposite, target genomic DNA strand | | | | | |
| pWISE | ID NO: | G.−9 | G.1 | G.3 | G.4 | G.7 | G.17 |
| pWISE121 | 56 | 0.007% | 0.164% | 0.108% | 0.102% | 0.194% | 0.052% |
| pWISE1921 | 428 | 0.054% | 0.020% | 0.027% | 0.016% | 0.008% | 0.016% |
| pWISE1925 | 429 | 0.004% | 0.014% | 0.001% | 0.018% | 0.010% | 0.002% |
| pWISE4332 | 430 | 0.043% | 0.019% | 0.001% | 0.034% | 0.001% | 0.006% |
| pWISE4339 | 431 | 0.003% | 0.041% | 0.046% | 0.043% | 0.003% | 0.020% |
| pWISE6852 | 432 | 0.008% | 0.008% | 0.017% | 0.002% | 0.001% | 0.019% |
| pWISE6853 | 433 | 0.058% | 0.010% | 0.008% | 0.002% | 0.000% | 0.001% |
| pWISE9117 | 450 | No data | No data | No data | No data | No data | No data |
| pWISE9118 | 451 | 0.015% | 0.028% | 0.005% | 0.007% | 0.003% | 0.019% |
| pWISE9119 | 452 | 0.002% | 0.015% | 0.003% | 0.004% | 0.007% | 0.189% |
| pWISE9120 | 453 | 0.091% | 0.031% | 0.058% | 0.046% | 0.005% | 1.054% |
| pWISE9121 | 454 | 0.019% | 0.022% | 0.019% | 0.029% | 0.008% | 0.003% |
| pWISE9122 | 455 | 0.016% | 0.005% | 0.019% | 0.031% | 0.027% | 0.195% |
| pWISE9123 | 456 | 0.021% | 0.009% | 0.027% | 0.041% | 0.012% | 0.079% |
| pWISE9124 | 457 | 0.001% | 0.018% | 0.002% | 0.001% | 0.006% | 0.130% |
| pWISE9125 | 458 | No data | No data | No data | No data | No data | No data |
| pWISE9126 | 459 | 0.003% | 0.003% | 0.008% | 0.000% | 0.001% | 0.275% |
| pWISE9127 | 460 | 0.018% | 0.013% | 0.011% | 0.001% | 0.002% | 0.054% |
| pWISE9128 | 461 | 0.021% | 0.013% | 0.017% | 0.012% | 0.023% | 0.054% |
| pWISE9134 | 426 | 0.059% | 0.004% | 0.009% | 0.020% | 0.003% | 0.009% |
| pWISE9135 | 427 | 0.010% | 0.006% | 0.001% | 0.024% | 0.012% | 0.131% |

| | | Standard deviation of the average G to A editing percent at the | | | | |
|---|---|---|---|---|---|---|
| | | nucleotides numbered according to the position in the PWsp449 | | | | |
| | Protein SEQ | spacer sequence on the opposite, target genomic DNA strand | | | | |
| pWISE | ID NO: | G.21 | G.24 | G.26 | G.28 | G.31 |
| pWISE121 | 56 | 0.051% | 0.007% | 0.072% | 0.064% | 0.009% |
| pWISE1921 | 428 | 0.040% | 0.413% | 0.253% | 0.039% | 0.047% |
| pWISE1925 | 429 | 0.077% | 0.009% | 0.038% | 0.002% | 0.042% |
| pWISE4332 | 430 | 0.026% | 0.185% | 0.263% | 0.027% | 0.007% |
| pWISE4339 | 431 | 0.021% | 0.098% | 0.043% | 0.083% | 0.023% |
| pWISE6852 | 432 | 0.027% | 0.047% | 0.014% | 0.013% | 0.039% |
| pWISE6853 | 433 | 0.015% | 0.106% | 0.033% | 0.129% | 0.031% |
| pWISE9117 | 450 | No data | No data | No data | No data | No data |
| pWISE9118 | 451 | 0.302% | 0.268% | 0.266% | 0.086% | 0.101% |
| pWISE9119 | 452 | 0.177% | 0.223% | 0.724% | 0.003% | 0.108% |
| pWISE9120 | 453 | 0.798% | 0.592% | 0.548% | 0.241% | 0.101% |
| pWISE9121 | 454 | 0.101% | 0.226% | 0.129% | 0.029% | 0.039% |
| pWISE9122 | 455 | 0.343% | 0.822% | 0.713% | 0.382% | 0.529% |
| pWISE9123 | 456 | 0.353% | 0.184% | 0.308% | 0.017% | 0.145% |
| pWISE9124 | 457 | 0.041% | 0.021% | 0.102% | 0.034% | 0.084% |
| pWISE9125 | 458 | No data | No data | No data | No data | No data |
| pWISE9126 | 459 | 0.231% | 0.188% | 0.179% | 0.004% | 0.035% |
| pWISE9127 | 460 | 0.000% | 0.116% | 0.066% | 0.002% | 0.042% |
| pWISE9128 | 461 | 0.067% | 0.740% | 0.571% | 0.003% | 0.091% |
| pWISE9134 | 426 | 0.004% | 0.117% | 0.006% | 0.012% | 0.028% |
| pWISE9135 | 427 | 0.146% | 0.925% | 1.215% | 0.027% | 0.372% |

TABLE 14

| | | Average C to T editing percent at the nucleotides numbered according | | | | | | |
|---|---|---|---|---|---|---|---|---|
| | Protein | to the position in the PWsp135 spacer sequence on the non-target, | | | | | | |
| | SEQ ID | genomic DNA strand | | | | | | |
| pWISE | NO: | C.−8 | C.−6 | C.−1 | C.3 | C.4 | C.6 | C.8 |
| pWISE121 | 56 | 0.004% | 0.082% | 0.007% | 0.032% | 0.028% | 0.093% | 0.027% |
| pWISE1921 | 428 | 0.449% | 0.002% | 2.335% | 0.055% | 0.099% | 33.274% | 32.089% |
| pWISE1925 | 429 | 0.493% | 0.000% | 1.944% | 0.005% | 0.023% | 28.748% | 27.917% |
| pWISE4332 | 430 | 0.058% | 0.002% | 1.078% | 0.003% | 0.001% | 13.589% | 13.370% |
| pWISE4339 | 431 | 0.505% | 0.013% | 2.176% | 0.037% | 0.042% | 17.310% | 11.945% |
| pWISE6852 | 432 | 0.353% | 0.000% | 0.965% | 0.002% | 0.018% | 15.624% | 15.013% |
| pWISE6853 | 433 | 0.307% | 0.004% | 1.592% | 0.038% | 0.042% | 27.356% | 26.892% |
| pWISE9117 | 450 | 0.316% | 0.013% | 2.178% | 0.028% | 0.018% | 32.296% | 26.313% |
| pWISE9118 | 451 | 0.205% | 0.006% | 1.582% | 0.014% | 0.018% | 9.416% | 2.599% |
| pWISE9119 | 452 | 0.340% | 0.004% | 1.983% | 0.043% | 0.034% | 22.773% | 17.812% |
| pWISE9120 | 453 | 0.399% | 0.031% | 1.455% | 0.034% | 0.037% | 4.755% | 1.729% |
| pWISE9121 | 454 | 0.414% | 0.019% | 1.723% | 0.054% | 0.077% | 18.202% | 14.310% |
| pWISE9122 | 455 | 0.428% | 0.005% | 3.150% | 0.036% | 0.046% | 17.129% | 6.481% |
| pWISE9123 | 456 | 0.404% | 0.000% | 2.146% | 0.074% | 0.065% | 30.831% | 28.556% |
| pWISE9124 | 457 | 0.329% | 0.000% | 1.657% | 0.021% | 0.012% | 29.966% | 20.258% |
| pWISE9125 | 458 | 0.335% | 0.014% | 2.390% | 0.013% | 0.046% | 25.072% | 16.678% |
| pWISE9126 | 459 | 0.086% | 0.008% | 0.476% | 0.005% | 0.003% | 9.905% | 4.317% |
| pWISE9127 | 460 | 0.066% | 0.003% | 0.533% | 0.015% | 0.012% | 4.287% | 0.375% |
| pWISE9128 | 461 | 0.524% | 0.000% | 2.685% | 0.056% | 0.036% | 20.135% | 14.338% |
| pWISE9134 | 426 | 0.446% | 0.018% | 2.248% | 0.034% | 0.053% | 30.029% | 29.043% |
| pWISE9135 | 427 | 0.480% | 0.018% | 2.492% | 0.053% | 0.068% | 27.522% | 18.083% |

| | | Average C to T editing percent at the nucleotides numbered according | | | | | |
|---|---|---|---|---|---|---|---|
| | | to the position in the PWsp135 spacer sequence on the non-target, | | | | | |
| | Protein SEQ | genomic DNA strand | | | | | |
| pWISE | ID NO: | C.9 | C.10 | C.11 | C.13 | C.17 | C.18 |
| pWISE121 | 56 | 0.066% | 0.062% | 0.183% | 0.110% | 0.529% | 0.497% |
| pWISE1921 | 428 | 32.913% | 33.030% | 33.154% | 33.103% | 8.980% | 9.597% |
| pWISE1925 | 429 | 28.554% | 28.591% | 28.672% | 28.621% | 4.710% | 4.742% |
| pWISE4332 | 430 | 13.499% | 13.522% | 13.549% | 13.465% | 0.984% | 1.005% |
| pWISE4339 | 431 | 15.873% | 18.932% | 21.414% | 24.483% | 5.246% | 5.865% |
| pWISE6852 | 432 | 15.417% | 15.438% | 15.468% | 15.453% | 1.108% | 0.810% |
| pWISE6853 | 433 | 27.260% | 27.248% | 27.317% | 27.187% | 2.880% | 3.198% |
| pWISE9117 | 450 | 30.307% | 31.080% | 31.396% | 32.299% | 15.909% | 19.326% |
| pWISE9118 | 451 | 5.969% | 7.855% | 9.749% | 15.186% | 0.260% | 0.250% |
| pWISE9119 | 452 | 21.323% | 22.141% | 22.744% | 24.504% | 9.434% | 10.306% |
| pWISE9120 | 453 | 2.699% | 3.216% | 3.695% | 6.311% | 0.660% | 0.855% |
| pWISE9121 | 454 | 16.297% | 17.133% | 17.846% | 19.783% | 5.145% | 5.294% |
| pWISE9122 | 455 | 14.034% | 19.647% | 24.408% | 29.916% | 9.575% | 9.993% |
| pWISE9123 | 456 | 30.055% | 30.102% | 30.445% | 30.861% | 11.225% | 12.172% |
| pWISE9124 | 457 | 27.775% | 28.622% | 28.955% | 30.110% | 3.770% | 3.909% |
| pWISE9125 | 458 | 21.677% | 23.952% | 24.566% | 25.457% | 6.897% | 6.556% |
| pWISE9126 | 459 | 7.872% | 8.717% | 9.140% | 10.044% | 0.230% | 0.211% |
| pWISE9127 | 460 | 1.626% | 2.758% | 3.639% | 6.516% | 0.141% | 0.138% |
| pWISE9128 | 461 | 21.975% | 22.372% | 22.577% | 22.783% | 4.866% | 5.772% |
| pWISE9134 | 426 | 29.709% | 29.702% | 29.879% | 29.991% | 9.456% | 9.988% |
| pWISE9135 | 427 | 24.364% | 26.319% | 26.967% | 28.052% | 8.167% | 7.647% |

| | | Average C to T editing percent at the nucleotides | | | | | |
|---|---|---|---|---|---|---|---|
| | | numbered according to the position in the PWsp135 spacer | | | | | |
| | Protein SEQ | sequence on the non-target, genomic DNA strand | | | | | |
| pWISE | ID NO: | C.19 | C.22 | C.25 | C.27 | C.29 | C.33 |
| pWISE121 | 56 | 0.364% | 0.669% | 0.030% | 0.048% | 0.102% | 0.036% |
| pWISE1921 | 428 | 12.497% | 0.162% | 0.181% | 0.312% | 0.290% | 0.163% |
| pWISE1925 | 429 | 5.591% | 0.080% | 0.161% | 0.104% | 0.183% | 0.148% |
| pWISE4332 | 430 | 2.019% | 0.084% | 0.072% | 0.084% | 0.116% | 0.026% |
| pWISE4339 | 431 | 8.595% | 1.401% | 1.333% | 1.499% | 1.569% | 1.304% |
| pWISE6852 | 432 | 1.948% | 0.075% | 0.046% | 0.054% | 0.011% | 0.072% |
| pWISE6853 | 433 | 4.081% | 0.184% | 0.138% | 0.073% | 0.192% | 0.096% |
| pWISE9117 | 450 | 26.265% | 0.544% | 0.231% | 0.163% | 0.281% | 0.203% |
| pWISE9118 | 451 | 1.418% | 0.082% | 0.203% | 0.215% | 0.073% | 0.191% |
| pWISE9119 | 452 | 13.392% | 2.260% | 1.905% | 2.309% | 2.310% | 2.053% |
| pWISE9120 | 453 | 1.251% | 0.184% | 0.318% | 0.309% | 0.263% | 0.347% |
| pWISE9121 | 454 | 6.573% | 0.172% | 0.183% | 0.191% | 0.205% | 0.168% |
| pWISE9122 | 455 | 13.863% | 1.587% | 1.614% | 1.930% | 2.198% | 2.088% |
| pWISE9123 | 456 | 18.386% | 0.243% | 0.270% | 0.374% | 0.223% | 0.412% |
| pWISE9124 | 457 | 6.291% | 0.102% | 0.089% | 0.076% | 0.106% | 0.068% |
| pWISE9125 | 458 | 6.007% | 0.276% | 0.244% | 0.283% | 0.261% | 0.267% |
| pWISE9126 | 459 | 0.326% | 0.013% | 0.045% | 0.040% | 0.043% | 0.040% |
| pWISE9127 | 460 | 0.212% | 0.044% | 0.045% | 0.067% | 0.059% | 0.042% |
| pWISE9128 | 461 | 7.406% | 0.330% | 0.245% | 0.341% | 0.367% | 0.132% |
| pWISE9134 | 426 | 12.707% | 0.289% | 0.184% | 0.185% | 0.167% | 0.098% |
| pWISE9135 | 427 | 7.159% | 0.181% | 0.261% | 0.218% | 0.260% | 0.256% |

TABLE 15

| | | Standard Deviation of the average editing percent at the nucleotides | | | | | | |
|---|---|---|---|---|---|---|---|---|
| | | numbered according to the position in the PWsp135 spacer sequence on | | | | | | |
| | Protein SEQ | DNA strand | | | | | | |
| pWISE | ID NO: | C.−8 | C.−6 | C.−1 | C.3 | C.4 | C.6 | C.8 |
| pWISE121 | 56 | 0.001% | 0.108% | 0.003% | 0.041% | 0.040% | 0.065% | 0.021% |
| pWISE1921 | 428 | 0.074% | 0.003% | 0.036% | 0.047% | 0.023% | 1.238% | 1.067% |
| pWISE1925 | 429 | 0.171% | 0.000% | 0.116% | 0.008% | 0.029% | 1.587% | 1.720% |
| pWISE4332 | 430 | 0.002% | 0.003% | 0.116% | 0.005% | 0.002% | 2.423% | 2.430% |
| pWISE4339 | 431 | 0.143% | 0.019% | 1.182% | 0.043% | 0.041% | 4.776% | 11.772% |
| pWISE6852 | 432 | 0.220% | 0.000% | 0.807% | 0.002% | 0.012% | 1.785% | 2.187% |
| pWISE6853 | 433 | No data | No data | No data | No data | No data | No data | No data |
| pWISE9117 | 450 | No data | No data | No data | No data | No data | No data | No data |
| pWISE9118 | 451 | 0.281% | 0.006% | 0.040% | 0.015% | 0.025% | 1.257% | 0.862% |
| pWISE9119 | 452 | 0.148% | 0.006% | 1.289% | 0.051% | 0.049% | 16.715% | 15.751% |
| pWISE9120 | 453 | 0.109% | 0.022% | 0.134% | 0.001% | 0.014% | 1.765% | 2.017% |
| pWISE9121 | 454 | 0.048% | 0.028% | 0.567% | 0.071% | 0.075% | 14.980% | 18.648% |
| pWISE9122 | 455 | 0.025% | 0.007% | 0.351% | 0.052% | 0.061% | 3.792% | 4.350% |
| pWISE9123 | 456 | No data | No data | No data | No data | No data | No data | No data |
| pWISE9124 | 457 | 0.351% | 0.000% | 0.482% | 0.030% | 0.013% | 1.052% | 0.356% |
| pWISE9125 | 458 | 0.181% | 0.015% | 0.128% | 0.016% | 0.041% | 0.961% | 0.502% |
| pWISE9126 | 459 | No data | No data | No data | No data | No data | No data | No data |
| pWISE9127 | 460 | No data | No data | No data | No data | No data | No data | No data |
| pWISE9128 | 461 | 0.043% | 0.000% | 0.020% | 0.019% | 0.047% | 0.826% | 0.511% |
| pWISE9134 | 426 | 0.027% | 0.022% | 0.503% | 0.048% | 0.074% | 1.078% | 1.393% |
| pWISE9135 | 427 | 0.169% | 0.001% | 0.110% | 0.004% | 0.050% | 1.443% | 0.999% |

| | | Standard Deviation of the average editing percent at the | | | | | |
|---|---|---|---|---|---|---|---|
| | | nucleotides numbered according to the position in the PWsp135 | | | | | |
| | Protein SEQ | spacer sequence on the non-target, genomic DNA strand | | | | | |
| pWISE | ID NO: | C.9 | C.10 | C.11 | C.13 | C.17 | C.18 |
| pWISE121 | 56 | 0.003% | 0.022% | 0.063% | 0.014% | 0.017% | 0.133% |
| pWISE1921 | 428 | 1.221% | 1.174% | 1.196% | 1.330% | 0.141% | 0.204% |
| pWISE1925 | 429 | 1.657% | 1.659% | 1.652% | 1.536% | 0.111% | 0.181% |
| pWISE4332 | 430 | 2.437% | 2.546% | 2.513% | 2.576% | 0.285% | 0.116% |
| pWISE4339 | 431 | 6.733% | 2.515% | 0.991% | 5.345% | 5.006% | 5.854% |
| pWISE6852 | 432 | 1.898% | 1.938% | 1.973% | 1.967% | 0.074% | 0.657% |
| pWISE6853 | 433 | No data | No data | No data | No data | No data | No data |
| pWISE9117 | 450 | No data | No data | No data | No data | No data | No data |
| pWISE9118 | 451 | 0.189% | 0.802% | 1.211% | 2.879% | 0.363% | 0.342% |
| pWISE9119 | 452 | 18.009% | 17.499% | 17.553% | 16.536% | 8.552% | 9.523% |
| pWISE9120 | 453 | 2.301% | 2.266% | 2.227% | 1.528% | 0.782% | 0.872% |
| pWISE9121 | 454 | 17.261% | 15.941% | 15.310% | 12.699% | 5.648% | 6.513% |
| pWISE9122 | 455 | 3.392% | 1.522% | 0.672% | 2.655% | 1.097% | 1.092% |
| pWISE9123 | 456 | No data | No data | No data | No data | No data | No data |
| pWISE9124 | 457 | 0.673% | 0.902% | 0.794% | 0.910% | 0.118% | 0.075% |
| pWISE9125 | 458 | 0.475% | 0.622% | 0.892% | 0.916% | 0.003% | 0.004% |
| pWISE9126 | 459 | No data | No data | No data | No data | No data | No data |
| pWISE9127 | 460 | No data | No data | No data | No data | No data | No data |
| pWISE9128 | 461 | 0.713% | 0.690% | 0.795% | 0.620% | 0.894% | 0.613% |
| pWISE9134 | 426 | 0.950% | 1.111% | 0.937% | 1.095% | 0.065% | 0.252% |
| pWISE9135 | 427 | 1.275% | 1.333% | 1.204% | 1.394% | 0.114% | 0.182% |

| | | Standard Deviation of the average editing percent at the | | | | | |
|---|---|---|---|---|---|---|---|
| | | nucleotides numbered according to the position in the PWsp135 | | | | | |
| | Protein SEQ | spacer sequence on the non-target, genomic DNA strand | | | | | |
| pWISE | ID NO: | C.19 | C.22 | C.25 | C.27 | C.29 | C.33 |
| pWISE121 | 56 | 0.188% | 0.023% | 0.040% | 0.065% | 0.064% | 0.043% |
| pWISE1921 | 428 | 0.160% | 0.004% | 0.088% | 0.112% | 0.161% | 0.033% |
| pWISE1925 | 429 | 0.288% | 0.013% | 0.060% | 0.000% | 0.009% | 0.098% |
| pWISE4332 | 430 | 0.292% | 0.051% | 0.065% | 0.048% | 0.121% | 0.003% |
| pWISE4339 | 431 | 8.805% | 1.879% | 1.667% | 2.018% | 2.112% | 1.741% |
| pWISE6852 | 432 | 1.310% | 0.102% | 0.059% | 0.076% | 0.012% | 0.092% |
| pWISE6853 | 433 | No data | No data | No data | No data | No data | No data |
| pWISE9117 | 450 | No data | No data | No data | No data | No data | No data |
| pWISE9118 | 451 | 0.222% | 0.114% | 0.076% | 0.114% | 0.103% | 0.131% |
| pWISE9119 | 452 | 12.311% | 2.064% | 1.683% | 2.184% | 2.135% | 1.832% |
| pWISE9120 | 453 | 1.057% | 0.042% | 0.113% | 0.001% | 0.004% | 0.045% |
| pWISE9121 | 454 | 8.103% | 0.170% | 0.136% | 0.153% | 0.151% | 0.075% |
| pWISE9122 | 455 | 3.246% | 0.051% | 0.122% | 0.318% | 0.502% | 0.833% |
| pWISE9123 | 456 | No data | No data | No data | No data | No data | No data |
| pWISE9124 | 457 | 0.160% | 0.007% | 0.063% | 0.041% | 0.091% | 0.041% |
| pWISE9125 | 458 | 0.390% | 0.025% | 0.132% | 0.043% | 0.167% | 0.066% |
| pWISE9126 | 459 | No data | No data | No data | No data | No data | No data |
| pWISE9127 | 460 | No data | No data | No data | No data | No data | No data |
| pWISE9128 | 461 | 0.899% | 0.162% | 0.191% | 0.052% | 0.295% | 0.050% |
| pWISE9134 | 426 | 0.159% | 0.012% | 0.055% | 0.058% | 0.066% | 0.125% |
| pWISE9135 | 427 | 0.370% | 0.004% | 0.084% | 0.054% | 0.004% | 0.158% |

TABLE 16

| | | Average G to A editing percent | | |
|---|---|---|---|---|
| | | at the nucleotides numbered | | |
| | | according to the position in the | | |
| | | PWsp135 spacer sequence on the | | |
| | Protein SEQ | opposite, target genomic DNA strand | | |
| pWISE | ID NO: | G.-4 | G.2 | G.16 |
| pWISE121 | 56 | 0.004% | 0.001% | 0.611% |
| pWISE1921 | 428 | 0.003% | 0.015% | 0.026% |
| pWISE1925 | 429 | 0.001% | 0.003% | 0.039% |
| pWISE4332 | 430 | 0.005% | 0.004% | 0.001% |
| pWISE4339 | 431 | 0.000% | 0.005% | 0.099% |
| pWISE6852 | 432 | 0.015% | 0.000% | 0.012% |
| pWISE6853 | 433 | 0.008% | 0.015% | 0.000% |
| pWISE9117 | 450 | 0.000% | 0.013% | 0.090% |
| pWISE9118 | 451 | 0.006% | 0.003% | 0.258% |
| pWISE9119 | 452 | 0.012% | 0.003% | 0.051% |
| pWISE9120 | 453 | 0.019% | 0.002% | 6.797% |
| pWISE9121 | 454 | 0.004% | 0.000% | 0.079% |
| pWISE9122 | 455 | 0.003% | 0.004% | 0.279% |
| pWISE9123 | 456 | 0.002% | 0.002% | 0.214% |
| pWISE9124 | 457 | 0.001% | 0.004% | 1.014% |
| pWISE9125 | 458 | 0.004% | 0.005% | 0.779% |
| pWISE9126 | 459 | 0.003% | 0.000% | 2.303% |
| pWISE9127 | 460 | 0.003% | 0.002% | 0.020% |
| pWISE9128 | 461 | 0.003% | 0.005% | 0.680% |
| pWISE9134 | 426 | 0.004% | 0.002% | 0.066% |
| pWISE9135 | 427 | 0.000% | 0.000% | 0.951% |

TABLE 17

| | | Standard deviation of the average G to | | |
|---|---|---|---|---|
| | | A editing percent at the nucleotides | | |
| | | numbered according to the position | | |
| | | in the PWsp135 spacer sequence on the | | |
| | Protein SEQ | opposite, target genomic DNA strand | | |
| pWISE | ID NO: | G.-4 | G.2 | G.16 |
| pWISE121 | 56 | 0.001% | 0.002% | 0.025% |
| pWISE1921 | 428 | 0.002% | 0.017% | 0.011% |
| pWISE1925 | 429 | 0.002% | 0.000% | 0.020% |
| pWISE4332 | 430 | 0.002% | 0.000% | 0.002% |
| pWISE4339 | 431 | 0.000% | 0.007% | 0.139% |
| pWISE6852 | 432 | 0.015% | 0.000% | 0.006% |
| pWISE6853 | 433 | No data | No data | No data |
| pWISE9117 | 450 | No data | No data | No data |
| pWISE9118 | 451 | 0.006% | 0.001% | 0.120% |
| pWISE9119 | 452 | 0.003% | 0.004% | 0.022% |
| pWISE9120 | 453 | 0.027% | 0.002% | 0.793% |
| pWISE9121 | 454 | 0.006% | 0.000% | 0.011% |
| pWISE9122 | 455 | 0.000% | 0.005% | 0.384% |
| pWISE9123 | 456 | No data | No data | No data |
| pWISE9124 | 457 | 0.002% | 0.002% | 0.081% |
| pWISE9125 | 458 | 0.000% | 0.004% | 0.095% |
| pWISE9126 | 459 | No data | No data | No data |
| pWISE9127 | 460 | No data | No data | No data |
| pWISE9128 | 461 | 0.001% | 0.001% | 0.026% |
| pWISE9134 | 426 | 0.005% | 0.002% | 0.037% |
| pWISE9135 | 427 | 0.000% | 0.000% | 0.094% |

TABLE 18

| | | Average C to T editing percent at the nucleotides numbered | | | | | |
|---|---|---|---|---|---|---|---|
| | Protein | according to the position in the PWsp389 spacer sequence on the | | | | | |
| | SEQ ID | non-target, genomic DNA strand | | | | | |
| pWISE | NO: | C.-5 | C.-1 | C.4 | C.6 | C.7 | C.11 |
| pWISE121 | 56 | 0.000% | 0.004% | 0.011% | 0.051% | 0.125% | 0.323% |
| pWISE1921 | 428 | 0.008% | 0.456% | 0.513% | 9.800% | 19.476% | 20.965% |
| pWISE1925 | 429 | 0.000% | 0.843% | 0.536% | 11.402% | 18.213% | 19.821% |
| pWISE4332 | 430 | 0.000% | 0.000% | 2.086% | 2.095% | 6.942% | 6.942% |
| pWISE4339 | 431 | 0.000% | 0.435% | 0.274% | 8.060% | 11.558% | 12.045% |
| pWISE6852 | 432 | 0.007% | 0.238% | 0.163% | 5.269% | 8.548% | 9.071% |
| pWISE6853 | 433 | 0.000% | 0.213% | 0.180% | 9.374% | 12.452% | 13.227% |
| pWISE9117 | 450 | 0.010% | 0.552% | 0.480% | 4.521% | 12.347% | 15.308% |
| pWISE9118 | 451 | 0.024% | 0.477% | 0.467% | 0.217% | 1.897% | 7.253% |
| pWISE9119 | 452 | 0.005% | 0.573% | 0.556% | 2.548% | 17.092% | 23.577% |
| pWISE9120 | 453 | 0.010% | 0.185% | 0.292% | 0.058% | 0.301% | 2.158% |
| pWISE9121 | 454 | 0.000% | 0.294% | 0.311% | 0.177% | 2.178% | 7.121% |
| pWISE9122 | 455 | 0.032% | 0.871% | 1.003% | 0.392% | 3.083% | 17.531% |
| pWISE9123 | 456 | 0.034% | 0.660% | 0.901% | 7.263% | 21.480% | 26.577% |
| pWISE9124 | 457 | 0.007% | 0.276% | 0.317% | 0.774% | 7.124% | 13.284% |
| pWISE9125 | 458 | 0.010% | 0.632% | 0.805% | 9.056% | 11.011% | 17.307% |
| pWISE9126 | 459 | 0.004% | 0.061% | 0.041% | 0.063% | 0.471% | 1.067% |
| pWISE9127 | 460 | 0.001% | 0.072% | 0.102% | 0.138% | 0.838% | 3.744% |
| pWISE9128 | 461 | 0.000% | 0.937% | 0.860% | 1.562% | 6.108% | 19.437% |
| pWISE9134 | 426 | 0.022% | 0.579% | 0.776% | 11.143% | 20.723% | 22.302% |
| pWISE9135 | 427 | 0.014% | 0.985% | 1.065% | 9.861% | 13.922% | 22.716% |

| | | Average C to T editing percent at the nucleotides numbered | | | | | |
|---|---|---|---|---|---|---|---|
| | Protein | according to the position in the PWsp389 spacer sequence on | | | | | |
| | SEQ ID | the non-target, genomic DNA strand | | | | | |
| pWISE | NO: | C.12 | C.16 | C.17 | C.18 | C.25 | C.26 |
| pWISE121 | 56 | 0.338% | 0.536% | 1.449% | 0.404% | 0.004% | 0.084% |
| pWISE1921 | 428 | 20.946% | 5.889% | 3.032% | 2.926% | 0.012% | 0.000% |
| pWISE1925 | 429 | 19.800% | 4.399% | 2.128% | 1.868% | 0.024% | 0.003% |
| pWISE4332 | 430 | 6.942% | 4.494% | 4.494% | 2.418% | 0.000% | 0.000% |
| pWISE4339 | 431 | 11.975% | 1.598% | 0.749% | 0.767% | 0.009% | 0.006% |
| pWISE6852 | 432 | 8.969% | 2.465% | 0.903% | 0.828% | 0.014% | 0.000% |
| pWISE6853 | 433 | 13.300% | 1.329% | 0.406% | 0.362% | 0.004% | 0.008% |
| pWISE9117 | 450 | 15.149% | 7.565% | 5.997% | 6.612% | 0.003% | 0.000% |
| pWISE9118 | 451 | 6.845% | 1.855% | 0.913% | 0.793% | 0.004% | 0.006% |
| pWISE9119 | 452 | 23.420% | 11.357% | 7.858% | 8.294% | 0.032% | 0.029% |
| pWISE9120 | 453 | 2.090% | 0.612% | 0.525% | 0.496% | 0.039% | 0.049% |
| pWISE9121 | 454 | 6.928% | 2.363% | 1.152% | 1.228% | 0.008% | 0.008% |
| pWISE9122 | 455 | 17.449% | 9.848% | 7.807% | 7.926% | 0.026% | 0.036% |
| pWISE9123 | 456 | 26.625% | 11.755% | 9.196% | 9.581% | 0.021% | 0.028% |
| pWISE9124 | 457 | 13.199% | 4.884% | 3.348% | 3.193% | 0.003% | 0.001% |
| pWISE9125 | 458 | 17.053% | 11.007% | 8.898% | 6.027% | 0.008% | 0.000% |
| pWISE9126 | 459 | 1.057% | 0.369% | 0.249% | 0.203% | 0.012% | 0.021% |
| pWISE9127 | 460 | 3.387% | 0.810% | 0.362% | 0.142% | 0.002% | 0.004% |
| pWISE9128 | 461 | 19.402% | 5.747% | 3.305% | 3.069% | 0.003% | 0.008% |
| pWISE9134 | 426 | 22.340% | 6.183% | 3.064% | 3.048% | 0.005% | 0.005% |
| pWISE9135 | 427 | 22.433% | 13.279% | 10.219% | 6.828% | 0.009% | 0.008% |

| | | Average C to T editing percent at the nucleotides | | |
|---|---|---|---|---|
| | | numbered according to the position in the PWsp389 | | |
| | Protein SEQ | spacer sequence on the non-target, genomic DNA strand | | |
| pWISE | ID NO: | C.27 | C.28 | C.30 |
| pWISE121 | 56 | 0.000% | 0.125% | 0.000% |
| pWISE1921 | 428 | 0.002% | 0.008% | 0.005% |
| pWISE1925 | 429 | 0.000% | 0.006% | 0.002% |
| pWISE4332 | 430 | 0.000% | 0.000% | 0.000% |
| pWISE4339 | 431 | 0.003% | 0.000% | 0.000% |
| pWISE6852 | 432 | 0.007% | 0.000% | 0.000% |
| pWISE6853 | 433 | 0.000% | 0.000% | 0.000% |
| pWISE9117 | 450 | 0.000% | 0.006% | 0.004% |
| pWISE9118 | 451 | 0.014% | 0.015% | 0.002% |
| pWISE9119 | 452 | 0.035% | 0.033% | 0.000% |
| pWISE9120 | 453 | 0.039% | 0.039% | 0.049% |
| pWISE9121 | 454 | 0.000% | 0.000% | 0.017% |
| pWISE9122 | 455 | 0.019% | 0.019% | 0.004% |
| pWISE9123 | 456 | 0.069% | 0.069% | 0.000% |
| pWISE9124 | 457 | 0.018% | 0.014% | 0.003% |
| pWISE9125 | 458 | 0.038% | 0.046% | 0.000% |
| pWISE9126 | 459 | 0.000% | 0.009% | 0.003% |
| pWISE9127 | 460 | 0.005% | 0.001% | 0.004% |
| pWISE9128 | 461 | 0.000% | 0.013% | 0.000% |
| pWISE9134 | 426 | 0.005% | 0.005% | 0.005% |
| pWISE9135 | 427 | 0.003% | 0.003% | 0.000% |

TABLE 19

| | | Standard Deviation of the average editing percent at the | | | | | |
|---|---|---|---|---|---|---|---|
| | | nucleotides numbered according to the position in the PWsp389 | | | | | |
| | Protein SEQ | spacer sequence on the non-target, genomic DNA strand | | | | | |
| pWISE | ID NO: | C.−5 | C.−1 | C.4 | C.6 | C.7 | C.11 |
| pWISE121 | 56 | No data | No data | No data | No data | No data | No data |
| pWISE1921 | 428 | 0.011% | 0.080% | 0.148% | 0.737% | 0.675% | 0.756% |
| pWISE1925 | 429 | 0.000% | 0.369% | 0.001% | 0.084% | 0.696% | 1.043% |
| pWISE4332 | 430 | No data | No data | No data | No data | No data | No data |
| pWISE4339 | 431 | No data | No data | No data | No data | No data | No data |
| pWISE6852 | 432 | No data | No data | No data | No data | No data | No data |
| pWISE6853 | 433 | 0.000% | 0.091% | 0.161% | 0.426% | 0.408% | 0.341% |
| pWISE9117 | 450 | 0.002% | 0.011% | 0.074% | 0.240% | 1.162% | 1.404% |
| pWISE9118 | 451 | 0.014% | 0.001% | 0.036% | 0.029% | 0.013% | 0.002% |
| pWISE9119 | 452 | 0.007% | 0.120% | 0.177% | 0.300% | 1.510% | 0.775% |
| pWISE9120 | 453 | No data | No data | No data | No data | No data | No data |
| pWISE9121 | 454 | No data | No data | No data | No data | No data | No data |
| pWISE9122 | 455 | 0.045% | 0.043% | 0.055% | 0.005% | 0.301% | 2.519% |
| pWISE9123 | 456 | No data | No data | No data | No data | No data | No data |
| pWISE9124 | 457 | 0.008% | 0.003% | 0.028% | 0.033% | 0.538% | 0.444% |
| pWISE9125 | 458 | 0.014% | 0.097% | 0.067% | 0.804% | 0.756% | 1.033% |
| pWISE9126 | 459 | 0.006% | 0.001% | 0.029% | 0.023% | 0.215% | 0.192% |
| pWISE9127 | 460 | 0.001% | 0.037% | 0.025% | 0.026% | 0.055% | 0.194% |
| pWISE9128 | 461 | 0.000% | 0.078% | 0.263% | 0.318% | 0.004% | 0.689% |
| pWISE9134 | 426 | No data | No data | No data | No data | No data | No data |
| pWISE9135 | 427 | 0.010% | 0.050% | 0.065% | 0.240% | 0.059% | 0.984% |
| | | Standard Deviation of the average editing percent at the | | | | | |
| | | nucleotides numbered according to the position in the PWsp389 | | | | | |
| | Protein SEQ | spacer sequence on the non-target, genomic DNA strand | | | | | |
| pWISE | ID NO: | C.12 | C.16 | C.17 | C.18 | C.25 | C.26 |
| pWISE121 | 56 | No data | No data | No data | No data | No data | No data |
| pWISE1921 | 428 | 0.624% | 0.062% | 0.095% | 0.255% | 0.016% | 0.000% |
| pWISE1925 | 429 | 1.003% | 0.146% | 0.365% | 0.218% | 0.024% | 0.005% |
| pWISE4332 | 430 | No data | No data | No data | No data | No data | No data |
| pWISE4339 | 431 | No data | No data | No data | No data | No data | No data |
| pWISE6852 | 432 | No data | No data | No data | No data | No data | No data |
| pWISE6853 | 433 | 0.285% | 0.189% | 0.244% | 0.072% | 0.006% | 0.000% |
| pWISE9117 | 450 | 1.499% | 1.089% | 0.807% | 0.861% | 0.004% | 0.000% |
| pWISE9118 | 451 | 0.051% | 0.271% | 0.033% | 0.028% | 0.005% | 0.002% |
| pWISE9119 | 452 | 0.892% | 0.129% | 0.138% | 0.557% | 0.038% | 0.021% |
| pWISE9120 | 453 | No data | No data | No data | No data | No data | No data |
| pWISE9121 | 454 | No data | No data | No data | No data | No data | No data |
| pWISE9122 | 455 | 2.503% | 1.873% | 0.988% | 1.246% | 0.015% | 0.029% |
| pWISE9123 | 456 | No data | No data | No data | No data | No data | No data |
| pWISE9124 | 457 | 0.519% | 0.663% | 0.419% | 0.299% | 0.005% | 0.001% |
| pWISE9125 | 458 | 1.169% | 0.697% | 0.796% | 0.795% | 0.002% | 0.000% |
| pWISE9126 | 459 | 0.169% | 0.174% | 0.107% | 0.098% | 0.008% | 0.017% |
| pWISE9127 | 460 | 0.195% | 0.071% | 0.035% | 0.026% | 0.003% | 0.005% |
| pWISE9128 | 461 | 0.712% | 0.438% | 0.123% | 0.174% | 0.004% | 0.002% |
| pWISE9134 | 426 | No data | No data | No data | No data | No data | No data |
| pWISE9135 | 427 | 0.932% | 0.240% | 0.136% | 0.160% | 0.009% | 0.002% |

| | | Standard Deviation of the average editing percent at the | | |
|---|---|---|---|---|
| | | nucleotides numbered according to the position in the PWsp389 | | |
| | Protein SEQ | spacer sequence on the non-target, genomic DNA strand | | |
| pWISE | ID NO: | C.27 | C.28 | C.30 |
| pWISE121 | 56 | No data | No data | No data |
| pWISE1921 | 428 | 0.003% | 0.011% | 0.007% |
| pWISE1925 | 429 | 0.000% | 0.003% | 0.002% |
| pWISE4332 | 430 | No data | No data | No data |
| pWISE4339 | 431 | No data | No data | No data |
| pWISE6852 | 432 | No data | No data | No data |
| pWISE6853 | 433 | 0.000% | 0.000% | 0.000% |
| pWISE9117 | 450 | 0.000% | 0.008% | 0.006% |
| pWISE9118 | 451 | 0.009% | 0.022% | 0.002% |
| pWISE9119 | 452 | 0.023% | 0.026% | 0.000% |
| pWISE9120 | 453 | No data | No data | No data |
| pWISE9121 | 454 | No data | No data | No data |
| pWISE9122 | 455 | 0.016% | 0.016% | 0.006% |
| pWISE9123 | 456 | No data | No data | No data |
| pWISE9124 | 457 | 0.011% | 0.016% | 0.004% |
| pWISE9125 | 458 | 0.054% | 0.028% | 0.000% |
| pWISE9126 | 459 | 0.000% | 0.012% | 0.005% |
| pWISE9127 | 460 | 0.003% | 0.001% | 0.004% |
| pWISE9128 | 461 | 0.000% | 0.009% | 0.000% |
| pWISE9134 | 426 | No data | No data | No data |
| pWISE9135 | 427 | 0.004% | 0.000% | 0.000% |

TABLE 20
numbered according to the position in the PWsp389 spacer

| | | Average G to A editing percent at the nucleotides | | | | |
|---|---|---|---|---|---|---|
| | | numbered according to the position in the PWsp389 spacer | | | | |
| | Protein SEQ | sequence on the opposite, target genomic DNA strand | | | | |
| pWISE | ID NO: | G.−9 | G.−8 | G.−7 | G.−6 | G.2 |
| pWISE121 | 56 | 0.004% | 0.000% | 0.000% | 0.011% | 0.154% |
| pWISE1921 | 428 | 0.054% | 0.005% | 0.005% | 0.000% | 0.000% |
| pWISE1925 | 429 | 0.030% | 0.005% | 0.002% | 0.007% | 0.022% |
| pWISE4332 | 430 | 0.000% | 0.000% | 0.000% | 0.000% | 0.000% |
| pWISE4339 | 431 | 0.003% | 0.003% | 0.000% | 0.003% | 0.009% |
| pWISE6852 | 432 | 0.068% | 0.000% | 0.007% | 0.007% | 0.068% |
| pWISE6853 | 433 | 0.004% | 0.004% | 0.000% | 0.008% | 0.000% |
| pWISE9117 | 450 | 0.038% | 0.034% | 0.003% | 0.000% | 0.000% |
| pWISE9118 | 451 | 0.078% | 0.053% | 0.001% | 0.000% | 0.008% |
| pWISE9119 | 452 | 0.039% | 0.006% | 0.002% | 0.013% | 0.002% |
| pWISE9120 | 453 | 0.000% | 0.000% | 0.000% | 0.000% | 0.000% |
| pWISE9121 | 454 | 0.008% | 0.008% | 0.000% | 0.000% | 0.000% |
| pWISE9122 | 455 | 0.006% | 0.011% | 0.002% | 0.005% | 0.018% |
| pWISE9123 | 456 | 0.021% | 0.007% | 0.000% | 0.000% | 0.007% |
| pWISE9124 | 457 | 0.007% | 0.029% | 0.014% | 0.004% | 0.006% |
| pWISE9125 | 458 | 0.000% | 0.003% | 0.000% | 0.000% | 0.005% |
| pWISE9126 | 459 | 0.004% | 0.000% | 0.000% | 0.000% | 0.000% |
| pWISE9127 | 460 | 0.002% | 0.006% | 0.001% | 0.001% | 0.004% |
| pWISE9128 | 461 | 0.016% | 0.000% | 0.000% | 0.005% | 0.013% |
| pWISE9134 | 426 | 0.060% | 0.000% | 0.005% | 0.000% | 0.000% |
| pWISE9135 | 427 | 0.023% | 0.005% | 0.008% | 0.003% | 0.005% |
| | | Average G to A editing percent at the nucleotides | | | | |
| | | numbered according to the position in the PWsp389 spacer | | | | |
| | Protein SEQ | sequence on the opposite, target genomic DNA strand | | | | |
| pWISE | ID NO: | G.14 | G.21 | G.23 | G.24 | G.32 |
| pWISE121 | 56 | 1.244% | 0.205% | 0.561% | 0.495% | 0.007% |
| pWISE1921 | 428 | 0.059% | 0.016% | 0.032% | 0.033% | 0.138% |
| pWISE1925 | 429 | 0.045% | 0.014% | 0.000% | 0.002% | 0.071% |
| pWISE4332 | 430 | 0.010% | 0.000% | 0.000% | 0.000% | 0.010% |
| pWISE4339 | 431 | 0.021% | 0.006% | 0.003% | 0.000% | 0.079% |
| pWISE6852 | 432 | 0.014% | 0.007% | 0.007% | 0.007% | 0.081% |
| pWISE6853 | 433 | 0.021% | 0.000% | 0.004% | 0.000% | 0.063% |
| pWISE9117 | 450 | 0.156% | 0.008% | 0.046% | 0.094% | 0.233% |
| pWISE9118 | 451 | 0.064% | 0.036% | 0.021% | 0.014% | 0.090% |
| pWISE9119 | 452 | 0.065% | 0.076% | 0.200% | 0.126% | 0.448% |
| pWISE9120 | 453 | 1.264% | 0.301% | 0.146% | 0.146% | 0.117% |
| pWISE9121 | 454 | 0.101% | 0.000% | 0.000% | 0.000% | 0.000% |
| pWISE9122 | 455 | 0.245% | 0.067% | 0.286% | 0.387% | 0.255% |
| pWISE9123 | 456 | 0.392% | 0.144% | 1.231% | 1.052% | 0.413% |
| pWISE9124 | 457 | 0.128% | 0.169% | 0.153% | 0.086% | 0.052% |
| pWISE9125 | 458 | 0.470% | 0.024% | 0.013% | 0.054% | 0.346% |
| pWISE9126 | 459 | 0.216% | 0.079% | 0.032% | 0.012% | 0.007% |
| pWISE9127 | 460 | 0.005% | 0.023% | 0.027% | 0.021% | 0.027% |
| pWISE9128 | 461 | 0.041% | 0.005% | 0.058% | 0.058% | 0.181% |
| pWISE9134 | 426 | 0.005% | 0.005% | 0.005% | 0.005% | 0.109% |
| pWISE9135 | 427 | 0.394% | 0.038% | 0.150% | 0.116% | 0.630% |

TABLE 21

| | | Standard deviation of the average G to A editing percent at the | | | | |
|---|---|---|---|---|---|---|
| | | nucleotides numbered according to the position in the PWsp389 | | | | |
| | Protein SEQ | spacer sequence on the opposite, target genomic DNA strand | | | | |
| pWISE | ID NO: | G.−9 | G.−8 | G.−7 | G.−6 | G.2 |
| pWISE121 | 56 | No data | No data | No data | No data | No data |
| pWISE1921 | 428 | 0.003% | 0.007% | 0.007% | 0.000% | 0.000% |
| pWISE1925 | 429 | 0.038% | 0.002% | 0.003% | 0.001% | 0.031% |
| pWISE4332 | 430 | No data | No data | No data | No data | No data |
| pWISE4339 | 431 | No data | No data | No data | No data | No data |
| pWISE6852 | 432 | No data | No data | No data | No data | No data |
| pWISE6853 | 433 | 0.006% | 0.006% | 0.000% | 0.000% | 0.000% |
| pWISE9117 | 450 | 0.018% | 0.024% | 0.004% | 0.000% | 0.000% |
| pWISE9118 | 451 | 0.029% | 0.055% | 0.001% | 0.000% | 0.002% |
| pWISE9119 | 452 | 0.013% | 0.002% | 0.003% | 0.012% | 0.003% |
| pWISE9120 | 453 | No data | No data | No data | No data | No data |
| pWISE9121 | 454 | No data | No data | No data | No data | No data |
| pWISE9122 | 455 | 0.003% | 0.004% | 0.002% | 0.007% | 0.026% |
| pWISE9123 | 456 | No data | No data | No data | No data | No data |
| pWISE9124 | 457 | 0.009% | 0.013% | 0.010% | 0.003% | 0.000% |
| pWISE9125 | 458 | 0.000% | 0.005% | 0.000% | 0.000% | 0.007% |
| pWISE9126 | 459 | 0.006% | 0.000% | 0.000% | 0.000% | 0.000% |
| pWISE9127 | 460 | 0.003% | 0.001% | 0.002% | 0.002% | 0.005% |
| pWISE9128 | 461 | 0.005% | 0.000% | 0.000% | 0.007% | 0.009% |
| pWISE9134 | 426 | No data | No data | No data | No data | No data |
| pWISE9135 | 427 | 0.029% | 0.002% | 0.002% | 0.000% | 0.002% |
| | | Standard deviation of the average G to A editing percent at the | | | | |
| | | nucleotides numbered according to the position in the PWsp389 | | | | |
| | Protein SEQ | spacer sequence on the opposite, target genomic DNA strand | | | | |
| pWISE | ID NO: | G.14 | G.21 | G.23 | G.24 | G.32 |
| pWISE121 | 56 | No data | No data | No data | No data | No data |
| pWISE1921 | 428 | 0.061% | 0.023% | 0.034% | 0.046% | 0.002% |
| pWISE1925 | 429 | 0.012% | 0.015% | 0.000% | 0.003% | 0.086% |
| pWISE4332 | 430 | No data | No data | No data | No data | No data |
| pWISE4339 | 431 | No data | No data | No data | No data | No data |
| pWISE6852 | 432 | No data | No data | No data | No data | No data |
| pWISE6853 | 433 | 0.029% | 0.000% | 0.006% | 0.000% | 0.016% |
| pWISE9117 | 450 | 0.076% | 0.012% | 0.041% | 0.069% | 0.046% |
| pWISE9118 | 451 | 0.068% | 0.021% | 0.019% | 0.020% | 0.001% |
| pWISE9119 | 452 | 0.024% | 0.014% | 0.137% | 0.052% | 0.058% |
| pWISE9120 | 453 | No data | No data | No data | No data | No data |
| pWISE9121 | 454 | No data | No data | No data | No data | No data |
| pWISE9122 | 455 | 0.134% | 0.028% | 0.013% | 0.145% | 0.053% |
| pWISE9123 | 456 | No data | No data | No data | No data | No data |
| pWISE9124 | 457 | 0.088% | 0.056% | 0.029% | 0.023% | 0.016% |
| pWISE9125 | 458 | 0.072% | 0.021% | 0.019% | 0.036% | 0.141% |
| pWISE9126 | 459 | 0.033% | 0.001% | 0.017% | 0.008% | 0.009% |
| pWISE9127 | 460 | 0.002% | 0.031% | 0.002% | 0.017% | 0.035% |
| pWISE9128 | 461 | 0.058% | 0.007% | 0.055% | 0.082% | 0.004% |
| pWISE9134 | 426 | No data | No data | No data | No data | No data |
| pWISE9135 | 427 | 0.085% | 0.005% | 0.062% | 0.068% | 0.181% |

TABLE 22

| | |
|---|---|
| | Average C to T editing percent at the nucleotides numbered |
| | according to the position in the PWsp390 spacer sequence on |
| Protein SEQ | the non-target, genomic DNA strand |

| pWISE | ID NO: | C.−5 | C.2 | C.6 | C.7 | C.8 | C.9 |
|---|---|---|---|---|---|---|---|
| pWISE121 | 56 | 0.001% | 0.036% | 0.204% | 0.243% | 0.271% | 0.306% |
| pWISE1921 | 428 | No data | No data | No data | No data | No data | No data |
| pWISE1925 | 429 | 0.004% | 4.734% | 16.811% | 16.945% | 17.111% | 17.252% |
| pWISE4332 | 430 | 0.007% | 1.615% | 9.902% | 10.126% | 10.478% | 10.624% |
| pWISE4339 | 431 | 0.021% | 1.945% | 12.224% | 12.230% | 12.411% | 12.516% |
| pWISE6852 | 432 | 0.006% | 1.154% | 6.962% | 7.048% | 7.260% | 7.323% |
| pWISE6853 | 433 | 0.006% | 1.719% | 12.179% | 12.324% | 12.571% | 12.862% |
| pWISE9117 | 450 | 0.041% | 1.451% | 10.661% | 11.872% | 12.949% | 13.410% |
| pWISE9118 | 451 | 0.004% | 1.054% | 1.660% | 2.057% | 3.233% | 4.731% |
| pWISE9119 | 452 | 0.025% | 2.798% | 14.296% | 16.976% | 18.713% | 19.812% |
| pWISE9120 | 453 | 0.046% | 0.381% | 0.494% | 0.702% | 0.894% | 1.114% |
| pWISE9121 | 454 | 0.056% | 1.364% | 2.252% | 2.827% | 4.559% | 6.308% |
| pWISE9122 | 455 | 0.016% | 2.888% | 4.090% | 5.096% | 7.686% | 9.968% |
| pWISE9123 | 456 | 0.035% | 1.986% | 13.883% | 15.149% | 16.342% | 17.234% |
| pWISE9124 | 457 | 0.049% | 1.177% | 3.785% | 4.339% | 6.071% | 7.191% |
| pWISE9125 | 458 | 0.025% | 2.081% | 15.070% | 15.595% | 17.056% | 17.938% |
| pWISE9126 | 459 | 0.000% | 0.532% | 0.662% | 0.713% | 1.069% | 1.196% |
| pWISE9127 | 460 | 0.021% | 0.468% | 1.118% | 1.168% | 2.348% | 3.537% |
| pWISE9128 | 461 | 0.008% | 2.151% | 7.752% | 8.531% | 11.355% | 14.796% |
| pWISE9134 | 426 | 0.020% | 4.185% | 16.492% | 16.768% | 17.044% | 17.304% |
| pWISE9135 | 427 | 0.061% | 2.445% | 15.598% | 16.287% | 17.347% | 18.365% |

| | | Average C to T editing percent at the nucleotides numbered | | | | | |
|---|---|---|---|---|---|---|---|
| | | according to the position in the PWsp390 spacer sequence on | | | | | |
| | Protein SEQ | the non-target, genomic DNA strand | | | | | |
| pWISE | ID NO: | C.11 | C.12 | C.14 | C.15 | C.16 | C.17 |
| pWISE121 | 56 | 0.308% | 0.392% | 0.187% | 0.728% | 1.621% | 2.842% |
| pWISE1921 | 428 | No data | No data | No data | No data | No data | No data |
| pWISE1925 | 429 | 17.241% | 17.406% | 8.709% | 9.728% | 9.726% | 9.821% |
| pWISE4332 | 430 | 10.649% | 10.694% | 6.360% | 6.625% | 6.376% | 6.485% |
| pWISE4339 | 431 | 12.478% | 12.483% | 8.800% | 9.120% | 8.912% | 8.841% |
| pWISE6852 | 432 | 7.427% | 7.392% | 4.040% | 4.316% | 4.333% | 4.436% |
| pWISE6853 | 433 | 12.750% | 12.862% | 8.214% | 8.590% | 8.483% | 8.209% |
| pWISE9117 | 450 | 13.765% | 13.761% | 3.804% | 5.090% | 5.741% | 7.314% |
| pWISE9118 | 451 | 9.524% | 9.034% | 0.662% | 1.272% | 1.622% | 1.975% |
| pWISE9119 | 452 | 20.612% | 20.307% | 8.860% | 11.277% | 11.756% | 12.877% |
| pWISE9120 | 453 | 1.990% | 1.966% | 0.322% | 0.637% | 0.731% | 0.770% |
| pWISE9121 | 454 | 12.786% | 12.213% | 0.915% | 1.715% | 2.034% | 2.295% |
| pWISE9122 | 455 | 16.937% | 17.006% | 6.783% | 9.797% | 10.570% | 11.929% |
| pWISE9123 | 456 | 17.516% | 17.518% | 7.876% | 9.803% | 10.296% | 10.554% |
| pWISE9124 | 457 | 8.179% | 8.052% | 2.610% | 4.134% | 4.586% | 5.049% |
| pWISE9125 | 458 | 19.331% | 19.180% | 7.894% | 9.511% | 10.417% | 10.657% |
| pWISE9126 | 459 | 1.681% | 1.681% | 0.317% | 0.662% | 0.724% | 0.738% |
| pWISE9127 | 460 | 7.514% | 7.037% | 0.309% | 0.619% | 0.850% | 1.043% |
| pWISE9128 | 461 | 15.344% | 15.376% | 5.060% | 5.483% | 5.811% | 6.444% |
| pWISE9134 | 426 | 17.281% | 17.332% | 7.595% | 8.535% | 8.609% | 8.941% |
| pWISE9135 | 427 | 19.656% | 19.504% | 6.609% | 8.847% | 10.648% | 10.740% |

| | | Average C to T editing percent at the nucleotides numbered | | | |
|---|---|---|---|---|---|
| | | according to the position in the PWsp390 spacer sequence on | | | |
| | Protein SEQ | the non-target, genomic DNA strand | | | |
| pWISE | ID NO: | C.19 | C.27 | C.28 | C.30 |
| pWISE121 | 56 | 1.681% | 0.001% | 0.000% | 0.071% |
| pWISE1921 | 428 | No data | No data | No data | No data |
| pWISE1925 | 429 | 3.939% | 0.014% | 0.035% | 0.002% |
| pWISE4332 | 430 | 1.635% | 0.000% | 0.005% | 0.000% |
| pWISE4339 | 431 | 2.099% | 0.000% | 0.000% | 0.004% |
| pWISE6852 | 432 | 1.429% | 0.006% | 0.000% | 0.000% |
| pWISE6853 | 433 | 2.441% | 0.011% | 0.011% | 0.006% |
| pWISE9117 | 450 | 5.321% | 0.007% | 0.011% | 0.035% |
| pWISE9118 | 451 | 0.982% | 0.021% | 0.025% | 0.004% |
| pWISE9119 | 452 | 7.808% | 0.018% | 0.014% | 0.027% |
| pWISE9120 | 453 | 0.854% | 0.056% | 0.049% | 0.009% |
| pWISE9121 | 454 | 0.872% | 0.000% | 0.006% | 0.000% |
| pWISE9122 | 455 | 7.986% | 0.036% | 0.020% | 0.003% |
| pWISE9123 | 456 | 6.218% | 0.016% | 0.024% | 0.014% |
| pWISE9124 | 457 | 2.675% | 0.008% | 0.016% | 0.013% |
| pWISE9125 | 458 | 3.721% | 0.004% | 0.004% | 0.004% |
| pWISE9126 | 459 | 0.680% | 0.000% | 0.000% | 0.008% |
| pWISE9127 | 460 | 0.364% | 0.002% | 0.000% | 0.000% |
| pWISE9128 | 461 | 2.416% | 0.020% | 0.016% | 0.008% |
| pWISE9134 | 426 | 3.120% | 0.008% | 0.037% | 0.000% |
| pWISE9135 | 427 | 5.677% | 0.006% | 0.030% | 0.000% |

TABLE 23

| | | Standard Deviation of the average editing percent at the | | | | | |
|---|---|---|---|---|---|---|---|
| | | nucleotides numbered according to the position in the PWsp390 | | | | | |
| | Protein SEQ | spacer sequence on the non-target, genomic DNA | | | | | |
| pWISE | ID NO: | C.−5 | C.2 | C.6 | C.7 | C.8 | C.9 |
| pWISE121 | 56 | No data | No data | No data | No data | No data | No data |
| pWISE1921 | 428 | No data | No data | No data | No data | No data | No data |
| pWISE1925 | 429 | 0.005% | 1.077% | 3.060% | 3.185% | 3.182% | 3.036% |
| pWISE4332 | 430 | 0.010% | 0.200% | 0.307% | 0.105% | 0.257% | 0.225% |
| pWISE4339 | 431 | 0.029% | 0.581% | 4.052% | 4.019% | 3.920% | 3.864% |
| pWISE6852 | 432 | No data | No data | No data | No data | No data | No data |
| pWISE6853 | 433 | No data | No data | No data | No data | No data | No data |
| pWISE9117 | 450 | 0.029% | 0.246% | 1.315% | 1.272% | 1.409% | 1.542% |
| pWISE9118 | 451 | 0.005% | 0.194% | 0.494% | 0.625% | 0.699% | 1.102% |
| pWISE9119 | 452 | 0.013% | 0.243% | 1.642% | 2.207% | 2.162% | 2.340% |
| pWISE9120 | 453 | 0.054% | 0.307% | 0.113% | 0.177% | 0.316% | 0.252% |
| pWISE9121 | 454 | 0.011% | 0.070% | 0.273% | 0.126% | 0.035% | 0.162% |
| pWISE9122 | 455 | 0.013% | 0.775% | 0.777% | 0.927% | 1.415% | 2.114% |
| pWISE9123 | 456 | 0.036% | 0.645% | 2.107% | 1.502% | 1.891% | 1.714% |
| pWISE9124 | 457 | 0.070% | 1.665% | 5.302% | 6.098% | 8.522% | 10.106% |
| pWISE9125 | 458 | 0.036% | 0.561% | 3.364% | 3.608% | 4.404% | 4.059% |
| pWISE9126 | 459 | 0.000% | 0.691% | 0.703% | 0.631% | 0.790% | 0.624% |
| pWISE9127 | 460 | 0.009% | 0.052% | 0.393% | 0.292% | 0.449% | 0.364% |
| pWISE9128 | 461 | 0.012% | 0.231% | 0.437% | 0.512% | 0.324% | 0.267% |
| pWISE9134 | 426 | 0.028% | 0.342% | 1.549% | 1.538% | 1.499% | 1.424% |
| pWISE9135 | 427 | 0.052% | 0.195% | 1.083% | 1.180% | 1.258% | 1.027% |

| | | Standard Deviation of the average editing percent at the | | | | | |
|---|---|---|---|---|---|---|---|
| | | nucleotides numbered according to the position in the PWsp390 | | | | | |
| | Protein SEQ | spacer sequence on the non-target, genomic DNA | | | | | |
| pWISE | ID NO: | C.11 | C.12 | C.14 | C.15 | C.16 | C.17 |
| pWISE121 | 56 | No data | No data | No data | No data | No data | No data |
| pWISE1921 | 428 | No data | No data | No data | No data | No data | No data |
| pWISE1925 | 429 | 3.064% | 3.114% | 2.169% | 2.252% | 2.163% | 2.319% |
| pWISE4332 | 430 | 0.101% | 0.125% | 0.758% | 0.973% | 0.448% | 0.629% |
| pWISE4339 | 431 | 3.885% | 3.818% | 3.164% | 3.477% | 3.289% | 2.889% |
| pWISE6852 | 432 | No data | No data | No data | No data | No data | No data |
| pWISE6853 | 433 | No data | No data | No data | No data | No data | No data |
| pWISE9117 | 450 | 1.797% | 1.705% | 0.779% | 0.954% | 0.996% | 1.255% |
| pWISE9118 | 451 | 0.978% | 0.885% | 0.004% | 0.032% | 0.094% | 0.472% |
| pWISE9119 | 452 | 2.413% | 2.310% | 1.219% | 1.622% | 1.354% | 1.236% |
| pWISE9120 | 453 | 0.354% | 0.320% | 0.135% | 0.184% | 0.162% | 0.150% |
| pWISE9121 | 454 | 1.081% | 0.999% | 0.159% | 0.115% | 0.159% | 0.082% |
| pWISE9122 | 455 | 4.434% | 4.445% | 1.720% | 2.239% | 2.319% | 2.600% |
| pWISE9123 | 456 | 1.446% | 1.443% | 0.636% | 0.994% | 1.467% | 1.832% |
| pWISE9124 | 457 | 11.490% | 11.311% | 3.692% | 5.847% | 6.473% | 7.128% |
| pWISE9125 | 458 | 3.820% | 4.046% | 2.077% | 2.700% | 2.260% | 1.981% |
| pWISE9126 | 459 | 0.953% | 0.953% | 0.346% | 0.703% | 0.628% | 0.596% |
| pWISE9127 | 460 | 0.632% | 0.340% | 0.036% | 0.112% | 0.090% | 0.153% |
| pWISE9128 | 461 | 0.374% | 0.502% | 0.323% | 0.143% | 0.061% | 0.488% |
| pWISE9134 | 426 | 1.352% | 1.384% | 0.284% | 0.366% | 0.309% | 0.484% |
| pWISE9135 | 427 | 1.182% | 1.295% | 1.011% | 1.241% | 1.609% | 1.257% |

| | | Standard Deviation of the average editing percent at the | | | |
|---|---|---|---|---|---|
| | | nucleotides numbered according to the position in the PWsp390 | | | |
| | Protein SEQ | spacer sequence on the non-target, genomic DNA strand | | | |
| pWISE | ID NO: | C.19 | C.27 | C.28 | C.30 |
| pWISE121 | 56 | No data | No data | No data | No data |
| pWISE1921 | 428 | No data | No data | No data | No data |
| pWISE1925 | 429 | 0.804% | 0.013% | 0.017% | 0.003% |
| pWISE4332 | 430 | 0.013% | 0.000% | 0.007% | 0.000% |
| pWISE4339 | 431 | 0.863% | 0.000% | 0.000% | 0.005% |
| pWISE6852 | 432 | No data | No data | No data | No data |
| pWISE6853 | 433 | No data | No data | No data | No data |
| pWISE9117 | 450 | 1.020% | 0.010% | 0.004% | 0.000% |
| pWISE9118 | 451 | 0.182% | 0.030% | 0.025% | 0.005% |
| pWISE9119 | 452 | 1.433% | 0.025% | 0.000% | 0.020% |
| pWISE9120 | 453 | 0.082% | 0.057% | 0.059% | 0.013% |
| pWISE9121 | 454 | 0.001% | 0.000% | 0.001% | 0.000% |
| pWISE9122 | 455 | 1.751% | 0.051% | 0.008% | 0.005% |
| pWISE9123 | 456 | 0.496% | 0.016% | 0.035% | 0.019% |
| pWISE9124 | 457 | 3.783% | 0.011% | 0.022% | 0.018% |
| pWISE9125 | 458 | 0.356% | 0.006% | 0.006% | 0.006% |
| pWISE9126 | 459 | 0.677% | 0.000% | 0.000% | 0.001% |
| pWISE9127 | 460 | 0.095% | 0.003% | 0.000% | 0.000% |
| pWISE9128 | 461 | 0.095% | 0.020% | 0.014% | 0.012% |
| pWISE9134 | 426 | 0.096% | 0.002% | 0.052% | 0.000% |
| pWISE9135 | 427 | 0.803% | 0.000% | 0.017% | 0.000% |

TABLE 24

| | | Average G to A editing percent at the nucleotides | | | | |
|---|---|---|---|---|---|---|
| | | numbered according to the position in the PWsp390 spacer | | | | |
| | Protein SEQ | sequence on the opposite, target genomic DNA strand | | | | |
| pWISE | ID NO: | G.−9 | G.−8 | G.4 | G.21 | G.23 |
| pWISE121 | 56 | 0.007% | 0.001% | 0.006% | 0.258% | 0.927% |
| pWISE1921 | 428 | No data | No data | No data | No data | No data |
| pWISE1925 | 429 | 0.027% | 0.025% | 0.149% | 0.019% | 0.099% |
| pWISE4332 | 430 | 0.000% | 0.052% | 0.168% | 0.002% | 0.036% |
| pWISE4339 | 431 | 0.000% | 0.000% | 0.080% | 0.034% | 0.069% |
| pWISE6852 | 432 | 0.006% | 0.000% | 0.121% | 0.011% | 0.000% |
| pWISE6853 | 433 | 0.067% | 0.045% | 0.101% | 0.028% | 0.000% |
| pWISE9117 | 450 | 0.026% | 0.044% | 0.046% | 0.052% | 0.058% |
| pWISE9118 | 451 | 0.016% | 0.000% | 0.128% | 0.040% | 0.109% |
| pWISE9119 | 452 | 0.002% | 0.010% | 0.006% | 0.018% | 0.435% |
| pWISE9120 | 453 | 0.008% | 0.004% | 0.077% | 0.762% | 0.688% |
| pWISE9121 | 454 | 0.050% | 0.071% | 0.081% | 0.034% | 0.180% |
| pWISE9122 | 455 | 0.003% | 0.006% | 0.035% | 0.077% | 0.509% |
| pWISE9123 | 456 | 0.019% | 0.000% | 0.019% | 0.014% | 0.100% |
| pWISE9124 | 457 | 0.021% | 0.010% | 0.010% | 0.153% | 0.355% |
| pWISE9125 | 458 | 0.029% | 0.000% | 0.029% | 0.008% | 0.034% |
| pWISE9126 | 459 | 0.000% | 0.000% | 0.022% | 0.079% | 0.050% |
| pWISE9127 | 460 | 0.008% | 0.008% | 0.004% | 0.004% | 0.093% |
| pWISE9128 | 461 | 0.006% | 0.003% | 0.008% | 0.030% | 0.046% |
| pWISE9134 | 426 | 0.026% | 0.026% | 0.028% | 0.047% | 0.086% |
| pWISE9135 | 427 | 0.000% | 0.000% | 0.015% | 0.036% | 0.152% |

| | | Average G to A editing percent at the nucleotides | | | |
|---|---|---|---|---|---|
| | | numbered according to the position in the PWsp390 spacer | | | |
| | Protein SEQ | sequence on the opposite, target genomic DNA strand | | | |
| pWISE | ID NO: | G.24 | G.25 | G.26 | G.33 |
| pWISE121 | 56 | 0.187% | 0.090% | 0.071% | 0.073% |
| pWISE1921 | 428 | No data | No data | No data | No data |
| pWISE1925 | 429 | 0.064% | 0.023% | 0.023% | 0.262% |
| pWISE4332 | 430 | 0.009% | 0.000% | 0.002% | 0.085% |
| pWISE4339 | 431 | 0.089% | 0.004% | 0.000% | 0.146% |
| pWISE6852 | 432 | 0.000% | 0.006% | 0.006% | 0.034% |
| pWISE6853 | 433 | 0.022% | 0.000% | 0.006% | 0.050% |
| pWISE9117 | 450 | 0.021% | 0.024% | 0.024% | 0.387% |
| pWISE9118 | 451 | 0.049% | 0.033% | 0.015% | 0.029% |
| pWISE9119 | 452 | 0.322% | 0.216% | 0.180% | 0.639% |
| pWISE9120 | 453 | 0.449% | 0.319% | 0.240% | 0.083% |
| pWISE9121 | 454 | 0.228% | 0.009% | 0.014% | 0.069% |
| pWISE9122 | 455 | 0.478% | 0.231% | 0.151% | 0.398% |
| pWISE9123 | 456 | 0.106% | 0.129% | 0.105% | 0.529% |
| pWISE9124 | 457 | 0.326% | 0.186% | 0.127% | 0.196% |
| pWISE9125 | 458 | 0.055% | 0.017% | 0.013% | 0.503% |
| pWISE9126 | 459 | 0.032% | 0.007% | 0.012% | 0.004% |
| pWISE9127 | 460 | 0.091% | 0.067% | 0.062% | 0.140% |
| pWISE9128 | 461 | 0.041% | 0.004% | 0.015% | 0.170% |
| pWISE9134 | 426 | 0.089% | 0.027% | 0.042% | 0.116% |
| pWISE9135 | 427 | 0.155% | 0.143% | 0.121% | 0.440% |

TABLE 25

| | | Standard deviation of the average G to A editing percent at the | | | | |
|---|---|---|---|---|---|---|
| | | nucleotides numbered according to the position in the PWsp390 | | | | |
| | Protein | spacer sequence on the opposite, target genomic DNA strand | | | | |
| pWISE | SEQ ID NO: | G.-9 | G.-8 | G.4 | G.21 | G.23 |
| pWISE121 | 56 | No data | No data | No data | No data | No data |
| pWISE1921 | 428 | No data | No data | No data | No data | No data |
| pWISE1925 | 429 | 0.026% | 0.029% | 0.048% | 0.027% | 0.011% |
| pWISE4332 | 430 | 0.000% | 0.074% | 0.131% | 0.003% | 0.050% |
| pWISE4339 | 431 | 0.000% | 0.000% | 0.103% | 0.048% | 0.086% |
| pWISE6852 | 432 | No data | No data | No data | No data | No data |
| pWISE6853 | 433 | No data | No data | No data | No data | No data |
| pWISE9117 | 450 | 0.037% | 0.062% | 0.046% | 0.074% | 0.042% |
| pWISE9118 | 451 | 0.002% | 0.000% | 0.036% | 0.057% | 0.032% |
| pWISE9119 | 452 | 0.002% | 0.005% | 0.002% | 0.016% | 0.095% |
| pWISE9120 | 453 | 0.011% | 0.006% | 0.057% | 0.104% | 0.042% |
| pWISE9121 | 454 | 0.042% | 0.071% | 0.008% | 0.009% | 0.022% |
| pWISE9122 | 455 | 0.004% | 0.001% | 0.030% | 0.022% | 0.150% |
| pWISE9123 | 456 | 0.020% | 0.000% | 0.020% | 0.019% | 0.135% |
| pWISE9124 | 457 | 0.029% | 0.015% | 0.001% | 0.216% | 0.464% |
| pWISE9125 | 458 | 0.042% | 0.000% | 0.030% | 0.012% | 0.048% |
| pWISE9126 | 459 | 0.000% | 0.000% | 0.031% | 0.112% | 0.071% |
| pWISE9127 | 460 | 0.001% | 0.001% | 0.005% | 0.006% | 0.036% |
| pWISE9128 | 461 | 0.009% | 0.004% | 0.012% | 0.042% | 0.030% |
| pWISE9134 | 426 | 0.017% | 0.017% | 0.001% | 0.000% | 0.026% |
| pWISE9135 | 427 | 0.000% | 0.000% | 0.022% | 0.026% | 0.111% |

| | | Standard deviation of the average G to A editing percent at the | | | |
|---|---|---|---|---|---|
| | | nucleotides numbered according to the position in the PWsp390 | | | |
| | Protein | spacer sequence on the opposite, target genomic DNA strand | | | |
| pWISE | SEQ ID NO: | G.24 | G.25 | G.26 | G.33 |
| pWISE121 | 56 | No data | No data | No data | No data |
| pWISE1921 | 428 | No data | No data | No data | No data |
| pWISE1925 | 429 | 0.060% | 0.032% | 0.032% | 0.050% |
| pWISE4332 | 430 | 0.013% | 0.000% | 0.003% | 0.106% |
| pWISE4339 | 431 | 0.035% | 0.005% | 0.000% | 0.152% |
| pWISE6852 | 432 | No data | No data | No data | No data |
| pWISE6853 | 433 | No data | No data | No data | No data |
| pWISE9117 | 450 | 0.029% | 0.034% | 0.034% | 0.052% |
| pWISE9118 | 451 | 0.003% | 0.016% | 0.021% | 0.041% |
| pWISE9119 | 452 | 0.018% | 0.045% | 0.067% | 0.001% |
| pWISE9120 | 453 | 0.105% | 0.144% | 0.091% | 0.049% |
| pWISE9121 | 454 | 0.002% | 0.012% | 0.020% | 0.057% |
| pWISE9122 | 455 | 0.049% | 0.030% | 0.076% | 0.032% |
| pWISE9123 | 456 | 0.150% | 0.021% | 0.056% | 0.149% |
| pWISE9124 | 457 | 0.461% | 0.263% | 0.179% | 0.264% |
| pWISE9125 | 458 | 0.077% | 0.012% | 0.006% | 0.094% |
| pWISE9126 | 459 | 0.046% | 0.010% | 0.004% | 0.005% |
| pWISE9127 | 460 | 0.040% | 0.063% | 0.018% | 0.030% |
| pWISE9128 | 461 | 0.011% | 0.006% | 0.003% | 0.059% |
| pWISE9134 | 426 | 0.022% | 0.029% | 0.021% | 0.044% |
| pWISE9135 | 427 | 0.081% | 0.116% | 0.077% | 0.029% |

### Example 5

EnAsCas12a circular permutants according to some embodiments of the present invention were tested. A glycine-serine linker was used to connect the native N- and C-termini of EnAsCas12a (SEQ ID NO:298). The glycine-serine linker had either 10 amino acids and a sequence of (GSS)ₙG (SEQ ID NO:36), wherein n is 3, or 16 amino acids and a sequence of (GSS)ₙG (SEQ ID NO:36), wherein n is 5. The circular permutants and control are listed in Table 26 and the targets are provided in Table 27.

TABLE 26
Tested EnAsCas12a constructs

| | Vector SEQ | DNA SEQ | | Protein |
|---|---|---|---|---|
| pWISE | ID NO: | ID NO: | Description | SEQ ID NO: |
| pWISE121 | 285 | 949 | EnAsCas12a | 298 |
| | | | nuclease(control) | |
| pWISE8345 | 286 | 327 | CP13_AsCas12a | 299 |
| pWISE8346 | 287 | 328 | CP14_AsCas12a | 300 |
| pWISE8347 | 288 | 329 | CP15_AsCas12a | 301 |
| pWISE8348 | 289 | 330 | CP16_AsCas12a | 302 |
| pWISE8349 | 290 | 331 | CP17_AsCas12a | 303 |
| pWISE8350 | 291 | 332 | CP18_AsCas12a | 304 |
| pWISE8351 | 292 | 333 | CP19_AsCas12a | 305 |
| pWISE8352 | 293 | 334 | CP20_AsCas12a | 306 |
| pWISE8353 | 294 | 335 | CP21_AsCas12a | 307 |
| pWISE8354 | 295 | 336 | CP22_AsCas12a | 308 |
| pWISE8355 | 296 | 337 | CP23_AsCas12a | 309 |
| pWISE8356 | 297 | 338 | CP24_AsCas12a | 310 |

TABLE 27
Targets

| Target | Target | | Spacer | | Vector |
|---|---|---|---|---|---|
| Nucleic | SEQ ID | | SEQ | | SEQ |
| Acid | NO: | Spacer | ID NO: | pWISE | ID NO: |
| human DNMT1 | 272 | PWsp1956 | 804 | pWISE5986 | 814 |
| human FANCF | 273 | PWsp1959 | 805 | pWISE5989 | 815 |
| human RNF2 | 274 | PWsp1960 | 806 | pWISE5990 | 816 |

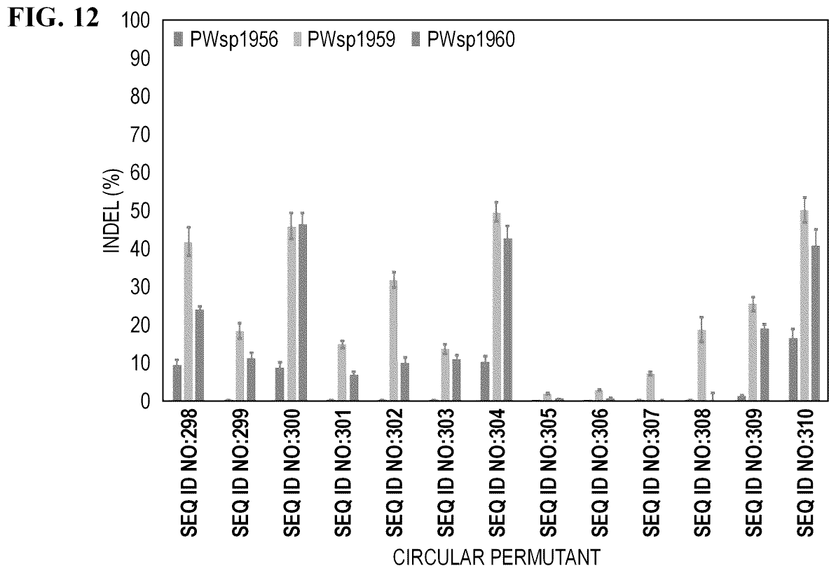

The circular permutants and control were tested for their ability to generate INDELS at three different sites across two target nucleic acids (SEQ ID NOs: 273-274) in HEK293T cells using three spacers (SEQ ID NOs: 804-806). Three biological replicates and two technical replicates were tested. HEK293T cells were seeded into 48-well collagen-coated plates (Corning) in the absence of an antibiotic using DMEM media. At 70-80% confluency, cells were transfected with 1.5 μL of LTX (ThermoFisher Scientific) using 500 ng of the control or circular permutant plasmid and 500 ng of guide RNA plasmid according to manufacturer's protocol. After 3 days, the cells were lysed with a crude extraction method using TritonX buffer. Each control or circular permutant was scored based on the indel placement percentage in the FANCF and RNF2 genes using the guide RNAs. The results are shown in FIG. 12. As seen in FIG. 12, several EnAsCas12a circular permutants worked well in achieving high INDEL percentages.

### Example 6

Circular permutants according to some embodiments were tested in soy by optimizing the codon usage, promoter sequence, terminator sequence, and nuclear localization sequences for expression in soy. The circular permutants and control are listed in Table 28 and the targets are listed in Table 29.

TABLE 28
Constructs tested in soy

| | Vector | DNA | Description of | Protein |
|---|---|---|---|---|
| | SEQ | SEQ | soy optimized | SEQ |
| pWISE | ID NO: | ID NO: | construct | ID NO: |
| pWISE3380 | 734 | 246 | LbCas12a (control) | 56 |
| pWISE9517 | 728 | 248 | CP02-LbCas12a | 235 |
| pWISE9519 | 729 | 250 | CP04-LbCas12a | 237 |
| pWISE9521 | 730 | 252 | CP06-LbCas12a | 239 |
| pWISE9523 | 731 | 254 | CP08-LbCas12a | 241 |
| pWISE9525 | 732 | 256 | CP10-LbCas12a | 243 |
| pWISE9529 | 733 | 258 | CP12-LbCas12a | 245 |

TABLE 29
Targets

| Target Nucleic | Target SEQ ID | | Spacer SEQ ID |
|---|---|---|---|
| Acid | NO: | Spacer | NO: |
| Soy AHK4 | 840 | PWsp1243 | 807 |
| Locus906 | | | |
| Soy AHK4 | 841 | PWsp2567 | 808 |
| Locus215 | | | |

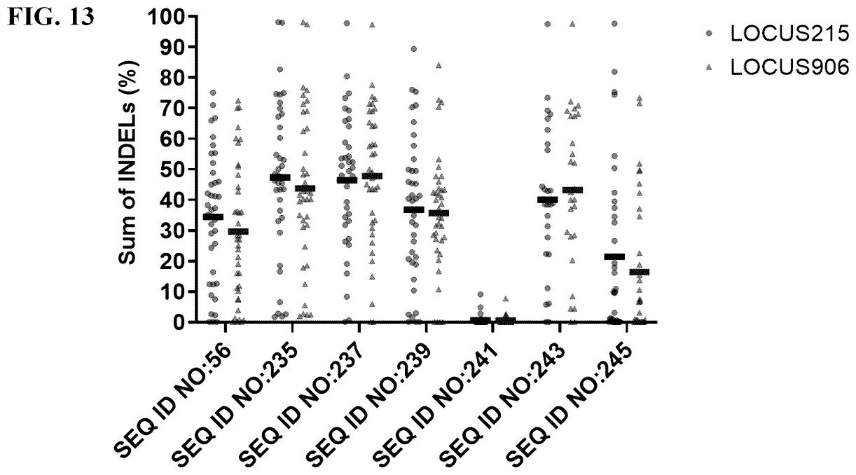

The circular permutants and control were tested for their ability to generate INDELS at two sites in the AHK4 gene (SEQ ID NOs: 840-841) in soy plants using two spacers (SEQ ID NOs: 807-808). 30-40 soy plants were recovered for each combination of locus and circular permutant. Each control or circular permutant was scored based on indel placement percentage in the AHK4 gene using the guide RNAs. The results are provided in FIG. 13. As seen in FIG. 13, several of the circular permutants that were optimized for rapid soy achieved high INDEL percentages and some had activity comparable to or better than LbCas 12a in rapid soy.

### Example 7

Additional circular permutants of LbCas12a other than those described in Example 1 were tested. The circular permutants and control (LbCas12a; SEQ ID NO:56) are listed in Table 30.

TABLE 30
Tested constructs.

| | Vector SEQ | DNA SEQ | Protein SEQ |
|---|---|---|---|
| pWISE | ID NO: | ID NO: | ID NO: |
| pWISE121 | 259 | 246 | 56 |
| pWISE8346 | 261 | 248 | 235 |
| pWISE10270 | 585 | 561 | 537 |
| pWISE10271 | 586 | 562 | 538 |
| pWISE10272 | 587 | 563 | 539 |
| pWISE10273 | 588 | 564 | 540 |
| pWISE8348 | 263 | 250 | 237 |
| pWISE10274 | 589 | 565 | 541 |
| pWISE10275 | 590 | 566 | 542 |
| pWISE10276 | 591 | 567 | 543 |
| pWISE10277 | 592 | 568 | 544 |
| pWISE8350 | 265 | 252 | 239 |
| pWISE10278 | 593 | 569 | 545 |
| pWISE10279 | 594 | 570 | 546 |
| pWISE10280 | 595 | 571 | 547 |
| pWISE10281 | 596 | 572 | 548 |
| pWISE8351 | 266 | 253 | 240 |
| pWISE10282 | 597 | 573 | 549 |
| pWISE10283 | 598 | 574 | 550 |
| pWISE10284 | 599 | 575 | 551 |
| pWISE10285 | 600 | 576 | 552 |
| pWISE8354 | 269 | 256 | 243 |
| pWISE10286 | 601 | 577 | 553 |
| pWISE10287 | 602 | 578 | 554 |
| pWISE10288 | 603 | 579 | 555 |
| pWISE10289 | 604 | 580 | 556 |
| pWISE8356 | 271 | 258 | 245 |
| pWISE10290 | 605 | 581 | 557 |
| pWISE10291 | 606 | 582 | 558 |
| pWISE10292 | 607 | 583 | 559 |
| pWISE10293 | 608 | 584 | 560 |

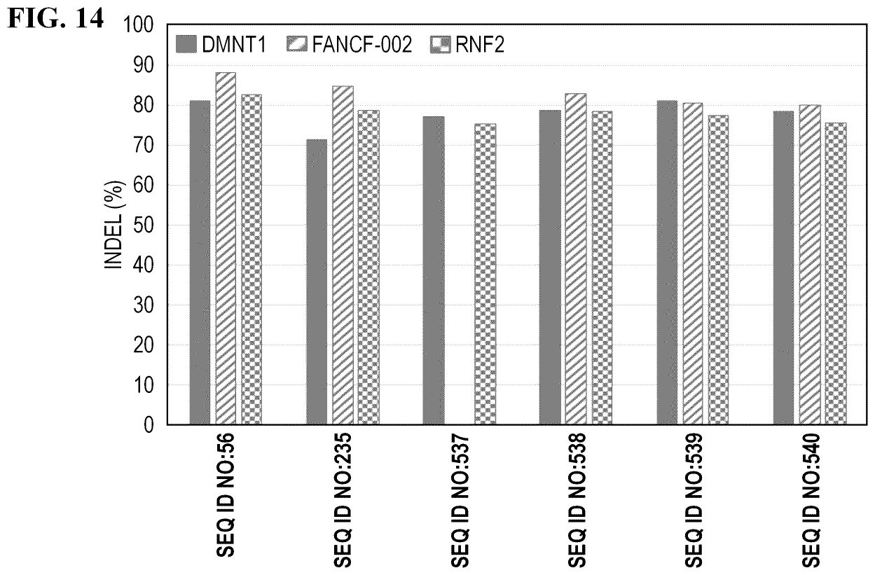

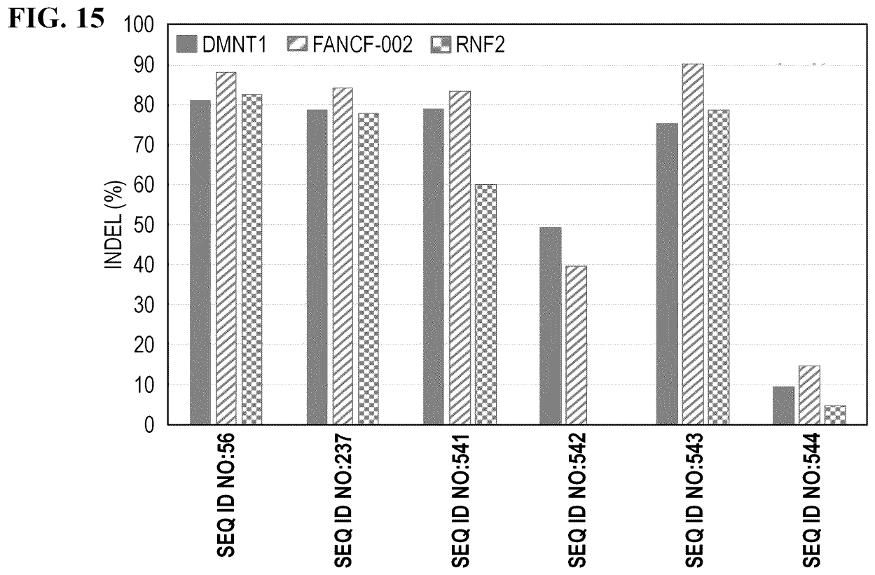

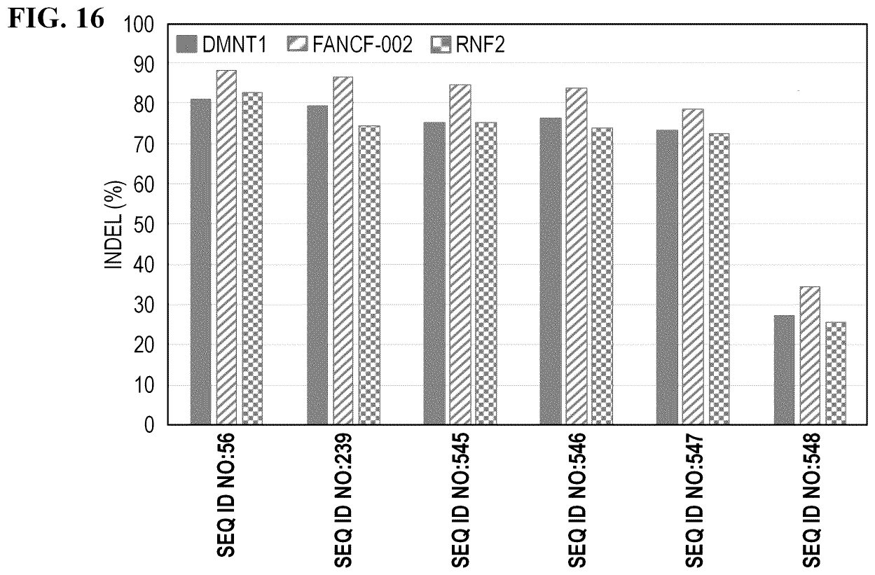

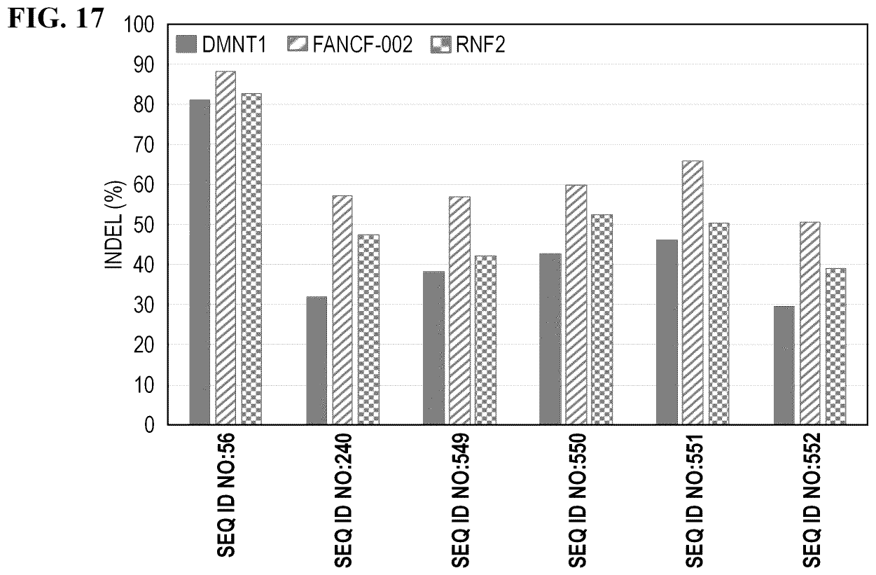

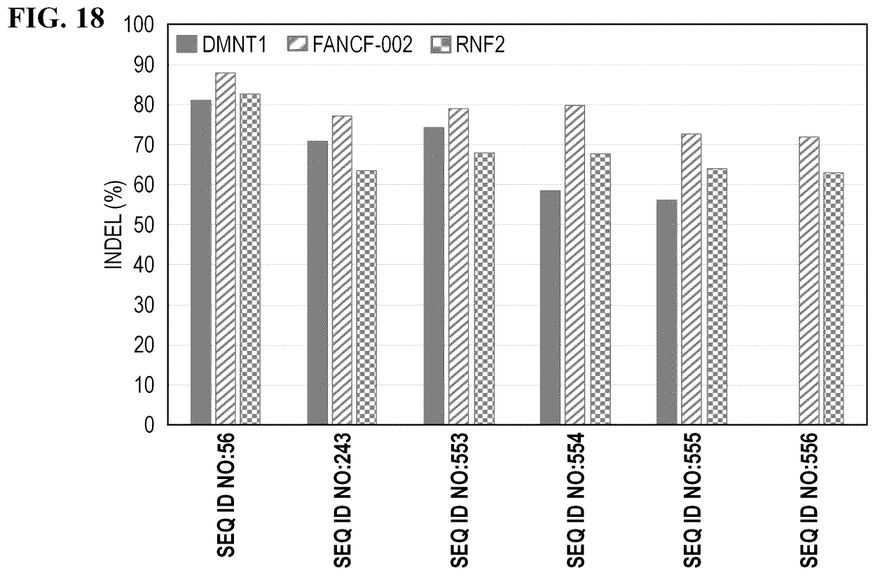

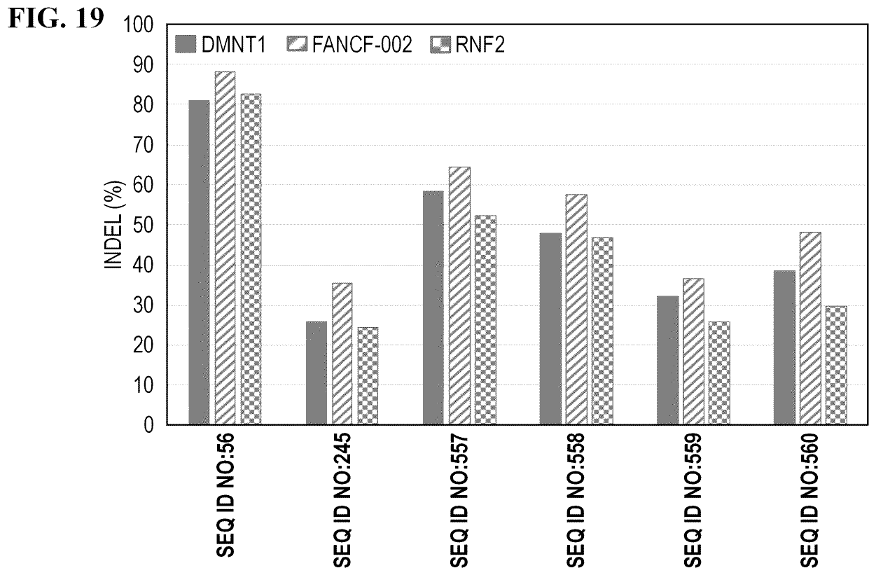

The circular permutants and control were tested for their ability to generate INDELS in the DNMT1, FANCF, and RNF2 genes (SEQ ID NOs: 272-274) in HEK293T cells using the spacers according to SEQ ID NOs: 275-277. Two technical replicates were tested. HEK293T cells were seeded into 48-well collagen-coated plates (Corning) in the absence of an antibiotic using DMEM media. At 70-80% confluency, cells were transfected with 1.5 μL of LTX (ThermoFisher Scientific) using 500 ng of the control or circular permutant plasmid and 500 ng of guide RNA plasmid according to manufacturer's protocol. After 3 days, the cells were lysed with a crude extraction method using TritonX buffer. Each control or circular permutant was scored based on the precise base pair editing and indel placement percentage in the DNMT1, FANCF, and RNF2 genes using the guide RNAs. Results are provided in FIGS. 14-19 and demonstrate that each of the circular permutants had measurable INDEL activity.

### Example 8

Arginine substitution variants of the soy optimized circular permutants described in Example 6 were tested in soy. The arginine mutations were analogous to a D156R mutation in LbCas12a. These circular permutants and the control are listed in Table 31. The arginine mutations are numbered according to the protein sequence listed in the fourth column of Table 31.

TABLE 31
Tested constructs.

| | Vector | DNA | Description of | Protein |
|---|---|---|---|---|
| | SEQ | SEQ | soy optimized | SEQ |
| pWISE | ID NO: | ID NO: | construct | ID NO: |
| pWISE9528 | 755 | 748 | LbCas12a + | 741 |
| | | | D156R (control) | |
| pWISE9516 | 749 | 742 | CP02 + D71R | 735 |
| pWISE9518 | 750 | 743 | CP04 + D1107R | 736 |
| pWISE9520 | 751 | 744 | CP06 + D243R | 737 |
| pWISE9522 | 752 | 745 | CP08 + D882R | 738 |
| pWISE9524 | 753 | 746 | CP10 + D957R | 739 |
| pWISE9526 | 754 | 747 | CP12 + D282R | 740 |

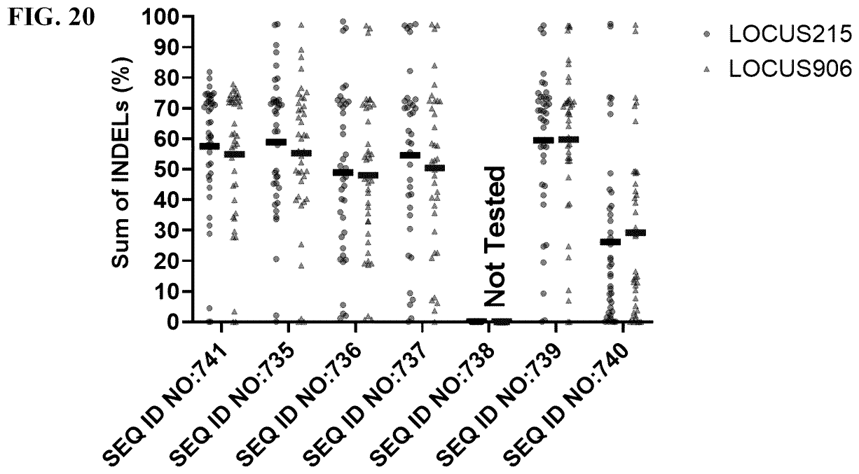

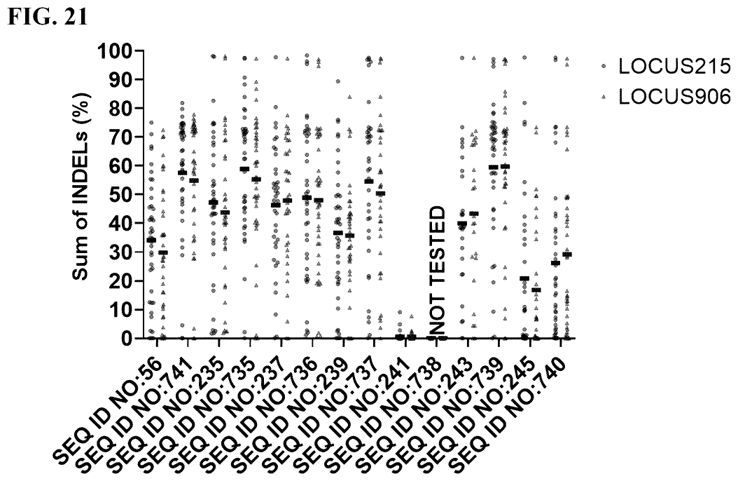

The circular permutants and control were tested for their ability to generate INDELS at two sites in the AHK4 gene (SEQ ID NOs: 840-841) in rapid soy plants using one spacer (SEQ ID NO: 807). 30-40 soy plants were transformed for each combination of locus and variant soy optimized circular permutant. Each control or circular permutant was scored based on indel placement percentage in the AHK4 gene using the guide RNAs. The results are provided in FIGS. 20 and 21 with FIG. 21 providing a comparison to the constructs described in Example 6. As seen in FIGS. 20 and 21, several of the circular permutants achieved high INDEL percentages and some had activity comparable to or better than LbCas12a and LbCas12a+D156R in rapid soy. For the majority of the circular permutants, the mutation had a similar effect to that seen for LbCas12a D156R relative to wild-type LbCas12a.

### Example 9

Circular permutants of a soy optimized engineered nuclease were tested in soy. These circular permutants (SEQ ID NOs: 390 and 757-760) and controls (SEQ ID NOs: 56 and 761) are listed in Table 32.

TABLE 32
Tested constructs.

| | Vector SEQ | DNA SEQ | Protein SEQ |
|---|---|---|---|
| pWISE | ID NO: | ID NO: | ID NO: |
| pWISE3380 | 734 | 246 | 56 |
| pWISE6345 | 773 | 767 | 761 |
| pWISE10428 | 768 | 402 | 390 |
| pWISE10429 | 769 | 763 | 757 |
| pWISE10430 | 770 | 764 | 758 |
| pWISE10431 | 771 | 765 | 759 |
| pWISE10432 | 772 | 766 | 760 |

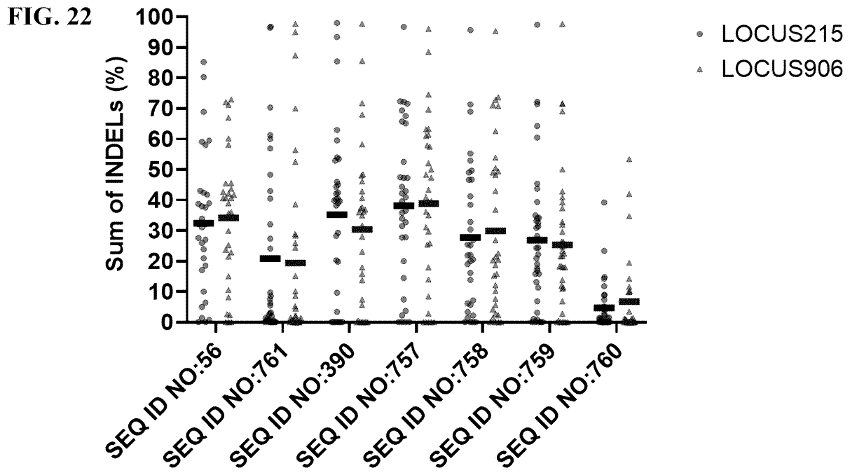

The circular permutants and controls were tested for their ability to generate INDELS at two sites in the AHK4 gene (SEQ ID NOs: 840-841) in rapid soy plants using one spacer (SEQ ID NO:807). 30-40 soy plants were transformed for each combination of locus and variant soy optimized circular permutant. Each control or circular permutant was scored based on indel placement percentage in the AHK4 gene using the guide RNAs. The results are provided in FIG. 22. Several of the circular permutants achieved high average INDEL percentages similar to LbCas12a (SEQ ID NO:56) and outperformed the soy optimized engineered nuclease (SEQ ID NO:761).

### Example 10

Circular permutants of an enzymatically inactive LbCas12a (dLbCas12a (SEQ ID NO: 59)) were tested for cytosine base editing by fusing a UGI to the C-terminus of the circular permutant and a cytosine deaminase to the N-terminus of the circular permutant with a linker between the circular permutant and cytosine deaminase to provide a fusion protein. The tested linkers between the circular permutant and cytosine deaminase included a GS-XTEN-GS linker (SEQ ID NO:30); an XTEN linker (SEQ ID NO:29); a six amino acid glycine serine (6X-GS) linker of (SGS)ₙ wherein n is 2 (SEQ ID NO:869); an eight amino acid glycine-serine (8X-GS) linker of (SGGS)ₙ wherein n is 2 (SEQ ID NO:870); and a twelve amino acid glycine-serine (12X-GS) linker of (SGGS)ₙ wherein n is 3 (SEQ ID NO:870). The fusion proteins (SEQ ID NOs: 456 and 624-628)—and control (SEQ ID NO:56) re listed in Table 33 and the targets are provided in Table 34.

TABLE 33
Tested constructs.

| | Vector SEQ | DNA SEQ | Protein SEQ |
|---|---|---|---|
| pWISE | ID NO: | ID NO: | ID NO: |
| pWISE121 | 259 | 246 | 56 |
| pWISE9123 | 480 | 468 | 456 |
| pWISE10643 | 686 | 655 | 624 |
| pWISE10644 | 687 | 656 | 625 |
| pWISE10645 | 688 | 657 | 626 |
| pWISE10646 | 689 | 658 | 627 |
| pWISE10647 | 690 | 659 | 628 |

TABLE 34
Targets

| Target | Target | | Spacer | | Vector |
|---|---|---|---|---|---|
| Nucleic | SEQ | | SEQ | | SEQ |
| Acid | ID NO: | Spacer | ID NO: | pWISE | ID NO: |
| human FANCF | 273 | PWsp132 | 799 | pWISE878 | 809 |
| human FANCF | 273 | PWsp449 | 276 | pWISE878 | 279 |
| human AAVS1 | 863 | PWsp390 | 802 | pWISE5986 | 812 |
| human RUNX1 | 864 | PWsp135 | 800 | pWISE5989 | 810 |

The fusion proteins and control were tested for their ability to perform base editing at five different sites in three target nucleic acids (SEQ ID NOs: 273, 863, and 864) in HEK293T cells using four spacers (SEQ ID NOs: 276, 799, 800, and 802). A single biological replicate and two technical replicates were tested. HEK293T cells were seeded into 48-well collagen-coated plates (Corning) in the absence of an antibiotic using DMEM media. At 70-80% confluency, cells were transfected with 1.5 μL of LTX (ThermoFisher Scientific) using 500 ng of the control or fusion protein plasmid and 500 ng of guide RNA plasmid according to manufacturer's protocol. After 3 days, the cells were lysed with a crude extraction method using TritonX buffer. Each control or fusion protein was scored based on the precise base pair editing in the FANCF, RUNX1, and AAVS1 genes using the guide RNAs (SEQ ID NOs: 276 and 799-802). Low, background levels of INDEL formation were seen for the fusion proteins, which was expected from using a dead LbCas12a. The results are provided in Tables 35-54. The values in Tables 35-54 that are below 0.1% are considered to be in the noise of the instrument (below the limit of detection) and are not indicative of editing. Values that are between 0.1% and 0.5% indicate that editing is present in the experiment at the specified location, but the assay is not sensitive enough to accurately quantify the amount of base editing. As seen in Tables 35-54, the fusion proteins provided efficient C to T editing over a wide editing window and changing the length and/or type of the linker between the cytosine deaminase and the circular permutant did not significantly modify the editing window or efficiency at positions shown to be highly edited with the control. In addition, the fusion proteins containing shorter linkers were found to have improved C to T editing towards the 3′ end of the spacer (e.g., at about the last 5-7 nucleotides of the spacer sequence).

TABLE 35

| | | Average C to T editing percent at the nucleotides numbered | | | | | | |
|---|---|---|---|---|---|---|---|---|
| | | according to the position in the PWsp132 spacer sequence on the | | | | | | |
| | Protein | non-target, genomic DNA strand | | | | | | |
| pWISE | SEQ ID NO: | C.-7 | C.-6 | C.-4 | C.-1 | C.2 | C.3 | C.11 |
| pWISE121 | 56 | 0.039% | 0.052% | 0.028% | 0.043% | 0.097% | 0.093% | 0.277% |
| pWISE9123 | 456 | 0.163% | 0.095% | 0.022% | 0.623% | 0.011% | 0.067% | 18.926% |
| pWISE10643 | 624 | 0.266% | 0.148% | 0.019% | 0.843% | 0.062% | 0.085% | 29.592% |
| pWISE10644 | 625 | 0.253% | 0.101% | 0.019% | 0.627% | 0.032% | 0.095% | 27.603% |
| pWISE10645 | 626 | 0.131% | 0.080% | 0.004% | 0.611% | 0.044% | 0.032% | 19.961% |
| pWISE10646 | 627 | 0.195% | 0.083% | 0.042% | 0.727% | 0.074% | 0.162% | 28.278% |
| pWISE10647 | 628 | 0.095% | 0.075% | 0.024% | 0.517% | 0.071% | 0.077% | 19.680% |

| | | Average C to T editing percent at the nucleotides numbered | | | | | |
|---|---|---|---|---|---|---|---|
| | | according to the position in the PWsp132 spacer sequence on the | | | | | |
| | Protein | non-target, genomic DNA strand | | | | | |
| pWISE | SEQ ID NO: | C.14 | C.17 | C.19 | C.21 | C.24 | C.28 |
| pWISE121 | 56 | 1.076% | 0.870% | 0.687% | 0.477% | 0.153% | 0.176% |
| pWISE9123 | 456 | 17.483% | 2.140% | 14.776% | 10.536% | 0.067% | 0.067% |
| pWISE10643 | 624 | 28.137% | 3.954% | 23.827% | 16.232% | 0.039% | 0.000% |
| pWISE10644 | 625 | 26.456% | 4.167% | 23.170% | 16.952% | 0.025% | 0.019% |
| pWISE10645 | 626 | 19.542% | 3.876% | 18.300% | 13.597% | 0.135% | 0.021% |
| pWISE10646 | 627 | 28.197% | 5.506% | 26.825% | 20.603% | 0.193% | 0.135% |
| pWISE10647 | 628 | 19.619% | 4.468% | 19.085% | 15.061% | 0.306% | 0.224% |

TABLE 36

| | | Standard Deviation of the average editing percent at the | | | | | | |
|---|---|---|---|---|---|---|---|---|
| | | nucleotides numbered according to the position in the PWsp132 | | | | | | |
| | Protein | spacer sequence on the non-target, genomic DNA strand | | | | | | |
| pWISE | SEQ ID NO: | C.-7 | C.-6 | C.-4 | C.-1 | C.2 | C.3 | C.11 |
| pWISE121 | 56 | 0.050% | 0.025% | 0.009% | 0.006% | 0.082% | 0.003% | 0.090% |
| pWISE9123 | 456 | No data | No data | No data | No data | No data | No data | No data |
| pWISE10643 | 624 | 0.007% | 0.104% | 0.016% | 0.096% | 0.034% | 0.046% | 0.346% |
| pWISE10644 | 625 | No data | No data | No data | No data | No data | No data | No data |
| pWISE10645 | 626 | 0.052% | 0.041% | 0.006% | 0.096% | 0.027% | 0.009% | 2.321% |
| pWISE10646 | 627 | 0.088% | 0.019% | 0.009% | 0.062% | 0.065% | 0.101% | 4.114% |
| pWISE10647 | 628 | 0.044% | 0.073% | 0.023% | 0.231% | 0.005% | 0.038% | 12.047% |

| | | Standard Deviation of the average editing percent at the | | | | | |
|---|---|---|---|---|---|---|---|
| | | nucleotides numbered according to the position in the PWsp132 | | | | | |
| | Protein | spacer sequence on the non-target, genomic DNA strand | | | | | |
| pWISE | SEQ ID NO: | C.14 | C.17 | C.19 | C.21 | C.24 | C.28 |
| pWISE121 | 56 | 0.130% | 0.481% | 0.258% | 0.094% | 0.003% | 0.121% |
| pWISE9123 | 456 | No data | No data | No data | No data | No data | No data |
| pWISE10643 | 624 | 0.455% | 0.450% | 0.190% | 0.857% | 0.023% | 0.000% |
| pWISE10644 | 625 | No data | No data | No data | No data | No data | No data |
| pWISE10645 | 626 | 2.262% | 0.363% | 2.534% | 1.382% | 0.131% | 0.012% |
| pWISE10646 | 627 | 3.968% | 0.479% | 3.774% | 2.106% | 0.102% | 0.145% |
| pWISE10647 | 628 | 12.044% | 2.759% | 11.764% | 9.300% | 0.270% | 0.219% |

TABLE 37

| | | Average G to A editing percent at the nucleotides numbered | | | | |
|---|---|---|---|---|---|---|
| | | according to the position in the PWsp132 spacer sequence on the | | | | |
| | Protein | opposite, target genomic DNA strand | | | | |
| pWISE | SEQ ID NO: | G.-5 | G.6 | G.7 | G.9 | G.12 |
| pWISE121 | 56 | 0.053% | 0.094% | 0.091% | 0.210% | 0.189% |
| pWISE9123 | 456 | 0.011% | 0.000% | 0.011% | 0.275% | 0.326% |
| pWISE10643 | 624 | 0.019% | 0.007% | 0.062% | 0.161% | 0.092% |
| pWISE10644 | 625 | 0.019% | 0.025% | 0.038% | 0.184% | 0.101% |
| pWISE10645 | 626 | 0.013% | 0.021% | 0.039% | 0.147% | 0.122% |
| pWISE10646 | 627 | 0.008% | 0.027% | 0.058% | 0.257% | 0.134% |
| pWISE10647 | 628 | 0.009% | 0.029% | 0.058% | 0.307% | 0.475% |
| | | Average G to A editing percent at the nucleotides numbered | | | | |
| | | according to the position in the PWsp132 spacer sequence on the | | | | |
| | Protein | opposite, target genomic DNA strand | | | | |
| pWISE | SEQ ID NO: | G.13 | G.15 | G.23 | G.25 | G.30 |
| pWISE121 | 56 | 0.282% | 0.462% | 0.899% | 0.299% | 0.113% |
| pWISE9123 | 456 | 0.337% | 0.854% | 1.179% | 1.196% | 4.150% |
| pWISE10643 | 624 | 0.043% | 0.704% | 1.113% | 2.039% | 7.594% |
| pWISE10644 | 625 | 0.044% | 0.709% | 1.089% | 2.242% | 7.181% |
| pWISE10645 | 626 | 0.096% | 0.727% | 1.246% | 2.223% | 4.849% |
| pWISE10646 | 627 | 0.110% | 1.106% | 1.910% | 3.507% | 7.690% |
| pWISE10647 | 628 | 0.464% | 1.130% | 1.526% | 2.811% | 5.804% |

TABLE 38

| | | Standard deviation of the average G to A editing percent at the | | | | |
|---|---|---|---|---|---|---|
| | | nucleotides numbered according to the position in the PWsp132 | | | | |
| | Protein | spacer sequence on the opposite, target genomic DNA strand | | | | |
| pWISE | SEQ ID NO: | G.-5 | G.6 | G.7 | G.9 | G.12 |
| pWISE121 | 56 | 0.075% | 0.078% | 0.074% | 0.102% | 0.026% |
| pWISE9123 | 456 | No data | No data | No data | No data | No data |
| pWISE10643 | 624 | 0.016% | 0.001% | 0.034% | 0.069% | 0.078% |
| pWISE10644 | 625 | No data | No data | No data | No data | No data |
| pWISE10645 | 626 | 0.018% | 0.012% | 0.008% | 0.006% | 0.065% |
| pWISE10646 | 627 | 0.001% | 0.008% | 0.036% | 0.001% | 0.008% |
| pWISE10647 | 628 | 0.012% | 0.017% | 0.001% | 0.173% | 0.305% |
| | | Standard deviation of the average G to A editing percent at the | | | | |
| | | nucleotides numbered according to the position in the PWsp132 | | | | |
| | Protein | spacer sequence on the opposite, target genomic DNA strand | | | | |
| pWISE | SEQ ID NO: | G.13 | G.15 | G.23 | G.25 | G.30 |
| pWISE121 | 56 | 0.052% | 0.055% | 0.291% | 0.014% | 0.077% |
| pWISE9123 | 456 | No data | No data | No data | No data | No data |
| pWISE10643 | 624 | 0.018% | 0.266% | 0.105% | 0.475% | 0.545% |
| pWISE10644 | 625 | No data | No data | No data | No data | No data |
| pWISE10645 | 626 | 0.001% | 0.133% | 0.249% | 0.611% | 1.151% |
| pWISE10646 | 627 | 0.031% | 0.025% | 0.216% | 0.364% | 0.954% |
| pWISE10647 | 628 | 0.273% | 0.187% | 0.647% | 1.725% | 3.185% |

TABLE 39

| | | Average C to T editing percent at the nucleotides numbered | | | | | |
|---|---|---|---|---|---|---|---|
| | | according to the position in the PWsp449 spacer sequence on the | | | | | |
| | Protein | non-target, genomic DNA strand | | | | | |
| pWISE | SEQ ID NO: | C.-5 | C.-1 | C.2 | C.10 | C.11 | C.15 |
| pWISE121 | 56 | 0.095% | 0.158% | 0.123% | 0.401% | 0.431% | 2.083% |
| pWISE9123 | 456 | 1.400% | 1.976% | 0.215% | 32.361% | 31.901% | 31.984% |
| pWISE10643 | 624 | 1.672% | 2.237% | 0.297% | 38.859% | 38.221% | 38.715% |
| pWISE10644 | 625 | 1.296% | 1.994% | 0.326% | 35.125% | 34.600% | 34.817% |
| pWISE10645 | 626 | 1.343% | 2.371% | 0.369% | 38.229% | 37.569% | 38.221% |
| pWISE10646 | 627 | 1.075% | 2.088% | 0.349% | 35.195% | 34.944% | 35.296% |
| pWISE10647 | 628 | 1.198% | 2.470% | 0.348% | 41.096% | 40.766% | 41.317% |

| | | Average C to T editing percent at the nucleotides numbered | | | | |
|---|---|---|---|---|---|---|
| | | according to the position in the PWsp449 spacer sequence on the | | | | |
| | Protein | non-target, genomic DNA strand | | | | |
| pWISE | SEQ ID NO: | C.20 | C.22 | C.30 | C.32 | C.33 |
| pWISE121 | 56 | 0.505% | 1.066% | 1.515% | 1.047% | 1.095% |
| pWISE9123 | 456 | 4.524% | 1.672% | 0.216% | 0.064% | 0.047% |
| pWISE10643 | 624 | 6.493% | 2.701% | 0.361% | 0.048% | 0.050% |
| pWISE10644 | 625 | 5.222% | 1.890% | 0.306% | 0.057% | 0.080% |
| pWISE10645 | 626 | 7.171% | 3.732% | 0.543% | 0.068% | 0.098% |
| pWISE10646 | 627 | 6.277% | 3.662% | 0.500% | 0.056% | 0.091% |
| pWISE10647 | 628 | 7.456% | 4.823% | 0.794% | 0.162% | 0.207% |

TABLE 40

| | | Standard Deviation of the average editing percent at the | | | | | |
|---|---|---|---|---|---|---|---|
| | | nucleotides numbered according to the position in the PWsp449 | | | | | |
| | Protein | spacer sequence on the non-target, genomic DNA strand | | | | | |
| pWISE | SEQ ID NO: | C.-5 | C.-1 | C.2 | C.10 | C.11 | C.15 |
| pWISE121 | 56 | 0.067% | 0.050% | 0.037% | 0.011% | 0.017% | 0.001% |
| pWISE9123 | 456 | 0.029% | 0.157% | 0.013% | 1.234% | 1.451% | 1.429% |
| pWISE10643 | 624 | 0.156% | 0.078% | 0.033% | 2.566% | 2.362% | 2.577% |
| pWISE10644 | 625 | 0.049% | 0.195% | 0.012% | 1.883% | 1.646% | 1.715% |
| pWISE10645 | 626 | 0.069% | 0.248% | 0.058% | 0.739% | 0.543% | 0.412% |
| pWISE10646 | 627 | 0.177% | 0.166% | 0.033% | 2.131% | 2.060% | 2.098% |
| pWISE10647 | 628 | 0.180% | 0.218% | 0.105% | 1.145% | 1.220% | 1.075% |

| | | Standard Deviation of the average editing percent at the | | | | |
|---|---|---|---|---|---|---|
| | | nucleotides numbered according to the position in the PWsp449 | | | | |
| | Protein | spacer sequence on the non-target, genomic DNA strand | | | | |
| pWISE | SEQ ID NO: | C.20 | C.22 | C.30 | C.32 | C.33 |
| pWISE121 | 56 | 0.035% | 0.223% | 0.938% | 0.690% | 0.714% |
| pWISE9123 | 456 | 0.318% | 0.139% | 0.010% | 0.002% | 0.042% |
| pWISE10643 | 624 | 0.431% | 0.270% | 0.225% | 0.052% | 0.048% |
| pWISE10644 | 625 | 0.231% | 0.136% | 0.131% | 0.014% | 0.013% |
| pWISE10645 | 626 | 0.081% | 0.220% | 0.266% | 0.028% | 0.000% |
| pWISE10646 | 627 | 0.309% | 0.016% | 0.048% | 0.010% | 0.022% |
| pWISE10647 | 628 | 0.012% | 0.339% | 0.033% | 0.014% | 0.022% |

TABLE 41

| | | Average G to A editing percent at the nucleotides numbered | | | | | |
|---|---|---|---|---|---|---|---|
| | | according to the position in the PWsp449 spacer sequence on the | | | | | |
| | Protein | opposite, target genomic DNA strand | | | | | |
| pWISE | SEQ ID NO: | G.-9 | G.1 | G.3 | G.4 | G.7 | G.17 |
| pWISE121 | 56 | 0.048% | 0.105% | 0.144% | 0.133% | 0.178% | 0.670% |
| pWISE9123 | 456 | 0.068% | 0.021% | 0.036% | 0.064% | 0.019% | 0.682% |
| pWISE10643 | 624 | 0.016% | 0.016% | 0.022% | 0.034% | 0.005% | 1.130% |
| pWISE10644 | 625 | 0.026% | 0.034% | 0.033% | 0.049% | 0.010% | 0.921% |
| pWISE10645 | 626 | 0.010% | 0.008% | 0.020% | 0.031% | 0.015% | 1.235% |
| pWISE10646 | 627 | 0.035% | 0.035% | 0.047% | 0.042% | 0.026% | 1.278% |
| pWISE10647 | 628 | 0.011% | 0.032% | 0.046% | 0.066% | 0.038% | 1.642% |

| | | Average G to A editing percent at the nucleotides numbered | | | | |
|---|---|---|---|---|---|---|
| | | according to the position in the PWsp449 spacer sequence on the | | | | |
| | Protein | opposite, target genomic DNA strand | | | | |
| pWISE | SEQ ID NO: | G.21 | G.24 | G.26 | G.28 | G.31 |
| pWISE121 | 56 | 0.425% | 0.732% | 0.656% | 0.146% | 0.194% |
| pWISE9123 | 456 | 1.387% | 7.126% | 6.427% | 0.711% | 1.559% |
| pWISE10643 | 624 | 2.098% | 10.400% | 9.246% | 1.087% | 2.132% |
| pWISE10644 | 625 | 2.210% | 9.591% | 8.436% | 1.185% | 2.234% |
| pWISE10645 | 626 | 2.959% | 13.013% | 11.749% | 2.111% | 3.075% |
| pWISE10646 | 627 | 2.993% | 11.783% | 10.963% | 1.986% | 2.821% |
| pWISE10647 | 628 | 4.148% | 15.842% | 14.591% | 3.042% | 4.037% |

TABLE 42

| | | Standard deviation of the average G to A editing percent at the | | | | | |
|---|---|---|---|---|---|---|---|
| | | nucleotides numbered according to the position in the PWsp449 | | | | | |
| | Protein | spacer sequence on the opposite, target genomic DNA strand | | | | | |
| pWISE | SEQ ID NO: | G.-9 | G.1 | G.3 | G.4 | G.7 | G.17 |
| pWISE121 | 56 | 0.024% | 0.042% | 0.018% | 0.021% | 0.018% | 0.152% |
| pWISE9123 | 456 | 0.043% | 0.017% | 0.027% | 0.028% | 0.020% | 0.137% |
| pWISE10643 | 624 | 0.007% | 0.015% | 0.008% | 0.033% | 0.008% | 0.088% |
| pWISE10644 | 625 | 0.037% | 0.026% | 0.017% | 0.004% | 0.008% | 0.045% |
| pWISE10645 | 626 | 0.015% | 0.002% | 0.021% | 0.016% | 0.021% | 0.251% |
| pWISE10646 | 627 | 0.034% | 0.019% | 0.018% | 0.029% | 0.016% | 0.020% |
| pWISE10647 | 628 | 0.003% | 0.039% | 0.046% | 0.018% | 0.011% | 0.093% |

| | | Standard deviation of the average G to A editing percent at the | | | | |
|---|---|---|---|---|---|---|
| | | nucleotides numbered according to the position in the PWsp449 | | | | |
| | Protein | spacer sequence on the opposite, target genomic DNA strand | | | | |
| pWISE | SEQ ID NO: | G.21 | G.24 | G.26 | G.28 | G.31 |
| pWISE121 | 56 | 0.144% | 0.201% | 0.297% | 0.083% | 0.045% |
| pWISE9123 | 456 | 0.061% | 0.422% | 0.566% | 0.083% | 0.033% |
| pWISE10643 | 624 | 0.134% | 0.774% | 0.628% | 0.087% | 0.435% |
| pWISE10644 | 625 | 0.028% | 0.419% | 0.469% | 0.081% | 0.123% |
| pWISE10645 | 626 | 0.564% | 0.378% | 0.411% | 0.232% | 0.158% |
| pWISE10646 | 627 | 0.055% | 0.029% | 0.044% | 0.170% | 0.080% |
| pWISE10647 | 628 | 0.130% | 0.097% | 0.086% | 0.202% | 0.008% |

TABLE 43

| | | Average C to T editing percent at the nucleotides numbered | | | | | | |
|---|---|---|---|---|---|---|---|---|
| | | according to the position in the PWsp135 spacer sequence on the | | | | | | |
| | Protein | non-target, genomic DNA strand | | | | | | |
| pWISE | SEQ ID NO: | C.-8 | C.-6 | C.-1 | C.3 | C.4 | C.6 | C.8 |
| pWISE121 | 56 | 0.012% | 0.011% | 0.054% | 0.017% | 0.030% | 0.342% | 0.327% |
| pWISE9123 | 456 | 0.234% | 0.009% | 1.556% | 0.027% | 0.064% | 16.533% | 15.417% |
| pWISE10643 | 624 | 0.244% | 0.002% | 1.736% | 0.017% | 0.041% | 21.563% | 20.357% |
| pWISE10644 | 625 | 0.287% | 0.008% | 2.409% | 0.039% | 0.047% | 27.834% | 26.754% |
| pWISE10645 | 626 | 0.211% | 0.026% | 2.402% | 0.059% | 0.088% | 29.354% | 28.047% |
| pWISE10646 | 627 | 0.304% | 0.014% | 2.513% | 0.122% | 0.127% | 30.168% | 29.065% |
| pWISE10647 | 628 | 0.254% | 0.005% | 2.280% | 0.082% | 0.073% | 30.279% | 29.132% |

| | | Average C to T editing percent at the nucleotides numbered | | | | | |
|---|---|---|---|---|---|---|---|
| | | according to the position in the PWsp135 spacer sequence on the | | | | | |
| | Protein | non-target, genomic DNA strand | | | | | |
| pWISE | SEQ ID NO: | C.9 | C.10 | C.11 | C.13 | C.17 | C.18 |
| pWISE121 | 56 | 0.368% | 0.359% | 0.352% | 0.382% | 0.512% | 0.540% |
| pWISE9123 | 456 | 16.268% | 16.358% | 16.514% | 16.635% | 5.964% | 6.610% |
| pWISE10643 | 624 | 21.206% | 21.283% | 21.423% | 21.650% | 8.925% | 10.127% |
| pWISE10644 | 625 | 27.616% | 27.715% | 27.786% | 27.959% | 13.392% | 14.599% |
| pWISE10645 | 626 | 29.037% | 29.233% | 29.436% | 29.733% | 19.309% | 20.354% |
| pWISE10646 | 627 | 30.163% | 30.294% | 30.483% | 30.583% | 21.660% | 22.612% |
| pWISE10647 | 628 | 30.141% | 30.210% | 30.396% | 30.585% | 22.017% | 22.833% |

| | | Average C to T editing percent at the nucleotides numbered | | | | | |
|---|---|---|---|---|---|---|---|
| | | according to the position in the PWsp135 spacer sequence on the | | | | | |
| | Protein | non-target, genomic DNA strand | | | | | |
| pWISE | SEQ ID NO: | C.19 | C.22 | C.25 | C.27 | C.29 | C.33 |
| pWISE121 | 56 | 0.501% | 0.378% | 0.115% | 0.085% | 0.123% | 0.057% |
| pWISE9123 | 456 | 9.843% | 0.173% | 0.198% | 0.225% | 0.201% | 0.226% |
| pWISE10643 | 624 | 14.063% | 0.349% | 0.253% | 0.324% | 0.352% | 0.265% |
| pWISE10644 | 625 | 19.052% | 0.465% | 0.398% | 0.439% | 0.403% | 0.339% |
| pWISE10645 | 626 | 23.992% | 1.459% | 1.081% | 1.189% | 1.207% | 0.972% |
| pWISE10646 | 627 | 25.918% | 1.725% | 1.205% | 1.383% | 1.321% | 1.103% |
| pWISE10647 | 628 | 26.311% | 1.921% | 1.331% | 1.408% | 1.379% | 1.111% |

TABLE 44

| | | Standard Deviation of the average editing percent at the nucleotides | | | | | | |
|---|---|---|---|---|---|---|---|---|
| | | numbered according to the position in the PWsp135 spacer sequence on | | | | | | |
| | Protein SEQ | the non-target, genomic DNA strand | | | | | | |
| pWISE | ID NO: | C.-8 | C.-6 | C.-1 | C.3 | C.4 | C.6 | C.8 |
| pWISE121 | 56 | 0.002% | 0.015% | 0.064% | 0.011% | 0.014% | 0.411% | 0.437% |
| pWISE9123 | 456 | 0.098% | 0.009% | 1.050% | 0.024% | 0.069% | 12.400% | 11.665% |
| pWISE10643 | 624 | 0.037% | 0.002% | 0.347% | 0.005% | 0.010% | 5.778% | 5.450% |
| pWISE10644 | 625 | 0.051% | 0.001% | 0.050% | 0.010% | 0.007% | 2.156% | 2.039% |
| pWISE10645 | 626 | No data | No data | No data | No data | No data | No data | No data |
| pWISE10646 | 627 | 0.057% | 0.014% | 0.043% | 0.032% | 0.025% | 0.711% | 0.374% |
| pWISE10647 | 628 | 0.048% | 0.008% | 0.275% | 0.046% | 0.015% | 0.860% | 1.143% |

| | |
|---|---|
| | Standard Deviation of the average editing percent at the nucleotides |
| | numbered according to the position in the PWsp135 spacer |
| Protein SEQ | sequence on the non-target, genomic DNA strand |

| pWISE | ID NO: | C.9 | C.10 | C.11 | C.13 | C.17 | C.18 |
|---|---|---|---|---|---|---|---|
| pWISE121 | 56 | 0.454% | 0.441% | 0.409% | 0.435% | 0.456% | 0.378% |
| pWISE9123 | 456 | 12.240% | 12.293% | 12.418% | 12.446% | 4.241% | 4.813% |
| pWISE10643 | 624 | 5.658% | 5.695% | 5.663% | 5.699% | 2.346% | 2.455% |
| pWISE10644 | 625 | 2.220% | 2.196% | 2.172% | 2.152% | 1.202% | 1.207% |
| pWISE10645 | 626 | No data | No data | No data | No data | No data | No data |
| pWISE10646 | 627 | 0.790% | 0.722% | 0.755% | 0.909% | 0.658% | 0.810% |
| pWISE10647 | 628 | 1.016% | 0.928% | 0.918% | 0.829% | 0.644% | 0.549% |

| | |
|---|---|
| | Standard Deviation of the average editing percent at the nucleotides |
| | numbered according to the position in the PWsp135 spacer |
| Protein SEQ | sequence on the non-target, genomic DNA strand |

| pWISE | ID NO: | C.19 | C.22 | C.25 | C.27 | C.29 | C.33 |
|---|---|---|---|---|---|---|---|
| pWISE121 | 56 | 0.467% | 0.079% | 0.073% | 0.021% | 0.076% | 0.056% |
| pWISE9123 | 456 | 7.183% | 0.087% | 0.136% | 0.110% | 0.162% | 0.122% |
| pWISE10643 | 624 | 3.867% | 0.123% | 0.126% | 0.173% | 0.153% | 0.099% |
| pWISE10644 | 625 | 1.510% | 0.085% | 0.064% | 0.029% | 0.037% | 0.023% |
| pWISE10645 | 626 | No data | No data | No data | No data | No data | No data |
| pWISE10646 | 627 | 0.704% | 0.106% | 0.183% | 0.007% | 0.006% | 0.095% |
| pWISE10647 | 628 | 1.205% | 0.083% | 0.184% | 0.067% | 0.047% | 0.180% |

TABLE 45

| | | Average G to A editing percent at the nucleotides | | |
|---|---|---|---|---|
| | | numbered according to the position in the PWsp135 spacer | | |
| | Protein SEQ | sequence on the opposite, target genomic DNA strand | | |
| pWISE | ID NO: | G.-4 | G.2 | G.16 |
| pWISE121 | 56 | 0.003% | 0.028% | 0.546% |
| pWISE9123 | 456 | 0.001% | 0.006% | 0.109% |
| pWISE10643 | 624 | 0.009% | 0.004% | 0.199% |
| pWISE10644 | 625 | 0.006% | 0.014% | 0.172% |
| pWISE10645 | 626 | 0.006% | 0.000% | 0.517% |
| pWISE10646 | 627 | 0.002% | 0.007% | 0.510% |
| pWISE10647 | 628 | 0.003% | 0.008% | 0.657% |

TABLE 46

| | | Standard deviation of the average G to A editing percent at the | | |
|---|---|---|---|---|
| | | nucleotides numbered according to the position in the PWsp135 | | |
| | Protein SEQ | spacer sequence on the opposite, target genomic DNA strand | | |
| pWISE | ID NO: | G.-4 | G.2 | G.16 |
| pWISE121 | 56 | 0.005% | 0.002% | 0.290% |
| pWISE9123 | 456 | 0.002% | 0.001% | 0.085% |
| pWISE10643 | 624 | 0.002% | 0.006% | 0.003% |
| pWISE10644 | 625 | 0.001% | 0.004% | 0.051% |
| pWISE10645 | 626 | No data | No data | No data |
| pWISE10646 | 627 | 0.002% | 0.010% | 0.060% |
| pWISE10647 | 628 | 0.004% | 0.007% | 0.111% |

TABLE 47

| | | Average C to T editing percent at the nucleotides numbered | | | | |
|---|---|---|---|---|---|---|
| | | according to the position in the PWsp389 spacer sequence on | | | | |
| | Protein SEQ | the non-target, genomic DNA strand | | | | |
| pWISE | ID NO: | C.-5 | C.-1 | C.4 | C.6 | C.7 |
| pWISE121 | 56 | 0.000% | 0.070% | 0.068% | 0.094% | 0.270% |
| pWISE9123 | 456 | 0.006% | 0.834% | 0.897% | 6.149% | 21.236% |
| pWISE10643 | 624 | 0.027% | 0.713% | 0.970% | 7.783% | 25.729% |
| pWISE10644 | 625 | 0.010% | 0.738% | 1.080% | 8.182% | 25.666% |
| pWISE10645 | 626 | 0.040% | 1.246% | 1.452% | 7.392% | 30.506% |
| pWISE10646 | 627 | 0.022% | 0.914% | 1.349% | 5.465% | 31.416% |
| pWISE10647 | 628 | 0.008% | 1.057% | 1.285% | 4.425% | 30.272% |

| | | Average C to T editing percent at the nucleotides numbered | | | | |
|---|---|---|---|---|---|---|
| | | according to the position in the PWsp389 spacer sequence on | | | | |
| | Protein SEQ | the non-target, genomic DNA strand | | | | |
| pWISE | ID NO: | C.11 | C.12 | C.16 | C.17 | C.18 |
| pWISE121 | 56 | 0.168% | 0.254% | 0.726% | 0.805% | 0.793% |
| pWISE9123 | 456 | 27.208% | 27.249% | 11.176% | 8.338% | 8.484% |
| pWISE10643 | 624 | 30.862% | 31.021% | 15.051% | 11.834% | 11.892% |
| pWISE10644 | 625 | 29.986% | 30.157% | 17.982% | 15.163% | 14.980% |
| pWISE10645 | 626 | 36.301% | 36.625% | 27.609% | 25.063% | 24.404% |
| pWISE10646 | 627 | 37.283% | 37.658% | 29.920% | 27.394% | 26.508% |
| pWISE10647 | 628 | 35.584% | 36.017% | 29.926% | 27.729% | 26.942% |
| | | Average C to T editing percent at the nucleotides numbered | | | | |
| | | according to the position in the PWsp389 spacer sequence on | | | | |
| | Protein SEQ | the non-target, genomic DNA strand | | | | |
| pWISE | ID NO: | C.25 | C.26 | C.27 | C.28 | C.30 |
| pWISE121 | 56 | 0.052% | 0.120% | 0.087% | 0.175% | 0.082% |
| pWISE9123 | 456 | 0.039% | 0.026% | 0.040% | 0.046% | 0.023% |
| pWISE10643 | 624 | 0.021% | 0.037% | 0.072% | 0.076% | 0.050% |
| pWISE10644 | 625 | 0.058% | 0.061% | 0.110% | 0.111% | 0.052% |
| pWISE10645 | 626 | 0.209% | 0.229% | 0.380% | 0.393% | 0.192% |
| pWISE10646 | 627 | 0.194% | 0.200% | 0.397% | 0.363% | 0.140% |
| pWISE10647 | 628 | 0.219% | 0.208% | 0.393% | 0.401% | 0.132% |

TABLE 48

| | | Standard Deviation of the average editing percent at the | | | | |
|---|---|---|---|---|---|---|
| | | nucleotides numbered according to the position in the PWsp389 | | | | |
| | Protein SEQ | spacer sequence on the non-target, genomic DNA strand | | | | |
| pWISE | ID NO: | C.-5 | C.-1 | C.4 | C.6 | C.7 |
| pWISE121 | 56 | 0.000% | 0.077% | 0.005% | 0.012% | 0.004% |
| pWISE9123 | 456 | 0.008% | 0.235% | 0.032% | 0.179% | 0.060% |
| pWISE10643 | 624 | 0.013% | 0.227% | 0.030% | 0.375% | 0.041% |
| pWISE10644 | 625 | 0.003% | 0.052% | 0.193% | 0.995% | 1.587% |
| pWISE10645 | 626 | 0.039% | 0.026% | 0.017% | 0.089% | 0.371% |
| pWISE10646 | 627 | 0.031% | 0.172% | 0.143% | 0.088% | 0.140% |
| pWISE10647 | 628 | 0.002% | 0.001% | 0.012% | 0.274% | 0.571% |
| | | Standard Deviation of the average editing percent at the | | | | |
| | | nucleotides numbered according to the position in the PWsp389 | | | | |
| | Protein SEQ | spacer sequence on the non-target, genomic DNA strand | | | | |
| pWISE | ID NO: | C.11 | C.12 | C.16 | C.17 | C.18 |
| pWISE121 | 56 | 0.040% | 0.101% | 0.050% | 0.043% | 0.083% |
| pWISE9123 | 456 | 0.040% | 0.050% | 0.170% | 0.138% | 0.045% |
| pWISE10643 | 624 | 0.075% | 0.150% | 0.002% | 0.359% | 0.227% |
| pWISE10644 | 625 | 1.207% | 1.313% | 1.135% | 1.335% | 1.398% |
| pWISE10645 | 626 | 0.600% | 0.475% | 0.381% | 0.427% | 0.726% |
| pWISE10646 | 627 | 0.038% | 0.029% | 0.138% | 0.336% | 0.437% |
| pWISE10647 | 628 | 1.054% | 1.034% | 0.768% | 0.695% | 0.618% |
| | | Standard Deviation of the average editing percent at the | | | | |
| | | nucleotides numbered according to the position in the PWsp389 | | | | |
| | Protein SEQ | spacer sequence on the non-target, genomic DNA strand | | | | |
| pWISE | ID NO: | C.25 | C.26 | C.27 | C.28 | C.30 |
| pWISE121 | 56 | 0.028% | 0.012% | 0.029% | 0.078% | 0.028% |
| pWISE9123 | 456 | 0.026% | 0.029% | 0.041% | 0.057% | 0.025% |
| pWISE10643 | 624 | 0.020% | 0.022% | 0.022% | 0.058% | 0.021% |
| pWISE10644 | 625 | 0.028% | 0.032% | 0.090% | 0.081% | 0.062% |
| pWISE10645 | 626 | 0.004% | 0.004% | 0.095% | 0.078% | 0.012% |
| pWISE10646 | 627 | 0.066% | 0.065% | 0.032% | 0.057% | 0.032% |
| pWISE10647 | 628 | 0.011% | 0.008% | 0.043% | 0.018% | 0.014% |

TABLE 49

| | | Average G to A editing percent at the nucleotides numbered | | | | |
|---|---|---|---|---|---|---|
| | | according to the position in the PWsp389 spacer sequence on the | | | | |
| | Protein SEQ | opposite, target genomic DNA strand | | | | |
| pWISE | ID NO: | G.-9 | G.-8 | G.-7 | G.-6 | G.2 |
| pWISE121 | 56 | 0.008% | 0.033% | 0.022% | 0.023% | 0.094% |
| pWISE9123 | 456 | 0.071% | 0.008% | 0.008% | 0.003% | 0.008% |
| pWISE10643 | 624 | 0.064% | 0.009% | 0.010% | 0.025% | 0.022% |
| pWISE10644 | 625 | 0.013% | 0.003% | 0.010% | 0.016% | 0.000% |
| pWISE10645 | 626 | 0.033% | 0.002% | 0.002% | 0.015% | 0.005% |
| pWISE10646 | 627 | 0.015% | 0.000% | 0.000% | 0.006% | 0.008% |
| pWISE10647 | 628 | 0.044% | 0.016% | 0.003% | 0.003% | 0.005% |
| | | Average G to A editing percent at the nucleotides numbered | | | | |
| | | according to the position in the PWsp389 spacer sequence on the | | | | |
| | Protein SEQ | opposite, target genomic DNA strand | | | | |
| pWISE | ID NO: | G.14 | G.21 | G.23 | G.24 | G.32 |
| pWISE121 | 56 | 0.491% | 0.310% | 0.243% | 0.248% | 0.358% |
| pWISE9123 | 456 | 0.278% | 0.229% | 1.639% | 1.345% | 0.227% |
| pWISE10643 | 624 | 0.601% | 0.230% | 2.273% | 1.943% | 0.277% |
| pWISE10644 | 625 | 0.651% | 0.425% | 3.452% | 2.995% | 0.421% |
| pWISE10645 | 626 | 0.887% | 0.696% | 7.875% | 7.992% | 0.501% |
| pWISE10646 | 627 | 1.158% | 0.667% | 9.351% | 9.530% | 0.566% |
| pWISE10647 | 628 | 1.353% | 0.790% | 10.882% | 11.041% | 0.536% |

TABLE 50

| | | Standard deviation of the average G to A editing percent at the | | | | |
|---|---|---|---|---|---|---|
| | | nucleotides numbered according to the position in the PWsp389 | | | | |
| | Protein SEQ | spacer sequence on the opposite, target genomic DNA strand | | | | |
| pWISE | ID NO: | G.-9 | G.-8 | G.-7 | G.-6 | G.2 |
| pWISE121 | 56 | 0.011% | 0.015% | 0.001% | 0.013% | 0.019% |
| pWISE9123 | 456 | 0.035% | 0.003% | 0.004% | 0.004% | 0.004% |
| pWISE10643 | 624 | 0.010% | 0.003% | 0.006% | 0.006% | 0.011% |
| pWISE10644 | 625 | 0.007% | 0.004% | 0.003% | 0.012% | 0.000% |
| pWISE10645 | 626 | 0.002% | 0.003% | 0.003% | 0.013% | 0.007% |
| pWISE10646 | 627 | 0.021% | 0.000% | 0.000% | 0.001% | 0.012% |
| pWISE10647 | 628 | 0.029% | 0.023% | 0.005% | 0.005% | 0.007% |
| | | Standard deviation of the average G to A editing percent at the | | | | |
| | | nucleotides numbered according to the position in the PWsp389 | | | | |
| | Protein SEQ | spacer sequence on the opposite, target genomic DNA strand | | | | |
| pWISE | ID NO: | G.14 | G.21 | G.23 | G.24 | G.32 |
| pWISE121 | 56 | 0.002% | 0.092% | 0.047% | 0.081% | 0.074% |
| pWISE9123 | 456 | 0.094% | 0.107% | 0.176% | 0.179% | 0.061% |
| pWISE10643 | 624 | 0.112% | 0.115% | 0.212% | 0.209% | 0.031% |
| pWISE10644 | 625 | 0.023% | 0.102% | 0.577% | 0.626% | 0.002% |
| pWISE10645 | 626 | 0.056% | 0.167% | 0.108% | 0.255% | 0.150% |
| pWISE10646 | 627 | 0.346% | 0.001% | 0.853% | 1.067% | 0.063% |
| pWISE10647 | 628 | 0.111% | 0.239% | 0.075% | 0.090% | 0.034% |

TABLE 51

| | | Average C to T editing percent at the nucleotides numbered | | | | |
|---|---|---|---|---|---|---|
| | | according to the position in the PWsp390 spacer sequence on the | | | | |
| | Protein SEQ | non-target, genomic DNA strand | | | | |
| pWISE | ID NO: | C.-5 | C.2 | C.6 | C.7 | C.8 |
| pWISE121 | 56 | 0.008% | 0.040% | 0.048% | 0.079% | 0.063% |
| pWISE9123 | 456 | 0.043% | 2.125% | 16.387% | 17.060% | 18.221% |
| pWISE10643 | 624 | 0.013% | 2.497% | 20.555% | 21.406% | 22.889% |
| pWISE10644 | 625 | 0.021% | 3.107% | 23.432% | 24.235% | 25.764% |
| pWISE10645 | 626 | 0.039% | 4.129% | 24.324% | 26.202% | 28.604% |
| pWISE10646 | 627 | 0.019% | 3.839% | 23.946% | 26.784% | 29.869% |
| pWISE10647 | 628 | 0.038% | 3.506% | 22.643% | 26.039% | 29.620% |

| | | Average C to T editing percent at the nucleotides numbered | | | | |
|---|---|---|---|---|---|---|
| | | according to the position in the PWsp390 spacer sequence on | | | | |
| | Protein SEQ | the non-target, genomic DNA strand | | | | |
| pWISE | ID NO: | C.9 | C.11 | C.12 | C.14 | C.15 |
| pWISE121 | 56 | 0.079% | 0.103% | 0.143% | 0.254% | 0.531% |
| pWISE9123 | 456 | 18.939% | 19.166% | 19.204% | 9.327% | 11.582% |
| pWISE10643 | 624 | 23.621% | 23.776% | 23.698% | 14.265% | 16.642% |
| pWISE10644 | 625 | 26.395% | 26.496% | 26.499% | 18.960% | 21.075% |
| pWISE10645 | 626 | 29.522% | 29.674% | 29.709% | 23.030% | 25.752% |
| pWISE10646 | 627 | 30.631% | 30.793% | 30.841% | 25.672% | 27.864% |
| pWISE10647 | 628 | 30.324% | 30.456% | 30.474% | 25.511% | 27.839% |

| | | Average C to T editing percent at the nucleotides numbered according | | | | | |
|---|---|---|---|---|---|---|---|
| | | to the position in the PWsp390 spacer sequence on the non-target, | | | | | |
| | Protein SEQ | genomic DNA strand | | | | | |
| pWISE | ID NO: | C.16 | C.17 | C.19 | C.27 | C.28 | C.30 |
| pWISE121 | 56 | 1.428% | 3.824% | 1.475% | 0.159% | 0.151% | 0.008% |
| pWISE9123 | 456 | 12.384% | 12.723% | 7.551% | 0.019% | 0.040% | 0.011% |
| pWISE10643 | 624 | 17.310% | 17.746% | 9.845% | 0.019% | 0.030% | 0.029% |
| pWISE10644 | 625 | 21.633% | 21.838% | 11.311% | 0.034% | 0.031% | 0.028% |
| pWISE10645 | 626 | 26.111% | 26.337% | 13.150% | 0.059% | 0.078% | 0.020% |
| pWISE10646 | 627 | 28.139% | 28.158% | 13.430% | 0.072% | 0.119% | 0.042% |
| pWISE10647 | 628 | 28.147% | 28.259% | 12.442% | 0.130% | 0.161% | 0.091% |

TABLE 52

| | | Standard Deviation of the average editing percent at the | | | | |
|---|---|---|---|---|---|---|
| | | nucleotides numbered according to the position in the PWsp390 | | | | |
| | Protein SEQ | spacer sequence on the non-target, genomic DNA strand | | | | |
| pWISE | ID NO: | C.-5 | C.2 | C.6 | C.7 | C.8 |
| pWISE121 | 56 | No data | No data | No data | No data | No data |
| pWISE9123 | 456 | 0.002% | 1.173% | 9.154% | 9.487% | 10.206% |
| pWISE10643 | 624 | 0.007% | 1.127% | 9.924% | 10.450% | 11.200% |
| pWISE10644 | 625 | 0.022% | 0.090% | 0.551% | 0.566% | 1.128% |
| pWISE10645 | 626 | 0.056% | 0.439% | 1.920% | 2.056% | 2.079% |
| pWISE10646 | 627 | 0.006% | 0.224% | 0.594% | 0.857% | 0.630% |
| pWISE10647 | 628 | 0.025% | 0.760% | 1.077% | 1.039% | 1.047% |

| | | Standard Deviation of the average editing percent at the | | | | |
|---|---|---|---|---|---|---|
| | | nucleotides numbered according to the position in the PWsp390 | | | | |
| | Protein SEQ | spacer sequence on the non-target, genomic DNA strand | | | | |
| pWISE | ID NO: | C.9 | C.11 | C.12 | C.14 | C.15 |
| pWISE121 | 56 | No data | No data | No data | No data | No data |
| pWISE9123 | 456 | 10.685% | 10.749% | 10.873% | 5.486% | 6.549% |
| pWISE10643 | 624 | 11.618% | 11.767% | 11.682% | 6.806% | 7.940% |
| pWISE10644 | 625 | 1.410% | 1.369% | 1.232% | 0.291% | 0.591% |
| pWISE10645 | 626 | 1.898% | 1.950% | 1.867% | 1.962% | 2.056% |
| pWISE10646 | 627 | 0.633% | 0.727% | 0.703% | 0.875% | 0.885% |
| pWISE10647 | 628 | 1.123% | 1.035% | 1.069% | 1.508% | 1.402% |

| | | Standard Deviation of the average editing percent at the nucleotides | | | | | |
|---|---|---|---|---|---|---|---|
| | | numbered according to the position in the PWsp390 spacer sequence on | | | | | |
| | Protein SEQ | the non-target, genomic DNA strand | | | | | |
| pWISE | ID NO: | C.16 | C.17 | C.19 | C.27 | C.28 | C.30 |
| pWISE121 | 56 | No data | No data | No data | No data | No data | No data |
| pWISE9123 | 456 | 6.855% | 7.160% | 4.258% | 0.015% | 0.014% | 0.016% |
| pWISE10643 | 624 | 8.276% | 8.408% | 4.985% | 0.019% | 0.016% | 0.040% |
| pWISE10644 | 625 | 0.473% | 0.641% | 0.345% | 0.033% | 0.028% | 0.032% |
| pWISE10645 | 626 | 1.928% | 1.809% | 1.322% | 0.006% | 0.034% | 0.028% |
| pWISE10646 | 627 | 0.819% | 0.836% | 0.337% | 0.060% | 0.007% | 0.038% |
| pWISE10647 | 628 | 1.423% | 1.512% | 0.617% | 0.025% | 0.029% | 0.059% |

TABLE 53

| | | Average G to A editing percent at the nucleotides numbered | | | | |
|---|---|---|---|---|---|---|
| | | according to the position in the PWsp390 spacer sequence on the | | | | |
| | Protein SEQ | opposite, target genomic DNA strand | | | | |
| pWISE | ID NO: | G.-9 | G.-8 | G.4 | G.21 | G.23 |
| pWISE121 | 56 | 0.008% | 0.000% | 0.008% | 0.635% | 1.015% |
| pWISE9123 | 456 | 0.008% | 0.000% | 0.027% | 0.056% | 0.238% |
| pWISE10643 | 624 | 0.003% | 0.006% | 0.014% | 0.073% | 0.349% |
| pWISE10644 | 625 | 0.037% | 0.000% | 0.019% | 0.064% | 0.438% |
| pWISE10645 | 626 | 0.015% | 0.000% | 0.055% | 0.031% | 0.897% |
| pWISE10646 | 627 | 0.011% | 0.000% | 0.004% | 0.198% | 1.031% |
| pWISE10647 | 628 | 0.011% | 0.000% | 0.028% | 0.168% | 1.369% |

| | | Average G to A editing percent at the nucleotides | | | |
|---|---|---|---|---|---|
| | | numbered according to the position in the PWsp390 spacer | | | |
| | Protein SEQ | sequence on the opposite, target genomic DNA strand | | | |
| pWISE | ID NO: | G.24 | G.25 | G.26 | G.33 |
| pWISE121 | 56 | 0.206% | 0.294% | 0.087% | 0.103% |
| pWISE9123 | 456 | 0.277% | 0.177% | 0.071% | 0.411% |
| pWISE10643 | 624 | 0.376% | 0.271% | 0.153% | 0.510% |
| pWISE10644 | 625 | 0.474% | 0.305% | 0.244% | 0.502% |
| pWISE10645 | 626 | 0.853% | 0.583% | 0.439% | 0.692% |
| pWISE10646 | 627 | 1.093% | 0.747% | 0.540% | 0.937% |
| pWISE10647 | 628 | 1.625% | 1.176% | 0.829% | 1.299% |

TABLE 54

| | | Standard deviation of the average G to A editing percent at the | | | | |
|---|---|---|---|---|---|---|
| | | nucleotides numbered according to the position in the PWsp390 | | | | |
| | Protein SEQ | spacer on the opposite, target genomic DNA strand | | | | |
| pWISE | ID NO: | G.-9 | G.-8 | G.4 | G.21 | G.23 |
| pWISE121 | 56 | No data | No data | No data | No data | No data |
| pWISE9123 | 456 | 0.001% | 0.000% | 0.003% | 0.067% | 0.185% |
| pWISE10643 | 624 | 0.004% | 0.009% | 0.003% | 0.024% | 0.186% |
| pWISE10644 | 625 | 0.002% | 0.000% | 0.004% | 0.012% | 0.182% |
| pWISE10645 | 626 | 0.022% | 0.000% | 0.032% | 0.033% | 0.140% |
| pWISE10646 | 627 | 0.006% | 0.000% | 0.005% | 0.001% | 0.237% |
| pWISE10647 | 628 | 0.005% | 0.000% | 0.030% | 0.040% | 0.158% |

| | | Standard deviation of the average G to A editing percent at the | | | |
|---|---|---|---|---|---|
| | | nucleotides numbered according to the position in the PWsp390 | | | |
| | Protein SEQ | spacer sequence on the opposite, target genomic DNA strand | | | |
| pWISE | ID NO: | G.24 | G.25 | G.26 | G.33 |
| pWISE121 | 56 | No data | No data | No data | No data |
| pWISE9123 | 456 | 0.182% | 0.052% | 0.066% | 0.359% |
| pWISE10643 | 624 | 0.171% | 0.066% | 0.084% | 0.236% |
| pWISE10644 | 625 | 0.100% | 0.105% | 0.002% | 0.033% |
| pWISE10645 | 626 | 0.078% | 0.003% | 0.084% | 0.107% |
| pWISE10646 | 627 | 0.304% | 0.257% | 0.115% | 0.008% |
| pWISE10647 | 628 | 0.184% | 0.093% | 0.161% | 0.039% |

### Example 11

Circular permutants of an enzymatically inactive LbCas12a (dLbCas12a (SEQ ID NO: 59)) or Cas12a comprising a R1138A mutation (nCas12a), which is a non-target strand catalytic nickase mutation, were fused to a cytosine deaminase at the N-terminus of the circular permutant using a linker and a UGI was fused to the C-terminus of the circular permutant to provide a fusion protein. These fusion proteins were tested both for C to T base editing on the target strand and G to A editing on the non-target strand. The tested linkers between the cytosine deaminase and circular permutant included a GS-XTEN-GS linker (SEQ ID NO:30); an XTEN linker (SEQ ID NO:29); a four amino acid glycine-serine (4XGS) linker of SGGS (SEQ ID NO:26); a six amino acid glycine serine (6XGS) linker of (SGS)ₙ wherein n is 2 (SEQ ID NO:869); an eight amino acid glycine-serine (8XGS) linker of (SGGS)ₙ wherein n is 2 (SEQ ID NO:870); and a twelve amino acid glycine-serine (12XGS) linker of (SGGS)ₙ wherein n is 3. The fusion proteins are listed in Table 55 and the targets are listed in Table 56.

TABLE 55
Tested constructs.

| | Vector SEQ | DNA SEQ | Protein SEQ |
|---|---|---|---|
| pWISE | ID NO: | ID NO: | ID NO: |
| pWISE9120 | 477 | 465 | 453 |
| pWISE10648 | 691 | 660 | 629 |
| pWISE10649 | 692 | 661 | 630 |
| pWISE10650 | 693 | 662 | 631 |
| pWISE10651 | 694 | 663 | 632 |
| pWISE10652 | 695 | 664 | 633 |
| pWISE10658 | 701 | 670 | 639 |
| pWISE10653 | 696 | 665 | 634 |
| pWISE10654 | 697 | 666 | 635 |
| pWISE10655 | 698 | 667 | 636 |
| pWISE10656 | 699 | 668 | 637 |
| pWISE10657 | 700 | 669 | 638 |

TABLE 56
Targets

| Target Nucleic | Target SEQ | | Spacer SEQ | | Vector SEQ |
|---|---|---|---|---|---|
| Acid | ID NO: | Spacer | ID NO: | pWISE | ID NO: |
| human FANCF | 273 | PWsp132 | 799 | pWISE878 | 809 |
| human FANCF | 273 | PWsp449 | 276 | pWISE878 | 279 |
| human AAVS1 | 863 | PWsp389 | 801 | pWISE5986 | 811 |
| human AAVS1 | 863 | PWsp390 | 802 | pWISE5986 | 812 |
| human RUNX1 | 864 | PWsp135 | 800 | pWISE5989 | 810 |
| human RUNX1 | 864 | PWsp133 | 873 | pWISE254 | 878 |
| human TRAC | 871 | PWsp3627 | 874 | pWISE10674 | 879 |
| human TRAC | 871 | PWsp3628 | 875 | pWISE10675 | 880 |
| human AAVS1 | 273 | PWsp3629 | 876 | pWISE10676 | 881 |
| human HEK2 | 872 | PWsp3630 | 877 | pWISE10677 | 882 |

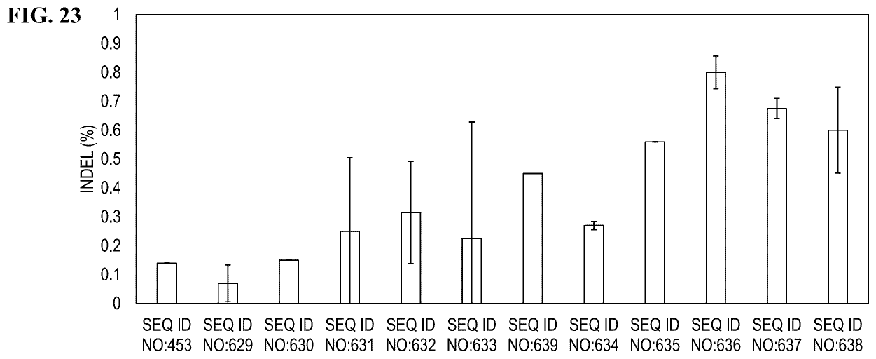

The fusion proteins were tested for their ability to perform base editing on both the target and non-target strands at ten different sites in five target nucleic acids (SEQ ID NOs: 273, 863, 864, 871, and 872) in HEK293T cells using ten spacers (SEQ ID NOs: 276, 799-802, and 873-877). A single biological replicate and two technical replicates or three biological replicates and two technical replicates were tested. HEK293T cells were seeded into 48-well collagen-coated plates (Corning) in the absence of an antibiotic using DMEM media. At 70-80% confluency, cells were transfected with 1.5 L of LTX (ThermoFisher Scientific) using 500 ng of the fusion protein plasmid and 500 ng of guide RNA plasmid according to manufacturer's protocol. After 3 days, the cells were lysed with a crude extraction method using TritonX buffer. Each fusion protein was scored based on the precise base pair editing in the FANCF, RUNX1, AAVS1, TRAC, and HEK2 genes using the guide RNAs (SEQ ID NOs: 276, 799-802, and 873-877). Low, background levels of INDEL formation were seen, which was expected. The INDEL percentages for fusion proteins including a circular permutant comprising an inactivating mutation (SEQ ID NOs: 453 and 629-633) or a catalytic nickase mutation (SEQ ID NOs: 634-639) are shown in FIG. 23. For the tested constructs, an increase in INDEL percentage was observed for the fusion proteins that included a circular permutant having a catalytic nickase mutation (SEQ ID NOs: 634-639) compared to the fusion proteins including a circular permutant having an inactivating mutation (SEQ ID NOs: 629-633). The results for the tested fusion proteins are provided in Tables 57-96. The values in Tables 57-96 that are below 0.1% are considered to be in the noise of the instrument (below the limit of detection) and are not indicative of editing. Values that are between 0.1% and 0.5% indicate that editing is present in the experiment at the specified location, but the assay is not sensitive enough to accurately quantify the amount of base editing. As seen in Tables 57-96, fusion proteins showed both efficient target and non-target strand base editing. Specifically, fusion proteins including a circular permutant of dLbCas 12a had an efficiency of up to about 10% and fusion proteins including a circular permutant of nCas 12a had an efficiency of up to about 25%. The window of editing appears to be particularly beneficial between about positions 9-23 of the target spacer sequence. Similar to what was shown in Example 10, the shorter linkers were found to have improved editing towards the 3′ end of the spacer for both the target and non-target strand editing.

TABLE 57

| | | Average C to T editing percent at the nucleotides numbered | | | | | | |
|---|---|---|---|---|---|---|---|---|
| | | according to the position in the PWsp132 spacer sequence on the | | | | | | |
| | Protein SEQ | non-target, genomic DNA strand | | | | | | |
| pWISE | ID NO: | C.-7 | C.-6 | C.-4 | C.-1 | C.2 | C.3 | C.11 |
| pWISE121 | 56 | 0.039% | 0.052% | 0.028% | 0.043% | 0.097% | 0.093% | 0.277% |
| pWISE9120 | 453 | 0.131% | 0.074% | 0.006% | 0.308% | 0.006% | 0.051% | 1.108% |
| pWISE10648 | 629 | 0.172% | 0.159% | 0.030% | 0.275% | 0.035% | 0.045% | 0.611% |
| pWISE10649 | 630 | 0.179% | 0.118% | 0.000% | 0.269% | 0.022% | 0.030% | 0.451% |
| pWISE10650 | 631 | 0.280% | 0.225% | 0.061% | 0.409% | 0.010% | 0.052% | 0.648% |
| pWISE10651 | 632 | 0.302% | 0.203% | 0.044% | 0.541% | 0.049% | 0.091% | 0.591% |
| pWISE10652 | 633 | 0.073% | 0.058% | 0.011% | 0.186% | 0.010% | 0.026% | 0.494% |
| pWISE10658 | 639 | 0.213% | 0.179% | 0.044% | 0.362% | 0.071% | 0.114% | 1.434% |
| pWISE10653 | 634 | 0.140% | 0.122% | 0.043% | 0.341% | 0.064% | 0.068% | 0.720% |
| pWISE10654 | 635 | 0.185% | 0.138% | 0.057% | 0.425% | 0.054% | 0.132% | 0.944% |
| pWISE10655 | 636 | 0.135% | 0.076% | 0.038% | 0.306% | 0.018% | 0.053% | 0.712% |
| pWISE10656 | 637 | 0.208% | 0.132% | 0.036% | 0.331% | 0.069% | 0.079% | 0.660% |
| pWISE10657 | 638 | 0.124% | 0.084% | 0.006% | 0.238% | 0.006% | 0.016% | 0.556% |

| | | Average C to T editing percent at the nucleotides numbered | | | | | |
|---|---|---|---|---|---|---|---|
| | | according to the position in the PWsp132 spacer sequence on the | | | | | |
| | Protein SEQ | non-target, genomic DNA strand | | | | | |
| pWISE | ID NO: | C.14 | C.17 | C.19 | C.21 | C.24 | C.28 |
| pWISE121 | 56 | 1.076% | 0.870% | 0.687% | 0.477% | 0.153% | 0.176% |
| pWISE9120 | 453 | 0.720% | 0.251% | 2.011% | 2.102% | 0.794% | 0.326% |
| pWISE10648 | 629 | 0.373% | 0.091% | 1.242% | 1.252% | 0.550% | 0.020% |
| pWISE10649 | 630 | 0.495% | 0.209% | 3.356% | 3.622% | 1.946% | 0.289% |
| pWISE10650 | 631 | 0.657% | 0.189% | 4.384% | 4.777% | 2.522% | 0.380% |
| pWISE10651 | 632 | 0.651% | 0.199% | 4.697% | 4.967% | 2.608% | 0.356% |
| pWISE10652 | 633 | 0.459% | 0.073% | 3.167% | 3.481% | 2.003% | 0.280% |
| pWISE10658 | 639 | 0.722% | 0.281% | 3.091% | 3.705% | 0.998% | 0.069% |
| pWISE10653 | 634 | 0.429% | 0.159% | 1.826% | 1.882% | 0.703% | 0.068% |
| pWISE10654 | 635 | 0.639% | 0.464% | 4.466% | 5.076% | 2.270% | 0.301% |
| pWISE10655 | 636 | 0.575% | 0.186% | 4.885% | 5.470% | 2.219% | 0.267% |
| pWISE10656 | 637 | 0.706% | 0.208% | 4.496% | 5.168% | 2.199% | 0.299% |
| pWISE10657 | 638 | 0.513% | 0.051% | 4.023% | 4.924% | 1.510% | 0.182% |

TABLE 58

| | | Standard Deviation of the average editing percent at the nucleotides | | | | | | |
|---|---|---|---|---|---|---|---|---|
| | | numbered according to the position in the PWsp132 spacer sequence | | | | | | |
| | Protein SEQ | on the non-target, genomic DNA strand | | | | | | |
| pWISE | ID NO: | C.-7 | C.-6 | C.-4 | C.-1 | C.2 | C.3 | C.11 |
| pWISE121 | 56 | 0.050% | 0.025% | 0.009% | 0.006% | 0.082% | 0.003% | 0.090% |
| pWISE9120 | 453 | No data | No data | No data | No data | No data | No data | No data |
| pWISE10648 | 629 | 0.012% | 0.016% | 0.000% | 0.007% | 0.007% | 0.001% | 0.070% |
| pWISE10649 | 630 | No data | No data | No data | No data | No data | No data | No data |
| pWISE10650 | 631 | 0.012% | 0.013% | 0.041% | 0.035% | 0.001% | 0.028% | 0.089% |
| pWISE10651 | 632 | 0.036% | 0.092% | 0.049% | 0.116% | 0.055% | 0.087% | 0.001% |
| pWISE10652 | 633 | 0.050% | 0.020% | 0.007% | 0.111% | 0.015% | 0.037% | 0.064% |
| pWISE10658 | 639 | 0.057% | 0.029% | 0.035% | 0.031% | 0.019% | 0.033% | 0.206% |
| pWISE10653 | 634 | 0.027% | 0.002% | 0.027% | 0.040% | 0.018% | 0.017% | 0.089% |
| pWISE10654 | 635 | 0.060% | 0.063% | 0.074% | 0.050% | 0.027% | 0.089% | 0.218% |
| pWISE10655 | 636 | 0.011% | 0.015% | 0.038% | 0.072% | 0.009% | 0.026% | 0.111% |
| pWISE10656 | 637 | 0.034% | 0.008% | 0.002% | 0.014% | 0.090% | 0.090% | 0.182% |
| pWISE10657 | 638 | 0.067% | 0.032% | 0.008% | 0.034% | 0.008% | 0.006% | 0.037% |

| | | Standard Deviation of the average editing percent at the nucleotides | | | | | |
|---|---|---|---|---|---|---|---|
| | | numbered according to the position in the PWsp132 spacer | | | | | |
| | Protein SEQ | sequence on the non-target, genomic DNA strand | | | | | |
| pWISE | ID NO: | C.14 | C.17 | C.19 | C.21 | C.24 | C.28 |
| pWISE121 | 56 | 0.130% | 0.481% | 0.258% | 0.094% | 0.003% | 0.121% |
| pWISE9120 | 453 | No data | No data | No data | No data | No data | No data |
| pWISE10648 | 629 | 0.073% | 0.034% | 0.097% | 0.061% | 0.042% | 0.000% |
| pWISE10649 | 630 | No data | No data | No data | No data | No data | No data |
| pWISE10650 | 631 | 0.101% | 0.017% | 0.306% | 0.240% | 0.469% | 0.073% |
| pWISE10651 | 632 | 0.020% | 0.046% | 0.029% | 0.091% | 0.110% | 0.034% |
| pWISE10652 | 633 | 0.121% | 0.103% | 0.321% | 0.309% | 0.069% | 0.007% |
| pWISE10658 | 639 | 0.053% | 0.134% | 0.162% | 0.367% | 0.052% | 0.044% |
| pWISE10653 | 634 | 0.091% | 0.065% | 0.274% | 0.200% | 0.050% | 0.005% |
| pWISE10654 | 635 | 0.051% | 0.005% | 0.163% | 0.081% | 0.203% | 0.028% |
| pWISE10655 | 636 | 0.074% | 0.062% | 0.318% | 0.373% | 0.030% | 0.085% |
| pWISE10656 | 637 | 0.180% | 0.167% | 1.135% | 1.379% | 0.714% | 0.184% |
| pWISE10657 | 638 | 0.016% | 0.014% | 0.014% | 0.143% | 0.328% | 0.034% |

TABLE 59

| | | Average G to A editing percent at the nucleotides numbered | | | | |
|---|---|---|---|---|---|---|
| | | according to the position in the PWsp132 spacer sequence on the | | | | |
| | Protein SEQ | opposite, target genomic DNA strand | | | | |
| pWISE | ID NO: | G.-5 | G.6 | G.7 | G.9 | G.12 |
| pWISE121 | 56 | 0.053% | 0.094% | 0.091% | 0.210% | 0.189% |
| pWISE9120 | 453 | 0.023% | 0.080% | 0.131% | 2.016% | 5.724% |
| pWISE10648 | 629 | 0.013% | 0.078% | 0.124% | 1.258% | 5.045% |
| pWISE10649 | 630 | 0.000% | 0.047% | 0.118% | 1.006% | 6.560% |
| pWISE10650 | 631 | 0.008% | 0.074% | 0.165% | 1.198% | 6.271% |
| pWISE10651 | 632 | 0.017% | 0.103% | 0.205% | 1.063% | 4.607% |
| pWISE10652 | 633 | 0.000% | 0.095% | 0.163% | 0.363% | 1.474% |
| pWISE10658 | 639 | 0.006% | 0.100% | 0.239% | 3.398% | 11.948% |
| pWISE10653 | 634 | 0.018% | 0.061% | 0.103% | 1.484% | 8.508% |
| pWISE10654 | 635 | 0.034% | 0.061% | 0.147% | 1.771% | 8.709% |
| pWISE10655 | 636 | 0.020% | 0.102% | 0.216% | 2.025% | 8.551% |
| pWISE10656 | 637 | 0.033% | 0.117% | 0.222% | 1.748% | 5.471% |
| pWISE10657 | 638 | 0.000% | 0.056% | 0.282% | 1.063% | 2.384% |

| | | Average G to A editing percent at the nucleotides numbered | | | | |
|---|---|---|---|---|---|---|
| | | according to the position in the PWsp132 spacer sequence on the | | | | |
| | Protein SEQ | opposite, target genomic DNA strand | | | | |
| pWISE | ID NO: | G.13 | G.15 | G.23 | G.25 | G.30 |
| pWISE121 | 56 | 0.282% | 0.462% | 0.899% | 0.299% | 0.113% |
| pWISE9120 | 453 | 5.547% | 7.409% | 2.285% | 0.765% | 2.016% |
| pWISE10648 | 629 | 4.807% | 6.238% | 2.183% | 0.770% | 1.476% |
| pWISE10649 | 630 | 6.379% | 9.240% | 4.353% | 1.643% | 3.276% |
| pWISE10650 | 631 | 6.577% | 9.906% | 5.875% | 2.173% | 3.961% |
| pWISE10651 | 632 | 5.432% | 10.521% | 5.881% | 2.171% | 4.274% |
| pWISE10652 | 633 | 2.019% | 7.001% | 5.017% | 2.129% | 3.116% |
| pWISE10658 | 639 | 11.329% | 15.258% | 3.875% | 1.277% | 3.300% |
| pWISE10653 | 634 | 8.251% | 10.985% | 2.802% | 0.922% | 2.407% |
| pWISE10654 | 635 | 8.183% | 12.532% | 5.417% | 1.888% | 4.190% |
| pWISE10655 | 636 | 8.506% | 14.415% | 6.372% | 2.480% | 5.135% |
| pWISE10656 | 637 | 5.970% | 12.706% | 6.151% | 2.501% | 4.557% |
| pWISE10657 | 638 | 3.250% | 13.476% | 6.261% | 2.242% | 5.242% |

TABLE 60

| | | Standard deviation of the average G to A editing percent at the | | | | |
|---|---|---|---|---|---|---|
| | | nucleotides numbered according to the position in the PWsp132 | | | | |
| | Protein SEQ | spacer sequence on the opposite, target genomic DNA strand | | | | |
| pWISE | ID NO: | G.-5 | G.6 | G.7 | G.9 | G.12 |
| pWISE121 | 56 | 0.075% | 0.078% | 0.074% | 0.102% | 0.026% |
| pWISE9120 | 453 | No data | No data | No data | No data | No data |
| pWISE10648 | 629 | 0.018% | 0.019% | 0.009% | 0.075% | 0.281% |
| pWISE10649 | 630 | No data | No data | No data | No data | No data |
| pWISE10650 | 631 | 0.002% | 0.028% | 0.027% | 0.087% | 0.414% |
| pWISE10651 | 632 | 0.003% | 0.083% | 0.011% | 0.136% | 0.007% |
| pWISE10652 | 633 | 0.000% | 0.009% | 0.047% | 0.058% | 0.019% |
| pWISE10658 | 639 | 0.002% | 0.022% | 0.053% | 0.020% | 0.035% |
| pWISE10653 | 634 | 0.003% | 0.001% | 0.021% | 0.161% | 0.102% |
| pWISE10654 | 635 | 0.008% | 0.040% | 0.070% | 0.324% | 0.265% |
| pWISE10655 | 636 | 0.004% | 0.006% | 0.005% | 0.220% | 0.712% |
| pWISE10656 | 637 | 0.002% | 0.016% | 0.021% | 0.047% | 0.364% |
| pWISE10657 | 638 | 0.000% | 0.007% | 0.075% | 0.024% | 0.211% |
| | | Standard deviation of the average G to A editing percent at the | | | | |
| | | nucleotides numbered according to the position in the PWsp132 | | | | |
| | Protein SEQ | spacer sequence on the opposite, target genomic DNA strand | | | | |
| pWISE | ID NO: | G.13 | G.15 | G.23 | G.25 | G.30 |
| pWISE121 | 56 | 0.052% | 0.055% | 0.291% | 0.014% | 0.077% |
| pWISE9120 | 453 | No data | No data | No data | No data | No data |
| pWISE10648 | 629 | 0.250% | 0.477% | 0.231% | 0.078% | 0.176% |
| pWISE10649 | 630 | No data | No data | No data | No data | No data |
| pWISE10650 | 631 | 0.346% | 0.467% | 0.239% | 0.027% | 0.030% |
| pWISE10651 | 632 | 0.103% | 0.090% | 0.183% | 0.043% | 0.164% |
| pWISE10652 | 633 | 0.083% | 0.056% | 0.613% | 0.454% | 0.312% |
| pWISE10658 | 639 | 0.016% | 0.065% | 0.144% | 0.005% | 0.234% |
| pWISE10653 | 634 | 0.210% | 0.069% | 0.041% | 0.163% | 0.037% |
| pWISE10654 | 635 | 0.064% | 0.244% | 0.253% | 0.002% | 0.274% |
| pWISE10655 | 636 | 0.558% | 0.755% | 0.567% | 0.106% | 0.249% |
| pWISE10656 | 637 | 0.251% | 1.415% | 1.042% | 0.621% | 0.909% |
| pWISE10657 | 638 | 0.034% | 0.695% | 0.226% | 0.095% | 0.327% |

TABLE 61

| | | Average C to T editing percent at the nucleotides numbered | | | | | |
|---|---|---|---|---|---|---|---|
| | | according to the position in the PWsp449 spacer sequence on the | | | | | |
| | Protein SEQ | non-target, genomic DNA strand | | | | | |
| pWISE | ID NO: | C.-5 | C.-1 | C.2 | C.10 | C.11 | C.15 |
| pWISE121 | 56 | 0.095% | 0.158% | 0.123% | 0.401% | 0.431% | 2.083% |
| pWISE9120 | 453 | 0.333% | 0.491% | 0.138% | 4.611% | 4.642% | 3.777% |
| pWISE10648 | 629 | 0.204% | 0.214% | 0.074% | 1.536% | 1.513% | 1.659% |
| pWISE10649 | 630 | 0.234% | 0.235% | 0.097% | 1.368% | 1.244% | 1.649% |
| pWISE10650 | 631 | 0.316% | 0.420% | 0.111% | 2.774% | 2.600% | 3.411% |
| pWISE10651 | 632 | 0.308% | 0.327% | 0.080% | 2.629% | 2.437% | 3.317% |
| pWISE10652 | 633 | 0.245% | 0.337% | 0.076% | 2.137% | 1.855% | 2.639% |
| pWISE10658 | 639 | 0.578% | 0.833% | 0.287% | 7.718% | 7.601% | 4.955% |
| pWISE10653 | 634 | 0.348% | 0.345% | 0.138% | 3.499% | 3.361% | 2.385% |
| pWISE10654 | 635 | 0.417% | 0.628% | 0.253% | 4.403% | 3.868% | 3.202% |
| pWISE10655 | 636 | 0.463% | 0.646% | 0.254% | 4.931% | 4.539% | 4.151% |
| pWISE10656 | 637 | 0.394% | 0.563% | 0.148% | 4.459% | 4.095% | 4.047% |
| pWISE10657 | 638 | 0.316% | 0.526% | 0.155% | 4.514% | 3.949% | 4.070% |

| | | Average C to T editing percent at the nucleotides numbered | | | | |
|---|---|---|---|---|---|---|
| | | according to the position in the PWsp449 spacer sequence on the | | | | |
| | Protein SEQ | non-target, genomic DNA strand | | | | |
| pWISE | ID NO: | C.20 | C.22 | C.30 | C.32 | C.33 |
| pWISE121 | 56 | 0.505% | 1.066% | 1.515% | 1.047% | 1.095% |
| pWISE9120 | 453 | 0.890% | 1.111% | 0.128% | 0.015% | 0.026% |
| pWISE10648 | 629 | 0.522% | 0.628% | 0.148% | 0.059% | 0.064% |
| pWISE10649 | 630 | 0.779% | 0.840% | 0.101% | 0.030% | 0.028% |
| pWISE10650 | 631 | 1.906% | 2.081% | 0.212% | 0.055% | 0.066% |
| pWISE10651 | 632 | 1.726% | 1.713% | 0.225% | 0.012% | 0.019% |
| pWISE10652 | 633 | 1.735% | 1.671% | 0.167% | 0.008% | 0.020% |
| pWISE10658 | 639 | 1.169% | 1.357% | 0.142% | 0.015% | 0.004% |
| pWISE10653 | 634 | 0.722% | 0.830% | 0.113% | 0.002% | 0.007% |
| pWISE10654 | 635 | 1.552% | 1.744% | 0.192% | 0.028% | 0.023% |
| pWISE10655 | 636 | 1.630% | 1.907% | 0.157% | 0.014% | 0.036% |
| pWISE10656 | 637 | 2.020% | 2.107% | 0.214% | 0.006% | 0.027% |
| pWISE10657 | 638 | 1.879% | 2.018% | 0.171% | 0.031% | 0.027% |

TABLE 62

| | | Standard Deviation of the average editing percent at the | | | | | |
|---|---|---|---|---|---|---|---|
| | | nucleotides numbered according to the position in the PWsp449 | | | | | |
| | Protein SEQ | spacer sequence on the non-target, genomic DNA strand | | | | | |
| pWISE | ID NO: | C.-5 | C.-1 | C.2 | C.10 | C.11 | C.15 |
| pWISE121 | 56 | 0.067% | 0.050% | 0.037% | 0.011% | 0.017% | 0.001% |
| pWISE9120 | 453 | No data | No data | No data | No data | No data | No data |
| pWISE10648 | 629 | 0.086% | 0.114% | 0.062% | 0.358% | 0.369% | 0.288% |
| pWISE10649 | 630 | 0.057% | 0.091% | 0.024% | 0.033% | 0.083% | 0.100% |
| pWISE10650 | 631 | 0.037% | 0.011% | 0.083% | 0.109% | 0.215% | 0.378% |
| pWISE10651 | 632 | 0.040% | 0.052% | 0.011% | 0.864% | 0.724% | 0.877% |
| pWISE10652 | 633 | 0.019% | 0.019% | 0.040% | 0.082% | 0.010% | 0.126% |
| pWISE10658 | 639 | 0.024% | 0.100% | 0.054% | 0.877% | 0.857% | 0.413% |
| pWISE10653 | 634 | 0.156% | 0.098% | 0.052% | 0.657% | 0.605% | 0.443% |
| pWISE10654 | 635 | No data | No data | No data | No data | No data | No data |
| pWISE10655 | 636 | 0.151% | 0.192% | 0.098% | 0.353% | 0.241% | 0.441% |
| pWISE10656 | 637 | 0.057% | 0.003% | 0.013% | 0.165% | 0.075% | 0.022% |
| pWISE10657 | 638 | 0.140% | 0.122% | 0.036% | 0.675% | 0.444% | 0.492% |

| | | Standard Deviation of the average editing percent at the | | | | |
|---|---|---|---|---|---|---|
| | | nucleotides numbered according to the position in the PWsp449 | | | | |
| | Protein SEQ | spacer sequence on the non-target, genomic DNA strand | | | | |
| pWISE | ID NO: | C.20 | C.22 | C.30 | C.32 | C.33 |
| pWISE121 | 56 | 0.035% | 0.223% | 0.938% | 0.690% | 0.714% |
| pWISE9120 | 453 | No data | No data | No data | No data | No data |
| pWISE10648 | 629 | 0.193% | 0.223% | 0.153% | 0.062% | 0.035% |
| pWISE10649 | 630 | 0.010% | 0.117% | 0.030% | 0.018% | 0.008% |
| pWISE10650 | 631 | 0.194% | 0.219% | 0.005% | 0.045% | 0.021% |
| pWISE10651 | 632 | 0.501% | 0.344% | 0.048% | 0.002% | 0.012% |
| pWISE10652 | 633 | 0.069% | 0.052% | 0.048% | 0.005% | 0.004% |
| pWISE10658 | 639 | 0.115% | 0.139% | 0.006% | 0.006% | 0.005% |
| pWISE10653 | 634 | 0.033% | 0.098% | 0.011% | 0.003% | 0.003% |
| pWISE10654 | 635 | No data | No data | No data | No data | No data |
| pWISE10655 | 636 | 0.075% | 0.255% | 0.012% | 0.011% | 0.051% |
| pWISE10656 | 637 | 0.013% | 0.152% | 0.046% | 0.009% | 0.014% |
| pWISE10657 | 638 | 0.134% | 0.003% | 0.091% | 0.004% | 0.008% |

TABLE 63

| | | Average G to A editing percent at the nucleotides numbered | | | | | |
|---|---|---|---|---|---|---|---|
| | | according to the position in the PWsp449 spacer sequence | | | | | |
| | Protein | on the opposite, target genomic DNA strand | | | | | |
| pWISE | SEQ ID NO: | G.-9 | G.1 | G.3 | G.4 | G.7 | G.17 |
| pWISE121 | 56 | 0.048% | 0.105% | 0.144% | 0.133% | 0.178% | 0.670% |
| pWISE9120 | 453 | 0.005% | 0.061% | 0.056% | 0.123% | 0.015% | 5.788% |
| pWISE10648 | 629 | 0.002% | 0.037% | 0.094% | 0.094% | 0.022% | 3.324% |
| pWISE10649 | 630 | 0.008% | 0.029% | 0.032% | 0.057% | 0.013% | 4.321% |
| pWISE10650 | 631 | 0.016% | 0.010% | 0.033% | 0.097% | 0.028% | 7.663% |
| pWISE10651 | 632 | 0.014% | 0.005% | 0.037% | 0.084% | 0.009% | 7.590% |
| pWISE10652 | 633 | 0.012% | 0.057% | 0.094% | 0.107% | 0.036% | 6.836% |
| pWISE10658 | 639 | 0.021% | 0.043% | 0.039% | 0.051% | 0.014% | 10.694% |
| pWISE10653 | 634 | 0.035% | 0.041% | 0.056% | 0.058% | 0.027% | 7.636% |
| pWISE10654 | 635 | 0.019% | 0.000% | 0.070% | 0.103% | 0.005% | 10.854% |
| pWISE10655 | 636 | 0.022% | 0.039% | 0.050% | 0.064% | 0.014% | 12.072% |
| pWISE10656 | 637 | 0.021% | 0.022% | 0.042% | 0.058% | 0.016% | 11.537% |
| pWISE10657 | 638 | 0.016% | 0.037% | 0.072% | 0.089% | 0.016% | 11.411% |

| | | Average G to A editing percent at the nucleotides numbered | | | | |
|---|---|---|---|---|---|---|
| | | according to the position in the PWsp449 spacer sequence | | | | |
| | Protein | on the opposite, target genomic DNA strand | | | | |
| pWISE | SEQ ID NO: | G.21 | G.24 | G.26 | G.28 | G.31 |
| pWISE121 | 56 | 0.425% | 0.732% | 0.656% | 0.146% | 0.194% |
| pWISE9120 | 453 | 4.637% | 5.737% | 4.330% | 0.517% | 0.512% |
| pWISE10648 | 629 | 2.675% | 3.271% | 2.485% | 0.392% | 0.395% |
| pWISE10649 | 630 | 3.838% | 4.557% | 3.560% | 0.613% | 0.542% |
| pWISE10650 | 631 | 7.215% | 8.293% | 6.719% | 1.160% | 0.975% |
| pWISE10651 | 632 | 7.273% | 8.207% | 6.554% | 1.527% | 1.221% |
| pWISE10652 | 633 | 6.741% | 7.520% | 6.055% | 1.546% | 1.293% |
| pWISE10658 | 639 | 7.782% | 9.590% | 7.451% | 0.889% | 0.838% |
| pWISE10653 | 634 | 5.570% | 6.517% | 4.768% | 0.623% | 0.491% |
| pWISE10654 | 635 | 9.209% | 10.512% | 7.994% | 1.238% | 0.952% |
| pWISE10655 | 636 | 10.655% | 11.870% | 9.214% | 1.592% | 1.320% |
| pWISE10656 | 637 | 10.228% | 11.666% | 9.072% | 1.626% | 1.455% |
| pWISE10657 | 638 | 10.249% | 11.805% | 9.203% | 1.686% | 1.228% |

TABLE 64

| | | Standard deviation of the average G to A editing percent at the | | | | | |
|---|---|---|---|---|---|---|---|
| | | nucleotides numbered according to the position in the PWsp449 | | | | | |
| | Protein | spacer sequence on the opposite, target genomic DNA strand | | | | | |
| pWISE | SEQ ID NO: | G.-9 | G.1 | G.3 | G.4 | G.7 | G.17 |
| pWISE121 | 56 | 0.024% | 0.042% | 0.018% | 0.021% | 0.018% | 0.152% |
| pWISE9120 | 453 | No data | No data | No data | No data | No data | No data |
| pWISE10648 | 629 | 0.004% | 0.003% | 0.035% | 0.042% | 0.010% | 0.234% |
| pWISE10649 | 630 | 0.005% | 0.001% | 0.003% | 0.015% | 0.018% | 0.931% |
| pWISE10650 | 631 | 0.002% | 0.006% | 0.006% | 0.018% | 0.007% | 1.232% |
| pWISE10651 | 632 | 0.006% | 0.000% | 0.038% | 0.009% | 0.006% | 1.423% |
| pWISE10652 | 633 | 0.008% | 0.007% | 0.059% | 0.028% | 0.042% | 0.854% |
| pWISE10658 | 639 | 0.009% | 0.008% | 0.009% | 0.018% | 0.019% | 2.101% |
| pWISE10653 | 634 | 0.006% | 0.017% | 0.032% | 0.035% | 0.024% | 1.647% |
| pWISE10654 | 635 | No data | No data | No data | No data | No data | No data |
| pWISE10655 | 636 | 0.013% | 0.011% | 0.044% | 0.030% | 0.011% | 1.303% |
| pWISE10656 | 637 | 0.007% | 0.013% | 0.029% | 0.016% | 0.001% | 1.100% |
| pWISE10657 | 638 | 0.010% | 0.013% | 0.023% | 0.040% | 0.010% | 1.823% |

| | | Standard deviation of the average G to A editing percent at the | | | | |
|---|---|---|---|---|---|---|
| | | nucleotides numbered according to the position in the PWsp449 | | | | |
| | Protein | spacer sequence on the opposite, target genomic DNA strand | | | | |
| pWISE | SEQ ID NO: | G.21 | G.24 | G.26 | G.28 | G.31 |
| pWISE121 | 56 | 0.144% | 0.201% | 0.297% | 0.083% | 0.045% |
| pWISE9120 | 453 | No data | No data | No data | No data | No data |
| pWISE10648 | 629 | 0.220% | 0.454% | 0.454% | 0.015% | 0.100% |
| pWISE10649 | 630 | 0.844% | 1.031% | 0.814% | 0.182% | 0.074% |
| pWISE10650 | 631 | 1.016% | 1.311% | 0.767% | 0.099% | 0.097% |
| pWISE10651 | 632 | 1.209% | 1.490% | 1.466% | 0.132% | 0.139% |
| pWISE10652 | 633 | 0.548% | 0.748% | 0.397% | 0.067% | 0.173% |
| pWISE10658 | 639 | 1.395% | 1.580% | 1.440% | 0.133% | 0.183% |
| pWISE10653 | 634 | 1.457% | 1.543% | 1.096% | 0.230% | 0.010% |
| pWISE10654 | 635 | No data | No data | No data | No data | No data |
| pWISE10655 | 636 | 1.497% | 1.660% | 1.492% | 0.279% | 0.033% |
| pWISE10656 | 637 | 1.011% | 1.437% | 1.161% | 0.227% | 0.037% |
| pWISE10657 | 638 | 1.854% | 1.870% | 1.597% | 0.396% | 0.272% |

TABLE 65

| | | Average C to T editing percent at the nucleotides numbered | | | | | | |
|---|---|---|---|---|---|---|---|---|
| | | according to the position in the PWsp135 spacer sequence | | | | | | |
| | Protein | on the non-target, genomic DNA strand | | | | | | |
| pWISE | SEQ ID NO: | C.-8 | C.-6 | C.-1 | C.3 | C.4 | C.6 | C.8 |
| pWISE121 | 56 | 0.012% | 0.011% | 0.054% | 0.017% | 0.030% | 0.342% | 0.327% |
| pWISE9120 | 453 | 0.073% | 0.003% | 0.441% | 0.009% | 0.007% | 2.246% | 0.283% |
| pWISE10648 | 629 | 0.104% | 0.005% | 0.441% | 0.021% | 0.006% | 1.364% | 0.058% |
| pWISE10649 | 630 | 0.180% | 0.004% | 0.629% | 0.019% | 0.006% | 1.385% | 0.045% |
| pWISE10650 | 631 | 0.213% | 0.013% | 0.706% | 0.008% | 0.026% | 1.618% | 0.072% |
| pWISE10651 | 632 | 0.214% | 0.001% | 0.694% | 0.014% | 0.015% | 1.580% | 0.068% |
| pWISE10652 | 633 | 0.180% | 0.016% | 0.633% | 0.029% | 0.002% | 1.190% | 0.102% |
| pWISE10658 | 639 | 0.215% | 0.015% | 0.912% | 0.006% | 0.011% | 3.424% | 0.166% |
| pWISE10653 | 634 | 0.214% | 0.013% | 0.673% | 0.010% | 0.034% | 2.026% | 0.076% |
| pWISE10654 | 635 | 0.116% | 0.006% | 0.619% | 0.006% | 0.012% | 1.734% | 0.083% |
| pWISE10655 | 636 | 0.134% | 0.007% | 0.633% | 0.007% | 0.010% | 1.883% | 0.067% |
| pWISE10656 | 637 | 0.192% | 0.000% | 0.644% | 0.023% | 0.026% | 2.051% | 0.096% |
| pWISE10657 | 638 | 0.160% | 0.016% | 0.639% | 0.018% | 0.012% | 1.781% | 0.037% |

| | | Average C to T editing percent at the nucleotides numbered | | | | | |
|---|---|---|---|---|---|---|---|
| | | according to the position in the PWsp135 spacer sequence | | | | | |
| | Protein | on the non-target, genomic DNA strand | | | | | |
| pWISE | SEQ ID NO: | C.9 | C.10 | C.11 | C.13 | C.17 | C.18 |
| pWISE121 | 56 | 0.368% | 0.359% | 0.352% | 0.382% | 0.512% | 0.540% |
| pWISE9120 | 453 | 0.540% | 1.085% | 1.322% | 3.764% | 0.217% | 0.374% |
| pWISE10648 | 629 | 0.227% | 0.465% | 0.687% | 2.623% | 0.061% | 0.302% |
| pWISE10649 | 630 | 0.221% | 0.453% | 0.633% | 3.790% | 0.337% | 1.053% |
| pWISE10650 | 631 | 0.288% | 0.529% | 0.714% | 3.783% | 0.382% | 1.100% |
| pWISE10651 | 632 | 0.224% | 0.394% | 0.607% | 4.165% | 0.375% | 1.402% |
| pWISE10652 | 633 | 0.208% | 0.433% | 0.659% | 2.821% | 0.275% | 0.925% |
| pWISE10658 | 639 | 0.860% | 1.776% | 2.233% | 6.015% | 0.146% | 0.386% |
| pWISE10653 | 634 | 0.414% | 0.751% | 1.028% | 4.129% | 0.169% | 0.760% |
| pWISE10654 | 635 | 0.271% | 0.558% | 0.713% | 3.967% | 0.438% | 1.411% |
| pWISE10655 | 636 | 0.214% | 0.506% | 0.684% | 5.548% | 0.754% | 2.321% |
| pWISE10656 | 637 | 0.254% | 0.551% | 0.807% | 5.158% | 0.485% | 1.716% |
| pWISE10657 | 638 | 0.242% | 0.446% | 0.664% | 5.200% | 0.269% | 1.160% |
| | | Average C to T editing percent at the nucleotides numbered | | | | | |
| | | according to the position in the PWsp135 spacer sequence | | | | | |
| | Protein | on the non-target, genomic DNA strand | | | | | |
| pWISE | SEQ ID NO: | C.19 | C.22 | C.25 | C.27 | C.29 | C.33 |
| pWISE121 | 56 | 0.501% | 0.378% | 0.115% | 0.085% | 0.123% | 0.057% |
| pWISE9120 | 453 | 0.909% | 0.172% | 0.273% | 0.260% | 0.248% | 0.246% |
| pWISE10648 | 629 | 0.625% | 0.222% | 0.282% | 0.261% | 0.277% | 0.235% |
| pWISE10649 | 630 | 1.766% | 1.032% | 0.795% | 0.964% | 1.013% | 0.978% |
| pWISE10650 | 631 | 1.725% | 1.211% | 1.049% | 1.275% | 1.301% | 1.220% |
| pWISE10651 | 632 | 2.292% | 1.701% | 1.484% | 1.628% | 1.595% | 1.569% |
| pWISE10652 | 633 | 1.513% | 1.196% | 0.887% | 1.194% | 1.195% | 1.201% |
| pWISE10658 | 639 | 1.025% | 0.404% | 0.451% | 0.402% | 0.492% | 0.417% |
| pWISE10653 | 634 | 1.484% | 0.538% | 0.494% | 0.593% | 0.597% | 0.540% |
| pWISE10654 | 635 | 2.207% | 1.243% | 1.078% | 1.198% | 1.252% | 1.150% |
| pWISE10655 | 636 | 3.457% | 2.444% | 1.948% | 2.362% | 2.346% | 1.977% |
| pWISE10656 | 637 | 2.681% | 1.942% | 1.522% | 1.835% | 1.833% | 1.617% |
| pWISE10657 | 638 | 2.127% | 1.728% | 1.424% | 1.633% | 1.598% | 1.289% |

TABLE 66

| | | Standard Deviation of the average editing percent at the | | | | | | |
|---|---|---|---|---|---|---|---|---|
| | | nucleotides numbered according to the position in the PWsp135 | | | | | | |
| | Protein | spacer sequence on the non-target, genomic DNA strand | | | | | | |
| pWISE | SEQ ID NO: | C.-8 | C.-6 | C.-1 | C.3 | C.4 | C.6 | C.8 |
| pWISE121 | 56 | 0.002% | 0.015% | 0.064% | 0.011% | 0.014% | 0.411% | 0.437% |
| pWISE9120 | 453 | 0.092% | 0.004% | 0.238% | 0.007% | 0.002% | 0.988% | 0.400% |
| pWISE10648 | 629 | 0.001% | 0.003% | 0.131% | 0.002% | 0.004% | 0.188% | 0.046% |
| pWISE10649 | 630 | 0.017% | 0.001% | 0.109% | 0.022% | 0.002% | 0.226% | 0.008% |
| pWISE10650 | 631 | 0.112% | 0.009% | 0.054% | 0.007% | 0.024% | 0.209% | 0.046% |
| pWISE10651 | 632 | 0.046% | 0.002% | 0.133% | 0.014% | 0.000% | 0.023% | 0.009% |
| pWISE10652 | 633 | 0.085% | 0.008% | 0.003% | 0.041% | 0.002% | 0.009% | 0.058% |
| pWISE10658 | 639 | 0.084% | 0.014% | 0.379% | 0.002% | 0.003% | 0.283% | 0.034% |
| pWISE10653 | 634 | 0.025% | 0.019% | 0.036% | 0.014% | 0.010% | 0.049% | 0.017% |
| pWISE10654 | 635 | 0.039% | 0.008% | 0.205% | 0.004% | 0.013% | 0.819% | 0.056% |
| pWISE10655 | 636 | 0.053% | 0.009% | 0.003% | 0.009% | 0.009% | 0.024% | 0.015% |
| pWISE10656 | 637 | 0.008% | 0.000% | 0.023% | 0.012% | 0.012% | 0.363% | 0.033% |
| pWISE10657 | 638 | 0.091% | 0.006% | 0.329% | 0.004% | 0.017% | 0.316% | 0.013% |

| | |
|---|---|
| | Standard Deviation of the average editing percent at the |
| | nucleotides numbered according to the position in the PWsp135 |
| Protein | spacer sequence on the non-target, genomic DNA strand |

| pWISE | SEQ ID NO: | C.9 | C.10 | C.11 | C.13 | C.17 | C.18 |
|---|---|---|---|---|---|---|---|
| pWISE121 | 56 | 0.454% | 0.441% | 0.409% | 0.435% | 0.456% | 0.378% |
| pWISE9120 | 453 | 0.763% | 0.710% | 0.909% | 1.971% | 0.295% | 0.333% |
| pWISE10648 | 629 | 0.030% | 0.128% | 0.114% | 0.112% | 0.031% | 0.030% |
| pWISE10649 | 630 | 0.062% | 0.180% | 0.184% | 0.595% | 0.131% | 0.283% |
| pWISE10650 | 631 | 0.018% | 0.021% | 0.075% | 0.562% | 0.071% | 0.321% |
| pWISE10651 | 632 | 0.006% | 0.071% | 0.086% | 0.609% | 0.103% | 0.437% |
| pWISE10652 | 633 | 0.000% | 0.039% | 0.002% | 0.007% | 0.024% | 0.102% |
| pWISE10658 | 639 | 0.074% | 0.073% | 0.181% | 0.175% | 0.038% | 0.087% |
| pWISE10653 | 634 | 0.068% | 0.034% | 0.050% | 0.062% | 0.039% | 0.018% |
| pWISE10654 | 635 | 0.097% | 0.232% | 0.288% | 2.243% | 0.085% | 0.524% |
| pWISE10655 | 636 | 0.072% | 0.006% | 0.008% | 0.308% | 0.036% | 0.071% |
| pWISE10656 | 637 | 0.023% | 0.120% | 0.224% | 0.670% | 0.026% | 0.020% |
| pWISE10657 | 638 | 0.007% | 0.104% | 0.246% | 0.940% | 0.023% | 0.287% |

| | |
|---|---|
| | Standard Deviation of the average editing percent at the |
| | nucleotides numbered according to the position in the PWsp135 |
| Protein | spacer sequence on the non-target, genomic DNA strand |

| pWISE | SEQ ID NO: | C.19 | C.22 | C.25 | C.27 | C.29 | C.33 |
|---|---|---|---|---|---|---|---|
| pWISE121 | 56 | 0.467% | 0.079% | 0.073% | 0.021% | 0.076% | 0.056% |
| pWISE9120 | 453 | 0.349% | 0.065% | 0.024% | 0.178% | 0.173% | 0.211% |
| pWISE10648 | 629 | 0.045% | 0.018% | 0.074% | 0.041% | 0.018% | 0.059% |
| pWISE10649 | 630 | 0.344% | 0.334% | 0.217% | 0.193% | 0.257% | 0.243% |
| pWISE10650 | 631 | 0.336% | 0.243% | 0.254% | 0.277% | 0.291% | 0.303% |
| pWISE10651 | 632 | 0.560% | 0.407% | 0.348% | 0.342% | 0.393% | 0.402% |
| pWISE10652 | 633 | 0.017% | 0.124% | 0.095% | 0.117% | 0.095% | 0.052% |
| pWISE10658 | 639 | 0.148% | 0.015% | 0.044% | 0.043% | 0.074% | 0.052% |
| pWISE10653 | 634 | 0.039% | 0.011% | 0.025% | 0.009% | 0.025% | 0.051% |
| pWISE10654 | 635 | 0.972% | 0.499% | 0.472% | 0.555% | 0.601% | 0.480% |
| pWISE10655 | 636 | 0.153% | 0.009% | 0.067% | 0.031% | 0.058% | 0.045% |
| pWISE10656 | 637 | 0.238% | 0.345% | 0.258% | 0.335% | 0.259% | 0.324% |
| pWISE10657 | 638 | 0.296% | 0.214% | 0.250% | 0.224% | 0.297% | 0.264% |

TABLE 67

| | | Average G to A editing percent at the nucleotides numbered | | |
|---|---|---|---|---|
| | | according to the position in the PWsp135 spacer sequence on | | |
| | Protein SEQ | the opposite, target genomic DNA strand | | |
| pWISE | ID NO: | G.-4 | G.2 | G.16 |
| pWISE121 | 56 | 0.003% | 0.028% | 0.546% |
| pWISE9120 | 453 | 0.005% | 0.006% | 5.479% |
| pWISE10648 | 629 | 0.016% | 0.008% | 5.415% |
| pWISE10649 | 630 | 0.007% | 0.014% | 8.924% |
| pWISE10650 | 631 | 0.010% | 0.020% | 10.706% |
| pWISE10651 | 632 | 0.011% | 0.012% | 10.714% |
| pWISE10652 | 633 | 0.018% | 0.014% | 7.916% |
| pWISE10658 | 639 | 0.021% | 0.005% | 8.970% |
| pWISE10653 | 634 | 0.003% | 0.005% | 9.975% |
| pWISE10654 | 635 | 0.016% | 0.015% | 10.168% |
| pWISE10655 | 636 | 0.007% | 0.009% | 14.117% |
| pWISE10656 | 637 | 0.030% | 0.015% | 13.045% |
| pWISE10657 | 638 | 0.005% | 0.018% | 10.448% |

TABLE 68

| | | Standard deviation of the average G to A editing percent at the | | |
|---|---|---|---|---|
| | | nucleotides numbered according to the position in the PWsp135 | | |
| | Protein SEQ | spacer sequence on the opposite, target genomic DNA strand | | |
| pWISE | ID NO: | G.-4 | G.2 | G.16 |
| pWISE121 | 56 | 0.005% | 0.002% | 0.290% |
| pWISE9120 | 453 | 0.001% | 0.004% | 1.864% |
| pWISE10648 | 629 | 0.013% | 0.007% | 1.070% |
| pWISE10649 | 630 | 0.010% | 0.015% | 1.642% |
| pWISE10650 | 631 | 0.005% | 0.014% | 0.576% |
| pWISE10651 | 632 | 0.012% | 0.005% | 2.047% |
| pWISE10652 | 633 | 0.020% | 0.015% | 0.034% |
| pWISE10658 | 639 | 0.026% | 0.000% | 0.597% |
| pWISE10653 | 634 | 0.004% | 0.007% | 0.725% |
| pWISE10654 | 635 | 0.011% | 0.017% | 5.446% |
| pWISE10655 | 636 | 0.004% | 0.009% | 1.165% |
| pWISE10656 | 637 | 0.007% | 0.013% | 1.794% |
| pWISE10657 | 638 | 0.003% | 0.004% | 1.601% |

TABLE 69

| | | Average C to T editing percent at the nucleotides numbered | | | | |
|---|---|---|---|---|---|---|
| | | according to the position in the PWsp389 spacer sequence | | | | |
| | Protein | on the non-target, genomic DNA strand | | | | |
| pWISE | SEQ ID NO: | C.-5 | C.-1 | C.4 | C.6 | C.7 |
| pWISE121 | 56 | 0.000% | 0.070% | 0.068% | 0.094% | 0.270% |
| pWISE9120 | 453 | 0.014% | 0.191% | 0.231% | 0.036% | 0.390% |
| pWISE10648 | 629 | 0.005% | 0.063% | 0.072% | 0.000% | 0.120% |
| pWISE10649 | 630 | 0.004% | 0.052% | 0.025% | 0.022% | 0.098% |
| pWISE10650 | 631 | 0.023% | 0.160% | 0.165% | 0.059% | 0.260% |
| pWISE10651 | 632 | 0.010% | 0.109% | 0.145% | 0.042% | 0.203% |
| pWISE10652 | 633 | 0.023% | 0.103% | 0.092% | 0.031% | 0.086% |
| pWISE10658 | 639 | 0.003% | 0.324% | 0.259% | 0.128% | 0.397% |
| pWISE10653 | 634 | 0.010% | 0.148% | 0.172% | 0.072% | 0.154% |
| pWISE10654 | 635 | 0.013% | 0.213% | 0.151% | 0.057% | 0.180% |
| pWISE10655 | 636 | 0.008% | 0.174% | 0.178% | 0.073% | 0.179% |
| pWISE10656 | 637 | 0.017% | 0.146% | 0.191% | 0.038% | 0.216% |
| pWISE10657 | 638 | 0.026% | 0.201% | 0.207% | 0.044% | 0.165% |
| | | Average C to T editing percent at the nucleotides numbered | | | | |
| | | according to the position in the PWsp389 spacer sequence | | | | |
| | Protein | on the non-target, genomic DNA strand | | | | |
| pWISE | SEQ ID NO: | C.11 | C.12 | C.16 | C.17 | C.18 |
| pWISE121 | 56 | 0.168% | 0.254% | 0.726% | 0.805% | 0.793% |
| pWISE9120 | 453 | 1.885% | 1.898% | 0.572% | 0.359% | 0.357% |
| pWISE10648 | 629 | 0.525% | 0.487% | 0.212% | 0.125% | 0.111% |
| pWISE10649 | 630 | 0.546% | 0.504% | 0.154% | 0.117% | 0.127% |
| pWISE10650 | 631 | 1.226% | 1.139% | 0.525% | 0.377% | 0.380% |
| pWISE10651 | 632 | 1.372% | 1.256% | 0.538% | 0.422% | 0.436% |
| pWISE10652 | 633 | 0.594% | 0.592% | 0.344% | 0.251% | 0.257% |
| pWISE10658 | 639 | 2.299% | 2.086% | 0.433% | 0.187% | 0.217% |
| pWISE10653 | 634 | 1.022% | 0.953% | 0.257% | 0.137% | 0.106% |
| pWISE10654 | 635 | 1.214% | 1.107% | 0.260% | 0.170% | 0.198% |
| pWISE10655 | 636 | 1.333% | 1.139% | 0.265% | 0.149% | 0.198% |
| pWISE10656 | 637 | 1.187% | 0.964% | 0.261% | 0.139% | 0.108% |
| pWISE10657 | 638 | 1.170% | 1.025% | 0.270% | 0.128% | 0.112% |
| | | Average C to T editing percent at the nucleotides numbered | | | | |
| | | according to the position in the PWsp389 spacer sequence | | | | |
| | Protein | on the non-target, genomic DNA strand | | | | |
| pWISE | SEQ ID NO: | C.25 | C.26 | C.27 | C.28 | C.30 |
| pWISE121 | 56 | 0.052% | 0.120% | 0.087% | 0.175% | 0.082% |
| pWISE9120 | 453 | 0.040% | 0.031% | 0.051% | 0.074% | 0.058% |
| pWISE10648 | 629 | 0.053% | 0.087% | 0.125% | 0.183% | 0.246% |
| pWISE10649 | 630 | 0.019% | 0.021% | 0.022% | 0.032% | 0.017% |
| pWISE10650 | 631 | 0.055% | 0.066% | 0.056% | 0.077% | 0.030% |
| pWISE10651 | 632 | 0.049% | 0.037% | 0.043% | 0.037% | 0.039% |
| pWISE10652 | 633 | 0.045% | 0.040% | 0.040% | 0.047% | 0.004% |
| pWISE10658 | 639 | 0.005% | 0.008% | 0.022% | 0.017% | 0.005% |
| pWISE10653 | 634 | 0.003% | 0.003% | 0.003% | 0.010% | 0.003% |
| pWISE10654 | 635 | 0.031% | 0.021% | 0.021% | 0.040% | 0.017% |
| pWISE10655 | 636 | 0.017% | 0.033% | 0.047% | 0.029% | 0.016% |
| pWISE10656 | 637 | 0.024% | 0.017% | 0.028% | 0.024% | 0.014% |
| pWISE10657 | 638 | 0.031% | 0.034% | 0.032% | 0.032% | 0.003% |

TABLE 70

| | | Standard Deviation of the average editing percent at the | | | | |
|---|---|---|---|---|---|---|
| | | nucleotides numbered according to the position in the PWsp389 | | | | |
| | Protein | spacer sequence on the non-target, genomic DNA strand | | | | |
| pWISE | SEQ ID NO: | C.-5 | C.-1 | C.4 | C.6 | C.7 |
| pWISE121 | 56 | 0.000% | 0.077% | 0.005% | 0.012% | 0.004% |
| pWISE9120 | 453 | 0.019% | 0.090% | 0.057% | 0.026% | 0.056% |
| pWISE10648 | 629 | No data | No data | No data | No data | No data |
| pWISE10649 | 630 | 0.006% | 0.003% | 0.012% | 0.007% | 0.015% |
| pWISE10650 | 631 | 0.013% | 0.037% | 0.063% | 0.008% | 0.009% |
| pWISE10651 | 632 | 0.003% | 0.053% | 0.002% | 0.015% | 0.063% |
| pWISE10652 | 633 | 0.009% | 0.041% | 0.057% | 0.003% | 0.008% |
| pWISE10658 | 639 | 0.004% | 0.004% | 0.058% | 0.027% | 0.004% |
| pWISE10653 | 634 | 0.005% | 0.035% | 0.001% | 0.062% | 0.081% |
| pWISE10654 | 635 | 0.009% | 0.051% | 0.080% | 0.036% | 0.049% |
| pWISE10655 | 636 | 0.011% | 0.105% | 0.111% | 0.046% | 0.119% |
| pWISE10656 | 637 | 0.025% | 0.010% | 0.015% | 0.025% | 0.020% |
| pWISE10657 | 638 | 0.009% | 0.011% | 0.020% | 0.006% | 0.070% |
| | | Standard Deviation of the average editing percent at the | | | | |
| | | nucleotides numbered according to the position in the PWsp389 | | | | |
| | Protein | spacer sequence on the non-target, genomic DNA strand | | | | |
| pWISE | SEQ ID NO: | C.11 | C.12 | C.16 | C.17 | C.18 |
| pWISE121 | 56 | 0.040% | 0.101% | 0.050% | 0.043% | 0.083% |
| pWISE9120 | 453 | 0.470% | 0.501% | 0.028% | 0.098% | 0.010% |
| pWISE10648 | 629 | No data | No data | No data | No data | No data |
| pWISE10649 | 630 | 0.053% | 0.054% | 0.088% | 0.082% | 0.080% |
| pWISE10650 | 631 | 0.012% | 0.078% | 0.090% | 0.003% | 0.012% |
| pWISE10651 | 632 | 0.175% | 0.169% | 0.189% | 0.116% | 0.153% |
| pWISE10652 | 633 | 0.330% | 0.320% | 0.227% | 0.160% | 0.186% |
| pWISE10658 | 639 | 0.316% | 0.283% | 0.003% | 0.033% | 0.042% |
| pWISE10653 | 634 | 0.040% | 0.099% | 0.042% | 0.067% | 0.043% |
| pWISE10654 | 635 | 0.063% | 0.019% | 0.082% | 0.034% | 0.083% |
| pWISE10655 | 636 | 0.033% | 0.073% | 0.055% | 0.019% | 0.034% |
| pWISE10656 | 637 | 0.152% | 0.064% | 0.025% | 0.049% | 0.015% |
| pWISE10657 | 638 | 0.198% | 0.061% | 0.068% | 0.024% | 0.008% |
| | | Standard Deviation of the average editing percent at the | | | | |
| | | nucleotides numbered according to the position in the PWsp389 | | | | |
| | Protein | spacer sequence on the non-target, genomic DNA strand | | | | |
| pWISE | SEQ ID NO: | C.25 | C.26 | C.27 | C.28 | C.30 |
| pWISE121 | 56 | 0.028% | 0.012% | 0.029% | 0.078% | 0.028% |
| pWISE9120 | 453 | 0.043% | 0.031% | 0.002% | 0.007% | 0.008% |
| pWISE10648 | 629 | No data | No data | No data | No data | No data |
| pWISE10649 | 630 | 0.020% | 0.029% | 0.016% | 0.037% | 0.024% |
| pWISE10650 | 631 | 0.002% | 0.009% | 0.013% | 0.005% | 0.014% |
| pWISE10651 | 632 | 0.011% | 0.006% | 0.005% | 0.006% | 0.013% |
| pWISE10652 | 633 | 0.031% | 0.025% | 0.025% | 0.027% | 0.006% |
| pWISE10658 | 639 | 0.007% | 0.003% | 0.006% | 0.010% | 0.007% |
| pWISE10653 | 634 | 0.005% | 0.005% | 0.005% | 0.014% | 0.005% |
| pWISE10654 | 635 | 0.044% | 0.029% | 0.029% | 0.021% | 0.015% |
| pWISE10655 | 636 | 0.002% | 0.028% | 0.008% | 0.002% | 0.010% |
| pWISE10656 | 637 | 0.015% | 0.025% | 0.039% | 0.034% | 0.020% |
| pWISE10657 | 638 | 0.003% | 0.020% | 0.019% | 0.019% | 0.005% |

TABLE 71

| | | Average G to A editing percent at the nucleotides numbered | | | | |
|---|---|---|---|---|---|---|
| | | according to the position in the PWsp389 spacer sequence | | | | |
| | Protein | on the opposite, target genomic DNA strand | | | | |
| pWISE | SEQ ID NO: | G.-9 | G.-8 | G.-7 | G.-6 | G.2 |
| pWISE121 | 56 | 0.008% | 0.033% | 0.022% | 0.023% | 0.094% |
| pWISE9120 | 453 | 0.029% | 0.031% | 0.000% | 0.000% | 0.007% |
| pWISE10648 | 629 | 0.014% | 0.000% | 0.010% | 0.000% | 0.010% |
| pWISE10649 | 630 | 0.014% | 0.016% | 0.008% | 0.004% | 0.000% |
| pWISE10650 | 631 | 0.040% | 0.017% | 0.017% | 0.020% | 0.008% |
| pWISE10651 | 632 | 0.033% | 0.009% | 0.023% | 0.000% | 0.016% |
| pWISE10652 | 633 | 0.035% | 0.004% | 0.004% | 0.000% | 0.008% |
| pWISE10658 | 639 | 0.091% | 0.023% | 0.033% | 0.011% | 0.009% |
| pWISE10653 | 634 | 0.086% | 0.044% | 0.031% | 0.007% | 0.021% |
| pWISE10654 | 635 | 0.093% | 0.026% | 0.013% | 0.013% | 0.007% |
| pWISE10655 | 636 | 0.046% | 0.029% | 0.015% | 0.005% | 0.011% |
| pWISE10656 | 637 | 0.035% | 0.010% | 0.010% | 0.010% | 0.007% |
| pWISE10657 | 638 | 0.040% | 0.023% | 0.008% | 0.010% | 0.032% |
| | | Average G to A editing percent at the nucleotides numbered | | | | |
| | | according to the position in the PWsp389 spacer sequence | | | | |
| | Protein | on the opposite, target genomic DNA strand | | | | |
| pWISE | SEQ ID NO: | G.14 | G.21 | G.23 | G.24 | G.32 |
| pWISE121 | 56 | 0.491% | 0.310% | 0.243% | 0.248% | 0.358% |
| pWISE9120 | 453 | 0.690% | 0.193% | 0.117% | 0.077% | 0.136% |
| pWISE10648 | 629 | 0.352% | 0.077% | 0.135% | 0.092% | 0.222% |
| pWISE10649 | 630 | 0.301% | 0.139% | 0.106% | 0.101% | 0.055% |
| pWISE10650 | 631 | 0.567% | 0.296% | 0.303% | 0.211% | 0.116% |
| pWISE10651 | 632 | 1.042% | 0.527% | 0.518% | 0.437% | 0.128% |
| pWISE10652 | 633 | 0.603% | 0.427% | 0.415% | 0.331% | 0.116% |
| pWISE10658 | 639 | 1.085% | 0.480% | 0.232% | 0.139% | 0.087% |
| pWISE10653 | 634 | 0.885% | 0.391% | 0.144% | 0.075% | 0.051% |
| pWISE10654 | 635 | 0.833% | 0.524% | 0.281% | 0.235% | 0.146% |
| pWISE10655 | 636 | 0.989% | 0.718% | 0.446% | 0.313% | 0.112% |
| pWISE10656 | 637 | 0.756% | 0.540% | 0.366% | 0.244% | 0.108% |
| pWISE10657 | 638 | 0.904% | 0.745% | 0.680% | 0.477% | 0.163% |

TABLE 72

| | | Standard deviation of the average G to A editing percent at the | | | | |
|---|---|---|---|---|---|---|
| | | nucleotides numbered according to the position in the PWsp389 | | | | |
| | Protein | spacer sequence on the opposite, target genomic DNA strand | | | | |
| pWISE | SEQ ID NO: | G.-9 | G.-8 | G.-7 | G.-6 | G.2 |
| pWISE121 | 56 | 0.011% | 0.015% | 0.001% | 0.013% | 0.019% |
| pWISE9120 | 453 | 0.017% | 0.031% | 0.000% | 0.000% | 0.010% |
| pWISE10648 | 629 | No data | No data | No data | No data | No data |
| pWISE10649 | 630 | 0.019% | 0.000% | 0.011% | 0.006% | 0.000% |
| pWISE10650 | 631 | 0.057% | 0.024% | 0.024% | 0.028% | 0.011% |
| pWISE10651 | 632 | 0.012% | 0.012% | 0.009% | 0.000% | 0.023% |
| pWISE10652 | 633 | 0.033% | 0.006% | 0.006% | 0.000% | 0.012% |
| pWISE10658 | 639 | 0.027% | 0.018% | 0.013% | 0.001% | 0.013% |
| pWISE10653 | 634 | 0.043% | 0.043% | 0.005% | 0.010% | 0.010% |
| pWISE10654 | 635 | 0.092% | 0.017% | 0.018% | 0.009% | 0.010% |
| pWISE10655 | 636 | 0.020% | 0.002% | 0.021% | 0.006% | 0.016% |
| pWISE10656 | 637 | 0.010% | 0.005% | 0.005% | 0.005% | 0.010% |
| pWISE10657 | 638 | 0.016% | 0.023% | 0.002% | 0.014% | 0.036% |
| | | Standard deviation of the average G to A editing percent at the | | | | |
| | | nucleotides numbered according to the position in the PWsp389 | | | | |
| | Protein | spacer sequence on the opposite, target genomic DNA strand | | | | |
| pWISE | SEQ ID NO: | G.14 | G.21 | G.23 | G.24 | G.32 |
| pWISE121 | 56 | 0.002% | 0.092% | 0.047% | 0.081% | 0.074% |
| pWISE9120 | 453 | 0.190% | 0.124% | 0.082% | 0.064% | 0.117% |
| pWISE10648 | 629 | No data | No data | No data | No data | No data |
| pWISE10649 | 630 | 0.119% | 0.079% | 0.080% | 0.025% | 0.040% |
| pWISE10650 | 631 | 0.054% | 0.060% | 0.107% | 0.100% | 0.041% |
| pWISE10651 | 632 | 0.098% | 0.009% | 0.003% | 0.038% | 0.023% |
| pWISE10652 | 633 | 0.457% | 0.402% | 0.384% | 0.315% | 0.116% |
| pWISE10658 | 639 | 0.115% | 0.092% | 0.083% | 0.026% | 0.005% |
| pWISE10653 | 634 | 0.090% | 0.052% | 0.001% | 0.001% | 0.004% |
| pWISE10654 | 635 | 0.101% | 0.037% | 0.013% | 0.010% | 0.019% |
| pWISE10655 | 636 | 0.252% | 0.125% | 0.201% | 0.154% | 0.012% |
| pWISE10656 | 637 | 0.202% | 0.281% | 0.231% | 0.098% | 0.054% |
| pWISE10657 | 638 | 0.352% | 0.182% | 0.117% | 0.090% | 0.027% |

TABLE 73

| | | Average C to T editing percent at the nucleotides | | | | | |
|---|---|---|---|---|---|---|---|
| | | numbered according to the position in the PWsp390 | | | | | |
| | Protein | spacer sequence on the non-target, genomic DNA strand | | | | | |
| pWISE | SEQ ID NO: | C.-5 | C.2 | C.6 | C.7 | C.8 | C.9 |
| pWISE121 | 56 | 0.008% | 0.040% | 0.048% | 0.079% | 0.063% | 0.079% |
| pWISE9120 | 453 | 0.026% | 0.677% | 0.722% | 0.774% | 1.116% | 1.690% |
| pWISE10648 | 629 | 0.000% | 0.348% | 0.306% | 0.278% | 0.550% | 0.752% |
| pWISE10649 | 630 | 0.022% | 0.274% | 0.193% | 0.162% | 0.282% | 0.453% |
| pWISE10650 | 631 | 0.018% | 0.358% | 0.398% | 0.391% | 0.552% | 0.843% |
| pWISE10651 | 632 | 0.032% | 0.421% | 0.423% | 0.334% | 0.583% | 0.888% |
| pWISE10652 | 633 | 0.018% | 0.437% | 0.357% | 0.266% | 0.377% | 0.673% |
| pWISE10658 | 639 | 0.052% | 0.650% | 0.482% | 0.402% | 0.774% | 1.384% |
| pWISE10653 | 634 | 0.040% | 0.358% | 0.213% | 0.181% | 0.326% | 0.640% |
| pWISE10654 | 635 | No data | No data | No data | No data | No data | No data |
| pWISE10655 | 636 | 0.029% | 0.722% | 0.483% | 0.294% | 0.473% | 0.904% |
| pWISE10656 | 637 | 0.027% | 0.647% | 0.335% | 0.204% | 0.308% | 0.782% |
| pWISE10657 | 638 | 0.007% | 0.581% | 0.323% | 0.176% | 0.288% | 0.685% |

| | | Average C to T editing percent at the nucleotides | | | | |
|---|---|---|---|---|---|---|
| | | numbered according to the position in the PWsp390 | | | | |
| | Protein | spacer sequence on the non-target, genomic DNA strand | | | | |
| pWISE | SEQ ID NO: | C.11 | C.12 | C.14 | C.15 | C.16 |
| pWISE121 | 56 | 0.103% | 0.143% | 0.254% | 0.531% | 1.428% |
| pWISE9120 | 453 | 3.444% | 3.405% | 0.413% | 0.658% | 0.838% |
| pWISE10648 | 629 | 1.692% | 1.622% | 0.237% | 0.313% | 0.341% |
| pWISE10649 | 630 | 1.488% | 1.413% | 0.097% | 0.200% | 0.303% |
| pWISE10650 | 631 | 2.411% | 2.336% | 0.269% | 0.516% | 0.646% |
| pWISE10651 | 632 | 2.591% | 2.490% | 0.265% | 0.449% | 0.642% |
| pWISE10652 | 633 | 1.927% | 1.718% | 0.236% | 0.410% | 0.580% |
| pWISE10658 | 639 | 3.816% | 3.626% | 0.055% | 0.095% | 0.171% |
| pWISE10653 | 634 | 2.042% | 1.859% | 0.058% | 0.080% | 0.130% |
| pWISE10654 | 635 | No data | No data | No data | No data | No data |
| pWISE10655 | 636 | 3.942% | 3.676% | 0.070% | 0.088% | 0.196% |
| pWISE10656 | 637 | 3.596% | 3.163% | 0.061% | 0.124% | 0.192% |
| pWISE10657 | 638 | 3.217% | 2.969% | 0.042% | 0.098% | 0.163% |
| | | Average C to T editing percent at the nucleotides | | | | |
| | | numbered according to the position in the PWsp390 | | | | |
| | Protein | spacer sequence on the non-target, genomic DNA strand | | | | |
| pWISE | SEQ ID NO: | C.17 | C.19 | C.27 | C.28 | C.30 |
| pWISE121 | 56 | 3.824% | 1.475% | 0.159% | 0.151% | 0.008% |
| pWISE9120 | 453 | 0.942% | 1.142% | 0.052% | 0.052% | 0.039% |
| pWISE10648 | 629 | 0.411% | 0.801% | 0.021% | 0.007% | 0.000% |
| pWISE10649 | 630 | 0.343% | 0.769% | 0.026% | 0.030% | 0.018% |
| pWISE10650 | 631 | 0.700% | 1.537% | 0.022% | 0.051% | 0.015% |
| pWISE10651 | 632 | 0.736% | 1.643% | 0.019% | 0.016% | 0.009% |
| pWISE10652 | 633 | 0.671% | 1.339% | 0.043% | 0.039% | 0.004% |
| pWISE10658 | 639 | 0.250% | 0.630% | 0.000% | 0.028% | 0.012% |
| pWISE10653 | 634 | 0.149% | 0.465% | 0.015% | 0.008% | 0.004% |
| pWISE10654 | 635 | No data | No data | No data | No data | No data |
| pWISE10655 | 636 | 0.289% | 1.306% | 0.018% | 0.042% | 0.000% |
| pWISE10656 | 637 | 0.274% | 1.254% | 0.014% | 0.013% | 0.010% |
| pWISE10657 | 638 | 0.248% | 1.040% | 0.008% | 0.015% | 0.000% |

TABLE 74

| | | Standard Deviation of the average editing percent at the | | | | | |
|---|---|---|---|---|---|---|---|
| | | nucleotides numbered according to the position in the PWsp390 | | | | | |
| | Protein | spacer sequence on the non-target, genomic DNA strand | | | | | |
| pWISE | SEQ ID NO: | C.-5 | C.2 | C.6 | C.7 | C.8 | C.9 |
| pWISE121 | 56 | No data | No data | No data | No data | No data | No data |
| pWISE9120 | 453 | No data | No data | No data | No data | No data | No data |
| pWISE10648 | 629 | No data | No data | No data | No data | No data | No data |
| pWISE10649 | 630 | 0.031% | 0.103% | 0.050% | 0.046% | 0.028% | 0.031% |
| pWISE10650 | 631 | 0.026% | 0.121% | 0.148% | 0.137% | 0.128% | 0.135% |
| pWISE10651 | 632 | 0.037% | 0.096% | 0.194% | 0.127% | 0.254% | 0.270% |
| pWISE10652 | 633 | 0.026% | 0.251% | 0.160% | 0.135% | 0.208% | 0.313% |
| pWISE10658 | 639 | 0.035% | 0.014% | 0.194% | 0.189% | 0.107% | 0.126% |
| pWISE10653 | 634 | 0.018% | 0.010% | 0.011% | 0.017% | 0.007% | 0.039% |
| pWISE10654 | 635 | No data | No data | No data | No data | No data | No data |
| pWISE10655 | 636 | 0.008% | 0.218% | 0.110% | 0.041% | 0.134% | 0.098% |
| pWISE10656 | 637 | 0.011% | 0.141% | 0.017% | 0.021% | 0.029% | 0.114% |
| pWISE10657 | 638 | 0.001% | 0.062% | 0.132% | 0.020% | 0.017% | 0.036% |

| | | Standard Deviation of the average editing percent at the | | | | |
|---|---|---|---|---|---|---|
| | | nucleotides numbered according to the position in the PWsp390 | | | | |
| | Protein | spacer sequence on the non-target, genomic DNA strand | | | | |
| pWISE | SEQ ID NO: | C.11 | C.12 | C.14 | C.15 | C.16 |
| pWISE121 | 56 | No data | No data | No data | No data | No data |
| pWISE9120 | 453 | No data | No data | No data | No data | No data |
| pWISE10648 | 629 | No data | No data | No data | No data | No data |
| pWISE10649 | 630 | 0.170% | 0.145% | 0.066% | 0.089% | 0.062% |
| pWISE10650 | 631 | 0.196% | 0.198% | 0.143% | 0.096% | 0.132% |
| pWISE10651 | 632 | 0.443% | 0.467% | 0.030% | 0.065% | 0.171% |
| pWISE10652 | 633 | 0.570% | 0.483% | 0.114% | 0.161% | 0.202% |
| pWISE10658 | 639 | 0.145% | 0.029% | 0.019% | 0.007% | 0.004% |
| pWISE10653 | 634 | 0.052% | 0.068% | 0.044% | 0.025% | 0.018% |
| pWISE10654 | 635 | No data | No data | No data | No data | No data |
| pWISE10655 | 636 | 0.521% | 0.431% | 0.000% | 0.004% | 0.090% |
| pWISE10656 | 637 | 0.321% | 0.324% | 0.023% | 0.003% | 0.007% |
| pWISE10657 | 638 | 0.165% | 0.151% | 0.027% | 0.052% | 0.090% |
| | | Standard Deviation of the average editing percent at the | | | | |
| | | nucleotides numbered according to the position in the PWsp390 | | | | |
| | Protein | spacer sequence on the non-target, genomic DNA strand | | | | |
| pWISE | SEQ ID NO: | C.17 | C.19 | C.27 | C.28 | C.30 |
| pWISE121 | 56 | No data | No data | No data | No data | No data |
| pWISE9120 | 453 | No data | No data | No data | No data | No data |
| pWISE10648 | 629 | No data | No data | No data | No data | No data |
| pWISE10649 | 630 | 0.078% | 0.110% | 0.025% | 0.019% | 0.025% |
| pWISE10650 | 631 | 0.139% | 0.136% | 0.021% | 0.012% | 0.021% |
| pWISE10651 | 632 | 0.233% | 0.409% | 0.015% | 0.011% | 0.013% |
| pWISE10652 | 633 | 0.290% | 0.355% | 0.023% | 0.029% | 0.005% |
| pWISE10658 | 639 | 0.048% | 0.074% | 0.000% | 0.029% | 0.016% |
| pWISE10653 | 634 | 0.054% | 0.035% | 0.008% | 0.001% | 0.006% |
| pWISE10654 | 635 | No data | No data | No data | No data | No data |
| pWISE10655 | 636 | 0.166% | 0.096% | 0.025% | 0.059% | 0.000% |
| pWISE10656 | 637 | 0.040% | 0.062% | 0.010% | 0.001% | 0.004% |
| pWISE10657 | 638 | 0.047% | 0.015% | 0.011% | 0.012% | 0.000% |

TABLE 75

| | | Average G to A editing percent at the nucleotides | | | | |
|---|---|---|---|---|---|---|
| | | numbered according to the position in the PWsp390 spacer | | | | |
| | Protein | sequence on the opposite, target genomic DNA strand | | | | |
| pWISE | SEQ ID NO: | G.-9 | G.-8 | G.4 | G.21 | G.23 |
| pWISE121 | 56 | 0.008% | 0.000% | 0.008% | 0.635% | 1.015% |
| pWISE9120 | 453 | 0.045% | 0.039% | 0.232% | 1.019% | 0.806% |
| pWISE10648 | 629 | 0.014% | 0.014% | 0.160% | 0.717% | 0.654% |
| pWISE10649 | 630 | 0.035% | 0.018% | 0.070% | 0.707% | 0.667% |
| pWISE10650 | 631 | 0.004% | 0.007% | 0.118% | 1.208% | 1.080% |
| pWISE10651 | 632 | 0.027% | 0.003% | 0.088% | 1.378% | 1.425% |
| pWISE10652 | 633 | 0.009% | 0.014% | 0.054% | 1.281% | 1.151% |
| pWISE10658 | 639 | 0.070% | 0.061% | 0.030% | 0.576% | 0.891% |
| pWISE10653 | 634 | 0.112% | 0.087% | 0.069% | 0.639% | 0.554% |
| pWISE10654 | 635 | No data | No data | No data | No data | No data |
| pWISE10655 | 636 | 0.033% | 0.047% | 0.124% | 1.279% | 1.343% |
| pWISE10656 | 637 | 0.013% | 0.007% | 0.124% | 1.081% | 1.160% |
| pWISE10657 | 638 | 0.021% | 0.013% | 0.093% | 0.932% | 1.152% |

| | | Average G to A editing percent at the nucleotides | | | |
|---|---|---|---|---|---|
| | | numbered according to the position in the PWsp390 spacer | | | |
| | Protein | sequence on the opposite, target genomic DNA strand | | | |
| pWISE | SEQ ID NO: | G.24 | G.25 | G.26 | G.33 |
| pWISE121 | 56 | 0.206% | 0.294% | 0.087% | 0.103% |
| pWISE9120 | 453 | 0.535% | 0.316% | 0.206% | 0.064% |
| pWISE10648 | 629 | 0.480% | 0.320% | 0.244% | 0.104% |
| pWISE10649 | 630 | 0.431% | 0.256% | 0.167% | 0.227% |
| pWISE10650 | 631 | 0.790% | 0.447% | 0.195% | 0.232% |
| pWISE10651 | 632 | 0.901% | 0.623% | 0.453% | 0.268% |
| pWISE10652 | 633 | 0.723% | 0.450% | 0.346% | 0.313% |
| pWISE10658 | 639 | 0.615% | 0.271% | 0.165% | 0.168% |
| pWISE10653 | 634 | 0.333% | 0.218% | 0.112% | 0.093% |
| pWISE10654 | 635 | No data | No data | No data | No data |
| pWISE10655 | 636 | 0.901% | 0.348% | 0.213% | 0.212% |
| pWISE10656 | 637 | 0.773% | 0.360% | 0.237% | 0.260% |
| pWISE10657 | 638 | 0.751% | 0.354% | 0.221% | 0.152% |

TABLE 76

| | | Standard deviation of the average G to A editing percent at the | | | | |
|---|---|---|---|---|---|---|
| | | nucleotides numbered according to the position in the PWsp390 | | | | |
| | Protein | spacer sequence on the opposite, target genomic DNA strand | | | | |
| pWISE | SEQ ID NO: | G.-9 | G.-8 | G.4 | G.21 | G.23 |
| pWISE121 | 56 | No data | No data | No data | No data | No data |
| pWISE9120 | 453 | No data | No data | No data | No data | No data |
| pWISE10648 | 629 | No data | No data | No data | No data | No data |
| pWISE10649 | 630 | 0.002% | 0.025% | 0.003% | 0.079% | 0.045% |
| pWISE10650 | 631 | 0.005% | 0.010% | 0.031% | 0.215% | 0.113% |
| pWISE10651 | 632 | 0.021% | 0.004% | 0.006% | 0.118% | 0.042% |
| pWISE10652 | 633 | 0.013% | 0.019% | 0.013% | 0.054% | 0.289% |
| pWISE10658 | 639 | 0.020% | 0.066% | 0.012% | 0.076% | 0.020% |
| pWISE10653 | 634 | 0.007% | 0.016% | 0.029% | 0.028% | 0.161% |
| pWISE10654 | 635 | No data | No data | No data | No data | No data |
| pWISE10655 | 636 | 0.013% | 0.023% | 0.056% | 0.312% | 0.093% |
| pWISE10656 | 637 | 0.009% | 0.010% | 0.103% | 0.328% | 0.261% |
| pWISE10657 | 638 | 0.013% | 0.008% | 0.010% | 0.059% | 0.164% |

| | | Standard deviation of the average G to A editing percent at the | | | |
|---|---|---|---|---|---|
| | | nucleotides numbered according to the position in the PWsp390 | | | |
| | Protein | spacer sequence on the opposite, target genomic DNA strand | | | |
| pWISE | SEQ ID NO: | G.24 | G.25 | G.26 | G.33 |
| pWISE121 | 56 | No data | No data | No data | No data |
| pWISE9120 | 453 | No data | No data | No data | No data |
| pWISE10648 | 629 | No data | No data | No data | No data |
| pWISE10649 | 630 | 0.022% | 0.015% | 0.080% | 0.046% |
| pWISE10650 | 631 | 0.138% | 0.058% | 0.048% | 0.087% |
| pWISE10651 | 632 | 0.193% | 0.132% | 0.093% | 0.093% |
| pWISE10652 | 633 | 0.038% | 0.040% | 0.008% | 0.056% |
| pWISE10658 | 639 | 0.042% | 0.029% | 0.027% | 0.001% |
| pWISE10653 | 634 | 0.121% | 0.007% | 0.007% | 0.044% |
| pWISE10654 | 635 | No data | No data | No data | No data |
| pWISE10655 | 636 | 0.173% | 0.063% | 0.036% | 0.057% |
| pWISE10656 | 637 | 0.071% | 0.027% | 0.028% | 0.040% |
| pWISE10657 | 638 | 0.117% | 0.045% | 0.008% | 0.024% |

TABLE 77

| | | Average C to T editing percent at the nucleotides | | | | | | |
|---|---|---|---|---|---|---|---|---|
| | | numbered according to the position in the PWsp133 | | | | | | |
| | Protein | spacer sequence on the non-target, genomic DNA strand | | | | | | |
| pWISE | SEQ ID NO: | C.-5 | C.1 | C.11 | C.17 | C.20 | C.24 | C.27 |
| pWISE121 | 56 | 0.019% | 0.124% | 0.171% | 0.797% | 0.316% | 0.268% | 0.051% |
| pWISE9120 | 453 | 0.030% | 0.166% | 0.814% | 0.125% | 2.567% | 0.070% | 0.015% |
| pWISE10648 | 629 | 0.009% | 0.133% | 0.148% | 0.079% | 1.611% | 0.068% | 0.015% |
| pWISE10649 | 630 | 0.021% | 0.174% | 0.113% | 0.066% | 2.203% | 0.196% | 0.030% |
| pWISE10650 | 631 | 0.021% | 0.224% | 0.190% | 0.130% | 4.514% | 0.618% | 0.060% |
| pWISE10651 | 632 | 0.024% | 0.243% | 0.168% | 0.109% | 4.555% | 0.734% | 0.088% |
| pWISE10652 | 633 | 0.034% | 0.177% | 0.167% | 0.087% | 3.410% | 0.687% | 0.065% |
| pWISE10658 | 639 | 0.018% | 0.236% | 0.276% | 0.062% | 4.315% | 0.102% | 0.030% |
| pWISE10653 | 634 | 0.008% | 0.125% | 0.125% | 0.047% | 2.812% | 0.132% | 0.008% |
| pWISE10654 | 635 | 0.017% | 0.253% | 0.128% | 0.065% | 5.429% | 0.450% | 0.067% |
| pWISE10655 | 636 | 0.017% | 0.245% | 0.149% | 0.077% | 6.715% | 0.691% | 0.102% |
| pWISE10656 | 637 | 0.007% | 0.222% | 0.129% | 0.037% | 6.144% | 0.752% | 0.088% |
| pWISE10657 | 638 | 0.009% | 0.156% | 0.117% | 0.030% | 4.792% | 0.764% | 0.077% |

TABLE 78

| | | Standard Deviation of the average editing percent at the | | | | | | |
|---|---|---|---|---|---|---|---|---|
| | | nucleotides numbered according to the position in the PWsp133 | | | | | | |
| | Protein | spacer sequence on the non-target, genomic DNA strand | | | | | | |
| pWISE | SEQ ID NO: | C.-5 | C.1 | C.11 | C.17 | C.20 | C.24 | C.27 |
| pWISE121 | 56 | 0.022% | 0.043% | 0.049% | 0.184% | 0.080% | 0.076% | 0.028% |
| pWISE9120 | 453 | 0.020% | 0.063% | 0.220% | 0.025% | 0.379% | 0.029% | 0.008% |
| pWISE10648 | 629 | 0.010% | 0.049% | 0.029% | 0.038% | 0.168% | 0.032% | 0.020% |
| pWISE10649 | 630 | 0.017% | 0.056% | 0.029% | 0.010% | 0.306% | 0.087% | 0.019% |
| pWISE10650 | 631 | 0.019% | 0.078% | 0.077% | 0.031% | 0.565% | 0.135% | 0.025% |
| pWISE10651 | 632 | 0.030% | 0.063% | 0.034% | 0.011% | 0.740% | 0.152% | 0.056% |
| pWISE10652 | 633 | 0.023% | 0.037% | 0.051% | 0.050% | 0.541% | 0.200% | 0.030% |
| pWISE10658 | 639 | 0.011% | 0.064% | 0.025% | 0.020% | 0.496% | 0.023% | 0.019% |
| pWISE10653 | 634 | 0.010% | 0.035% | 0.046% | 0.026% | 0.533% | 0.038% | 0.013% |
| pWISE10654 | 635 | 0.012% | 0.050% | 0.056% | 0.025% | 0.567% | 0.055% | 0.025% |
| pWISE10655 | 636 | 0.013% | 0.073% | 0.035% | 0.055% | 1.195% | 0.137% | 0.036% |
| pWISE10656 | 637 | 0.007% | 0.025% | 0.035% | 0.018% | 0.818% | 0.209% | 0.025% |
| pWISE10657 | 638 | 0.008% | 0.055% | 0.045% | 0.020% | 0.939% | 0.249% | 0.044% |

TABLE 79

| | | Average G to A editing percent at the nucleotides | | | | | | |
|---|---|---|---|---|---|---|---|---|
| | | numbered according to the position in the PWsp133 spacer | | | | | | |
| | Protein | sequence on the opposite, target genomic DNA strand | | | | | | |
| pWISE | SEQ ID NO: | G.-9 | G.-8 | G.-6 | G.3 | G.4 | G.6 | G.7 |
| pWISE121 | 56 | 0.006% | 0.020% | 0.022% | 0.128% | 0.127% | 0.173% | 0.131% |
| pWISE9120 | 453 | 0.004% | 0.004% | 0.007% | 0.033% | 0.044% | 0.013% | 0.045% |
| pWISE10648 | 629 | 0.003% | 0.005% | 0.002% | 0.030% | 0.044% | 0.013% | 0.044% |
| pWISE10649 | 630 | 0.003% | 0.005% | 0.004% | 0.034% | 0.057% | 0.019% | 0.065% |
| pWISE10650 | 631 | 0.001% | 0.004% | 0.015% | 0.016% | 0.064% | 0.005% | 0.055% |
| pWISE10651 | 632 | 0.004% | 0.006% | 0.018% | 0.037% | 0.064% | 0.016% | 0.068% |
| pWISE10652 | 633 | 0.002% | 0.005% | 0.003% | 0.013% | 0.026% | 0.013% | 0.049% |
| pWISE10658 | 639 | 0.008% | 0.001% | 0.008% | 0.013% | 0.033% | 0.011% | 0.040% |
| pWISE10653 | 634 | 0.002% | 0.004% | 0.014% | 0.011% | 0.042% | 0.009% | 0.061% |
| pWISE10654 | 635 | 0.006% | 0.010% | 0.009% | 0.036% | 0.068% | 0.005% | 0.039% |
| pWISE10655 | 636 | 0.004% | 0.002% | 0.007% | 0.016% | 0.031% | 0.009% | 0.043% |
| pWISE10656 | 637 | 0.003% | 0.006% | 0.010% | 0.018% | 0.032% | 0.004% | 0.034% |
| pWISE10657 | 638 | 0.007% | 0.004% | 0.007% | 0.015% | 0.020% | 0.005% | 0.031% |

| | | Average G to A editing percent at the nucleotides | | | | | |
|---|---|---|---|---|---|---|---|
| | | numbered according to the position in the PWsp133 spacer | | | | | |
| | Protein | sequence on the opposite, target genomic DNA strand | | | | | |
| pWISE | SEQ ID NO: | G.10 | G.12 | G.15 | G.16 | G.22 | G.26 |
| pWISE121 | 56 | 0.171% | 0.263% | 0.737% | 1.561% | 0.321% | 0.065% |
| pWISE9120 | 453 | 3.694% | 4.314% | 2.470% | 2.091% | 2.252% | 0.280% |
| pWISE10648 | 629 | 1.359% | 2.899% | 2.006% | 1.700% | 1.659% | 0.246% |
| pWISE10649 | 630 | 1.344% | 3.271% | 2.224% | 1.861% | 2.403% | 0.298% |
| pWISE10650 | 631 | 1.453% | 3.751% | 2.751% | 2.324% | 3.657% | 0.492% |
| pWISE10651 | 632 | 1.089% | 2.386% | 1.794% | 1.512% | 3.057% | 0.473% |
| pWISE10652 | 633 | 0.626% | 1.462% | 1.169% | 0.980% | 2.523% | 0.486% |
| pWISE10658 | 639 | 6.851% | 7.752% | 5.354% | 4.701% | 4.565% | 0.483% |
| pWISE10653 | 634 | 3.298% | 6.114% | 4.332% | 3.691% | 3.513% | 0.252% |
| pWISE10654 | 635 | 4.019% | 8.284% | 5.971% | 5.023% | 5.746% | 0.644% |
| pWISE10655 | 636 | 3.151% | 6.759% | 4.916% | 4.235% | 5.713% | 0.610% |
| pWISE10656 | 637 | 2.221% | 4.167% | 3.230% | 2.818% | 4.490% | 0.448% |
| pWISE10657 | 638 | 1.446% | 2.598% | 2.309% | 2.002% | 3.808% | 0.437% |

TABLE 80

| | | Standard deviation of the average G to A editing percent at the | | | | | | |
|---|---|---|---|---|---|---|---|---|
| | | nucleotides numbered according to the position in the PWsp133 | | | | | | |
| | Protein SEQ | spacer sequence on the opposite, target genomic DNA strand | | | | | | |
| pWISE | ID NO: | G.-9 | G.-8 | G.-6 | G.3 | G.4 | G.6 | G.7 |
| pWISE121 | 56 | 0.004% | 0.016% | 0.011% | 0.023% | 0.041% | 0.044% | 0.044% |
| pWISE9120 | 453 | 0.005% | 0.003% | 0.006% | 0.023% | 0.035% | 0.009% | 0.022% |
| pWISE10648 | 629 | 0.005% | 0.005% | 0.001% | 0.025% | 0.025% | 0.013% | 0.032% |
| pWISE10649 | 630 | 0.003% | 0.005% | 0.003% | 0.048% | 0.040% | 0.016% | 0.030% |
| pWISE10650 | 631 | 0.002% | 0.002% | 0.011% | 0.009% | 0.039% | 0.005% | 0.026% |
| pWISE10651 | 632 | 0.004% | 0.004% | 0.016% | 0.025% | 0.027% | 0.022% | 0.028% |
| pWISE10652 | 633 | 0.002% | 0.006% | 0.005% | 0.015% | 0.014% | 0.015% | 0.027% |
| pWISE10658 | 639 | 0.007% | 0.002% | 0.004% | 0.014% | 0.017% | 0.009% | 0.030% |
| pWISE10653 | 634 | 0.003% | 0.002% | 0.016% | 0.009% | 0.030% | 0.005% | 0.014% |
| pWISE10654 | 635 | 0.014% | 0.013% | 0.009% | 0.032% | 0.054% | 0.007% | 0.035% |
| pWISE10655 | 636 | 0.002% | 0.004% | 0.007% | 0.013% | 0.027% | 0.007% | 0.026% |
| pWISE10656 | 637 | 0.002% | 0.005% | 0.008% | 0.017% | 0.011% | 0.004% | 0.031% |
| pWISE10657 | 638 | 0.008% | 0.004% | 0.006% | 0.014% | 0.022% | 0.003% | 0.021% |

| | |
|---|---|
| | Standard deviation of the average G to A editing percent at the |
| | nucleotides numbered according to the position in the PWsp133 |
| Protein SEQ | spacer sequence on the opposite, target genomic DNA strand |

| pWISE | ID NO: | G.10 | G.12 | G.15 | G.16 | G.22 | G.26 |
|---|---|---|---|---|---|---|---|
| pWISE121 | 56 | 0.043% | 0.062% | 0.111% | 0.146% | 0.068% | 0.028% |
| pWISE9120 | 453 | 0.396% | 0.421% | 0.142% | 0.103% | 0.360% | 0.074% |
| pWISE10648 | 629 | 0.202% | 0.235% | 0.141% | 0.136% | 0.178% | 0.119% |
| pWISE10649 | 630 | 0.204% | 0.327% | 0.315% | 0.393% | 0.386% | 0.085% |
| pWISE10650 | 631 | 0.317% | 0.582% | 0.423% | 0.350% | 0.561% | 0.071% |
| pWISE10651 | 632 | 0.144% | 0.261% | 0.249% | 0.250% | 0.394% | 0.100% |
| pWISE10652 | 633 | 0.096% | 0.149% | 0.162% | 0.124% | 0.394% | 0.204% |
| pWISE10658 | 639 | 1.011% | 1.134% | 0.645% | 0.606% | 0.543% | 0.120% |
| pWISE10653 | 634 | 0.342% | 0.648% | 0.341% | 0.375% | 0.507% | 0.080% |
| pWISE10654 | 635 | 0.468% | 1.037% | 0.684% | 0.570% | 0.753% | 0.192% |
| pWISE10655 | 636 | 0.426% | 0.900% | 0.696% | 0.657% | 1.047% | 0.148% |
| pWISE10656 | 637 | 0.253% | 0.662% | 0.464% | 0.381% | 0.534% | 0.098% |
| pWISE10657 | 638 | 0.118% | 0.344% | 0.416% | 0.346% | 0.705% | 0.134% |

TABLE 81

| | | Average C to T editing percent at the nucleotides numbered | | | | | |
|---|---|---|---|---|---|---|---|
| | | according to the position in the PWsp3627 spacer sequence on the | | | | | |
| | Protein SEQ | non-target, genomic DNA strand | | | | | |
| pWISE | ID NO: | C.-7 | C.-6 | C.-4 | C.-1 | C.13 | C.18 |
| pWISE121 | 56 | 0.026% | 0.032% | 0.013% | 0.037% | 0.392% | 2.274% |
| pWISE9120 | 453 | 0.007% | 0.002% | 0.000% | 0.290% | 3.725% | 1.194% |
| pWISE10648 | 629 | 0.003% | 0.007% | 0.004% | 0.124% | 0.888% | 0.312% |
| pWISE10649 | 630 | 0.003% | 0.003% | 0.002% | 0.095% | 0.592% | 0.450% |
| pWISE10650 | 631 | 0.006% | 0.004% | 0.005% | 0.163% | 0.828% | 0.639% |
| pWISE10651 | 632 | 0.003% | 0.007% | 0.010% | 0.152% | 1.096% | 0.939% |
| pWISE10652 | 633 | 0.001% | 0.003% | 0.003% | 0.281% | 0.966% | 0.540% |
| pWISE10658 | 639 | 0.003% | 0.006% | 0.003% | 0.258% | 1.580% | 0.542% |
| pWISE10653 | 634 | 0.003% | 0.012% | 0.003% | 0.190% | 1.137% | 0.712% |
| pWISE10654 | 635 | 0.004% | 0.005% | 0.005% | 0.181% | 0.927% | 1.052% |
| pWISE10655 | 636 | 0.002% | 0.007% | 0.004% | 0.286% | 1.137% | 1.809% |
| pWISE10656 | 637 | 0.007% | 0.008% | 0.001% | 0.250% | 1.092% | 1.698% |
| pWISE10657 | 638 | 0.002% | 0.004% | 0.000% | 0.244% | 1.298% | 1.160% |

| | | Average C to T editing percent at the nucleotides | | | | |
|---|---|---|---|---|---|---|
| | | numbered according to the position in the PWsp3627 | | | | |
| | Protein SEQ | spacer sequence on the non-target, genomic DNA strand | | | | |
| pWISE | ID NO: | C.19 | C.20 | C.24 | C.26 | C.30 |
| pWISE121 | 56 | 1.031% | 0.457% | 0.374% | 0.460% | 0.156% |
| pWISE9120 | 453 | 1.581% | 2.294% | 0.840% | 0.103% | 0.024% |
| pWISE10648 | 629 | 0.607% | 0.858% | 0.583% | 0.080% | 0.009% |
| pWISE10649 | 630 | 0.915% | 1.540% | 1.144% | 0.350% | 0.153% |
| pWISE10650 | 631 | 1.349% | 2.144% | 1.592% | 0.392% | 0.072% |
| pWISE10651 | 632 | 1.895% | 3.064% | 2.514% | 0.699% | 0.107% |
| pWISE10652 | 633 | 1.234% | 2.433% | 1.942% | 0.608% | 0.073% |
| pWISE10658 | 639 | 1.201% | 1.704% | 0.904% | 0.094% | 0.038% |
| pWISE10653 | 634 | 1.280% | 1.727% | 0.856% | 0.167% | 0.038% |
| pWISE10654 | 635 | 1.819% | 2.501% | 1.521% | 0.354% | 0.076% |
| pWISE10655 | 636 | 2.850% | 3.592% | 2.392% | 0.616% | 0.122% |
| pWISE10656 | 637 | 2.612% | 3.246% | 2.164% | 0.680% | 0.152% |
| pWISE10657 | 638 | 1.827% | 3.074% | 1.804% | 0.503% | 0.119% |

TABLE 82

| | | Standard Deviation of the average editing percent at the | | | | | |
|---|---|---|---|---|---|---|---|
| | | nucleotides numbered according to the position in the PWsp3627 | | | | | |
| | Protein SEQ | spacer sequence on the non-target, genomic DNA strand | | | | | |
| pWISE | ID NO: | C.-7 | C.-6 | C.-4 | C.-1 | C.13 | C.18 |
| pWISE121 | 56 | 0.029% | 0.031% | 0.016% | 0.029% | 0.120% | 0.782% |
| pWISE9120 | 453 | 0.005% | 0.002% | 0.001% | 0.158% | 1.204% | 0.422% |
| pWISE10648 | 629 | 0.002% | 0.007% | 0.007% | 0.055% | 0.446% | 0.194% |
| pWISE10649 | 630 | 0.002% | 0.003% | 0.004% | 0.065% | 0.254% | 0.276% |
| pWISE10650 | 631 | 0.005% | 0.005% | 0.004% | 0.077% | 0.335% | 0.355% |
| pWISE10651 | 632 | 0.003% | 0.007% | 0.013% | 0.106% | 0.301% | 0.430% |
| pWISE10652 | 633 | 0.002% | 0.002% | 0.002% | 0.099% | 0.584% | 0.295% |
| pWISE10658 | 639 | 0.004% | 0.003% | 0.005% | 0.132% | 0.513% | 0.241% |
| pWISE10653 | 634 | 0.004% | 0.014% | 0.004% | 0.110% | 0.362% | 0.293% |
| pWISE10654 | 635 | 0.004% | 0.003% | 0.006% | 0.086% | 0.414% | 0.666% |
| pWISE10655 | 636 | 0.002% | 0.005% | 0.008% | 0.175% | 0.393% | 0.721% |
| pWISE10656 | 637 | 0.010% | 0.012% | 0.003% | 0.106% | 0.365% | 0.987% |
| pWISE10657 | 638 | 0.002% | 0.006% | 0.001% | 0.125% | 0.510% | 0.674% |

| | | Standard Deviation of the average editing percent at the | | | | |
|---|---|---|---|---|---|---|
| | | nucleotides numbered according to the position in the PWsp3627 | | | | |
| | Protein SEQ | spacer sequence on the non-target, genomic DNA strand | | | | |
| pWISE | ID NO: | C.19 | C.20 | C.24 | C.26 | C.30 |
| pWISE121 | 56 | 0.374% | 0.189% | 0.139% | 0.170% | 0.097% |
| pWISE9120 | 453 | 0.562% | 0.874% | 0.407% | 0.063% | 0.021% |
| pWISE10648 | 629 | 0.356% | 0.485% | 0.362% | 0.056% | 0.009% |
| pWISE10649 | 630 | 0.591% | 0.777% | 0.513% | 0.178% | 0.219% |
| pWISE10650 | 631 | 0.778% | 1.206% | 0.880% | 0.223% | 0.045% |
| pWISE10651 | 632 | 0.842% | 1.280% | 1.181% | 0.371% | 0.083% |
| pWISE10652 | 633 | 0.798% | 1.418% | 1.200% | 0.470% | 0.050% |
| pWISE10658 | 639 | 0.540% | 0.827% | 0.456% | 0.062% | 0.018% |
| pWISE10653 | 634 | 0.440% | 0.623% | 0.305% | 0.069% | 0.030% |
| pWISE10654 | 635 | 1.082% | 1.501% | 0.910% | 0.214% | 0.084% |
| pWISE10655 | 636 | 1.232% | 1.597% | 1.170% | 0.275% | 0.060% |
| pWISE10656 | 637 | 1.415% | 1.700% | 1.149% | 0.378% | 0.094% |
| pWISE10657 | 638 | 1.065% | 1.823% | 1.029% | 0.295% | 0.083% |

TABLE 83

| | | Average G to A editing percent at the nucleotides numbered | | | | | | |
|---|---|---|---|---|---|---|---|---|
| | | according to the position in the PWsp3627 spacer sequence on the | | | | | | |
| | Protein SEQ | opposite, target genomic DNA strand | | | | | | |
| pWISE | ID NO: | G.-9 | G.-8 | G.2 | G.3 | G.5 | G.6 | G.8 |
| pWISE121 | 56 | 0.018% | 0.036% | 0.043% | 0.070% | 0.062% | 0.078% | 0.183% |
| pWISE9120 | 453 | 0.030% | 0.020% | 0.016% | 0.020% | 0.021% | 0.043% | 0.325% |
| pWISE10648 | 629 | 0.035% | 0.023% | 0.023% | 0.075% | 0.032% | 0.081% | 0.302% |
| pWISE10649 | 630 | 0.026% | 0.010% | 0.020% | 0.029% | 0.017% | 0.028% | 0.414% |
| pWISE10650 | 631 | 0.033% | 0.008% | 0.043% | 0.070% | 0.049% | 0.078% | 0.888% |
| pWISE10651 | 632 | 0.023% | 0.009% | 0.045% | 0.055% | 0.040% | 0.129% | 0.846% |
| pWISE10652 | 633 | 0.022% | 0.005% | 0.059% | 0.063% | 0.068% | 0.102% | 0.527% |
| pWISE10658 | 639 | 0.059% | 0.025% | 0.044% | 0.036% | 0.018% | 0.083% | 0.591% |
| pWISE10653 | 634 | 0.029% | 0.025% | 0.017% | 0.032% | 0.021% | 0.069% | 0.620% |
| pWISE10654 | 635 | 0.038% | 0.003% | 0.057% | 0.078% | 0.047% | 0.072% | 1.219% |
| pWISE10655 | 636 | 0.045% | 0.009% | 0.030% | 0.049% | 0.038% | 0.099% | 1.977% |
| pWISE10656 | 637 | 0.021% | 0.006% | 0.042% | 0.050% | 0.034% | 0.079% | 1.496% |
| pWISE10657 | 638 | 0.034% | 0.025% | 0.023% | 0.026% | 0.031% | 0.064% | 1.147% |

| | | Average G to A editing percent at the nucleotides numbered | | | | | |
|---|---|---|---|---|---|---|---|
| | | according to the position in the PWsp3627 spacer sequence on the | | | | | |
| | Protein SEQ | opposite, target genomic DNA strand | | | | | |
| pWISE | ID NO: | G.9 | G.14 | G.15 | G.28 | G.32 | G.33 |
| pWISE121 | 56 | 0.112% | 0.431% | 1.056% | 0.287% | 0.193% | 0.061% |
| pWISE9120 | 453 | 2.809% | 9.481% | 9.716% | 5.186% | 0.450% | 0.275% |
| pWISE10648 | 629 | 1.317% | 6.833% | 6.872% | 3.786% | 0.368% | 0.211% |
| pWISE10649 | 630 | 1.126% | 8.696% | 8.858% | 5.633% | 0.574% | 0.343% |
| pWISE10650 | 631 | 1.590% | 11.884% | 12.370% | 8.012% | 0.864% | 0.477% |
| pWISE10651 | 632 | 1.768% | 14.517% | 15.365% | 10.011% | 1.160% | 0.816% |
| pWISE10652 | 633 | 1.114% | 9.031% | 9.961% | 7.787% | 0.791% | 0.399% |
| pWISE10658 | 639 | 1.944% | 15.885% | 16.085% | 7.953% | 0.892% | 0.538% |
| pWISE10653 | 634 | 1.495% | 14.954% | 15.111% | 7.053% | 0.522% | 0.301% |
| pWISE10654 | 635 | 2.040% | 16.807% | 17.108% | 9.331% | 1.005% | 0.527% |
| pWISE10655 | 636 | 2.917% | 23.470% | 24.197% | 13.347% | 1.191% | 0.744% |
| pWISE10656 | 637 | 2.195% | 18.804% | 19.936% | 10.528% | 0.990% | 0.554% |
| pWISE10657 | 638 | 1.850% | 15.529% | 17.076% | 10.371% | 0.970% | 0.513% |

TABLE 84

| | | Standard deviation of the average G to A editing percent at the | | | | | | |
|---|---|---|---|---|---|---|---|---|
| | | nucleotides numbered according to the position in the PWsp3627 | | | | | | |
| | Protein SEQ | spacer sequence on the opposite, target genomic DNA strand | | | | | | |
| pWISE | ID NO: | G.-9 | G.-8 | G.2 | G.3 | G.5 | G.6 | G.8 |
| pWISE121 | 56 | 0.021% | 0.030% | 0.027% | 0.048% | 0.041% | 0.038% | 0.101% |
| pWISE9120 | 453 | 0.018% | 0.010% | 0.017% | 0.019% | 0.009% | 0.035% | 0.121% |
| pWISE10648 | 629 | 0.015% | 0.015% | 0.023% | 0.038% | 0.024% | 0.050% | 0.157% |
| pWISE10649 | 630 | 0.019% | 0.006% | 0.022% | 0.023% | 0.013% | 0.024% | 0.324% |
| pWISE10650 | 631 | 0.022% | 0.003% | 0.046% | 0.070% | 0.041% | 0.051% | 0.596% |
| pWISE10651 | 632 | 0.013% | 0.010% | 0.048% | 0.045% | 0.052% | 0.122% | 0.289% |
| pWISE10652 | 633 | 0.011% | 0.004% | 0.071% | 0.072% | 0.073% | 0.068% | 0.317% |
| pWISE10658 | 639 | 0.067% | 0.022% | 0.062% | 0.021% | 0.017% | 0.037% | 0.227% |
| pWISE10653 | 634 | 0.020% | 0.017% | 0.007% | 0.021% | 0.021% | 0.033% | 0.221% |
| pWISE10654 | 635 | 0.033% | 0.003% | 0.041% | 0.057% | 0.048% | 0.042% | 0.608% |
| pWISE10655 | 636 | 0.026% | 0.010% | 0.022% | 0.029% | 0.026% | 0.038% | 0.924% |
| pWISE10656 | 637 | 0.024% | 0.005% | 0.030% | 0.030% | 0.028% | 0.036% | 0.674% |
| pWISE10657 | 638 | 0.028% | 0.040% | 0.016% | 0.019% | 0.027% | 0.030% | 0.659% |

| | |
|---|---|
| | Standard deviation of the average G to A editing percent at the |
| | nucleotides numbered according to the position in the PWsp3627 |
| Protein SEQ | spacer sequence on the opposite, target genomic DNA strand |

| pWISE | ID NO: | G.9 | G.14 | G.15 | G.28 | G.32 | G.33 |
|---|---|---|---|---|---|---|---|
| pWISE121 | 56 | 0.028% | 0.162% | 0.390% | 0.159% | 0.051% | 0.047% |
| pWISE9120 | 453 | 1.161% | 4.267% | 4.398% | 2.476% | 0.248% | 0.180% |
| pWISE10648 | 629 | 0.765% | 3.843% | 3.847% | 2.320% | 0.338% | 0.180% |
| pWISE10649 | 630 | 0.802% | 4.336% | 4.682% | 2.972% | 0.470% | 0.280% |
| pWISE10650 | 631 | 0.930% | 6.925% | 7.214% | 4.923% | 0.670% | 0.386% |
| pWISE10651 | 632 | 0.693% | 6.888% | 7.233% | 4.865% | 0.834% | 0.902% |
| pWISE10652 | 633 | 0.625% | 5.069% | 5.715% | 4.656% | 0.587% | 0.331% |
| pWISE10658 | 639 | 0.836% | 8.405% | 8.517% | 4.361% | 0.665% | 0.418% |
| pWISE10653 | 634 | 0.580% | 6.482% | 6.546% | 3.282% | 0.309% | 0.214% |
| pWISE10654 | 635 | 1.047% | 10.674% | 10.877% | 6.122% | 0.845% | 0.459% |
| pWISE10655 | 636 | 1.199% | 10.541% | 10.733% | 6.567% | 0.820% | 0.483% |
| pWISE10656 | 637 | 0.968% | 9.861% | 10.487% | 6.010% | 0.703% | 0.396% |
| pWISE10657 | 638 | 1.035% | 8.674% | 9.631% | 6.156% | 0.724% | 0.386% |

TABLE 85

| | | Average C to T editing percent at the nucleotides numbered | | | | | |
|---|---|---|---|---|---|---|---|
| | | according to the position in the PWsp3628 spacer sequence on the | | | | | |
| | Protein SEQ | non-target, genomic DNA strand | | | | | |
| pWISE | ID NO: | C.-9 | C.-7 | C.-1 | C.1 | C.2 | C.3 |
| pWISE121 | 56 | 0.016% | 0.024% | 0.025% | 0.014% | 0.023% | 0.105% |
| pWISE9120 | 453 | 0.014% | 0.027% | 0.031% | 0.015% | 0.016% | 0.038% |
| pWISE10648 | 629 | 0.004% | 0.008% | 0.006% | 0.003% | 0.007% | 0.064% |
| pWISE10649 | 630 | 0.008% | 0.028% | 0.012% | 0.011% | 0.010% | 0.096% |
| pWISE10650 | 631 | 0.007% | 0.050% | 0.009% | 0.006% | 0.005% | 0.053% |
| pWISE10651 | 632 | 0.007% | 0.033% | 0.050% | 0.029% | 0.019% | 0.076% |
| pWISE10652 | 633 | 0.006% | 0.026% | 0.039% | 0.004% | 0.006% | 0.060% |
| pWISE10658 | 639 | 0.012% | 0.018% | 0.008% | 0.004% | 0.001% | 0.093% |
| pWISE10653 | 634 | 0.003% | 0.013% | 0.014% | 0.012% | 0.009% | 0.060% |
| pWISE10654 | 635 | 0.002% | 0.017% | 0.022% | 0.004% | 0.005% | 0.015% |
| pWISE10655 | 636 | 0.012% | 0.033% | 0.025% | 0.008% | 0.005% | 0.060% |
| pWISE10656 | 637 | 0.002% | 0.025% | 0.021% | 0.007% | 0.004% | 0.062% |
| pWISE10657 | 638 | 0.006% | 0.020% | 0.010% | 0.007% | 0.002% | 0.060% |

| | | Average C to T editing percent at the nucleotides numbered | | | | |
|---|---|---|---|---|---|---|
| | | according to the position in the PWsp3628 spacer sequence on | | | | |
| | Protein SEQ | the non-target, genomic DNA strand | | | | |
| pWISE | ID NO: | C.8 | C.13 | C.17 | C.31 | C.32 |
| pWISE121 | 56 | 0.068% | 0.146% | 1.093% | 0.147% | 0.104% |
| pWISE9120 | 453 | 1.396% | 1.449% | 0.407% | 0.004% | 0.023% |
| pWISE10648 | 629 | 0.039% | 0.081% | 0.038% | 0.001% | 0.023% |
| pWISE10649 | 630 | 0.020% | 0.058% | 0.019% | 0.013% | 0.032% |
| pWISE10650 | 631 | 0.027% | 0.057% | 0.015% | 0.006% | 0.010% |
| pWISE10651 | 632 | 0.042% | 0.089% | 0.048% | 0.006% | 0.021% |
| pWISE10652 | 633 | 0.018% | 0.031% | 0.008% | 0.003% | 0.033% |
| pWISE10658 | 639 | 0.069% | 0.138% | 0.051% | 0.005% | 0.036% |
| pWISE10653 | 634 | 0.068% | 0.089% | 0.087% | 0.003% | 0.023% |
| pWISE10654 | 635 | 0.056% | 0.066% | 0.090% | 0.005% | 0.034% |
| pWISE10655 | 636 | 0.018% | 0.070% | 0.030% | 0.003% | 0.054% |
| pWISE10656 | 637 | 0.045% | 0.053% | 0.021% | 0.004% | 0.030% |
| pWISE10657 | 638 | 0.017% | 0.060% | 0.005% | 0.002% | 0.065% |

TABLE 86

| | | Standard Deviation of the average editing percent at the | | | | | |
|---|---|---|---|---|---|---|---|
| | | nucleotides numbered according to the position in the PWsp3628 | | | | | |
| | Protein SEQ | spacer sequence on the non-target, genomic DNA strand | | | | | |
| pWISE | ID NO: | C.-9 | C.-7 | C.-1 | C.1 | C.2 | C.3 |
| pWISE121 | 56 | 0.016% | 0.021% | 0.030% | 0.013% | 0.018% | 0.072% |
| pWISE9120 | 453 | 0.020% | 0.020% | 0.018% | 0.016% | 0.018% | 0.031% |
| pWISE10648 | 629 | 0.004% | 0.007% | 0.006% | 0.003% | 0.005% | 0.113% |
| pWISE10649 | 630 | 0.014% | 0.021% | 0.013% | 0.016% | 0.016% | 0.128% |
| pWISE10650 | 631 | 0.007% | 0.056% | 0.009% | 0.005% | 0.004% | 0.090% |
| pWISE10651 | 632 | 0.008% | 0.036% | 0.044% | 0.025% | 0.016% | 0.093% |
| pWISE10652 | 633 | 0.005% | 0.030% | 0.049% | 0.004% | 0.004% | 0.077% |
| pWISE10658 | 639 | 0.012% | 0.009% | 0.008% | 0.003% | 0.001% | 0.107% |
| pWISE10653 | 634 | 0.003% | 0.011% | 0.018% | 0.008% | 0.009% | 0.097% |
| pWISE10654 | 635 | 0.002% | 0.018% | 0.024% | 0.004% | 0.004% | 0.014% |
| pWISE10655 | 636 | 0.017% | 0.032% | 0.026% | 0.008% | 0.004% | 0.072% |
| pWISE10656 | 637 | 0.002% | 0.037% | 0.013% | 0.005% | 0.003% | 0.063% |
| pWISE10657 | 638 | 0.006% | 0.018% | 0.014% | 0.011% | 0.002% | 0.078% |

| | | Standard Deviation of the average editing percent at the | | | | |
|---|---|---|---|---|---|---|
| | | nucleotides numbered according to the position in the PWsp3628 | | | | |
| | Protein SEQ | spacer sequence on the non-target, genomic DNA strand | | | | |
| pWISE | ID NO: | C.8 | C.13 | C.17 | C.31 | C.32 |
| pWISE121 | 56 | 0.076% | 0.131% | 0.740% | 0.112% | 0.090% |
| pWISE9120 | 453 | 0.423% | 0.458% | 0.139% | 0.004% | 0.020% |
| pWISE10648 | 629 | 0.027% | 0.042% | 0.028% | 0.001% | 0.025% |
| pWISE10649 | 630 | 0.010% | 0.055% | 0.020% | 0.027% | 0.024% |
| pWISE10650 | 631 | 0.025% | 0.029% | 0.008% | 0.006% | 0.012% |
| pWISE10651 | 632 | 0.033% | 0.053% | 0.036% | 0.008% | 0.017% |
| pWISE10652 | 633 | 0.020% | 0.030% | 0.006% | 0.004% | 0.053% |
| pWISE10658 | 639 | 0.033% | 0.118% | 0.029% | 0.005% | 0.028% |
| pWISE10653 | 634 | 0.036% | 0.062% | 0.035% | 0.003% | 0.018% |
| pWISE10654 | 635 | 0.036% | 0.047% | 0.064% | 0.005% | 0.033% |
| pWISE10655 | 636 | 0.009% | 0.035% | 0.038% | 0.003% | 0.043% |
| pWISE10656 | 637 | 0.021% | 0.025% | 0.024% | 0.005% | 0.033% |
| pWISE10657 | 638 | 0.015% | 0.044% | 0.005% | 0.003% | 0.085% |

TABLE 87

| | | Average G to A editing percent at the nucleotides numbered | | | | | |
|---|---|---|---|---|---|---|---|
| | | according to the position in the PWsp3628 spacer sequence on the | | | | | |
| | Protein SEQ | opposite, target genomic DNA strand | | | | | |
| pWISE | ID NO: | G.-6 | G.-5 | G.6 | G.7 | G.10 | G.12 |
| pWISE121 | 56 | 0.008% | 0.011% | 0.083% | 0.042% | 0.118% | 0.082% |
| pWISE9120 | 453 | 0.010% | 0.003% | 0.003% | 0.002% | 2.155% | 1.054% |
| pWISE10648 | 629 | 0.012% | 0.003% | 0.006% | 0.006% | 0.819% | 0.545% |
| pWISE10649 | 630 | 0.004% | 0.002% | 0.002% | 0.002% | 1.133% | 0.747% |
| pWISE10650 | 631 | 0.006% | 0.002% | 0.003% | 0.012% | 1.107% | 0.699% |
| pWISE10651 | 632 | 0.004% | 0.003% | 0.006% | 0.003% | 1.400% | 0.686% |
| pWISE10652 | 633 | 0.012% | 0.003% | 0.003% | 0.005% | 0.908% | 0.405% |
| pWISE10658 | 639 | 0.010% | 0.006% | 0.006% | 0.007% | 3.508% | 1.566% |
| pWISE10653 | 634 | 0.006% | 0.002% | 0.002% | 0.003% | 1.609% | 0.921% |
| pWISE10654 | 635 | 0.005% | 0.006% | 0.004% | 0.007% | 2.585% | 1.535% |
| pWISE10655 | 636 | 0.019% | 0.004% | 0.006% | 0.003% | 2.102% | 1.079% |
| pWISE10656 | 637 | 0.009% | 0.004% | 0.003% | 0.013% | 1.866% | 0.763% |
| pWISE10657 | 638 | 0.013% | 0.002% | 0.004% | 0.006% | 1.295% | 0.385% |
| | | Average G to A editing percent at the nucleotides numbered | | | | | |
| | | according to the position in the PWsp3628 spacer sequence on the | | | | | |
| | Protein SEQ | opposite, target genomic DNA strand | | | | | |
| pWISE | ID NO: | G.15 | G.16 | G.19 | G.20 | G.21 | G.23 |
| pWISE121 | 56 | 0.250% | 0.227% | 0.211% | 0.333% | 0.416% | 0.334% |
| pWISE9120 | 453 | 0.289% | 0.251% | 0.369% | 0.263% | 0.283% | 0.274% |
| pWISE10648 | 629 | 0.255% | 0.239% | 0.300% | 0.228% | 0.286% | 0.233% |
| pWISE10649 | 630 | 0.201% | 0.169% | 0.237% | 0.154% | 0.193% | 0.155% |
| pWISE10650 | 631 | 0.153% | 0.132% | 0.169% | 0.117% | 0.191% | 0.130% |
| pWISE10651 | 632 | 0.149% | 0.129% | 0.195% | 0.116% | 0.172% | 0.177% |
| pWISE10652 | 633 | 0.060% | 0.042% | 0.082% | 0.057% | 0.139% | 0.082% |
| pWISE10658 | 639 | 0.665% | 0.606% | 0.595% | 0.418% | 0.396% | 0.470% |
| pWISE10653 | 634 | 0.407% | 0.378% | 0.423% | 0.315% | 0.347% | 0.298% |
| pWISE10654 | 635 | 0.585% | 0.528% | 0.618% | 0.442% | 0.441% | 0.410% |
| pWISE10655 | 636 | 0.498% | 0.428% | 0.453% | 0.339% | 0.330% | 0.325% |
| pWISE10656 | 637 | 0.312% | 0.302% | 0.411% | 0.253% | 0.207% | 0.291% |
| pWISE10657 | 638 | 0.304% | 0.308% | 0.316% | 0.243% | 0.265% | 0.233% |
| | | Average G to A editing percent at the nucleotides numbered | | | | | |
| | | according to the position in the PWsp3628 spacer sequence on the | | | | | |
| | Protein SEQ | opposite, target genomic DNA strand | | | | | |
| pWISE | ID NO: | G.24 | G.25 | G.26 | G.27 | G.29 | G.30 |
| pWISE121 | 56 | 0.272% | 0.214% | 0.177% | 0.206% | 0.336% | 0.129% |
| pWISE9120 | 453 | 0.260% | 0.266% | 0.283% | 0.366% | 0.081% | 0.055% |
| pWISE10648 | 629 | 0.234% | 0.234% | 0.251% | 0.337% | 0.122% | 0.091% |
| pWISE10649 | 630 | 0.144% | 0.145% | 0.152% | 0.206% | 0.061% | 0.042% |
| pWISE10650 | 631 | 0.129% | 0.135% | 0.163% | 0.202% | 0.074% | 0.054% |
| pWISE10651 | 632 | 0.161% | 0.167% | 0.192% | 0.266% | 0.077% | 0.071% |
| pWISE10652 | 633 | 0.078% | 0.082% | 0.088% | 0.102% | 0.057% | 0.054% |
| pWISE10658 | 639 | 0.403% | 0.394% | 0.413% | 0.504% | 0.148% | 0.131% |
| pWISE10653 | 634 | 0.279% | 0.282% | 0.298% | 0.331% | 0.100% | 0.048% |
| pWISE10654 | 635 | 0.392% | 0.387% | 0.399% | 0.507% | 0.147% | 0.097% |
| pWISE10655 | 636 | 0.288% | 0.289% | 0.311% | 0.387% | 0.097% | 0.062% |
| pWISE10656 | 637 | 0.243% | 0.222% | 0.268% | 0.340% | 0.100% | 0.068% |
| pWISE10657 | 638 | 0.209% | 0.204% | 0.246% | 0.289% | 0.124% | 0.109% |

TABLE 88

| | | Standard deviation of the average G to A editing percent at the | | | | | |
|---|---|---|---|---|---|---|---|
| | | nucleotides numbered according to the position in the PWsp3628 | | | | | |
| | Protein SEQ | spacer sequence on the opposite, target genomic DNA strand | | | | | |
| pWISE | ID NO: | G.-6 | G.-5 | G.6 | G.7 | G.10 | G.12 |
| pWISE121 | 56 | 0.012% | 0.023% | 0.077% | 0.058% | 0.087% | 0.082% |
| pWISE9120 | 453 | 0.010% | 0.005% | 0.002% | 0.003% | 1.025% | 0.513% |
| pWISE10648 | 629 | 0.016% | 0.003% | 0.010% | 0.003% | 0.334% | 0.228% |
| pWISE10649 | 630 | 0.005% | 0.003% | 0.001% | 0.002% | 0.579% | 0.388% |
| pWISE10650 | 631 | 0.004% | 0.002% | 0.003% | 0.019% | 0.960% | 0.621% |
| pWISE10651 | 632 | 0.005% | 0.005% | 0.005% | 0.003% | 0.621% | 0.352% |
| pWISE10652 | 633 | 0.022% | 0.002% | 0.002% | 0.006% | 0.457% | 0.239% |
| pWISE10658 | 639 | 0.012% | 0.006% | 0.004% | 0.003% | 1.589% | 0.627% |
| pWISE10653 | 634 | 0.006% | 0.003% | 0.004% | 0.004% | 0.904% | 0.464% |
| pWISE10654 | 635 | 0.004% | 0.006% | 0.004% | 0.009% | 1.276% | 0.649% |
| pWISE10655 | 636 | 0.030% | 0.003% | 0.005% | 0.004% | 1.112% | 0.581% |
| pWISE10656 | 637 | 0.004% | 0.004% | 0.003% | 0.015% | 0.933% | 0.418% |
| pWISE10657 | 638 | 0.016% | 0.002% | 0.004% | 0.006% | 0.792% | 0.259% |
| | | Standard deviation of the average G to A editing percent at the | | | | | |
| | | nucleotides numbered according to the position in the PWsp3628 | | | | | |
| | Protein SEQ | spacer sequence on the opposite, target genomic DNA strand | | | | | |
| pWISE | ID NO: | G.15 | G.16 | G.19 | G.20 | G.21 | G.23 |
| pWISE121 | 56 | 0.167% | 0.161% | 0.161% | 0.232% | 0.242% | 0.219% |
| pWISE9120 | 453 | 0.128% | 0.094% | 0.131% | 0.075% | 0.077% | 0.066% |
| pWISE10648 | 629 | 0.081% | 0.087% | 0.131% | 0.094% | 0.168% | 0.100% |
| pWISE10649 | 630 | 0.055% | 0.041% | 0.060% | 0.031% | 0.120% | 0.023% |
| pWISE10650 | 631 | 0.132% | 0.116% | 0.148% | 0.109% | 0.151% | 0.127% |
| pWISE10651 | 632 | 0.098% | 0.081% | 0.123% | 0.059% | 0.133% | 0.133% |
| pWISE10652 | 633 | 0.043% | 0.050% | 0.058% | 0.041% | 0.108% | 0.063% |
| pWISE10658 | 639 | 0.327% | 0.323% | 0.214% | 0.203% | 0.184% | 0.214% |
| pWISE10653 | 634 | 0.190% | 0.181% | 0.187% | 0.127% | 0.077% | 0.119% |
| pWISE10654 | 635 | 0.230% | 0.185% | 0.236% | 0.141% | 0.132% | 0.121% |
| pWISE10655 | 636 | 0.261% | 0.226% | 0.244% | 0.153% | 0.068% | 0.158% |
| pWISE10656 | 637 | 0.216% | 0.206% | 0.300% | 0.160% | 0.132% | 0.185% |
| pWISE10657 | 638 | 0.218% | 0.232% | 0.223% | 0.185% | 0.166% | 0.183% |
| | | Standard deviation of the average G to A editing percent at the | | | | | |
| | | nucleotides numbered according to the position in the PWsp3628 | | | | | |
| | Protein SEQ | spacer sequence on the opposite, target genomic DNA strand | | | | | |
| pWISE | ID NO: | G.24 | G.25 | G.26 | G.27 | G.29 | G.30 |
| pWISE121 | 56 | 0.196% | 0.155% | 0.155% | 0.129% | 0.243% | 0.127% |
| pWISE9120 | 453 | 0.064% | 0.062% | 0.078% | 0.113% | 0.025% | 0.032% |
| pWISE10648 | 629 | 0.103% | 0.106% | 0.103% | 0.173% | 0.059% | 0.062% |
| pWISE10649 | 630 | 0.019% | 0.023% | 0.028% | 0.057% | 0.018% | 0.012% |
| pWISE10650 | 631 | 0.122% | 0.120% | 0.186% | 0.183% | 0.040% | 0.027% |
| pWISE10651 | 632 | 0.105% | 0.100% | 0.152% | 0.180% | 0.032% | 0.041% |
| pWISE10652 | 633 | 0.063% | 0.063% | 0.061% | 0.079% | 0.052% | 0.053% |
| pWISE10658 | 639 | 0.135% | 0.137% | 0.153% | 0.167% | 0.051% | 0.114% |
| pWISE10653 | 634 | 0.103% | 0.103% | 0.098% | 0.101% | 0.058% | 0.051% |
| pWISE10654 | 635 | 0.100% | 0.097% | 0.105% | 0.160% | 0.064% | 0.036% |
| pWISE10655 | 636 | 0.138% | 0.143% | 0.162% | 0.202% | 0.049% | 0.035% |
| pWISE10656 | 637 | 0.178% | 0.164% | 0.214% | 0.261% | 0.087% | 0.061% |
| pWISE10657 | 638 | 0.151% | 0.151% | 0.215% | 0.209% | 0.111% | 0.091% |

TABLE 89

| | | Average C to T editing percent at the nucleotides numbered | | | | | | |
|---|---|---|---|---|---|---|---|---|
| | | according to the position in the PWsp3629 spacer sequence on the | | | | | | |
| | Protein SEQ | non-target, genomic DNA strand | | | | | | |
| pWISE | ID NO: | C.-7 | C.1 | C.4 | C.5 | C.7 | C.8 | C.15 |
| pWISE121 | 56 | 0.035% | 0.048% | 0.287% | 0.061% | 0.095% | 0.184% | 1.003% |
| pWISE9120 | 453 | 0.006% | 0.005% | 0.006% | 0.005% | 2.400% | 2.466% | 0.840% |
| pWISE10648 | 629 | 0.004% | 0.004% | 0.003% | 0.008% | 1.142% | 1.219% | 0.329% |
| pWISE10649 | 630 | 0.007% | 0.002% | 0.002% | 0.006% | 0.681% | 0.683% | 0.201% |
| pWISE10650 | 631 | 0.004% | 0.018% | 0.015% | 0.015% | 1.678% | 1.742% | 0.634% |
| pWISE10651 | 632 | 0.005% | 0.014% | 0.004% | 0.005% | 1.589% | 1.704% | 0.689% |
| pWISE10652 | 633 | 0.006% | 0.006% | 0.010% | 0.008% | 1.163% | 1.258% | 0.438% |
| pWISE10658 | 639 | 0.004% | 0.005% | 0.010% | 0.004% | 4.049% | 4.367% | 0.754% |
| pWISE10653 | 634 | 0.004% | 0.007% | 0.020% | 0.012% | 2.062% | 2.117% | 0.382% |
| pWISE10654 | 635 | 0.007% | 0.009% | 0.008% | 0.014% | 1.999% | 2.085% | 0.406% |
| pWISE10655 | 636 | 0.013% | 0.011% | 0.014% | 0.018% | 2.000% | 1.869% | 0.530% |
| pWISE10656 | 637 | 0.003% | 0.002% | 0.009% | 0.016% | 1.865% | 1.862% | 0.545% |
| pWISE10657 | 638 | 0.006% | 0.004% | 0.008% | 0.005% | 1.897% | 1.949% | 0.485% |

| | | Average C to T editing percent at the nucleotides numbered | | | | | |
|---|---|---|---|---|---|---|---|
| | | according to the position in the PWsp3629 spacer sequence on the | | | | | |
| | Protein SEQ | non-target, genomic DNA strand | | | | | |
| pWISE | ID NO: | C.16 | C.22 | C.23 | C.24 | C.25 | C.29 |
| pWISE121 | 56 | 0.590% | 0.658% | 0.550% | 0.418% | 0.207% | 0.132% |
| pWISE9120 | 453 | 0.666% | 0.201% | 0.179% | 0.165% | 0.184% | 0.018% |
| pWISE10648 | 629 | 0.149% | 0.121% | 0.098% | 0.090% | 0.118% | 0.026% |
| pWISE10649 | 630 | 0.094% | 0.265% | 0.203% | 0.186% | 0.181% | 0.035% |
| pWISE10650 | 631 | 0.334% | 0.751% | 0.685% | 0.678% | 0.662% | 0.084% |
| pWISE10651 | 632 | 0.407% | 0.991% | 0.907% | 0.881% | 0.922% | 0.128% |
| pWISE10652 | 633 | 0.243% | 0.820% | 0.643% | 0.632% | 0.628% | 0.096% |
| pWISE10658 | 639 | 0.385% | 0.311% | 0.204% | 0.202% | 0.220% | 0.036% |
| pWISE10653 | 634 | 0.154% | 0.200% | 0.132% | 0.125% | 0.141% | 0.023% |
| pWISE10654 | 635 | 0.185% | 0.575% | 0.433% | 0.402% | 0.398% | 0.060% |
| pWISE10655 | 636 | 0.237% | 0.876% | 0.750% | 0.695% | 0.709% | 0.088% |
| pWISE10656 | 637 | 0.252% | 0.688% | 0.610% | 0.580% | 0.607% | 0.073% |
| pWISE10657 | 638 | 0.192% | 0.790% | 0.648% | 0.652% | 0.676% | 0.090% |

TABLE 90

| | | Standard Deviation of the average editing percent at the | | | | | | |
|---|---|---|---|---|---|---|---|---|
| | | nucleotides numbered according to the position in the PWsp3629 | | | | | | |
| | Protein SEQ | spacer sequence on the non-target, genomic DNA strand | | | | | | |
| pWISE | ID NO: | C.-7 | C.1 | C.4 | C.5 | C.7 | C.8 | C.15 |
| pWISE121 | 56 | 0.027% | 0.059% | 0.190% | 0.046% | 0.106% | 0.134% | 0.563% |
| pWISE9120 | 453 | 0.004% | 0.001% | 0.006% | 0.004% | 1.047% | 1.007% | 0.367% |
| pWISE10648 | 629 | 0.005% | 0.004% | 0.002% | 0.005% | 0.436% | 0.427% | 0.109% |
| pWISE10649 | 630 | 0.004% | 0.001% | 0.002% | 0.010% | 0.276% | 0.252% | 0.080% |
| pWISE10650 | 631 | 0.004% | 0.024% | 0.023% | 0.030% | 0.628% | 0.575% | 0.266% |
| pWISE10651 | 632 | 0.005% | 0.014% | 0.005% | 0.009% | 0.293% | 0.316% | 0.167% |
| pWISE10652 | 633 | 0.003% | 0.005% | 0.008% | 0.011% | 0.477% | 0.447% | 0.305% |
| pWISE10658 | 639 | 0.003% | 0.002% | 0.009% | 0.005% | 1.381% | 1.313% | 0.245% |
| pWISE10653 | 634 | 0.007% | 0.008% | 0.014% | 0.014% | 0.894% | 0.909% | 0.182% |
| pWISE10654 | 635 | 0.005% | 0.006% | 0.007% | 0.011% | 0.638% | 0.638% | 0.218% |
| pWISE10655 | 636 | 0.020% | 0.017% | 0.019% | 0.017% | 0.768% | 0.508% | 0.232% |
| pWISE10656 | 637 | 0.004% | 0.003% | 0.010% | 0.010% | 0.864% | 0.715% | 0.248% |
| pWISE10657 | 638 | 0.005% | 0.007% | 0.012% | 0.004% | 0.610% | 0.603% | 0.241% |

| | |
|---|---|
| | Standard Deviation of the average editing percent at the |
| | nucleotides numbered according to the position in the PWsp3629 |
| Protein SEQ | spacer sequence on the non-target, genomic DNA strand |

| pWISE | ID NO: | C.16 | C.22 | C.23 | C.24 | C.25 | C.29 |
|---|---|---|---|---|---|---|---|
| pWISE121 | 56 | 0.388% | 0.382% | 0.285% | 0.262% | 0.150% | 0.142% |
| pWISE9120 | 453 | 0.252% | 0.103% | 0.105% | 0.104% | 0.108% | 0.023% |
| pWISE10648 | 629 | 0.071% | 0.067% | 0.067% | 0.070% | 0.093% | 0.026% |
| pWISE10649 | 630 | 0.025% | 0.167% | 0.118% | 0.113% | 0.102% | 0.030% |
| pWISE10650 | 631 | 0.116% | 0.456% | 0.413% | 0.413% | 0.402% | 0.056% |
| pWISE10651 | 632 | 0.074% | 0.600% | 0.554% | 0.539% | 0.513% | 0.068% |
| pWISE10652 | 633 | 0.162% | 0.573% | 0.618% | 0.592% | 0.573% | 0.055% |
| pWISE10658 | 639 | 0.184% | 0.179% | 0.133% | 0.131% | 0.126% | 0.027% |
| pWISE10653 | 634 | 0.043% | 0.125% | 0.107% | 0.103% | 0.116% | 0.024% |
| pWISE10654 | 635 | 0.086% | 0.350% | 0.267% | 0.264% | 0.224% | 0.070% |
| pWISE10655 | 636 | 0.109% | 0.576% | 0.516% | 0.464% | 0.447% | 0.074% |
| pWISE10656 | 637 | 0.096% | 0.553% | 0.526% | 0.496% | 0.496% | 0.063% |
| pWISE10657 | 638 | 0.046% | 0.499% | 0.420% | 0.411% | 0.429% | 0.066% |

TABLE 91

| | | Average G to A editing percent at the nucleotides numbered | | | | | | |
|---|---|---|---|---|---|---|---|---|
| | | according to the position in the PWsp3629 spacer sequence on the | | | | | | |
| | Protein SEQ | opposite, target genomic DNA strand | | | | | | |
| pWISE | ID NO: | G.-8 | G.-4 | G.-1 | G.3 | G.10 | G.11 | G.12 |
| pWISE121 | 56 | 0.007% | 0.027% | 0.025% | 0.032% | 0.099% | 0.082% | 0.135% |
| pWISE9120 | 453 | 0.008% | 0.005% | 0.004% | 0.014% | 3.714% | 3.744% | 3.934% |
| pWISE10648 | 629 | 0.005% | 0.013% | 0.007% | 0.010% | 2.207% | 2.220% | 2.310% |
| pWISE10649 | 630 | 0.002% | 0.013% | 0.002% | 0.006% | 1.599% | 1.620% | 1.645% |
| pWISE10650 | 631 | 0.010% | 0.002% | 0.009% | 0.011% | 3.606% | 3.766% | 3.918% |
| pWISE10651 | 632 | 0.005% | 0.025% | 0.023% | 0.033% | 3.767% | 3.939% | 4.154% |
| pWISE10652 | 633 | 0.006% | 0.005% | 0.003% | 0.011% | 2.778% | 2.908% | 3.103% |
| pWISE10658 | 639 | 0.009% | 0.003% | 0.004% | 0.010% | 6.408% | 6.407% | 6.532% |
| pWISE10653 | 634 | 0.005% | 0.004% | 0.004% | 0.014% | 3.136% | 3.182% | 3.288% |
| pWISE10654 | 635 | 0.004% | 0.001% | 0.002% | 0.009% | 4.984% | 5.029% | 5.090% |
| pWISE10655 | 636 | 0.003% | 0.009% | 0.002% | 0.014% | 4.913% | 5.047% | 5.176% |
| pWISE10656 | 637 | 0.002% | 0.005% | 0.008% | 0.012% | 4.152% | 4.333% | 4.435% |
| pWISE10657 | 638 | 0.007% | 0.005% | 0.003% | 0.007% | 3.300% | 3.415% | 3.529% |
| | | Average G to A editing percent at the nucleotides numbered | | | | | | |
| | | according to the position in the PWsp3629 spacer sequence on the | | | | | | |
| | Protein SEQ | opposite, target genomic DNA strand | | | | | | |
| pWISE | ID NO: | G.18 | G.20 | G.26 | G.28 | G.31 | G.32 | G.33 |
| pWISE121 | 56 | 0.315% | 0.260% | 0.108% | 0.081% | 0.083% | 0.109% | 0.102% |
| pWISE9120 | 453 | 1.002% | 0.758% | 1.077% | 0.308% | 0.160% | 0.129% | 0.148% |
| pWISE10648 | 629 | 0.630% | 0.545% | 0.812% | 0.244% | 0.151% | 0.133% | 0.150% |
| pWISE10649 | 630 | 0.746% | 0.821% | 1.278% | 0.334% | 0.249% | 0.193% | 0.195% |
| pWISE10650 | 631 | 2.774% | 2.729% | 3.918% | 1.299% | 0.774% | 0.638% | 0.680% |
| pWISE10651 | 632 | 2.961% | 2.980% | 4.709% | 1.595% | 0.955% | 0.717% | 0.752% |
| pWISE10652 | 633 | 2.698% | 2.628% | 4.418% | 1.626% | 0.945% | 0.726% | 0.737% |
| pWISE10658 | 639 | 1.737% | 1.383% | 1.677% | 0.312% | 0.200% | 0.133% | 0.179% |
| pWISE10653 | 634 | 0.746% | 0.583% | 0.849% | 0.170% | 0.153% | 0.139% | 0.134% |
| pWISE10654 | 635 | 2.501% | 2.471% | 3.210% | 0.986% | 0.579% | 0.424% | 0.513% |
| pWISE10655 | 636 | 2.995% | 3.089% | 4.137% | 1.262% | 0.734% | 0.595% | 0.635% |
| pWISE10656 | 637 | 2.526% | 2.418% | 3.720% | 1.085% | 0.770% | 0.561% | 0.590% |
| pWISE10657 | 638 | 2.638% | 2.441% | 4.667% | 1.312% | 0.855% | 0.647% | 0.681% |

TABLE 92

| | | Standard deviation of the average G to A editing percent at the | | | | | | |
|---|---|---|---|---|---|---|---|---|
| | | nucleotides numbered according to the position in the PWsp3629 | | | | | | |
| | Protein SEQ | spacer sequence on the opposite, target genomic DNA strand | | | | | | |
| pWISE | ID NO: | G.-8 | G.-4 | G.-1 | G.3 | G.10 | G.11 | G.12 |
| pWISE121 | 56 | 0.011% | 0.032% | 0.025% | 0.018% | 0.055% | 0.045% | 0.126% |
| pWISE9120 | 453 | 0.006% | 0.005% | 0.006% | 0.011% | 1.750% | 1.758% | 1.783% |
| pWISE10648 | 629 | 0.005% | 0.018% | 0.006% | 0.012% | 1.001% | 1.004% | 1.075% |
| pWISE10649 | 630 | 0.002% | 0.016% | 0.002% | 0.004% | 0.628% | 0.624% | 0.636% |
| pWISE10650 | 631 | 0.012% | 0.002% | 0.012% | 0.010% | 1.573% | 1.725% | 1.837% |
| pWISE10651 | 632 | 0.005% | 0.032% | 0.027% | 0.016% | 1.099% | 1.193% | 1.263% |
| pWISE10652 | 633 | 0.007% | 0.005% | 0.003% | 0.006% | 0.801% | 0.818% | 0.852% |
| pWISE10658 | 639 | 0.016% | 0.001% | 0.006% | 0.004% | 2.460% | 2.474% | 2.515% |
| pWISE10653 | 634 | 0.009% | 0.003% | 0.002% | 0.010% | 1.150% | 1.207% | 1.260% |
| pWISE10654 | 635 | 0.002% | 0.002% | 0.003% | 0.007% | 2.056% | 2.092% | 2.113% |
| pWISE10655 | 636 | 0.004% | 0.004% | 0.003% | 0.018% | 2.008% | 2.086% | 2.133% |
| pWISE10656 | 637 | 0.003% | 0.005% | 0.006% | 0.011% | 1.833% | 1.983% | 2.034% |
| pWISE10657 | 638 | 0.011% | 0.006% | 0.002% | 0.006% | 0.999% | 1.051% | 1.101% |
| | | Standard deviation of the average G to A editing percent at the | | | | | | |
| | | nucleotides numbered according to the position in the PWsp3629 | | | | | | |
| | Protein SEQ | spacer sequence on the opposite, target genomic DNA strand | | | | | | |
| pWISE | ID NO: | G.18 | G.20 | G.26 | G.28 | G.31 | G.32 | G.33 |
| pWISE121 | 56 | 0.214% | 0.175% | 0.097% | 0.086% | 0.050% | 0.080% | 0.078% |
| pWISE9120 | 453 | 0.540% | 0.494% | 0.606% | 0.215% | 0.077% | 0.062% | 0.066% |
| pWISE10648 | 629 | 0.345% | 0.336% | 0.521% | 0.206% | 0.111% | 0.099% | 0.114% |
| pWISE10649 | 630 | 0.284% | 0.334% | 0.563% | 0.155% | 0.139% | 0.102% | 0.107% |
| pWISE10650 | 631 | 1.528% | 1.590% | 2.484% | 0.895% | 0.506% | 0.420% | 0.449% |
| pWISE10651 | 632 | 1.156% | 1.424% | 2.613% | 1.049% | 0.561% | 0.464% | 0.466% |
| pWISE10652 | 633 | 1.120% | 1.185% | 2.406% | 1.064% | 0.596% | 0.460% | 0.457% |
| pWISE10658 | 639 | 0.861% | 0.757% | 0.912% | 0.195% | 0.114% | 0.083% | 0.094% |
| pWISE10653 | 634 | 0.459% | 0.317% | 0.469% | 0.158% | 0.124% | 0.122% | 0.106% |
| pWISE10654 | 635 | 1.245% | 1.374% | 1.960% | 0.725% | 0.382% | 0.281% | 0.370% |
| pWISE10655 | 636 | 1.580% | 1.718% | 2.443% | 0.834% | 0.447% | 0.366% | 0.406% |
| pWISE10656 | 637 | 1.412% | 1.550% | 2.849% | 0.976% | 0.644% | 0.451% | 0.477% |
| pWISE10657 | 638 | 1.176% | 1.167% | 2.630% | 0.832% | 0.516% | 0.381% | 0.397% |

TABLE 93

| | | Average C to T editing percent at the nucleotides numbered | | | | | |
|---|---|---|---|---|---|---|---|
| | | according to the position in the PWsp3630 spacer sequence on the | | | | | |
| | Protein SEQ | non-target, genomic DNA strand | | | | | |
| pWISE | ID NO: | C.-7 | C.-6 | C.-1 | C.1 | C.6 | C.11 |
| pWISE121 | 56 | 0.016% | 0.018% | 0.018% | 0.023% | 0.051% | 0.145% |
| pWISE9120 | 453 | 0.059% | 0.044% | 0.114% | 0.056% | 1.177% | 1.749% |
| pWISE10648 | 629 | 0.069% | 0.046% | 0.101% | 0.052% | 0.476% | 1.144% |
| pWISE10649 | 630 | 0.055% | 0.024% | 0.057% | 0.029% | 0.166% | 0.390% |
| pWISE10650 | 631 | 0.097% | 0.063% | 0.130% | 0.087% | 0.386% | 1.020% |
| pWISE10651 | 632 | 0.071% | 0.057% | 0.093% | 0.062% | 0.292% | 0.837% |
| pWISE10652 | 633 | 0.074% | 0.052% | 0.093% | 0.045% | 0.298% | 0.855% |
| pWISE10658 | 639 | 0.046% | 0.033% | 0.111% | 0.051% | 0.489% | 0.940% |
| pWISE10653 | 634 | 0.052% | 0.041% | 0.095% | 0.048% | 0.489% | 0.973% |
| pWISE10654 | 635 | 0.080% | 0.065% | 0.127% | 0.068% | 0.295% | 0.705% |
| pWISE10655 | 636 | 0.056% | 0.027% | 0.070% | 0.035% | 0.278% | 0.599% |
| pWISE10656 | 637 | 0.032% | 0.022% | 0.055% | 0.024% | 0.201% | 0.557% |
| pWISE10657 | 638 | 0.043% | 0.039% | 0.076% | 0.060% | 0.227% | 0.590% |
| | | Average C to T editing percent at the nucleotides numbered | | | | | |
| | | according to the position in the PWsp3630 spacer sequence on the | | | | | |
| | Protein SEQ | non-target, genomic DNA strand | | | | | |
| pWISE | ID NO: | C.12 | C.15 | C.19 | C.30 | C.31 | C.33 |
| pWISE121 | 56 | 0.145% | 0.992% | 0.336% | 0.394% | 0.201% | 0.166% |
| pWISE9120 | 453 | 2.255% | 2.946% | 0.558% | 0.002% | 0.007% | 0.003% |
| pWISE10648 | 629 | 1.591% | 2.669% | 0.909% | 0.005% | 0.011% | 0.023% |
| pWISE10649 | 630 | 0.700% | 1.893% | 0.821% | 0.010% | 0.025% | 0.011% |
| pWISE10650 | 631 | 1.545% | 4.167% | 2.020% | 0.016% | 0.025% | 0.007% |
| pWISE10651 | 632 | 1.359% | 3.838% | 2.030% | 0.010% | 0.017% | 0.010% |
| pWISE10652 | 633 | 1.352% | 4.082% | 2.138% | 0.011% | 0.034% | 0.006% |
| pWISE10658 | 639 | 1.525% | 1.985% | 0.411% | 0.003% | 0.008% | 0.003% |
| pWISE10653 | 634 | 1.411% | 2.498% | 0.804% | 0.005% | 0.031% | 0.015% |
| pWISE10654 | 635 | 1.277% | 3.042% | 1.376% | 0.013% | 0.018% | 0.002% |
| pWISE10655 | 636 | 1.021% | 2.645% | 1.272% | 0.009% | 0.024% | 0.007% |
| pWISE10656 | 637 | 0.904% | 2.375% | 1.088% | 0.002% | 0.015% | 0.012% |
| pWISE10657 | 638 | 0.961% | 2.547% | 0.818% | 0.010% | 0.012% | 0.005% |

TABLE 94

| | | Standard Deviation of the average editing percent at the nucleotides | | | | | |
|---|---|---|---|---|---|---|---|
| | | numbered according to the position in the PWsp3630 spacer sequence | | | | | |
| | Protein SEQ | on the non-target, genomic DNA strand | | | | | |
| pWISE | ID NO: | C.-7 | C.-6 | C.-1 | C.1 | C.6 | C.11 |
| pWISE121 | 56 | 0.018% | 0.018% | 0.023% | 0.029% | 0.037% | 0.061% |
| pWISE9120 | 453 | 0.010% | 0.015% | 0.044% | 0.012% | 0.613% | 0.889% |
| pWISE10648 | 629 | 0.025% | 0.022% | 0.031% | 0.013% | 0.224% | 0.556% |
| pWISE10649 | 630 | 0.026% | 0.014% | 0.015% | 0.016% | 0.108% | 0.162% |
| pWISE10650 | 631 | 0.049% | 0.017% | 0.048% | 0.029% | 0.125% | 0.396% |
| pWISE10651 | 632 | 0.022% | 0.013% | 0.021% | 0.019% | 0.073% | 0.313% |
| pWISE10652 | 633 | 0.038% | 0.027% | 0.044% | 0.022% | 0.129% | 0.303% |
| pWISE10658 | 639 | 0.055% | 0.033% | 0.113% | 0.055% | 0.449% | 0.858% |
| pWISE10653 | 634 | 0.024% | 0.010% | 0.044% | 0.024% | 0.349% | 0.573% |
| pWISE10654 | 635 | 0.061% | 0.057% | 0.084% | 0.058% | 0.204% | 0.493% |
| pWISE10655 | 636 | 0.048% | 0.028% | 0.056% | 0.025% | 0.222% | 0.486% |
| pWISE10656 | 637 | 0.018% | 0.018% | 0.051% | 0.019% | 0.156% | 0.429% |
| pWISE10657 | 638 | 0.036% | 0.038% | 0.065% | 0.055% | 0.175% | 0.463% |

| | |
|---|---|
| | Standard Deviation of the average editing percent at the nucleotides |
| | numbered according to the position in the PWsp3630 spacer |
| Protein SEQ | sequence on the non-target, genomic DNA strand |

| pWISE | ID NO: | C.12 | C.15 | C.19 | C.30 | C.31 | C.33 |
|---|---|---|---|---|---|---|---|
| pWISE121 | 56 | 0.076% | 0.137% | 0.143% | 0.153% | 0.074% | 0.019% |
| pWISE9120 | 453 | 1.170% | 1.475% | 0.231% | 0.004% | 0.006% | 0.003% |
| pWISE10648 | 629 | 0.735% | 1.070% | 0.372% | 0.004% | 0.016% | 0.025% |
| pWISE10649 | 630 | 0.345% | 0.892% | 0.379% | 0.009% | 0.013% | 0.006% |
| pWISE10650 | 631 | 0.556% | 1.615% | 0.730% | 0.013% | 0.021% | 0.007% |
| pWISE10651 | 632 | 0.477% | 1.388% | 0.711% | 0.010% | 0.010% | 0.010% |
| pWISE10652 | 633 | 0.470% | 1.450% | 0.717% | 0.014% | 0.015% | 0.008% |
| pWISE10658 | 639 | 1.417% | 1.837% | 0.390% | 0.002% | 0.009% | 0.003% |
| pWISE10653 | 634 | 0.734% | 1.315% | 0.337% | 0.004% | 0.031% | 0.019% |
| pWISE10654 | 635 | 0.902% | 1.978% | 0.902% | 0.014% | 0.027% | 0.002% |
| pWISE10655 | 636 | 0.834% | 2.170% | 1.008% | 0.007% | 0.021% | 0.010% |
| pWISE10656 | 637 | 0.717% | 1.896% | 0.860% | 0.002% | 0.011% | 0.008% |
| pWISE10657 | 638 | 0.764% | 2.033% | 0.635% | 0.012% | 0.009% | 0.004% |

TABLE 95

| | | Average G to A editing percent at the nucleotides numbered | | | | |
|---|---|---|---|---|---|---|
| | | according to the position in the PWsp3630 spacer sequence on | | | | |
| | Protein | the opposite, target genomic DNA strand | | | | |
| pWISE | SEQ ID NO: | G.-4 | G.8 | G.9 | G.10 | G.14 |
| pWISE121 | 56 | 0.015% | 0.083% | 0.081% | 0.073% | 0.160% |
| pWISE9120 | 453 | 0.002% | 0.185% | 0.528% | 0.772% | 0.945% |
| pWISE10648 | 629 | 0.012% | 0.079% | 0.128% | 0.196% | 1.284% |
| pWISE10649 | 630 | 0.004% | 0.060% | 0.084% | 0.147% | 0.942% |
| pWISE10650 | 631 | 0.003% | 0.154% | 0.181% | 0.282% | 1.976% |
| pWISE10651 | 632 | 0.002% | 0.166% | 0.200% | 0.257% | 1.813% |
| pWISE10652 | 633 | 0.011% | 0.107% | 0.142% | 0.236% | 1.721% |
| pWISE10658 | 639 | 0.004% | 0.242% | 0.750% | 1.400% | 1.650% |
| pWISE10653 | 634 | 0.002% | 0.191% | 0.248% | 0.400% | 2.330% |
| pWISE10654 | 635 | 0.006% | 0.167% | 0.282% | 0.516% | 2.486% |
| pWISE10655 | 636 | 0.002% | 0.215% | 0.287% | 0.464% | 2.080% |
| pWISE10656 | 637 | 0.003% | 0.126% | 0.201% | 0.391% | 1.718% |
| pWISE10657 | 638 | 0.002% | 0.096% | 0.129% | 0.324% | 1.252% |
| | | Average G to A editing percent at the nucleotides numbered | | | | |
| | | according to the position in the PWsp3630 spacer sequence on | | | | |
| | Protein | the opposite, target genomic DNA strand | | | | |
| pWISE | SEQ ID NO: | G.16 | G.17 | G.18 | G.21 | G.22 |
| pWISE121 | 56 | 0.528% | 0.436% | 1.115% | 0.329% | 0.504% |
| pWISE9120 | 453 | 0.604% | 0.420% | 0.378% | 0.388% | 0.400% |
| pWISE10648 | 629 | 1.015% | 0.718% | 0.652% | 0.689% | 0.685% |
| pWISE10649 | 630 | 0.881% | 0.718% | 0.677% | 0.689% | 0.708% |
| pWISE10650 | 631 | 2.177% | 1.772% | 1.659% | 1.555% | 1.594% |
| pWISE10651 | 632 | 2.184% | 1.835% | 1.727% | 1.668% | 1.688% |
| pWISE10652 | 633 | 2.467% | 1.967% | 1.894% | 1.810% | 1.878% |
| pWISE10658 | 639 | 1.618% | 1.071% | 0.874% | 0.724% | 0.727% |
| pWISE10653 | 634 | 2.462% | 1.828% | 1.671% | 1.259% | 1.266% |
| pWISE10654 | 635 | 2.789% | 2.265% | 2.148% | 1.966% | 2.040% |
| pWISE10655 | 636 | 2.721% | 2.162% | 2.045% | 1.887% | 1.940% |
| pWISE10656 | 637 | 2.459% | 1.969% | 1.854% | 1.738% | 1.867% |
| pWISE10657 | 638 | 2.236% | 1.673% | 1.529% | 1.423% | 1.571% |

TABLE 96

| | | Standard deviation of the average G to A editing percent at the | | | | |
|---|---|---|---|---|---|---|
| | | nucleotides numbered according to the position in the PWsp3630 | | | | |
| | Protein | spacer sequence on the opposite, target genomic DNA strand | | | | |
| pWISE | SEQ ID NO: | G.-4 | G.8 | G.9 | G.10 | G.14 |
| pWISE121 | 56 | 0.023% | 0.032% | 0.019% | 0.026% | 0.059% |
| pWISE9120 | 453 | 0.002% | 0.156% | 0.322% | 0.411% | 0.503% |
| pWISE10648 | 629 | 0.014% | 0.053% | 0.067% | 0.086% | 0.614% |
| pWISE10649 | 630 | 0.001% | 0.030% | 0.022% | 0.054% | 0.381% |
| pWISE10650 | 631 | 0.003% | 0.055% | 0.071% | 0.113% | 0.712% |
| pWISE10651 | 632 | 0.002% | 0.078% | 0.088% | 0.121% | 0.747% |
| pWISE10652 | 633 | 0.015% | 0.036% | 0.066% | 0.110% | 0.664% |
| pWISE10658 | 639 | 0.003% | 0.239% | 0.705% | 1.301% | 1.542% |
| pWISE10653 | 634 | 0.002% | 0.236% | 0.213% | 0.319% | 1.124% |
| pWISE10654 | 635 | 0.012% | 0.104% | 0.169% | 0.318% | 1.583% |
| pWISE10655 | 636 | 0.003% | 0.161% | 0.230% | 0.380% | 1.690% |
| pWISE10656 | 637 | 0.002% | 0.109% | 0.164% | 0.320% | 1.422% |
| pWISE10657 | 638 | 0.002% | 0.080% | 0.097% | 0.247% | 0.974% |
| | | Standard deviation of the average G to A editing percent at the | | | | |
| | | nucleotides numbered according to the position in the PWsp3630 | | | | |
| | Protein | spacer sequence on the opposite, target genomic DNA strand | | | | |
| pWISE | SEQ ID NO: | G.16 | G.17 | G.18 | G.21 | G.22 |
| pWISE121 | 56 | 0.099% | 0.128% | 0.212% | 0.151% | 0.180% |
| pWISE9120 | 453 | 0.318% | 0.213% | 0.188% | 0.186% | 0.192% |
| pWISE10648 | 629 | 0.477% | 0.295% | 0.268% | 0.304% | 0.322% |
| pWISE10649 | 630 | 0.371% | 0.297% | 0.263% | 0.270% | 0.265% |
| pWISE10650 | 631 | 0.757% | 0.588% | 0.548% | 0.506% | 0.533% |
| pWISE10651 | 632 | 0.891% | 0.750% | 0.699% | 0.588% | 0.589% |
| pWISE10652 | 633 | 0.787% | 0.629% | 0.622% | 0.622% | 0.619% |
| pWISE10658 | 639 | 1.486% | 0.987% | 0.806% | 0.663% | 0.672% |
| pWISE10653 | 634 | 1.169% | 0.857% | 0.725% | 0.543% | 0.553% |
| pWISE10654 | 635 | 1.726% | 1.359% | 1.301% | 1.286% | 1.303% |
| pWISE10655 | 636 | 2.219% | 1.743% | 1.647% | 1.501% | 1.551% |
| pWISE10656 | 637 | 2.024% | 1.616% | 1.530% | 1.413% | 1.538% |
| pWISE10657 | 638 | 1.793% | 1.343% | 1.230% | 1.127% | 1.256% |

### Example 12

Circular permutants of an enzymatically inactive LbCas 12a enzyme (dLbCas12a (SEQ ID NO: 59)) were tested for adenine base editing by fusing an adenine deaminase to either the N-terminus or C-terminus of the circular permutant using a linker to provide a fusion protein. The tested linkers included a GS-XTEN-GS linker (SEQ ID NO:30); an XTEN linker (SEQ ID NO: 29); a four amino acid glycine-serine (4XGS) linker of SGGS (SEQ ID NO:26); a six amino acid glycine serine (6XGS) linker of (SGS)ₙ wherein n is 2 (SEQ ID NO:869); an eight amino acid glycine-serine (8XGS) linker of (SGGS)ₙ wherein n is 2 (SEQ ID NO:870); and a twelve amino acid glycine-serine (12XGS) linker of (SGGS)ₙ wherein n is 3. The fusion proteins and control (SEQ ID NO:56) are listed in Table 97 and the targets are in Table 98.

TABLE 97
Tested constructs.

| | Vector SEQ | DNA SEQ | Protein SEQ |
|---|---|---|---|
| pWISE | ID NO: | ID NO: | ID NO: |
| pWISE121 | 259 | 246 | 56 |
| pWISE9205 | 532 | 515 | 498 |
| pWISE9193 | 520 | 503 | 486 |
| pWISE10616 | 671 | 640 | 609 |
| pWISE10617 | 672 | 641 | 610 |
| pWISE10618 | 673 | 642 | 611 |
| pWISE10619 | 674 | 643 | 612 |
| pWISE10620 | 675 | 644 | 613 |
| pWISE9199 | 526 | 509 | 492 |
| pWISE10621 | 676 | 645 | 614 |
| pWISE10622 | 677 | 646 | 615 |
| pWISE10623 | 678 | 647 | 616 |
| pWISE10624 | 679 | 648 | 617 |
| pWISE10625 | 680 | 649 | 618 |
| pWISE9201 | 528 | 511 | 494 |
| pWISE10626 | 681 | 650 | 619 |
| pWISE10627 | 682 | 651 | 620 |
| pWISE10628 | 683 | 652 | 621 |
| pWISE10629 | 684 | 653 | 622 |
| pWISE10630 | 685 | 654 | 623 |

TABLE 98
Targets

| | Target | | Spacer | | Vector |
|---|---|---|---|---|---|
| Target Nucleic | SEQ ID | | SEQ ID | | SEQ ID |
| Acid | NO: | Spacer | NO: | pWISE | NO: |
| human DNMT1 | 272 | PWsp143 | 275 | pWISE264 | 278 |
| human RNF2 | 274 | PWsp453 | 277 | pWISE882 | 280 |
| human RNF2 | 274 | PWsp454 | 865 | pWISE883 | 867 |
| human RNF2 | 274 | PWsp455 | 866 | pWISE884 | 868 |

The fusion proteins and control were tested for their ability to perform base editing at four different sites in two target nucleic acids (SEQ ID NOs: 272 and 274) in HEK293T cells using four spacers (SEQ ID NOs: 275, 277, 865, and 866). A single biological replicate and two technical replicates were tested. HEK293T cells were seeded into 48-well collagen-coated plates (Corning) in the absence of an antibiotic using DMEM media. At 70-80% confluency, 10 cells were transfected with 1.5 μL of LTX (ThermoFisher Scientific) using 500 ng of the control or fusion protein plasmid and 500 ng of guide RNA plasmid according to manufacturer's protocol. After 3 days, the cells were lysed with a crude extraction method using TritonX buffer. Each control or fusion protein was scored based on the precise base pair editing in the DNMT1 and RNF2 genes using the guide RNAs (SEQ ID NOs: 275, 277, 865, and 866). Low, background levels of INDEL formation were seen, which was expected from using a dead LbCas12a. The results are provided in Tables 99-106. The values in Tables 99-106 that are below 0.1% are considered to be in the noise of the instrument (below the limit of detection) and are not indicative of editing. Values that are between 0.1% and 0.5% indicate that editing is present in the experiment at the specified location, but the assay is not sensitive enough to accurately quantify the amount of base editing. Similar to what was demonstrated in Example 10 for cytosine base editing, as seen in Tables 99-106, the fusion proteins provided efficient A to G editing over a wide editing window and changing the length and/or type of the linker between the adenine deaminase and the circular permutant did not significantly modify the editing window or efficiency at positions shown to be highly edited with the control. In addition, the fusion proteins containing shorter linkers (e.g., 6XGS linker) were found to have improved A to G editing towards the 3′ end of the spacer (e.g., at about the last 11-23 nucleotides of the spacer sequence).

TABLE 99

| | | Average A to G editing percent at the nucleotides | | | |
|---|---|---|---|---|---|
| | | numbered according to the position in the PWsp143 spacer | | | |
| | Protein SEQ | sequence on the opposite, target genomic DNA strand | | | |
| pWISE | ID NO: | A.5 | A.8 | A.12 | A.21 |
| pWISE121 | 56 | 0.049% | 0.454% | 0.361% | 0.141% |
| pWISE9205 | 498 | 0.017% | 10.881% | 6.116% | 0.021% |
| pWISE9193 | 486 | 0.008% | 6.988% | 8.961% | 0.335% |
| pWISE10616 | 609 | 0.012% | 5.531% | 7.466% | 0.292% |
| pWISE10617 | 610 | 0.010% | 5.564% | 8.150% | 0.225% |
| pWISE10618 | 611 | 0.016% | 9.120% | 10.199% | 0.470% |
| pWISE10619 | 612 | 0.007% | 10.127% | 10.751% | 0.416% |
| pWISE10620 | 613 | No data | No data | No data | No data |
| pWISE9199 | 492 | 0.012% | 11.264% | 7.690% | 0.096% |
| pWISE10621 | 614 | 0.015% | 12.532% | 9.651% | 0.152% |
| pWISE10622 | 615 | 0.024% | 12.238% | 10.625% | 0.204% |
| pWISE10623 | 616 | 0.027% | 9.530% | 10.037% | 0.606% |
| pWISE10624 | 617 | 0.018% | 6.966% | 9.839% | 0.667% |
| pWISE10625 | 618 | 0.018% | 6.658% | 9.787% | 0.725% |
| pWISE9201 | 494 | 0.005% | 9.097% | 8.061% | 0.131% |
| pWISE10626 | 619 | 0.015% | 13.655% | 11.997% | 0.058% |
| pWISE10627 | 620 | 0.015% | 10.271% | 9.656% | 0.045% |
| pWISE10628 | 621 | 0.015% | 12.506% | 12.122% | 0.102% |
| pWISE10629 | 622 | 0.014% | 11.561% | 11.607% | 0.056% |
| pWISE10630 | 623 | 0.016% | 8.140% | 9.604% | 0.055% |

TABLE 100

| | | Standard deviation of the average A to G editing percent at the | | | |
|---|---|---|---|---|---|
| | | nucleotides numbered according to the position in the PWsp143 | | | |
| | Protein SEQ | spacer sequence on the opposite, target genomic DNA strand | | | |
| pWISE | ID NO: | A.5 | A.8 | A.12 | A.21 |
| pWISE121 | 56 | 0.019% | 0.539% | 0.180% | 0.098% |
| pWISE9205 | 498 | 0.000% | 2.037% | 1.549% | 0.009% |
| pWISE9193 | 486 | 0.000% | 1.580% | 2.439% | 0.170% |
| pWISE10616 | 609 | 0.000% | 0.515% | 1.132% | 0.032% |
| pWISE10617 | 610 | 0.004% | 2.072% | 2.689% | 0.051% |
| pWISE10618 | 611 | 0.001% | 1.999% | 2.508% | 0.154% |
| pWISE10619 | 612 | 0.005% | 1.253% | 1.351% | 0.235% |
| pWISE10620 | 613 | No data | No data | No data | No data |
| pWISE9199 | 492 | 0.007% | 1.294% | 1.424% | 0.041% |
| pWISE10621 | 614 | No data | No data | No data | No data |
| pWISE10622 | 615 | 0.047% | 0.070% | 0.428% | 0.077% |
| pWISE10623 | 616 | 0.006% | 0.490% | 0.628% | 0.164% |
| pWISE10624 | 617 | 0.006% | 0.258% | 0.734% | 0.124% |
| pWISE10625 | 618 | 0.021% | 0.114% | 0.193% | 0.029% |
| pWISE9201 | 494 | 0.001% | 0.459% | 0.216% | 0.004% |
| pWISE10626 | 619 | 0.024% | 0.428% | 0.010% | 0.099% |
| pWISE10627 | 620 | 0.017% | 0.026% | 0.048% | 0.008% |
| pWISE10628 | 621 | 0.007% | 1.453% | 1.913% | 0.025% |
| pWISE10629 | 622 | 0.004% | 0.131% | 0.187% | 0.034% |
| pWISE10630 | 623 | 0.004% | 0.062% | 0.413% | 0.053% |

TABLE 101

| | | Average A to G editing percent at the nucleotides numbered | | | | |
|---|---|---|---|---|---|---|
| | | according to the position in the PWsp453 spacer sequence on | | | | |
| | Protein | the opposite, target genomic DNA strand | | | | |
| pWISE | SEQ ID NO: | A.2 | A.5 | A.9 | A.11 | A.12 |
| pWISE121 | 56 | 0.138% | 0.035% | 1.161% | 0.878% | 0.777% |
| pWISE9205 | 498 | 0.127% | 0.012% | 14.129% | 7.618% | 1.198% |
| pWISE9193 | 486 | 0.012% | 0.018% | 11.629% | 8.829% | 3.176% |
| pWISE10616 | 609 | 0.010% | 0.015% | 6.160% | 5.140% | 2.609% |
| pWISE10617 | 610 | 0.012% | 0.021% | 12.117% | 10.179% | 4.984% |
| pWISE10618 | 611 | 0.034% | 0.007% | 12.573% | 11.037% | 7.749% |
| pWISE10619 | 612 | 0.018% | 0.019% | 14.205% | 12.317% | 9.538% |
| pWISE10620 | 613 | 0.015% | 0.005% | 13.150% | 11.442% | 8.542% |
| pWISE9199 | 492 | 0.022% | 0.009% | 19.128% | 13.423% | 6.854% |
| pWISE10621 | 614 | 0.007% | 0.010% | 13.483% | 9.736% | 5.018% |
| pWISE10622 | 615 | 0.054% | 0.005% | 15.184% | 12.268% | 7.328% |
| pWISE10623 | 616 | 0.043% | 0.000% | 14.828% | 11.765% | 7.585% |
| pWISE10624 | 617 | 0.023% | 0.000% | 13.658% | 10.912% | 7.263% |
| pWISE10625 | 618 | 0.024% | 0.012% | 14.390% | 11.538% | 7.500% |
| pWISE9201 | 494 | 0.014% | 0.014% | 12.161% | 8.762% | 3.396% |
| pWISE10626 | 619 | 0.019% | 0.015% | 15.661% | 12.756% | 6.276% |
| pWISE10627 | 620 | 0.018% | 0.024% | 10.200% | 9.008% | 4.639% |
| pWISE10628 | 621 | 0.000% | 0.000% | 14.353% | 12.995% | 6.304% |
| pWISE10629 | 622 | 0.044% | 0.015% | 16.251% | 14.846% | 7.550% |
| pWISE10630 | 623 | 0.028% | 0.009% | 15.393% | 14.333% | 6.270% |

| | |
|---|---|
| | Average A to G editing percent at the nucleotides |
| | numbered according to the position in the PWsp453 spacer |
| Protein | sequence on the opposite, target genomic DNA strand |

| pWISE | SEQ ID NO: | A.15 | A.16 | A.18 | A.23 |
|---|---|---|---|---|---|
| pWISE121 | 56 | 0.552% | 0.743% | 1.054% | 0.165% |
| pWISE9205 | 498 | 0.106% | 0.249% | 1.538% | 0.017% |
| pWISE9193 | 486 | 0.436% | 1.612% | 7.737% | 0.024% |
| pWISE10616 | 609 | 0.471% | 1.094% | 4.119% | 0.010% |
| pWISE10617 | 610 | 0.815% | 2.195% | 8.928% | 0.005% |
| pWISE10618 | 611 | 1.779% | 4.469% | 10.096% | 0.112% |
| pWISE10619 | 612 | 2.408% | 5.715% | 11.579% | 0.090% |
| pWISE10620 | 613 | 2.315% | 5.327% | 10.147% | 0.094% |
| pWISE9199 | 492 | 0.746% | 3.337% | 9.253% | 0.033% |
| pWISE10621 | 614 | 0.445% | 2.418% | 6.582% | 0.041% |
| pWISE10622 | 615 | 0.922% | 4.262% | 9.635% | 0.029% |
| pWISE10623 | 616 | 1.863% | 6.573% | 12.093% | 0.091% |
| pWISE10624 | 617 | 2.155% | 6.661% | 11.814% | 0.100% |
| pWISE10625 | 618 | 2.420% | 7.991% | 13.011% | 0.105% |
| pWISE9201 | 494 | 0.361% | 0.851% | 2.037% | 0.006% |
| pWISE10626 | 619 | 0.378% | 1.315% | 3.752% | 0.010% |
| pWISE10627 | 620 | 0.294% | 0.977% | 3.272% | 0.006% |
| pWISE10628 | 621 | 0.745% | 1.508% | 4.553% | 0.000% |
| pWISE10629 | 622 | 0.681% | 2.299% | 5.520% | 0.005% |
| pWISE10630 | 623 | 0.817% | 2.121% | 5.463% | 0.019% |

TABLE 102

| | | Standard deviation of the average A to G editing percent at the | | | | |
|---|---|---|---|---|---|---|
| | | nucleotides numbered according to the position in the PWsp453 | | | | |
| | Protein | spacer sequence on the opposite, target genomic DNA strand | | | | |
| pWISE | SEQ ID NO: | A.2 | A.5 | A.9 | A.11 | A.12 |
| pWISE121 | 56 | 0.042% | 0.014% | 1.113% | 1.125% | 0.864% |
| pWISE9205 | 498 | 0.152% | 0.006% | 5.123% | 3.289% | 1.052% |
| pWISE9193 | 486 | No data | No data | No data | No data | No data |
| pWISE10616 | 609 | No data | No data | No data | No data | No data |
| pWISE10617 | 610 | 0.010% | 0.009% | 0.247% | 0.109% | 0.208% |
| pWISE10618 | 611 | No data | No data | No data | No data | No data |
| pWISE10619 | 612 | 0.011% | 0.015% | 0.325% | 0.280% | 0.196% |
| pWISE10620 | 613 | 0.021% | 0.007% | 2.091% | 2.091% | 1.872% |
| pWISE9199 | 492 | 0.025% | 0.002% | 0.832% | 0.263% | 0.241% |
| pWISE10621 | 614 | 0.010% | 0.000% | 2.954% | 2.654% | 1.135% |
| pWISE10622 | 615 | No data | No data | No data | No data | No data |
| pWISE10623 | 616 | 0.010% | 0.000% | 0.400% | 0.848% | 0.242% |
| pWISE10624 | 617 | 0.026% | 0.000% | 0.037% | 0.239% | 0.253% |
| pWISE10625 | 618 | 0.013% | 0.003% | 0.836% | 0.760% | 0.983% |
| pWISE9201 | 494 | 0.002% | 0.002% | 0.423% | 0.252% | 0.793% |
| pWISE10626 | 619 | 0.018% | 0.015% | 0.492% | 0.168% | 0.206% |
| pWISE10627 | 620 | No data | No data | No data | No data | No data |
| pWISE10628 | 621 | No data | No data | No data | No data | No data |
| pWISE10629 | 622 | 0.049% | 0.008% | 2.848% | 2.553% | 1.124% |
| pWISE10630 | 623 | No data | No data | No data | No data | No data |

| | | Standard deviation of the average A to G editing percent at the | | | |
|---|---|---|---|---|---|
| | | nucleotides numbered according to the position in the PWsp453 | | | |
| | Protein | spacer sequence on the opposite, target genomic DNA strand | | | |
| pWISE | SEQ ID NO: | A.15 | A.16 | A.18 | A.23 |
| pWISE121 | 56 | 0.312% | 0.453% | 0.869% | 0.036% |
| pWISE9205 | 498 | 0.150% | 0.342% | 1.025% | 0.013% |
| pWISE9193 | 486 | No data | No data | No data | No data |
| pWISE10616 | 609 | No data | No data | No data | No data |
| pWISE10617 | 610 | 0.030% | 0.048% | 0.053% | 0.007% |
| pWISE10618 | 611 | No data | No data | No data | No data |
| pWISE10619 | 612 | 0.055% | 0.067% | 0.517% | 0.057% |
| pWISE10620 | 613 | 0.526% | 0.971% | 2.537% | 0.059% |
| pWISE9199 | 492 | 0.097% | 0.281% | 0.124% | 0.010% |
| pWISE10621 | 614 | 0.135% | 0.515% | 1.823% | 0.023% |
| pWISE10622 | 615 | No data | No data | No data | No data |
| pWISE10623 | 616 | 0.076% | 0.261% | 0.259% | 0.047% |
| pWISE10624 | 617 | 0.014% | 0.079% | 0.177% | 0.007% |
| pWISE10625 | 618 | 0.469% | 0.685% | 0.737% | 0.040% |
| pWISE9201 | 494 | 0.236% | 0.161% | 0.716% | 0.009% |
| pWISE10626 | 619 | 0.028% | 0.089% | 0.395% | 0.005% |
| pWISE10627 | 620 | No data | No data | No data | No data |
| pWISE10628 | 621 | No data | No data | No data | No data |
| pWISE10629 | 622 | 0.261% | 0.362% | 1.299% | 0.007% |
| pWISE10630 | 623 | No data | No data | No data | No data |

TABLE 103

| | | Average A to G editing percent at the nucleotides | | | |
|---|---|---|---|---|---|
| | | numbered according to the position in the PWsp454 spacer | | | |
| | Protein SEQ | sequence on the opposite, target genomic DNA strand | | | |
| pWISE | ID NO: | A.2 | A.9 | A.11 | A.23 |
| pWISE121 | 56 | 0.015% | 0.119% | 0.104% | 0.327% |
| pWISE9205 | 498 | 0.002% | 12.330% | 13.245% | 0.013% |
| pWISE9193 | 486 | 0.003% | 8.383% | 12.081% | 0.077% |
| pWISE10616 | 609 | 0.003% | 5.482% | 7.900% | 0.025% |
| pWISE10617 | 610 | 0.009% | 8.076% | 11.812% | 0.055% |
| pWISE10618 | 611 | 0.001% | 10.544% | 12.643% | 0.136% |
| pWISE10619 | 612 | 0.017% | 10.483% | 12.245% | 0.017% |
| pWISE10620 | 613 | 0.000% | 10.423% | 11.549% | 0.037% |
| pWISE9199 | 492 | 0.000% | 11.850% | 13.364% | 0.028% |
| pWISE10621 | 614 | 0.000% | 9.153% | 10.414% | 0.013% |
| pWISE10622 | 615 | 0.005% | 9.612% | 11.191% | 0.026% |
| pWISE10623 | 616 | 0.004% | 7.718% | 10.349% | 0.019% |
| pWISE10624 | 617 | 0.004% | 7.277% | 10.815% | 0.031% |
| pWISE10625 | 618 | 0.000% | 7.675% | 12.356% | 0.028% |
| pWISE9201 | 494 | 0.004% | 7.780% | 8.839% | 0.004% |
| pWISE10626 | 619 | 0.000% | 3.932% | 3.932% | 0.000% |
| pWISE10627 | 620 | No data | No data | No data | No data |
| pWISE10628 | 621 | 0.000% | 8.934% | 9.852% | 0.018% |
| pWISE10629 | 622 | 0.000% | 10.551% | 11.525% | 0.005% |
| pWISE10630 | 623 | 0.009% | 8.006% | 8.940% | 0.004% |

TABLE 104

| | | Standard deviation of the average A to G editing percent at the | | | |
|---|---|---|---|---|---|
| | | nucleotides numbered according to the position in the PWsp454 | | | |
| | Protein SEQ | spacer sequence on the opposite, target genomic DNA strand | | | |
| pWISE | ID NO: | A.2 | A.9 | A.11 | A.23 |
| pWISE121 | 56 | No data | No data | No data | No data |
| pWISE9205 | 498 | 0.003% | 0.209% | 0.283% | 0.009% |
| pWISE9193 | 486 | 0.005% | 0.529% | 0.512% | 0.071% |
| pWISE10616 | 609 | 0.004% | 0.425% | 0.728% | 0.008% |
| pWISE10617 | 610 | 0.006% | 1.459% | 1.746% | 0.020% |
| pWISE10618 | 611 | 0.002% | 0.580% | 0.671% | 0.062% |
| pWISE10619 | 612 | No data | No data | No data | No data |
| pWISE10620 | 613 | 0.000% | 0.897% | 0.685% | 0.011% |
| pWISE9199 | 492 | 0.000% | 0.013% | 0.030% | 0.005% |
| pWISE10621 | 614 | 0.000% | 1.657% | 1.924% | 0.006% |
| pWISE10622 | 615 | 0.008% | 1.191% | 1.694% | 0.001% |
| pWISE10623 | 616 | 0.006% | 1.038% | 1.414% | 0.016% |
| pWISE10624 | 617 | 0.006% | 0.629% | 0.796% | 0.010% |
| pWISE10625 | 618 | 0.000% | 0.135% | 0.211% | 0.019% |
| pWISE9201 | 494 | 0.006% | 1.276% | 0.968% | 0.006% |
| pWISE10626 | 619 | No data | No data | No data | No data |
| pWISE10627 | 620 | No data | No data | No data | No data |
| pWISE10628 | 621 | No data | No data | No data | No data |
| pWISE10629 | 622 | No data | No data | No data | No data |
| pWISE10630 | 623 | No data | No data | No data | No data |

TABLE 105

| | | Average A to G editing percent at the nucleotides numbered | | | | | |
|---|---|---|---|---|---|---|---|
| | | according to the position in the PWsp455 spacer sequence on | | | | | |
| | Protein | the opposite, target genomic DNA strand | | | | | |
| pWISE | SEQ ID NO: | A.2 | A.3 | A.8 | A.9 | A.10 | A.11 |
| pWISE121 | 56 | 0.166% | 0.275% | 0.544% | 1.043% | 0.179% | 0.326% |
| pWISE9205 | 498 | 0.005% | 0.012% | 7.282% | 5.673% | 2.411% | 4.140% |
| pWISE9193 | 486 | No data | No data | No data | No data | No data | No data |
| pWISE10616 | 609 | No data | No data | No data | No data | No data | No data |
| pWISE10617 | 610 | 0.017% | 0.025% | 7.855% | 5.408% | 4.640% | 10.816% |
| pWISE10618 | 611 | 0.008% | 0.008% | 10.980% | 9.523% | 7.365% | 12.119% |
| pWISE10619 | 612 | No data | No data | No data | No data | No data | No data |
| pWISE10620 | 613 | 0.011% | 0.057% | 15.583% | 12.660% | 10.711% | 15.713% |
| pWISE9199 | 492 | 0.012% | 0.000% | 10.433% | 8.274% | 3.945% | 7.834% |
| pWISE10621 | 614 | 0.003% | 0.000% | 9.054% | 7.638% | 3.775% | 7.118% |
| pWISE10622 | 615 | 0.002% | 0.002% | 9.651% | 8.163% | 4.494% | 8.119% |
| pWISE10623 | 616 | 0.000% | 0.014% | 5.673% | 4.674% | 2.372% | 5.963% |
| pWISE10624 | 617 | 0.018% | 0.012% | 4.368% | 3.541% | 2.043% | 5.177% |
| pWISE10625 | 618 | 0.016% | 0.005% | 3.903% | 3.005% | 1.708% | 4.842% |
| pWISE9201 | 494 | 0.000% | 0.009% | 10.929% | 7.710% | 4.491% | 8.909% |
| pWISE10626 | 619 | 0.015% | 0.000% | 12.765% | 10.769% | 7.658% | 11.621% |
| pWISE10627 | 620 | 0.007% | 0.003% | 8.499% | 8.510% | 6.716% | 9.321% |
| pWISE10628 | 621 | 0.005% | 0.010% | 9.154% | 10.779% | 8.955% | 11.099% |
| pWISE10629 | 622 | 0.037% | 0.004% | 7.896% | 11.576% | 10.312% | 11.996% |
| pWISE10630 | 623 | 0.008% | 0.012% | 3.815% | 9.166% | 8.909% | 9.687% |

| | | Average A to G editing percent at the nucleotides numbered | | | | | |
|---|---|---|---|---|---|---|---|
| | | according to the position in the PWsp455 spacer sequence on | | | | | |
| | Protein | the opposite, target genomic DNA strand | | | | | |
| pWISE | SEQ ID NO: | A.15 | A.16 | A.17 | A.19 | A.21 | A.22 |
| pWISE121 | 56 | 0.941% | 0.614% | 0.442% | 0.557% | 0.102% | 0.416% |
| pWISE9205 | 498 | 0.883% | 0.040% | 0.007% | 0.260% | 0.012% | 0.100% |
| pWISE9193 | 486 | No data | No data | No data | No data | No data | No data |
| pWISE10616 | 609 | No data | No data | No data | No data | No data | No data |
| pWISE10617 | 610 | 2.885% | 0.084% | 0.084% | 0.768% | 0.337% | 0.059% |
| pWISE10618 | 611 | 3.026% | 0.135% | 0.135% | 1.330% | 0.072% | 0.000% |
| pWISE10619 | 612 | No data | No data | No data | No data | No data | No data |
| pWISE10620 | 613 | 4.424% | 0.206% | 0.248% | 2.383% | 0.065% | 0.070% |
| pWISE9199 | 492 | 0.958% | 0.015% | 0.031% | 0.219% | 0.040% | 0.020% |
| pWISE10621 | 614 | 1.091% | 0.013% | 0.045% | 0.218% | 0.011% | 0.012% |
| pWISE10622 | 615 | 1.663% | 0.018% | 0.043% | 0.349% | 0.009% | 0.030% |
| pWISE10623 | 616 | 2.550% | 0.023% | 0.030% | 0.525% | 0.033% | 0.033% |
| pWISE10624 | 617 | 3.218% | 0.090% | 0.090% | 0.659% | 0.072% | 0.018% |
| pWISE10625 | 618 | 3.382% | 0.107% | 0.083% | 0.561% | 0.053% | 0.039% |
| pWISE9201 | 494 | 0.938% | 0.225% | 0.063% | 0.126% | 0.000% | 0.054% |
| pWISE10626 | 619 | 1.097% | 0.056% | 0.072% | 0.222% | 0.053% | 0.054% |
| pWISE10627 | 620 | 1.135% | 0.047% | 0.056% | 0.345% | 0.026% | 0.034% |
| pWISE10628 | 621 | 1.640% | 0.075% | 0.069% | 0.552% | 0.026% | 0.068% |
| pWISE10629 | 622 | 2.282% | 0.125% | 0.165% | 0.638% | 0.024% | 0.069% |
| pWISE10630 | 623 | 2.195% | 0.119% | 0.104% | 0.573% | 0.023% | 0.036% |

TABLE 106

| | | Standard deviation of the average A to G editing percent at the | | | | | |
|---|---|---|---|---|---|---|---|
| | | nucleotides numbered according to the position in the PWsp455 | | | | | |
| | Protein | spacer sequence on the opposite, target genomic DNA strand | | | | | |
| pWISE | SEQ ID NO: | A.2 | A.3 | A.8 | A.9 | A.10 | A.11 |
| pWISE121 | 56 | No data | No data | No data | No data | No data | No data |
| pWISE9205 | 498 | No data | No data | No data | No data | No data | No data |
| pWISE9193 | 486 | No data | No data | No data | No data | No data | No data |
| pWISE10616 | 609 | No data | No data | No data | No data | No data | No data |
| pWISE10617 | 610 | No data | No data | No data | No data | No data | No data |
| pWISE10618 | 611 | No data | No data | No data | No data | No data | No data |
| pWISE10619 | 612 | No data | No data | No data | No data | No data | No data |
| pWISE10620 | 613 | 0.007% | 0.001% | 0.510% | 0.374% | 0.563% | 0.648% |
| pWISE9199 | 492 | 0.006% | 0.000% | 0.185% | 0.115% | 0.495% | 0.260% |
| pWISE10621 | 614 | 0.005% | 0.000% | 0.698% | 0.477% | 0.379% | 0.613% |
| pWISE10622 | 615 | 0.003% | 0.003% | 0.708% | 0.663% | 0.703% | 1.611% |
| pWISE10623 | 616 | 0.000% | 0.004% | 0.097% | 0.096% | 0.339% | 0.362% |
| pWISE10624 | 617 | No data | No data | No data | No data | No data | No data |
| pWISE10625 | 618 | 0.001% | 0.007% | 0.002% | 0.037% | 0.048% | 0.079% |
| pWISE9201 | 494 | No data | No data | No data | No data | No data | No data |
| pWISE10626 | 619 | 0.011% | 0.000% | 0.739% | 0.239% | 0.334% | 0.282% |
| pWISE10627 | 620 | 0.001% | 0.005% | 0.332% | 0.222% | 0.114% | 0.209% |
| pWISE10628 | 621 | 0.007% | 0.006% | 0.453% | 0.698% | 0.519% | 0.284% |
| pWISE10629 | 622 | 0.007% | 0.006% | 0.089% | 0.483% | 0.440% | 0.148% |
| pWISE10630 | 623 | 0.000% | 0.005% | 0.417% | 0.019% | 0.051% | 0.260% |
| | | Standard deviation of the average A to G editing percent at the | | | | | |
| | | nucleotides numbered according to the position in the PWsp455 | | | | | |
| | Protein | spacer sequence on the opposite, target genomic DNA strand | | | | | |
| pWISE | SEQ ID NO: | A.15 | A.16 | A.17 | A.19 | A.21 | A.22 |
| pWISE121 | 56 | No data | No data | No data | No data | No data | No data |
| pWISE9205 | 498 | No data | No data | No data | No data | No data | No data |
| pWISE9193 | 486 | No data | No data | No data | No data | No data | No data |
| pWISE10616 | 609 | No data | No data | No data | No data | No data | No data |
| pWISE10617 | 610 | No data | No data | No data | No data | No data | No data |
| pWISE10618 | 611 | No data | No data | No data | No data | No data | No data |
| pWISE10619 | 612 | No data | No data | No data | No data | No data | No data |
| pWISE10620 | 613 | 0.074% | 0.107% | 0.052% | 0.182% | 0.001% | 0.027% |
| pWISE9199 | 492 | 0.032% | 0.021% | 0.013% | 0.037% | 0.035% | 0.018% |
| pWISE10621 | 614 | 0.078% | 0.018% | 0.016% | 0.068% | 0.004% | 0.007% |
| pWISE10622 | 615 | 0.019% | 0.014% | 0.011% | 0.082% | 0.000% | 0.004% |
| pWISE10623 | 616 | 0.095% | 0.020% | 0.018% | 0.030% | 0.022% | 0.038% |
| pWISE10624 | 617 | No data | No data | No data | No data | No data | No data |
| pWISE10625 | 618 | 0.004% | 0.020% | 0.032% | 0.108% | 0.011% | 0.009% |
| pWISE9201 | 494 | No data | No data | No data | No data | No data | No data |
| pWISE10626 | 619 | 0.206% | 0.007% | 0.014% | 0.042% | 0.002% | 0.029% |
| pWISE10627 | 620 | 0.093% | 0.009% | 0.013% | 0.063% | 0.002% | 0.037% |
| pWISE10628 | 621 | 0.095% | 0.030% | 0.022% | 0.032% | 0.012% | 0.040% |
| pWISE10629 | 622 | 0.085% | 0.024% | 0.080% | 0.016% | 0.034% | 0.008% |
| pWISE10630 | 623 | 0.126% | 0.009% | 0.024% | 0.135% | 0.001% | 0.017% |

### Example 13

Circular permutants of an enzymatically inactive LbCas 12a enzyme (dLbCas12a (SEQ ID NO: 59)) were tested for adenine base editing by fusing an adenine deaminase to either the N-terminus or C-terminus of the circular permutant using a linker (e.g., GS-XTEN-GS linker (SEQ ID NO:30)) to provide a fusion protein. The fusion proteins and control (SEQ ID NO: 56) are listed in Table 107 and the targets are in Table 108.

TABLE 107
Tested constructs.

| | Vector SEQ | DNA SEQ | Protein SEQ |
|---|---|---|---|
| pWISE | ID NO: | ID NO: | ID NO: |
| pWISE121 | 259 | 246 | 56 |
| pWISE2918 | 534 | 517 | 498 |
| pWISE4922 | 535 | 518 | 501 |
| pWISE6854 | 536 | 519 | 502 |
| pWISE9193 | 520 | 503 | 486 |
| pWISE9194 | 521 | 504 | 487 |
| pWISE9195 | 522 | 505 | 488 |
| pWISE9196 | 523 | 506 | 489 |
| pWISE9197 | 524 | 507 | 490 |
| pWISE9198 | 525 | 508 | 491 |
| pWISE9199 | 526 | 509 | 492 |
| pWISE9200 | 527 | 510 | 493 |
| pWISE9201 | 528 | 511 | 494 |
| pWISE9202 | 529 | 512 | 495 |
| pWISE9203 | 530 | 513 | 496 |
| pWISE9204 | 531 | 514 | 497 |
| pWISE9205 | 532 | 515 | 498 |
| pWISE9206 | 533 | 516 | 499 |

TABLE 108
Targets

| Target Nucleic | Target SEQ | | Spacer SEQ | | Vector SEQ |
|---|---|---|---|---|---|
| Acid | ID NO: | Spacer | ID NO: | pWISE | ID NO: |
| human DNMT1 | 272 | PWsp143 | 275 | pWISE264 | 278 |
| human RNF2 | 274 | PWsp453 | 277 | pWISE882 | 280 |
| human RNF2 | 274 | PWsp454 | 865 | pWISE883 | 867 |
| human RNF2 | 274 | PWsp455 | 866 | pWISE884 | 868 |

The fusion proteins and controls were tested for their ability to perform base editing at four different sites in two target nucleic acids (SEQ ID NOs: 272 and 274) in HEK293T cells using four spacers (SEQ ID NOs: 275, 277, 865, and 866). A single biological replicate and two technical replicates were tested. HEK293T cells were seeded into 48-well collagen-coated plates (Corning) in the absence of an antibiotic using DMEM media. At 70-80% confluency, cells were transfected with 1.5 μL of LTX (ThermoFisher Scientific) using 500 ng of the control or fusion protein plasmid and 500 ng of guide RNA plasmid according to manufacturer's protocol. After 3 days, the cells were lysed with a crude extraction method using TritonX buffer. Each control or fusion protein was scored based on the precise base pair editing in the DNMT1 and RNF2 genes using the guide RNAs (SEQ ID NOs: 275, 277, 865, and 866). Low, background levels of INDEL formation were seen, which was expected from using a dead LbCas12a. The results are provided in Tables 109-116. The values in Tables 109-116 that are below 0.1% are considered to be in the noise of the instrument (below the limit of detection) and are not indicative of editing. Values that are between 0.1% and 0.5% indicate that editing is present in the experiment at the specified location, but the assay is not sensitive enough to accurately quantify the amount of base editing. As seen in Tables 109-116, the fusion proteins provided efficient A to G editing over a wide window and specifically had improved editing at positions at about 11-23 of the target sequence (e.g., towards the 3′ end of the spacer sequence such as about nucleotides 11-23).

TABLE 109

| | | Average A to G editing percent at the nucleotides numbered | | | | | | |
|---|---|---|---|---|---|---|---|---|
| | | according to the position in the PWsp143 spacer sequence on the | | | | | | |
| | Protein | opposite, target genomic DNA strand | | | | | | |
| pWISE | SEQ ID NO: | A.-5 | A.-4 | A.5 | A.8 | A.12 | A.21 | A.29 |
| pWISE121 | 56 | 0.040% | 0.027% | 0.074% | 0.041% | 0.125% | 0.178% | 0.068% |
| pWISE2918 | 498 | 0.011% | 0.007% | 0.016% | 11.995% | 6.888% | 0.022% | 0.003% |
| pWISE4922 | 501 | 0.007% | 0.008% | 0.012% | 9.682% | 5.775% | 0.015% | 0.005% |
| pWISE6854 | 502 | 0.008% | 0.009% | 0.003% | 9.957% | 8.127% | 0.017% | 0.002% |
| pWISE9193 | 486 | 0.011% | 0.005% | 0.006% | 7.469% | 9.656% | 0.428% | 0.006% |
| pWISE9194 | 487 | 0.007% | 0.009% | 0.005% | 0.263% | 0.743% | 0.011% | 0.004% |
| pWISE9195 | 488 | 0.009% | 0.009% | 0.005% | 6.490% | 4.719% | 0.116% | 0.003% |
| pWISE9196 | 489 | 0.014% | 0.006% | 0.008% | 0.018% | 0.037% | 0.005% | 0.007% |
| pWISE9197 | 490 | 0.009% | 0.004% | 0.002% | 0.200% | 0.253% | 0.024% | 0.003% |
| pWISE9198 | 491 | 0.012% | 0.008% | 0.013% | 3.181% | 4.674% | 0.730% | 0.007% |
| pWISE9199 | 492 | 0.010% | 0.007% | 0.007% | 12.833% | 9.471% | 0.154% | 0.005% |
| pWISE9200 | 493 | 0.011% | 0.008% | 0.005% | 1.576% | 2.793% | 0.014% | 0.003% |
| pWISE9201 | 494 | 0.012% | 0.006% | 0.014% | 15.834% | 12.845% | 0.041% | 0.004% |
| pWISE9202 | 495 | 0.013% | 0.004% | 0.007% | 0.064% | 0.097% | 0.008% | 0.004% |
| pWISE9203 | 496 | 0.011% | 0.010% | 0.010% | 0.188% | 0.149% | 0.024% | 0.004% |
| pWISE9204 | 497 | 0.011% | 0.005% | 0.011% | 2.930% | 3.058% | 0.040% | 0.005% |
| pWISE9205 | 498 | 0.016% | 0.009% | 0.009% | 10.916% | 6.529% | 0.031% | 0.001% |
| pWISE9206 | 499 | 0.011% | 0.008% | 0.013% | 13.570% | 9.223% | 0.038% | 0.007% |

TABLE 110

| | | Standard deviation of the average A to G editing percent at the | | | | | | |
|---|---|---|---|---|---|---|---|---|
| | | nucleotides numbered according to the position in the PWsp143 spacer | | | | | | |
| | Protein | sequence on the opposite, target genomic DNA strand | | | | | | |
| pWISE | SEQ ID NO: | A.-5 | A.-4 | A.5 | A.8 | A.12 | A.21 | A.29 |
| pWISE121 | 56 | 0.029% | 0.025% | 0.012% | 0.020% | 0.024% | 0.030% | 0.010% |
| pWISE2918 | 498 | 0.001% | 0.001% | 0.012% | 1.008% | 0.942% | 0.001% | 0.003% |
| pWISE4922 | 501 | 0.004% | 0.001% | 0.007% | 0.064% | 0.037% | 0.005% | 0.005% |
| pWISE6854 | 502 | 0.001% | 0.005% | 0.003% | 0.131% | 0.071% | 0.001% | 0.000% |
| pWISE9193 | 486 | 0.001% | 0.001% | 0.002% | 0.223% | 0.265% | 0.068% | 0.006% |
| pWISE9194 | 487 | 0.002% | 0.006% | 0.001% | 0.021% | 0.033% | 0.003% | 0.003% |
| pWISE9195 | 488 | 0.006% | 0.010% | 0.005% | 0.043% | 0.010% | 0.013% | 0.004% |
| pWISE9196 | 489 | 0.004% | 0.001% | 0.000% | 0.005% | 0.025% | 0.000% | 0.005% |
| pWISE9197 | 490 | 0.000% | 0.002% | 0.000% | 0.119% | 0.001% | 0.027% | 0.000% |
| pWISE9198 | 491 | 0.004% | 0.001% | 0.005% | 0.119% | 0.061% | 0.004% | 0.004% |
| pWISE9199 | 492 | 0.003% | 0.001% | 0.005% | 0.557% | 0.386% | 0.030% | 0.000% |
| pWISE9200 | 493 | 0.005% | 0.002% | 0.001% | 0.041% | 0.310% | 0.005% | 0.003% |
| pWISE9201 | 494 | No data | No data | No data | No data | No data | No data | No data |
| pWISE9202 | 495 | 0.002% | 0.005% | 0.003% | 0.005% | 0.036% | 0.003% | 0.001% |
| pWISE9203 | 496 | 0.009% | 0.007% | 0.004% | 0.032% | 0.047% | 0.021% | 0.000% |
| pWISE9204 | 497 | 0.001% | 0.001% | 0.003% | 0.297% | 0.345% | 0.011% | 0.001% |
| pWISE9205 | 498 | 0.002% | 0.002% | 0.008% | 0.480% | 0.399% | 0.006% | 0.001% |
| pWISE9206 | 499 | 0.002% | 0.000% | 0.006% | 1.112% | 1.163% | 0.022% | 0.001% |

TABLE 111

| | | Average A to G editing percent at the nucleotides | | | | |
|---|---|---|---|---|---|---|
| | | numbered according to the position in the PWsp453 spacer | | | | |
| | Protein | sequence on the opposite, target genomic DNA strand | | | | |
| pWISE | SEQ ID NO: | A.-9 | A.-5 | A.-1 | A.2 | A.5 |
| pWISE121 | 56 | 0.010% | 0.023% | 0.066% | 0.070% | 0.044% |
| pWISE2918 | 498 | 0.013% | 0.010% | 0.062% | 0.069% | 0.008% |
| pWISE4922 | 501 | 0.007% | 0.008% | 0.036% | 0.031% | 0.006% |
| pWISE6854 | 502 | 0.012% | 0.005% | 0.018% | 0.013% | 0.012% |
| pWISE9193 | 486 | 0.005% | 0.010% | 0.026% | 0.027% | 0.011% |
| pWISE9194 | 487 | 0.009% | 0.008% | 0.005% | 0.008% | 0.009% |
| pWISE9195 | 488 | 0.007% | 0.006% | 0.020% | 0.016% | 0.013% |
| pWISE9196 | 489 | 0.006% | 0.011% | 0.007% | 0.002% | 0.010% |
| pWISE9197 | 490 | 0.008% | 0.004% | 0.007% | 0.015% | 0.008% |
| pWISE9198 | 491 | 0.008% | 0.008% | 0.014% | 0.013% | 0.005% |
| pWISE9199 | 492 | 0.007% | 0.008% | 0.028% | 0.016% | 0.009% |
| pWISE9200 | 493 | 0.012% | 0.008% | 0.011% | 0.005% | 0.011% |
| pWISE9201 | 494 | 0.012% | 0.011% | 0.017% | 0.015% | 0.012% |
| pWISE9202 | 495 | 0.009% | 0.009% | 0.008% | 0.003% | 0.012% |
| pWISE9203 | 496 | 0.008% | 0.009% | 0.010% | 0.004% | 0.003% |
| pWISE9204 | 497 | 0.007% | 0.009% | 0.011% | 0.006% | 0.009% |
| pWISE9205 | 498 | 0.002% | 0.007% | 0.029% | 0.035% | 0.004% |
| pWISE9206 | 499 | 0.011% | 0.011% | 0.033% | 0.018% | 0.006% |

| | | Average A to G editing percent at the nucleotides | | | | |
|---|---|---|---|---|---|---|
| | | numbered according to the position in the PWsp453 spacer | | | | |
| | Protein | sequence on the opposite, target genomic DNA strand | | | | |
| pWISE | SEQ ID NO: | A.9 | A.11 | A.12 | A.15 | A.16 |
| pWISE121 | 56 | 0.167% | 0.202% | 0.133% | 0.288% | 0.468% |
| pWISE2918 | 498 | 15.699% | 8.331% | 1.175% | 0.125% | 0.346% |
| pWISE4922 | 501 | 8.769% | 4.554% | 0.679% | 0.056% | 0.233% |
| pWISE6854 | 502 | 17.069% | 8.802% | 1.183% | 0.034% | 0.080% |
| pWISE9193 | 486 | 9.895% | 6.919% | 2.544% | 0.383% | 1.229% |
| pWISE9194 | 487 | 0.274% | 0.083% | 0.035% | 0.016% | 0.023% |
| pWISE9195 | 488 | 4.762% | 1.886% | 0.494% | 0.045% | 0.191% |
| pWISE9196 | 489 | 0.036% | 0.017% | 0.008% | 0.015% | 0.038% |
| pWISE9197 | 490 | 0.115% | 0.035% | 0.020% | 0.023% | 0.020% |
| pWISE9198 | 491 | 0.733% | 0.301% | 0.138% | 0.022% | 0.046% |
| pWISE9199 | 492 | 13.056% | 9.143% | 4.276% | 0.517% | 2.073% |
| pWISE9200 | 493 | 1.157% | 0.413% | 0.103% | 0.021% | 0.015% |
| pWISE9201 | 494 | 16.951% | 12.556% | 5.241% | 0.311% | 0.957% |
| pWISE9202 | 495 | 0.121% | 0.056% | 0.008% | 0.012% | 0.011% |
| pWISE9203 | 496 | 0.087% | 0.043% | 0.015% | 0.018% | 0.025% |
| pWISE9204 | 497 | 3.333% | 2.669% | 1.116% | 0.081% | 0.210% |
| pWISE9205 | 498 | 12.136% | 6.316% | 0.861% | 0.101% | 0.298% |
| pWISE9206 | 499 | 13.817% | 9.508% | 3.601% | 0.191% | 0.610% |

| | | Average A to G editing percent at the nucleotides | | | | |
|---|---|---|---|---|---|---|
| | | numbered according to the position in the PWsp453 spacer | | | | |
| | Protein | sequence on the opposite, target genomic DNA strand | | | | |
| pWISE | SEQ ID NO: | A.18 | A.23 | A.27 | A.28 | A.31 |
| pWISE121 | 56 | 0.320% | 0.271% | 0.081% | 0.061% | 0.048% |
| pWISE2918 | 498 | 1.967% | 0.009% | 0.009% | 0.005% | 0.006% |
| pWISE4922 | 501 | 1.082% | 0.010% | 0.013% | 0.006% | 0.006% |
| pWISE6854 | 502 | 0.334% | 0.007% | 0.011% | 0.008% | 0.004% |
| pWISE9193 | 486 | 6.193% | 0.015% | 0.020% | 0.007% | 0.010% |
| pWISE9194 | 487 | 0.021% | 0.010% | 0.009% | 0.009% | 0.007% |
| pWISE9195 | 488 | 0.938% | 0.027% | 0.007% | 0.007% | 0.010% |
| pWISE9196 | 489 | 0.011% | 0.008% | 0.011% | 0.006% | 0.010% |
| pWISE9197 | 490 | 0.009% | 0.012% | 0.009% | 0.007% | 0.004% |
| pWISE9198 | 491 | 0.200% | 0.014% | 0.013% | 0.006% | 0.006% |
| pWISE9199 | 492 | 6.227% | 0.021% | 0.009% | 0.006% | 0.006% |
| pWISE9200 | 493 | 0.043% | 0.008% | 0.010% | 0.003% | 0.005% |
| pWISE9201 | 494 | 3.006% | 0.014% | 0.010% | 0.009% | 0.007% |
| pWISE9202 | 495 | 0.012% | 0.013% | 0.011% | 0.006% | 0.004% |
| pWISE9203 | 496 | 0.019% | 0.010% | 0.005% | 0.003% | 0.017% |
| pWISE9204 | 497 | 0.832% | 0.009% | 0.013% | 0.005% | 0.008% |
| pWISE9205 | 498 | 1.500% | 0.006% | 0.008% | 0.002% | 0.007% |
| pWISE9206 | 499 | 2.100% | 0.007% | 0.009% | 0.002% | 0.003% |

TABLE 112

| | | Standard deviation of the average A to G editing percent at the | | | | |
|---|---|---|---|---|---|---|
| | | nucleotides numbered according to the position in the PWsp453 | | | | |
| | Protein | spacer sequence on the opposite, target genomic DNA strand | | | | |
| pWISE | SEQ ID NO: | A.-9 | A.-5 | A.-1 | A.2 | A.5 |
| pWISE121 | 56 | 0.010% | 0.003% | 0.016% | 0.016% | 0.011% |
| pWISE2918 | 498 | 0.007% | 0.002% | 0.010% | 0.027% | 0.002% |
| pWISE4922 | 501 | 0.002% | 0.005% | 0.018% | 0.009% | 0.001% |
| pWISE6854 | 502 | 0.002% | 0.000% | 0.008% | 0.006% | 0.002% |
| pWISE9193 | 486 | 0.000% | 0.004% | 0.024% | 0.014% | 0.005% |
| pWISE9194 | 487 | 0.005% | 0.001% | 0.000% | 0.002% | 0.005% |
| pWISE9195 | 488 | 0.004% | 0.008% | 0.005% | 0.023% | 0.012% |
| pWISE9196 | 489 | 0.003% | 0.006% | 0.004% | 0.000% | 0.005% |
| pWISE9197 | 490 | No data | No data | No data | No data | No data |
| pWISE9198 | 491 | No data | No data | No data | No data | No data |
| pWISE9199 | 492 | 0.001% | 0.003% | 0.003% | 0.006% | 0.008% |
| pWISE9200 | 493 | 0.005% | 0.002% | 0.007% | 0.005% | 0.003% |
| pWISE9201 | 494 | 0.001% | 0.002% | 0.011% | 0.007% | 0.003% |
| pWISE9202 | 495 | 0.001% | 0.000% | 0.004% | 0.002% | 0.006% |
| pWISE9203 | 496 | 0.003% | 0.005% | 0.005% | 0.004% | 0.004% |
| pWISE9204 | 497 | 0.001% | 0.001% | 0.001% | 0.002% | 0.004% |
| pWISE9205 | 498 | 0.003% | 0.003% | 0.009% | 0.017% | 0.006% |
| pWISE9206 | 499 | 0.004% | 0.009% | 0.004% | 0.011% | 0.006% |
| | | Standard deviation of the average A to G editing percent at the | | | | |
| | | nucleotides numbered according to the position in the PWsp453 | | | | |
| | Protein | spacer sequence on the opposite, target genomic DNA strand | | | | |
| pWISE | SEQ ID NO: | A.9 | A.11 | A.12 | A.15 | A.16 |
| pWISE121 | 56 | 0.016% | 0.024% | 0.028% | 0.004% | 0.052% |
| pWISE2918 | 498 | 0.046% | 0.208% | 0.070% | 0.039% | 0.008% |
| pWISE4922 | 501 | 0.321% | 0.246% | 0.065% | 0.018% | 0.075% |
| pWISE6854 | 502 | 0.718% | 0.327% | 0.092% | 0.007% | 0.004% |
| pWISE9193 | 486 | 0.147% | 0.005% | 0.197% | 0.104% | 0.127% |
| pWISE9194 | 487 | 0.031% | 0.029% | 0.003% | 0.013% | 0.004% |
| pWISE9195 | 488 | 0.364% | 0.015% | 0.014% | 0.001% | 0.019% |
| pWISE9196 | 489 | 0.007% | 0.003% | 0.005% | 0.001% | 0.036% |
| pWISE9197 | 490 | No data | No data | No data | No data | No data |
| pWISE9198 | 491 | No data | No data | No data | No data | No data |
| pWISE9199 | 492 | 0.053% | 0.152% | 0.130% | 0.030% | 0.216% |
| pWISE9200 | 493 | 0.105% | 0.033% | 0.027% | 0.005% | 0.008% |
| pWISE9201 | 494 | 0.917% | 0.569% | 0.626% | 0.013% | 0.050% |
| pWISE9202 | 495 | 0.037% | 0.002% | 0.000% | 0.004% | 0.000% |
| pWISE9203 | 496 | 0.000% | 0.001% | 0.013% | 0.009% | 0.001% |
| pWISE9204 | 497 | 0.335% | 0.427% | 0.192% | 0.017% | 0.085% |
| pWISE9205 | 498 | 0.213% | 0.104% | 0.309% | 0.031% | 0.044% |
| pWISE9206 | 499 | 1.010% | 0.918% | 0.320% | 0.043% | 0.038% |
| | | Standard deviation of the average A to G editing percent at the | | | | |
| | | nucleotides numbered according to the position in the PWsp453 | | | | |
| | Protein | spacer sequence on the opposite, target genomic DNA strand | | | | |
| pWISE | SEQ ID NO: | A.18 | A.23 | A.27 | A.28 | A.31 |
| pWISE121 | 56 | 0.098% | 0.047% | 0.013% | 0.001% | 0.008% |
| pWISE2918 | 498 | 0.061% | 0.001% | 0.000% | 0.006% | 0.000% |
| pWISE4922 | 501 | 0.094% | 0.006% | 0.003% | 0.001% | 0.001% |
| pWISE6854 | 502 | 0.020% | 0.003% | 0.001% | 0.002% | 0.004% |
| pWISE9193 | 486 | 0.011% | 0.003% | 0.001% | 0.003% | 0.002% |
| pWISE9194 | 487 | 0.003% | 0.000% | 0.000% | 0.005% | 0.001% |
| pWISE9195 | 488 | 0.022% | 0.002% | 0.010% | 0.002% | 0.003% |
| pWISE9196 | 489 | 0.000% | 0.004% | 0.000% | 0.000% | 0.001% |
| pWISE9197 | 490 | No data | No data | No data | No data | No data |
| pWISE9198 | 491 | No data | No data | No data | No data | No data |
| pWISE9199 | 492 | 0.018% | 0.007% | 0.003% | 0.002% | 0.004% |
| pWISE9200 | 493 | 0.001% | 0.003% | 0.003% | 0.000% | 0.001% |
| pWISE9201 | 494 | 0.358% | 0.008% | 0.004% | 0.002% | 0.001% |
| pWISE9202 | 495 | 0.001% | 0.001% | 0.000% | 0.002% | 0.004% |
| pWISE9203 | 496 | 0.000% | 0.003% | 0.002% | 0.004% | 0.011% |
| pWISE9204 | 497 | 0.067% | 0.001% | 0.008% | 0.001% | 0.007% |
| pWISE9205 | 498 | 0.043% | 0.001% | 0.003% | 0.003% | 0.002% |
| pWISE9206 | 499 | 0.111% | 0.002% | 0.004% | 0.002% | 0.001% |

TABLE 113

| | | Average A to G editing percent at the nucleotides | | | | | | | |
|---|---|---|---|---|---|---|---|---|---|
| | | numbered according to the position in the PWsp454 spacer | | | | | | | |
| | Protein | sequence on the opposite, target genomic DNA strand | | | | | | | |
| pWISE | SEQ ID NO: | A.-5 | A.-1 | A.2 | A.9 | A.11 | A.23 | A.27 | A.32 |
| pWISE121 | 56 | 0.014% | 0.025% | 0.042% | 0.102% | 0.063% | 0.188% | 0.161% | 0.060% |
| pWISE2918 | 498 | 0.002% | 0.014% | 0.002% | 10.929% | 11.829% | 0.011% | 0.015% | 0.003% |
| pWISE4922 | 501 | 0.004% | 0.004% | 0.002% | 7.989% | 8.529% | 0.005% | 0.005% | 0.004% |
| pWISE6854 | 502 | 0.002% | 0.006% | 0.005% | 7.648% | 8.799% | 0.010% | 0.004% | 0.010% |
| pWISE9193 | 486 | 0.007% | 0.003% | 0.002% | 7.465% | 10.715% | 0.023% | 0.003% | 0.011% |
| pWISE9194 | 487 | 0.006% | 0.004% | 0.004% | 0.096% | 0.404% | 0.012% | 0.008% | 0.007% |
| pWISE9195 | 488 | 0.003% | 0.003% | 0.009% | 5.314% | 7.055% | 0.024% | 0.005% | 0.005% |
| pWISE9196 | 489 | 0.007% | 0.003% | 0.003% | 0.018% | 0.018% | 0.008% | 0.004% | 0.005% |
| pWISE9197 | 490 | 0.006% | 0.001% | 0.005% | 0.093% | 0.388% | 0.012% | 0.005% | 0.005% |
| pWISE9198 | 491 | 0.010% | 0.003% | 0.007% | 0.743% | 2.436% | 0.039% | 0.020% | 0.004% |
| pWISE9199 | 492 | 0.004% | 0.000% | 0.007% | 7.638% | 8.686% | 0.010% | 0.002% | 0.007% |
| pWISE9200 | 493 | 0.005% | 0.002% | 0.002% | 0.827% | 2.586% | 0.012% | 0.003% | 0.007% |
| pWISE9201 | 494 | 0.005% | 0.002% | 0.005% | 9.689% | 10.575% | 0.022% | 0.002% | 0.005% |
| pWISE9202 | 495 | 0.004% | 0.001% | 0.006% | 0.038% | 0.137% | 0.010% | 0.005% | 0.005% |
| pWISE9203 | 496 | 0.009% | 0.004% | 0.000% | 0.054% | 0.091% | 0.006% | 0.003% | 0.005% |
| pWISE9204 | 497 | 0.004% | 0.003% | 0.008% | 2.223% | 2.468% | 0.012% | 0.003% | 0.012% |
| pWISE9205 | 498 | 0.000% | 0.003% | 0.001% | 8.947% | 9.565% | 0.012% | 0.005% | 0.003% |
| pWISE9206 | 499 | 0.005% | 0.003% | 0.003% | 9.336% | 10.616% | 0.005% | 0.003% | 0.003% |

TABLE 114

| | | Standard deviation of the average A to G editing percent at the nucleotides | | | | | | | |
|---|---|---|---|---|---|---|---|---|---|
| | | numbered according to the position in the PWsp454 spacer sequence on the | | | | | | | |
| | Protein | opposite, target genomic DNA strand | | | | | | | |
| pWISE | SEQ ID NO: | A.-5 | A.-1 | A.2 | A.9 | A.11 | A.23 | A.27 | A.32 |
| pWISE121 | 56 | 0.002% | 0.009% | 0.015% | 0.005% | 0.016% | 0.013% | 0.001% | 0.003% |
| pWISE2918 | 498 | No data | No data | No data | No data | No data | No data | No data | No data |
| pWISE4922 | 501 | 0.001% | 0.003% | 0.003% | 0.309% | 0.327% | 0.008% | 0.001% | 0.000% |
| pWISE6854 | 502 | 0.001% | 0.000% | 0.007% | 1.032% | 1.063% | 0.007% | 0.002% | 0.002% |
| pWISE9193 | 486 | 0.001% | 0.002% | 0.004% | 0.781% | 0.522% | 0.000% | 0.002% | 0.007% |
| pWISE9194 | 487 | 0.001% | 0.001% | 0.001% | 0.005% | 0.061% | 0.006% | 0.006% | 0.002% |
| pWISE9195 | 488 | 0.005% | 0.005% | 0.011% | 0.207% | 0.137% | 0.019% | 0.000% | 0.000% |
| pWISE9196 | 489 | 0.000% | 0.002% | 0.001% | 0.015% | 0.002% | 0.001% | 0.004% | 0.000% |
| pWISE9197 | 490 | 0.004% | 0.001% | 0.001% | 0.001% | 0.029% | 0.003% | 0.002% | 0.003% |
| pWISE9198 | 491 | No data | No data | No data | No data | No data | No data | No data | No data |
| pWISE9199 | 492 | 0.001% | 0.000% | 0.001% | 0.465% | 0.453% | 0.003% | 0.001% | 0.004% |
| pWISE9200 | 493 | 0.003% | 0.001% | 0.003% | 0.062% | 0.045% | 0.006% | 0.002% | 0.001% |
| pWISE9201 | 494 | 0.001% | 0.001% | 0.001% | 0.125% | 0.162% | 0.009% | 0.001% | 0.001% |
| pWISE9202 | 495 | 0.000% | 0.002% | 0.001% | 0.010% | 0.031% | 0.001% | 0.000% | 0.001% |
| pWISE9203 | 496 | 0.004% | 0.003% | 0.000% | 0.040% | 0.038% | 0.008% | 0.004% | 0.001% |
| pWISE9204 | 497 | 0.004% | 0.004% | 0.007% | 0.072% | 0.174% | 0.001% | 0.005% | 0.012% |
| pWISE9205 | 498 | No data | No data | No data | No data | No data | No data | No data | No data |
| pWISE9206 | 499 | No data | No data | No data | No data | No data | No data | No data | No data |

TABLE 115

| | |
|---|---|
| | Average A to G editing percent at the nucleotides numbered |
| | according to the position in the PWsp455 spacer sequence on the |
| Protein | opposite, target genomic DNA strand |

| pWISE | SEQ ID NO: | A.-8 | A.-6 | A.-1 | A.2 | A.3 | A.5 | A.8 |
|---|---|---|---|---|---|---|---|---|
| pWISE121 | 56 | 0.037% | 0.045% | 0.091% | 0.133% | 0.169% | 0.059% | 0.156% |
| pWISE2918 | 498 | 0.007% | 0.006% | 0.002% | 0.009% | 0.016% | 0.064% | 9.360% |
| pWISE4922 | 501 | 0.008% | 0.006% | 0.008% | 0.005% | 0.009% | 0.060% | 7.634% |
| pWISE6854 | 502 | 0.005% | 0.006% | 0.006% | 0.007% | 0.010% | 0.010% | 3.180% |
| pWISE9193 | 486 | 0.007% | 0.006% | 0.003% | 0.008% | 0.017% | 0.046% | 5.543% |
| pWISE9194 | 487 | 0.004% | 0.012% | 0.010% | 0.009% | 0.009% | 0.010% | 0.023% |
| pWISE9195 | 488 | 0.009% | 0.005% | 0.015% | 0.006% | 0.011% | 0.029% | 2.109% |
| pWISE9196 | 489 | 0.004% | 0.006% | 0.004% | 0.007% | 0.008% | 0.013% | 0.024% |
| pWISE9197 | 490 | 0.004% | 0.006% | 0.004% | 0.003% | 0.011% | 0.009% | 0.035% |
| pWISE9198 | 491 | 0.007% | 0.002% | 0.005% | 0.011% | 0.007% | 0.008% | 1.761% |
| pWISE9199 | 492 | 0.015% | 0.006% | 0.009% | 0.003% | 0.011% | 0.055% | 9.347% |
| pWISE9200 | 493 | 0.007% | 0.013% | 0.008% | 0.009% | 0.010% | 0.012% | 0.126% |
| pWISE9201 | 494 | 0.018% | 0.004% | 0.007% | 0.003% | 0.017% | 0.075% | 13.795% |
| pWISE9202 | 495 | 0.010% | 0.004% | 0.006% | 0.008% | 0.010% | 0.005% | 0.139% |
| pWISE9203 | 496 | 0.009% | 0.005% | 0.003% | 0.011% | 0.008% | 0.011% | 0.030% |
| pWISE9204 | 497 | 0.012% | 0.008% | 0.006% | 0.009% | 0.006% | 0.019% | 1.933% |
| pWISE9205 | 498 | 0.008% | 0.004% | 0.004% | 0.007% | 0.011% | 0.034% | 5.608% |
| pWISE9206 | 499 | 0.016% | 0.005% | 0.015% | 0.019% | 0.011% | 0.118% | 10.168% |

| | | Average A to G editing percent at the nucleotides numbered | | | | | | |
|---|---|---|---|---|---|---|---|---|
| | | according to the position in the PWsp455 spacer sequence on the | | | | | | |
| | Protein | opposite, target genomic DNA strand | | | | | | |
| pWISE | SEQ ID NO: | A.9 | A.10 | A.11 | A.15 | A.16 | A.17 | A.19 |
| pWISE121 | 56 | 0.323% | 0.304% | 0.261% | 1.356% | 1.577% | 1.071% | 0.355% |
| pWISE2918 | 498 | 6.675% | 2.657% | 4.690% | 1.004% | 0.024% | 0.025% | 0.182% |
| pWISE4922 | 501 | 5.449% | 2.362% | 3.960% | 0.726% | 0.024% | 0.025% | 0.144% |
| pWISE6854 | 502 | 2.123% | 0.749% | 1.482% | 0.339% | 0.026% | 0.018% | 0.076% |
| pWISE9193 | 486 | 3.610% | 2.551% | 6.447% | 1.400% | 0.035% | 0.030% | 0.456% |
| pWISE9194 | 487 | 0.020% | 0.014% | 0.033% | 0.013% | 0.017% | 0.012% | 0.009% |
| pWISE9195 | 488 | 1.524% | 1.506% | 4.362% | 1.156% | 0.028% | 0.011% | 0.230% |
| pWISE9196 | 489 | 0.022% | 0.013% | 0.032% | 0.014% | 0.012% | 0.016% | 0.016% |
| pWISE9197 | 490 | 0.041% | 0.014% | 0.041% | 0.003% | 0.008% | 0.014% | 0.005% |
| pWISE9198 | 491 | 1.202% | 1.744% | 5.841% | 0.386% | 0.028% | 0.034% | 0.228% |
| pWISE9199 | 492 | 7.609% | 3.561% | 6.885% | 0.797% | 0.019% | 0.027% | 0.139% |
| pWISE9200 | 493 | 0.085% | 0.052% | 0.158% | 0.041% | 0.008% | 0.005% | 0.012% |
| pWISE9201 | 494 | 11.036% | 6.672% | 12.328% | 0.951% | 0.041% | 0.026% | 0.221% |
| pWISE9202 | 495 | 0.075% | 0.046% | 0.319% | 0.029% | 0.028% | 0.025% | 0.038% |
| pWISE9203 | 496 | 0.028% | 0.018% | 0.022% | 0.015% | 0.008% | 0.008% | 0.011% |
| pWISE9204 | 497 | 1.699% | 1.520% | 2.776% | 0.404% | 0.040% | 0.026% | 0.178% |
| pWISE9205 | 498 | 3.956% | 1.720% | 2.781% | 0.606% | 0.020% | 0.010% | 0.091% |
| pWISE9206 | 499 | 7.342% | 3.719% | 7.520% | 0.550% | 0.024% | 0.037% | 0.181% |
| | | Average A to G editing percent at the nucleotides numbered | | | | | | |
| | | according to the position in the PWsp455 spacer sequence on the | | | | | | |
| | Protein | opposite, target genomic DNA strand | | | | | | |
| pWISE | SEQ ID NO: | A.21 | A.22 | A.24 | A.26 | A.28 | A.31 | A.32 |
| pWISE121 | 56 | 0.290% | 0.354% | 0.153% | 0.102% | 0.047% | 0.045% | 0.043% |
| pWISE2918 | 498 | 0.017% | 0.064% | 0.046% | 0.005% | 0.005% | 0.005% | 0.007% |
| pWISE4922 | 501 | 0.024% | 0.044% | 0.025% | 0.003% | 0.009% | 0.009% | 0.008% |
| pWISE6854 | 502 | 0.020% | 0.028% | 0.005% | 0.006% | 0.010% | 0.007% | 0.012% |
| pWISE9193 | 486 | 0.197% | 0.051% | 0.220% | 0.027% | 0.007% | 0.016% | 0.007% |
| pWISE9194 | 487 | 0.006% | 0.012% | 0.009% | 0.011% | 0.010% | 0.005% | 0.005% |
| pWISE9195 | 488 | 0.116% | 0.086% | 0.116% | 0.029% | 0.008% | 0.015% | 0.005% |
| pWISE9196 | 489 | 0.011% | 0.018% | 0.027% | 0.009% | 0.008% | 0.007% | 0.004% |
| pWISE9197 | 490 | 0.015% | 0.021% | 0.009% | 0.005% | 0.015% | 0.001% | 0.003% |
| pWISE9198 | 491 | 0.222% | 0.117% | 0.341% | 0.093% | 0.013% | 0.007% | 0.002% |
| pWISE9199 | 492 | 0.022% | 0.034% | 0.021% | 0.011% | 0.004% | 0.003% | 0.006% |
| pWISE9200 | 493 | 0.010% | 0.006% | 0.004% | 0.007% | 0.010% | 0.008% | 0.009% |
| pWISE9201 | 494 | 0.056% | 0.074% | 0.066% | 0.017% | 0.011% | 0.010% | 0.006% |
| pWISE9202 | 495 | 0.029% | 0.021% | 0.030% | 0.014% | 0.013% | 0.008% | 0.005% |
| pWISE9203 | 496 | 0.012% | 0.014% | 0.013% | 0.006% | 0.009% | 0.010% | 0.006% |
| pWISE9204 | 497 | 0.021% | 0.017% | 0.045% | 0.022% | 0.005% | 0.010% | 0.005% |
| pWISE9205 | 498 | 0.012% | 0.025% | 0.021% | 0.006% | 0.010% | 0.007% | 0.005% |
| pWISE9206 | 499 | 0.030% | 0.050% | 0.032% | 0.015% | 0.004% | 0.006% | 0.003% |

TABLE 116

| | | Standard deviation of the average A to G editing percent at the | | | | | | |
|---|---|---|---|---|---|---|---|---|
| | | nucleotides numbered according to the position in the PWsp455 | | | | | | |
| | Protein | spacer sequence on the opposite, target genomic DNA strand | | | | | | |
| pWISE | SEQ ID NO: | A.-8 | A.-6 | A.-1 | A.2 | A.3 | A.5 | A.8 |
| pWISE121 | 56 | 0.000% | 0.025% | 0.050% | 0.041% | 0.000% | 0.011% | 0.009% |
| pWISE2918 | 498 | No data | No data | No data | No data | No data | No data | No data |
| pWISE4922 | 501 | 0.004% | 0.001% | 0.007% | 0.005% | 0.001% | 0.021% | 1.149% |
| pWISE6854 | 502 | 0.001% | 0.005% | 0.000% | 0.008% | 0.001% | 0.006% | 0.301% |
| pWISE9193 | 486 | 0.001% | 0.003% | 0.002% | 0.002% | 0.002% | 0.024% | 0.837% |
| pWISE9194 | 487 | 0.002% | 0.002% | 0.002% | 0.004% | 0.002% | 0.009% | 0.006% |
| pWISE9195 | 488 | No data | No data | No data | No data | No data | No data | No data |
| pWISE9196 | 489 | 0.002% | 0.003% | 0.000% | 0.003% | 0.002% | 0.004% | 0.006% |
| pWISE9197 | 490 | 0.005% | 0.003% | 0.005% | 0.004% | 0.006% | 0.013% | 0.028% |
| pWISE9198 | 491 | 0.010% | 0.002% | 0.004% | 0.006% | 0.010% | 0.001% | 0.002% |
| pWISE9199 | 492 | 0.007% | 0.002% | 0.009% | 0.000% | 0.005% | 0.001% | 0.222% |
| pWISE9200 | 493 | 0.001% | 0.002% | 0.002% | 0.001% | 0.005% | 0.001% | 0.002% |
| pWISE9201 | 494 | 0.002% | 0.003% | 0.003% | 0.002% | 0.006% | 0.026% | 0.261% |
| pWISE9202 | 495 | 0.007% | 0.001% | 0.002% | 0.002% | 0.008% | 0.004% | 0.015% |
| pWISE9203 | 496 | 0.003% | 0.003% | 0.001% | 0.004% | 0.002% | 0.009% | 0.004% |
| pWISE9204 | 497 | 0.002% | 0.002% | 0.001% | 0.000% | 0.003% | 0.001% | 0.136% |
| pWISE9205 | 498 | 0.003% | 0.001% | 0.001% | 0.003% | 0.001% | 0.003% | 0.431% |
| pWISE9206 | 499 | 0.012% | 0.006% | 0.004% | 0.002% | 0.009% | 0.044% | 0.103% |
| | | Standard deviation of the average A to G editing percent at the | | | | | | |
| | | nucleotides numbered according to the position in the PWsp455 | | | | | | |
| | Protein | spacer sequence on the opposite, target genomic DNA strand | | | | | | |
| pWISE | SEQ ID NO: | A.9 | A.10 | A.11 | A.15 | A.16 | A.17 | A.19 |
| pWISE121 | 56 | 0.029% | 0.045% | 0.035% | 0.017% | 0.039% | 0.096% | 0.023% |
| pWISE2918 | 498 | No data | No data | No data | No data | No data | No data | No data |
| pWISE4922 | 501 | 0.778% | 0.267% | 0.559% | 0.074% | 0.005% | 0.012% | 0.038% |
| pWISE6854 | 502 | 0.163% | 0.059% | 0.284% | 0.064% | 0.004% | 0.007% | 0.002% |
| pWISE9193 | 486 | 0.837% | 0.486% | 1.261% | 0.322% | 0.015% | 0.022% | 0.133% |
| pWISE9194 | 487 | 0.007% | 0.007% | 0.012% | 0.001% | 0.003% | 0.004% | 0.004% |
| pWISE9195 | 488 | No data | No data | No data | No data | No data | No data | No data |
| pWISE9196 | 489 | 0.019% | 0.010% | 0.009% | 0.005% | 0.011% | 0.006% | 0.000% |
| pWISE9197 | 490 | 0.019% | 0.012% | 0.026% | 0.004% | 0.012% | 0.012% | 0.006% |
| pWISE9198 | 491 | 0.046% | 0.068% | 0.458% | 0.001% | 0.007% | 0.025% | 0.038% |
| pWISE9199 | 492 | 0.056% | 0.150% | 0.063% | 0.030% | 0.001% | 0.002% | 0.019% |
| pWISE9200 | 493 | 0.016% | 0.011% | 0.007% | 0.005% | 0.004% | 0.002% | 0.010% |
| pWISE9201 | 494 | 0.169% | 0.228% | 0.261% | 0.055% | 0.006% | 0.019% | 0.026% |
| pWISE9202 | 495 | 0.036% | 0.009% | 0.071% | 0.008% | 0.012% | 0.004% | 0.002% |
| pWISE9203 | 496 | 0.002% | 0.010% | 0.001% | 0.004% | 0.002% | 0.001% | 0.009% |
| pWISE9204 | 497 | 0.118% | 0.087% | 0.271% | 0.019% | 0.000% | 0.003% | 0.012% |
| pWISE9205 | 498 | 0.062% | 0.078% | 0.065% | 0.004% | 0.010% | 0.006% | 0.069% |
| pWISE9206 | 499 | 0.079% | 0.003% | 0.111% | 0.040% | 0.009% | 0.015% | 0.012% |
| | | Standard deviation of the average A to G editing percent at the | | | | | | |
| | | nucleotides numbered according to the position in the PWsp455 | | | | | | |
| | Protein | spacer sequence on the opposite, target genomic DNA strand | | | | | | |
| pWISE | SEQ ID NO: | A.21 | A.22 | A.24 | A.26 | A.28 | A.31 | A.32 |
| pWISE121 | 56 | 0.015% | 0.041% | 0.099% | 0.049% | 0.018% | 0.001% | 0.016% |
| pWISE2918 | 498 | No data | No data | No data | No data | No data | No data | No data |
| pWISE4922 | 501 | 0.000% | 0.005% | 0.013% | 0.002% | 0.001% | 0.003% | 0.003% |
| pWISE6854 | 502 | 0.005% | 0.012% | 0.006% | 0.000% | 0.001% | 0.003% | 0.011% |
| pWISE9193 | 486 | 0.033% | 0.017% | 0.023% | 0.010% | 0.002% | 0.002% | 0.003% |
| pWISE9194 | 487 | 0.004% | 0.000% | 0.004% | 0.008% | 0.004% | 0.002% | 0.000% |
| pWISE9195 | 488 | No data | No data | No data | No data | No data | No data | No data |
| pWISE9196 | 489 | 0.002% | 0.004% | 0.010% | 0.002% | 0.002% | 0.001% | 0.003% |
| pWISE9197 | 490 | 0.001% | 0.003% | 0.008% | 0.006% | 0.001% | 0.001% | 0.004% |
| pWISE9198 | 491 | 0.083% | 0.018% | 0.020% | 0.068% | 0.002% | 0.001% | 0.002% |
| pWISE9199 | 492 | 0.004% | 0.014% | 0.016% | 0.001% | 0.000% | 0.000% | 0.005% |
| pWISE9200 | 493 | 0.008% | 0.002% | 0.001% | 0.001% | 0.008% | 0.004% | 0.001% |
| pWISE9201 | 494 | 0.007% | 0.005% | 0.007% | 0.009% | 0.002% | 0.002% | 0.005% |
| pWISE9202 | 495 | 0.011% | 0.009% | 0.014% | 0.017% | 0.000% | 0.000% | 0.002% |
| pWISE9203 | 496 | 0.007% | 0.002% | 0.007% | 0.001% | 0.004% | 0.008% | 0.002% |
| pWISE9204 | 497 | 0.001% | 0.004% | 0.003% | 0.007% | 0.002% | 0.010% | 0.000% |
| pWISE9205 | 498 | 0.008% | 0.005% | 0.007% | 0.005% | 0.002% | 0.002% | 0.000% |
| pWISE9206 | 499 | 0.017% | 0.039% | 0.003% | 0.004% | 0.005% | 0.000% | 0.004% |

### Example 14

Arginine substitution variants of circular permutants described in Example 1 were tested in HEK293T cells to examine their ability to target different PAM site sequences. The arginine mutations were analogous to G532R and K595R mutations in LbCas12a. The circular permutants and the control are listed in Table 117. The arginine mutations are numbered according to the protein sequence listed in the fourth column of Table 31.

TABLE 117
Tested constructs.

| | Vector SEQ | DNA SEQ | | Protein SEQ |
|---|---|---|---|---|
| pWISE | ID NO: | ID NO: | Description | ID NO: |
| pWISE121 | 259 | 246 | WT − LbCas12a (control) | 56 |
| pWISE3559 | 713 | 709 | G532R + K595R − LbCas12a | 705 |
| pWISE10850 | 710 | 706 | G447R + K510R − CP02 | 702 |
| pWISE10851 | 711 | 707 | G619R + K682R − CP06 | 703 |
| pWISE10852 | 712 | 708 | G15R + K78R − CP08 | 704 |

TABLE 118

| | Target | | Spacer | | Vector | |
|---|---|---|---|---|---|---|
| Target | SEQ | | SEQ | | SEQ | PAM |
| Nucleic Acid | ID NO: | Spacer | ID NO: | pWISE | ID NO: | target |
| EMX1 | 887 | PWsp837 | 889 | pWISE2457 | 918 | ACCG |
| EMX1 | 887 | PWsp836 | 890 | pWISE2458 | 919 | ACCG |
| EMX1 | 887 | PWsp815 | 891 | pWISE2479 | 920 | ACCG |
| EMX1 | 887 | PWsp811 | 892 | pWISE2483 | 921 | CCCA |
| DNMT1 | 272 | PWsp786 | 893 | pWISE2508 | 922 | CCCA |
| DNMT1 | 272 | PWsp781 | 894 | pWISE2513 | 923 | CCCA |
| HEK2 | 872 | PWsp985 | 895 | pWISE2309 | 924 | GCCC |
| FANCF | 273 | PWsp972 | 896 | pWISE2322 | 925 | GCCC |
| FANCF | 273 | PWsp946 | 897 | pWISE2348 | 926 | GCCC |
| RNF2 | 274 | PWsp1292 | 898 | pWISE3611 | 927 | ACTA |
| HEK3 | 883 | PWsp1295 | 899 | pWISE3614 | 928 | ACTA |
| FANCF | 273 | PWsp1317 | 900 | pWISE3636 | 929 | ACTA |
| HEK3 | 883 | PWsp1298 | 901 | pWISE3617 | 930 | GTCG |
| FANCF | 273 | PWsp1326 | 902 | pWISE3645 | 931 | GTCG |
| FANCF | 273 | PWsp1341 | 903 | pWISE3658 | 932 | GTCG |
| DNMT1 | 272 | PWsp2381 | 904 | pWISE7545 | 933 | CTTA |
| AAVS1 | 863 | PWsp1940 | 905 | pWISE5966 | 934 | CTTA |
| AAVS1 | 863 | PWsp1943 | 906 | pWISE5969 | 935 | CTTA |
| FANCF | 273 | PWsp869 | 907 | pWISE2425 | 936 | GCCG |
| EMX1 | 887 | PWsp802 | 908 | pWISE2492 | 937 | GCCG |
| DNMT1 | 272 | PWsp766 | 909 | pWISE2529 | 938 | GCCG |
| DNMT1 | 272 | PWsp2463 | 910 | pWISE7860 | 939 | TTCA |
| FANCF | 273 | PWsp2464 | 911 | pWISE7861 | 940 | TTCA |
| HEK2 | 872 | PWsp2465 | 912 | pWISE7862 | 941 | TTCA |
| AAVS1 | 863 | PWsp2444 | 913 | pWISE7841 | 942 | CCCC |
| AAVS1 | 863 | PWsp2445 | 914 | pWISE7842 | 943 | CCCC |
| DNMT1 | 272 | PWsp2447 | 915 | pWISE7844 | 944 | CCCC |
| DNMT1 | 272 | PWsp143 | 275 | pWISE264 | 278 | TTTG |
| DNMT1 | 272 | PWsp137 | 916 | pWISE2360 | 945 | TTTC |
| DNMT1 | 272 | PWsp139 | 917 | pWISE258 | 946 | TTTC |

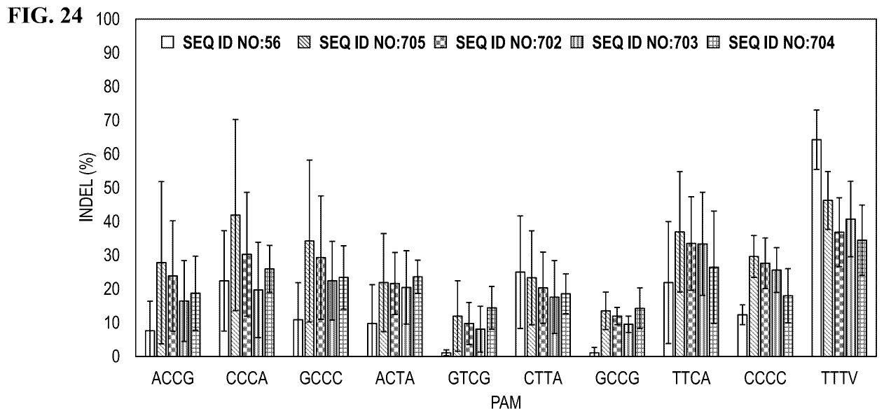

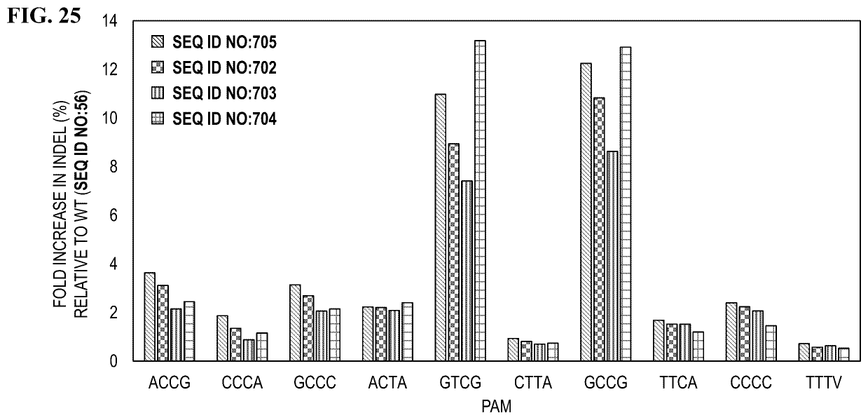

The circular permutants and control were tested for their ability to generate INDELS in the AAVS1, DNMT1, EMX1, FANCF, HEK2, HEK3, and RNF2 genes (SEQ ID NOs: 272-274, 863, 872, 887, and 888) in HEK293T cells using the spacers according to SEQ ID NOs: 275 and 889-917. Three biological replicates and one technical replicate were tested. HEK293T cells were seeded into 48-well collagen-coated plates (Corning) in the absence of an antibiotic using DMEM media. At 70-80% confluency, cells were transfected with 1.5 μL of LTX (ThermoFisher Scientific) using 500 ng of the control or circular permutant plasmid and 500 ng of guide RNA plasmid according to manufacturer's protocol. After 3 days, the cells were lysed with a crude extraction method using TritonX buffer. Each control or circular permutant was scored based on the precise base pair editing and indel placement percentage in the AAVS1, DNMT1, EMX1, FANCF, HEK2, HEK3, and RNF2 genes using the guide RNAs. Results are provided in FIGS. 24 and 25 and demonstrate that the two arginine mutations enabled recognition of non-canonical (e.g., non-TTTV) PAM sequences similar to the linear LbCas12a comprising the arginine mutations (SEQ ID NO:705).

The foregoing is illustrative of the present invention, and is not to be construed as limiting thereof. The invention is defined by the following claims, with equivalents of the claims to be included therein.

1. A polypeptide comprising a circular permutant of a Cas12a or an engineered protein that comprises a Cas12a polypeptide,
wherein the circular permutant binds to a nucleic acid.

2. The polypeptide of claim 1, wherein the circular permutant comprises a linker between two amino acid residues of the circular permutant.

3. The polypeptide of claim 2, wherein one of the two amino acid residues is located at the N-terminal end of the Cas12a or engineered protein and the other of the two amino acid residues is located at the C-terminal end of the Cas12a or engineered protein.

4. The polypeptide of claim 2, wherein the linker has a length of 1 amino acid to 20 amino acids.

5. (canceled)

6. The polypeptide of claim 1, wherein the circular permutant has a different editing window than that of the Cas12a or engineered protein.

7. The polypeptide of claim 1, wherein the circular permutant has increased nuclease activity compared to the Cas12a or engineered protein.

8.-9. (canceled)

10. The polypeptide of claim 1, wherein the circular permutant binds a target nucleic acid.

11. The polypeptide of claim 1, wherein the circular permutant has increased stability compared to the Cas12a or engineered protein, increased RNase activity compared to the Cas12a or engineered protein, modified crRNA binding compared to the Cas12a or engineered protein, and/or modified PAM access compared to the Cas12a or engineered protein.

12.-14. (canceled)

15. The polypeptide of claim 1, wherein the circular permutant has at least one improvement compared to a protein comprising an amino acid sequence of any one of SEQ ID NOs: 39-65, 69-132, 159-182, 281-284, 298, 400-401, 741, 761, and 850-861.

16. The polypeptide of claim 1, further comprising a cytosine deaminase.

17. The polypeptide of claim 1, further comprising a glycosylase inhibitor.

18. The polypeptide of claim 1, further comprising an adenine deaminase.

19. The polypeptide of claim 1, further comprising a reverse transcriptase.

20. The polypeptide of claim 1, wherein the polypeptide comprises having an amino acid sequence comprising at least 70% sequence identity to one or more of SEQ ID NOs: 234-245, 299-310, 351-362, 390-399, 450-461, 486-497, 537-560, 609-639, 702-704, 735-740, and 757-760.

21. A polypeptide comprising a first amino acid sequence having at least 70% sequence identity to a portion of one of SEQ ID NOs: 39-65, 69-132, 159-182, 281-284, 298, 400-401, 741, 761, and 850-861, wherein the portion of the one of SEQ ID NOs: 39-65, 69-132, 159-182, 281-284, 298, 400-401, 741, 761, and 850-861 includes the N-terminus of the one of SEQ ID NOs: 39-65, 69-132, 159-182, 281-284, 298, 400-401, 741, 761, and 850-861 and is less than the entire amino acid sequence of SEQ ID NOs: 39-65, 69-132, 159-182, 281-284, 298, 400-401, 741, 761, and 850-861 and the N-terminus of the one of SEQ ID NOs: 39-65, 69-132, 159-182, 281-284, 298, 400-401, 741, 761, and 850-861 is at or after amino acid residue 70 of the polypeptide.

22.-40. (canceled)

41. A complex comprising:
the polypeptide of claim 1; and
a guide nucleic acid.

42.-50. (canceled)

51. An expression cassette comprising:
a polynucleotide encoding a promoter sequence, and
a polynucleotide having at least 70% sequence identity to one or more of SEQ ID NOs: 247-258, 286-297, 327-338, 364-375, 402-411, 462-473, 503-514, 561-584, 640-670, 706-708, 742-747, 763-766, 784, and 787 and/or encoding a polypeptide that has at least 70% sequence identity to one or more of SEQ ID NOs: 234-245, 299-310, 351-362, 390-399, 450-461, 486-497, 537-560, 609-639, 702-704, 735-740, and 757-760,
optionally wherein the polynucleotide encoding the polypeptide is codon-optimized for expression in the organism.

52.-56. (canceled)

57. A method for producing the polypeptide of claim 1, the method comprising:
culturing a cell or group of cells that have been transformed with a nucleic acid encoding the polypeptide; and
isolating said polypeptide, thereby producing the polypeptide.

58. A method of modifying a target nucleic acid, the method comprising:
contacting the target nucleic acid with:
the polypeptide of claim 1; and
a guide nucleic acid, thereby modifying the target nucleic acid.

59.-60. (canceled)

61. The polypeptide of claim 1, wherein the circular permutant has nuclease activity.
